(19) United States
(12) Patent Application Publication
(10) Pub. No.: US 2003/0125315 A1
(43) Pub. Date: Jul. 3, 2003

(54) Probes, systems, and methods for drug discovery

(76) Inventors: Adnan M. M. Mjalli, Jamestown, NC (US); Chris Wysong, Winston-Salem, NC (US); Jerome Baudry, Champaign, IL (US); Thomas Scott Yokum, Greensboro, NC (US); Rob Andrews, Jamestown, NC (US); William K. Banner, Greensboro, NC (US)

Correspondence Address: Charles W. Calkins, Kilpatrick Stockton LLP, 1001 West Fourth Street, Winston-Salem, NC, 27101, US

(21) Appl. No.: 10/120,278

(22) Filed: Apr. 11, 2002

Related U.S. Application Data
(60) Provisional application No. 60/282,759, filed on Apr. 10, 2001.

(51) Int. Cl. G06G 7/48; G06G 7/58; G06F 19/00; G01N 33/48; G01N 33/50; A61K 31/397; A61K 31/53

(52) U.S. Cl. 514/210.01; 514/241; 514/649; 702/19; 703/11

(57) ABSTRACT

Aspects of the present invention include probes, methods, systems that have stand alone utility and may comprise features of a drug discovery system or method. The present invention also includes pharmaceutical compositions.

In more detail, the present invention provides molecular probes and methods for producing molecular probes. The present invention provides also provides systems and methods for new drug discovery. An embodiment of the present invention utilizes sets of probes of the present invention and a new approach to computational chemistry in a drug discovery method having increased focus in comparison to heretofore utilized combinatorial chemistry. The present invention also provides computer software and hardware tools useful in drug discovery systems. In an embodiment of a drug discovery method of the present invention in silico methods and in biologico screening methods are both utilized to maximize the probability of success while minimizing the time and number of wet laboratory steps necessary to achieve the success.

### STATEMENT OF RELATED APPLICATION

The present application claims priority under 35 USC 119 from U.S. Provisional Application Serial No. 60/282,759 filed Apr. 10, 2001, entitled “Method for Drug Discovery,” the disclosure of which is herein incorporated by reference.

### FIELD OF THE INVENTION

Aspects of the present invention include probes, methods, systems that have stand alone utility and may comprise features of a drug discovery system or method. The present invention also includes pharmaceutical compositions.

In more detail, the present invention provides molecular probes and methods for producing molecular probes. The present invention provides also provides systems and methods for new drug discovery. An embodiment of the present invention utilizes sets of probes of the present invention and a new approach to computational chemistry in a drug discovery method having increased focus in comparison to heretofore utilized combinatorial chemistry. The present invention also provides computer software and hardware tools useful in drug discovery systems. In an embodiment of a drug discovery method of the present invention in silico methods and in biologico screening methods are both utilized to maximize the probability of success while minimizing the time and number of wet laboratory steps necessary to achieve the success.

### BACKGROUND OF THE INVENTION

The discovery of chemical entities useful as drugs typically begins with the random screening of available chemical entities, usually from a given establishment's (company or university) chemical collection. Such an exercise, after considerable effort in data analysis, etc., may result in the discovery of some small number of active molecules termed “hits”. The systematic improvement of activity of such hits is often difficult in conventional methods due to such hits having different structural fingerprints thereby making an intuitively derived relationship between such molecules in terms of structure and their biological activity difficult.

The greater and greater chemical enablement of industry and academia allows the continued expansion of chemical diversity in an unordered way. Further, such continued practice of high throughput chemistry results often in larger and larger molecules which have limited usefulness as starting points for optimization, and further, one set of combinatorially derived molecules may not be easily relatable (via intuition or even computationally derived molecular descriptors) to another.

Thus, there is a need for a new approach to drug discovery.

### SUMMARY OF THE INVENTION

The present invention includes different aspects that have stand alone utility and also may comprise parts of a system for drug discovery.

In an aspect, the present invention provides molecular probes. The probes are useful in methods for drug discovery. The probes may also be useful in pharmaceutical compositions based on an association with a binding site of a therapeutic target.

In another aspect, the present invention provides chemical synthesis methods for producing probes. The methods may be used to prepare probes for biological screening.

In a further aspect, the present invention provides probe sets. The probe sets may comprise structurally nested probes. The probes sets are useful in systems and methods for drug discovery and may comprise computer representations and/or physical probes.

In an additional aspect, the present invention provides methods for producing probe sets. The methods may comprise the chemical synthesis methods of the present invention. The methods may alternatively, or additionally, comprise computer software and/or hardware methods for producing computer representations of probes.

The present invention also provides systems for drug discovery. The systems of the present invention may advantageously utilize probes, and/or probe sets, of the present invention, and/or may be performed with existing molecules.

The present invention further provides methods for drug discovery. The drug discovery methods may advantageously utilize probes, and/or probe sets, of the present invention.

Embodiments of the drug discovery systems and methods of the present invention may be performed in silico, or in biologico, or both. A feature of particular embodiments of the systems and methods of the present invention is that the methods comprise iterative steps for creating, evaluating, identifying and/or selecting probes.

In a still further aspect, the present invention provides pharmaceutical compositions. The pharmaceutical compositions may be identified through a drug discovery system or method of the present invention.

While features of the present invention are described with reference to the search for and identification of pharmacologically useful chemical compounds or drugs, features and aspects of the present invention are applicable to any attempt to search for an identify chemical compounds that have a desired physical characteristic.

An advantage of the present invention is that embodiments of the probes of the present invention may be utilized to explore the characteristics of a binding site of a target. Embodiments of the probes of the present invention have molecular weights sufficiently low, for example 1000 MW or below, to permit exploration of binding sites of smaller physical size than possible with other compositions.

Another advantage of the present invention is that embodiments of the probes of the present invention may be constructed in silico and/or in biologico.

A further advantage of the present invention is that embodiments of the systems and methods of the present invention provide a focused approach that permits a more rapid screening of probes with potential for association with a particular binding site with a higher likelihood of success.

Further details and advantages of aspects of the present invention are set forth in the following sections and the appended figures.

### BRIEF DESCRIPTION OF THE FIGURES

The present invention will be described with reference to the accompanying drawings, wherein:

FIG. 1 illustrates an exemplary environment for an embodiment of this invention.

FIG. 2 illustrates a multi-layer application framework in an embodiment of this invention.

FIG. 3 illustrates an embodiment of this invention as a 3-level structure of interrelated modules.

FIG. 4 illustrates the general process one embodiment of this invention utilizes in reference to the high-level modules of FIG. 3.

FIG. 5 illustrates the process implemented by the Protein Sequence Translation module in an embodiment of this invention.

FIG. 6 illustrates the binding site hypothesis process in an embodiment of this invention.

FIG. 7 illustrates the docking or screening process in an embodiment of this invention.

FIG. 8 illustrates the process implemented by the Selection and Analysis module in an embodiment of this invention.

FIG. 9 illustrates the general process of presenting and updating the user interface and scheduling and executing jobs in an embodiment of this invention.

FIG. 10 illustrates the search process in an embodiment of this invention.

FIG. 11 illustrates the general process of creating and executing jobs in an embodiment of this invention.

FIG. 12 illustrates utilizing templates and customized jobs in an embodiment of this invention.

FIG. 13 illustrates providing email notification of search results in an embodiment of this invention.

FIG. 14 illustrates providing modeling results via email in an embodiment of this invention.

FIG. 15 illustrates providing binding sites results via email in an embodiment of this invention.

FIG. 16 illustrates automated docking results via email in an embodiment of this invention.

FIG. 17 illustrates the creation and execution of a custom script for a commercial application component in an embodiment of this invention.

FIG. 18 illustrates the pre-paralellization process in an embodiment of this invention.

FIG. 19 illustrates the paralellization of a process in one embodiment of this invention.

FIG. 20 illustrates an exemplary environment for an embodiment of this invention.

FIG. 21a illustrates a process in an embodiment of this invention.

FIG. 21b is a screen shot of a logon screen in an embodiment of this invention.

FIG. 21c is a screen shot of a search screen in an embodiment of this invention.

FIG. 21d is a screen shot of a template creation and modification screen in an embodiment of this invention.

FIG. 21e is a screen shot of an assay data view in an embodiment of this invention.

FIG. 21f is a screen shot of a plotter view in an embodiment of this invention.

FIGS. 22-25 (except 23b) are process models of various embodiments of this invention.

FIG. 23b is a screen shot of a template view in an embodiment of this invention.

FIG. 26 is a block diagram of the method of drug discovery of the present invention.

FIG. 27 is a flow diagram depicting the operation of the in silico assay method.

FIG. 28 is a flow diagram depicting the operation of the in biologico assay method.

FIG. 29 is a flow diagram depiction the processing of a list of probes hits from the in silico assay method and the in biologico assay method.

FIG. 30 is a block flow diagram depicting the creation of a Probe Set and the location of a list of probes hits from the in silico assay method and the in biologico assay method.

FIG. 31 depicts a set of probes (Set I) displaying specific pharmacophoric features with variation of the distances between specific pharmacophoric features.

FIG. 32 depicts a set of probes (Set II) displaying specific pharmacophoric features with variation of the distances between specific pharmacophoric features.

FIG. 33 depicts a set of probes (Set III) displaying specific pharmacophoric features with variation of the distances between specific pharmacophoric features.

FIG. 34 depicts a set of probes (Set IV) displaying specific pharmacophoric features with variation of the distances between specific pharmacophoric features.

FIG. 35 is a graphical depiction of a set of recognition elements, binding sites, and frameworks.

FIG. 36 is a graphical depiction of a set of probes displaying various recognition elements and a hypothetical binding site of a target protein.

FIG. 37 is a graphical depiction of a hypothetical association of a probe and a binding site of a target protein.

FIG. 38 is a graphical depiction of a hypothetical association of a probe and a binding site of a target protein.

FIG. 39 is a graphical depiction of a hypothetical association of a probe and a binding site of a target protein.

FIG. 40 is a graphical depiction of a hypothetical association of a probe and a binding site of a target protein.

FIG. 41 is a graphical depiction of a combination of selected recognition elements and frameworks to yield a second generation probe.

FIG. 42 is a graphical depiction of a hypothetical association of a second generation probe with a target molecule.

### DETAILED DESCRIPTION OF THE INVENTION

As set forth above, the present invention provides probes, methods and systems, and also provides pharmacological compositions.

A probe comprises: a framework and an input fragment wherein the probe comprises a recognition element. In embodiments of the present invention the probe comprises a plurality of input fragments.

The probe may also comprise a plurality of recognition elements. The recognition element may be located on an input fragment or on the framework. An embodiment of a probe of the present invention that may be particularly useful in a drug discovery method comprises at least three input fragments and at least three recognition elements.

The probes of the present invention may be of any structure and/or size dictated by the selection of the framework and the input fragment. For use in a drug discovery method it may be advantageous to utilize probes of the present invention having a molecular weight less than 1000 MW. Smaller probes, for example having molecular weights less than 700 MW, or less than 500 MW may be even more advantageous.

The present invention also provides a method for producing a probe. The method may be performed in silico, or in biologico.

Further details relating to probes of the present invention, frameworks, input fragments and recognition elements, including chemical structures, are set forth below.

The present invention also provides pharmaceutical compositions.

A pharmaceutical composition comprises a probe of the present invention. The pharmaceutical composition may further comprise a pharmaceutically acceptable carrier and/or additional pharmacologically active ingredients.

Further details relating to pharmaceutical compositions of the present invention are set forth below.

The present invention further provides systems for drug discovery.

A system for drug discovery comprises:

a set of probes, each probe comprising a framework, an input fragment wherein the probe comprises a recognition element;

means for attempting to associate a probe from the set of probes with a binding site on a therapeutic target;

means for evaluating the association between the probe and the binding site; and

means for selecting probes with a desired association to the binding site.

The system for drug discovery may further comprise means for creating a pharmaceutical composition from a selected probe. The system for drug discovery may also further comprise means for creating a set of probes. Embodiments of probe sets suitable for use in a drug discovery system of the present invention include, but are not limited to, probe sets comprising probes of the present invention. Means for creating a set of probes include, but are not limited to, methods for producing probes of the present invention, including in silico and in biologico methods.

In an embodiment of a system for drug discovery of the present invention the means for attempting to associate a probe with a binding site may be performed in silico such that the means comprise computer software. Similarly, the means for evaluating the association between the probe and the binding site may be performed in silico such that the means comprise computer software. Further, the means for selecting probes with a desired association to the binding site may be performed in silico such that the means comprise computer software. In embodiments of the system of the present invention, one or all of these means may be performed in silico, while the remaining means, if any, are performed in biologico.

The present invention further provides a method for drug discovery utilizing a set of probes that comprises:

attempting to associate a probe from the set of probes with a binding site on a therapeutic target;

evaluating the association between the probe and the binding site; and

selecting probes with a desired association to the binding site.

The method for drug discovery may further comprise creating a pharmaceutical composition from a selected probe. The method for drug discovery may also further comprise means for creating a set of probes. Embodiments of probe sets suitable for use in a drug discovery method of the present invention include, but are not limited to, probe sets comprising probes of the present invention. Methods for creating a set of probes include, but are not limited to, methods for producing probes of the present invention, including in silico and in biologico methods.

In an embodiment of a method of the present invention the step of attempting to associate a probe with a binding site may be performed in silico such that the method comprises computer software. Similarly, the step of evaluating the association between the probe and the binding site may be performed in silico such that the method comprises computer software. Further, the step of selecting probes with a desired association to the binding site may be performed in silico such that the method comprises computer software. In embodiments of the system of the present invention, one or all of these means may be performed in silico, while the remaining means, if any, are performed in biologico.

The foregoing provides a general overview of aspects of the present invention. Further details on each aspect are set forth in the following sections.

The invention is directed to frameworks which when modified with input fragments, constitute probes which are useful molecules for screening against biological targets. The probe molecules are then studied for their potential interactions with biological targets.

The invention is also directed to a set of probes, a method for their synthesis, and a method for the selection of a subset of these probes for screening both computationally and biologically, and a method for iterative selection of further subsets of probes for secondary screening.

The probes of the present invention; a) may be synthesized, using solid phase or solution phase organic chemistry techniques, and then screened against biological targets using biochemical techniques known in the art, b) may be enumerated computationally, and then characterized computationally using a defined set of molecular descriptors, c) may be enumerated computationally and a three-dimensional structure or structures for each probe may be derived. Each probe may be examined computationally for its potential for association to a protein at one or more potential association sites, and each probe may be given a calculated score for its “fit” with the target protein. The steps a), b), and c) may be conducted simultaneously, independently, or employed iteratively in any sequence in selecting a hit molecule.

Therapeutic agents are chemical entities comprised of substructural moieties commonly known as pharmacophoric features. The types and geometric disposition of these features within a therapeutic molecule determine its binding affinity to a particular pharmacological target.

Medicinal chemists commonly recognize five pharmacophoric features: hydrophobes (H), hydrogen bond acceptors (A), hydrogen bond donors (D), negatively charged groups (N), and positively charged groups (P). Each feature can be represented by more than one chemical moiety. For example, a hydrophobic feature can correspond to an alkyl group, substituted or unsubstituted phenyl or thiophene rings, etc. A negatively charged feature could correspond to carboxylic, sulfonic, or other acid functionalities as well as tetrazole rings. A Feature Set comprises the five pharmacophoric featurs {H, A, D, N, P}. Many therapeutic agents are comprised of two to five features selected from this set.

The dependence of therapeutic effect on the type and geometric disposition of pharmacophoric features present in a therapeutic agent naturally leads to the concept of a Superset, intended to exhaust pharmacophore space. A Superset is defined as a set of probes that represents all possible combinations of pharmacophoric features, and, in which, every combination is represented by an ensemble of molecules that spans all possible reasonable geometries for that combination of pharmacophoric features. Reasonable geometries of pharmacophoric features can be inferred from known three-dimensional structures of pharmacological targets. Loading pharmacophoric features onto various frameworks enables the pharmacophoric features to adopt variable geometries, and enables the three-dimensional relationship between pharmacophoric features to span all reasonable geometries.

It should be noted that, in addition to constructing geometry spanning structures as described in the previous paragraph, conformational flexibility of a probe in the Superset represents an additional ensemble of thermally accessible geometries.

The Superset is expected to include compounds that are able to bind a broad diversity of pharmacological and therapeutic targets. Furthermore, due to the chemical degeneracy of each pharmacophoric feature, it is possible to construct several instances of the Superset. Each instance has a complete representation of a selected set of pharmacophoric features combinations and geometries. Different instances of a Superset differ in the specific chemical structural entities representing the individual pharmacophoric features.

Constructing a Superset starts with listing all possible combinations of pharmacophoric features selected from the Feature Set. An instance of the Superset is constructed by selecting chemical structural moieties to represent each selected member of the Feature Set. This is followed by constructing an ensemble of molecules for each combination of features such that distribution of feature geometries in the ensemble is uniformly distributed within the reasonable range. This process is illustrated below.

Table 1 shows a count of the number of possible combinations of features selected from the Feature Set for probes containing two to five features.

Tables 2, 3, 4, and 5 enumerate all combinations of 2, 3, 4, and 5 features, respectively, selected from the Feature Set

An instance of the Superset may comprise two A features, and one of each of H, P, D, and N features selected from the Feature Set. Chemical structures representing each these pharmacophoric features in this instance of the Superset are

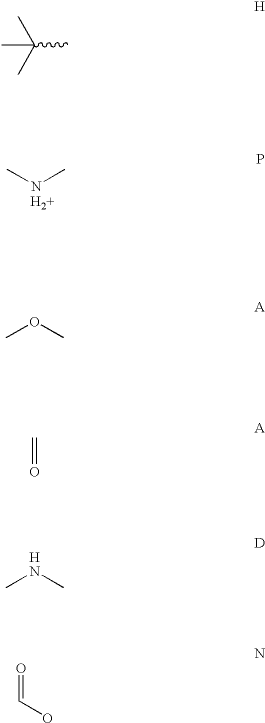

An alternative choice of chemical structural moieties to represent these six pharmacophoric features leads to an alternative instance of the Superset. Thus, utilizing phenyl ring to represent H and oxazole nitrogen or oxygen to represent the first, second, or both A's leads to an alternative instance of the Superset.

Constructing a complete Superset requires incorporating appropriate subsets of these six pharmacophoric features into molecules that represent every combination of pharmacophoric features enumerated in Tables 2-5. The discussion below illustrates the incorporation of a particular combination of five (H, P, A, A, D) of these six pharmacophoric features into one such molecule (Structure-I).

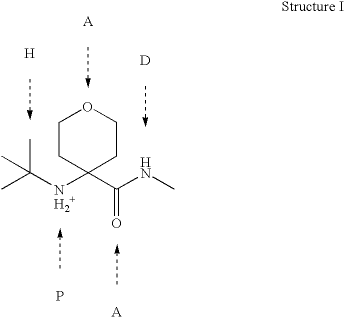

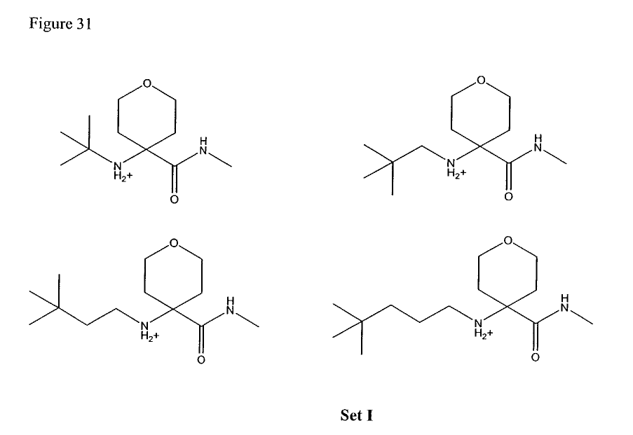

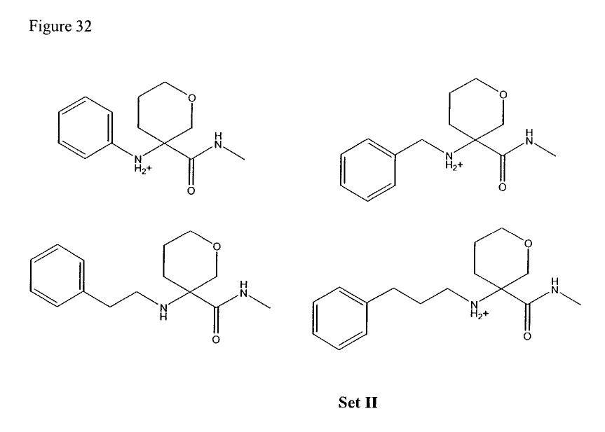

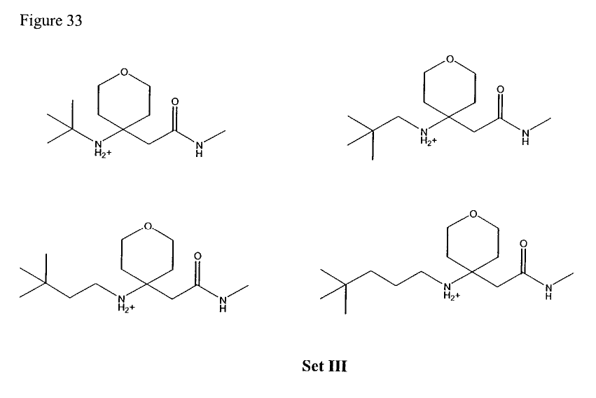

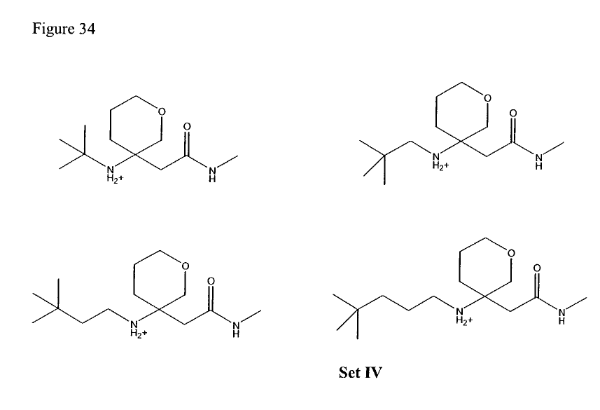

The follow discussion decribes the construction of an ensemble of “Structure—I”-type molecules. The structures in sets I, II, III, and IV are a subset of the ensemble of all reasonable geometries of H, P, A, A, D on a particular framework. These structures illustrate how a specific molecule, such as Structure—I, can be elaborated into an ensemble of reasonable geometries. The structures in sets I, II, III, IV (respective shown in FIGS. 31, 32, 33, and 34) constitute a subset of the ensemble of all reasonable geometries for this particular choice of pharmacophoric features in this instance of the Superset.

In Set I, the distances (geometry) between (P, A, A, D) are fixed relative to each other, while the distance between H and the (P, A, A, D) pharmacophoric features span reasonable geometries.

In Set II, the distances (geometry) between (P, A, A, D) are also fixed relative to each other, while the distance between H and the (P, A, A, D) pharmocophoric features span a reasonable range. Set II differs from Set I in that the distances between P and the other four pharmacophoric features are different from their corresponding values in Set I.

Sets III and IV are identical to Set I and II with the exception that the (A, D) features represented by (C(═O)—NH) are extended further away from A, P, and H.

TABLE 1
Number of combinations of two to five features
selected from the Feature Set

| Number of features | Number of combinations |
|---|---|
| 2 | 15 |
| 3 | 35 |
| 4 | 80 |
| 5 | 156 |

TABLE 2
All combinations of two features
selected from the Feature Set

| Combination # | Feature 1 | Feature 2 |
|---|---|---|
| 1 | H | D |
| 2 | H | A |
| 3 | H | N |
| 4 | H | P |
| 5 | D | A |
| 6 | D | N |
| 7 | D | P |
| 8 | A | N |
| 9 | A | P |
| 10 | N | P |
| 11 | H | H |
| 12 | D | D |
| 13 | A | A |
| 14 | N | N |
| 15 | P | P |

TABLE 3
All combinations of three features selected from the
Feature Set

| Combination # | Feature 1 | Feature 2 | Feature 3 |
|---|---|---|---|
| 1 | H | D | A |
| 2 | H | D | N |
| 3 | H | D | P |
| 4 | H | A | N |
| 5 | H | A | P |
| 6 | H | N | P |
| 7 | D | A | N |
| 8 | D | A | P |
| 9 | D | N | P |
| 10 | A | N | P |
| 11 | H | H | D |
| 12 | H | H | A |
| 13 | H | H | N |
| 14 | H | H | P |
| 15 | D | D | H |
| 16 | D | D | A |
| 17 | D | D | N |
| 18 | D | D | P |
| 19 | A | A | H |
| 20 | A | A | D |
| 21 | A | A | N |
| 22 | A | A | P |
| 23 | N | N | H |
| 24 | N | N | D |
| 25 | N | N | A |
| 26 | N | N | P |
| 27 | P | P | H |
| 28 | P | P | A |
| 29 | P | P | D |
| 30 | P | P | N |
| 31 | H | H | H |
| 32 | D | D | D |
| 33 | A | A | A |
| 34 | N | N | N |
| 35 | P | P | P |

TABLE 4
All combinations of four features selected from the Feature Set

| Combination # | Feature 1 | Feature 2 | Feature 3 | Feature 4 |
|---|---|---|---|---|
| 1 | H | D | A | N |
| 2 | H | D | A | P |
| 3 | H | D | N | P |
| 4 | H | A | N | P |
| 5 | D | A | N | P |
| 6 | H | H | D | A |
| 7 | H | H | D | N |
| 8 | H | H | D | P |
| 9 | H | H | A | N |
| 10 | H | H | A | P |
| 11 | H | H | N | P |
| 12 | D | D | H | A |
| 13 | D | D | H | N |
| 14 | D | D | H | P |
| 15 | D | D | A | N |
| 16 | D | D | A | P |
| 17 | D | D | N | P |
| 18 | A | A | H | D |
| 19 | A | A | H | N |
| 20 | A | A | H | P |
| 21 | A | A | D | N |
| 22 | A | A | D | P |
| 23 | A | A | N | P |
| 24 | N | N | D | H |
| 25 | N | N | D | A |
| 26 | N | N | D | P |
| 27 | N | N | H | A |
| 28 | N | N | H | P |
| 29 | N | N | A | P |
| 30 | P | P | H | D |
| 31 | P | P | H | A |
| 32 | P | P | H | N |
| 33 | P | P | D | A |
| 34 | P | P | D | N |
| 35 | P | P | A | N |
| 36 | H | H | D | D |
| 37 | H | H | A | A |
| 38 | H | H | N | N |
| 39 | H | H | P | P |
| 40 | D | D | H | H |
| 41 | D | D | A | A |
| 42 | D | D | N | N |
| 43 | D | D | P | P |
| 44 | A | A | H | H |
| 45 | A | A | D | D |
| 46 | A | A | N | N |
| 47 | A | A | P | P |
| 48 | N | N | D | D |
| 49 | N | N | H | H |
| 50 | N | N | A | A |
| 51 | N | N | P | P |
| 52 | P | P | H | H |
| 53 | P | P | D | D |
| 54 | P | P | A | A |
| 55 | P | P | N | N |
| 56 | H | H | H | D |
| 57 | H | H | H | A |
| 58 | H | H | H | N |
| 59 | H | H | H | P |
| 60 | D | D | D | H |
| 61 | D | D | D | A |
| 62 | D | D | D | N |
| 63 | D | D | D | P |
| 64 | A | A | A | H |
| 65 | A | A | A | D |
| 66 | A | A | A | N |
| 67 | A | A | A | P |
| 68 | N | N | N | D |
| 69 | N | N | N | H |
| 70 | N | N | N | A |
| 71 | N | N | N | P |
| 72 | P | P | P | H |
| 73 | P | P | P | D |
| 74 | P | P | P | A |
| 75 | P | P | P | N |
| 76 | H | H | H | H |
| 77 | D | D | D | D |
| 78 | A | A | A | A |
| 79 | N | N | N | N |
| 80 | P | P | P | P |

TABLE 5
All combinations of 5 features out of five

| Combination | | | | | |
|---|---|---|---|---|---|
| # | Feature 1 | Feature 2 | Feature 3 | Feature 4 | Feature 5 |
| 1 | H | D | A | N | P |
| 2 | H | H | D | A | N |
| 3 | H | H | D | A | P |
| 4 | H | H | D | N | P |
| 5 | H | H | A | N | P |
| 6 | D | D | H | A | N |
| 7 | D | D | H | A | P |
| 8 | D | D | H | N | P |
| 9 | D | D | A | N | P |
| 10 | A | A | H | D | N |
| 11 | A | A | H | D | P |
| 12 | A | A | H | N | P |
| 13 | A | A | D | N | P |
| 14 | N | N | D | H | A |
| 15 | N | N | D | H | P |
| 16 | N | N | D | A | P |
| 17 | N | N | H | A | P |
| 18 | P | P | H | D | A |
| 19 | P | P | H | D | N |
| 20 | P | P | H | A | N |
| 21 | P | P | D | A | N |
| 22 | H | H | H | D | A |
| 23 | H | H | H | D | N |
| 24 | H | H | H | D | P |
| 25 | H | H | H | A | N |
| 26 | H | H | H | A | P |
| 27 | H | H | H | N | P |
| 28 | D | D | D | H | A |
| 29 | D | D | D | H | N |
| 30 | D | D | D | H | P |
| 31 | D | D | D | A | N |
| 32 | D | D | D | A | P |
| 33 | D | D | D | N | P |
| 34 | A | A | A | H | D |
| 35 | A | A | A | H | N |
| 36 | A | A | A | H | P |
| 37 | A | A | A | D | N |
| 38 | A | A | A | D | P |
| 39 | A | A | A | N | P |
| 40 | N | N | N | D | H |
| 41 | N | N | N | D | A |
| 42 | N | N | N | D | P |
| 43 | N | N | N | H | A |
| 44 | N | N | N | H | P |
| 45 | N | N | N | A | P |
| 46 | P | P | P | H | D |
| 47 | P | P | P | H | A |
| 48 | P | P | P | H | N |
| 49 | P | P | P | D | A |
| 50 | P | P | P | D | N |
| 51 | P | P | P | A | N |
| 52 | H | H | H | H | H |
| 53 | D | D | D | D | D |
| 54 | N | N | N | N | N |
| 55 | A | A | A | A | A |
| 56 | P | P | P | P | P |
| 57 | H | H | D | D | A |
| 58 | H | H | D | D | N |
| 59 | H | H | D | D | P |
| 60 | H | H | A | A | D |
| 61 | H | H | A | A | N |
| 62 | H | H | A | A | P |
| 63 | H | H | N | N | D |
| 64 | H | H | N | N | A |
| 65 | H | H | N | N | P |
| 66 | H | H | P | P | D |
| 67 | H | H | P | P | A |
| 68 | H | H | P | P | P |
| 69 | D | D | H | H | A |
| 70 | D | D | H | H | N |
| 71 | D | D | H | H | P |
| 72 | D | D | A | A | H |
| 73 | D | D | A | A | N |
| 74 | D | D | A | A | P |
| 75 | D | D | N | N | H |
| 76 | D | D | N | N | A |
| 77 | D | D | N | N | P |
| 78 | D | D | P | P | H |
| 79 | D | D | P | P | A |
| 80 | D | D | P | P | P |
| 81 | A | A | H | H | D |
| 82 | A | A | H | H | N |
| 83 | A | A | H | H | P |
| 84 | A | A | D | D | H |
| 85 | A | A | D | D | N |
| 86 | A | A | D | D | P |
| 87 | A | A | N | N | H |
| 88 | A | A | N | N | D |
| 89 | A | A | N | N | P |
| 90 | A | A | P | P | H |
| 91 | A | A | P | P | D |
| 92 | A | A | P | P | P |
| 93 | N | N | D | D | H |
| 94 | N | N | D | D | A |
| 95 | N | N | D | D | P |
| 96 | N | N | H | H | D |
| 97 | N | N | H | H | A |
| 98 | N | N | H | H | P |
| 99 | N | N | A | A | D |
| 100 | N | N | A | A | H |
| 101 | N | N | A | A | P |
| 102 | N | N | P | P | D |
| 103 | N | N | P | P | H |
| 104 | N | N | P | P | P |
| 105 | P | P | H | H | D |
| 106 | P | P | H | H | A |
| 107 | P | P | H | H | N |
| 108 | P | P | D | D | H |
| 109 | P | P | D | D | A |
| 110 | P | P | D | D | N |
| 111 | P | P | A | A | H |
| 112 | P | P | A | A | D |
| 113 | P | P | A | A | N |
| 114 | P | P | N | N | H |
| 115 | P | P | N | N | D |
| 116 | P | P | N | N | N |
| 117 | H | H | D | D | D |
| 118 | H | H | A | A | A |
| 119 | H | H | N | N | N |
| 120 | H | H | P | P | P |
| 121 | D | D | H | H | H |
| 122 | D | D | A | A | A |
| 123 | D | D | N | N | N |
| 124 | D | D | P | P | P |
| 125 | A | A | H | H | H |
| 126 | A | A | D | D | D |
| 127 | A | A | N | N | N |
| 128 | A | A | P | P | P |
| 129 | N | N | D | D | D |
| 130 | N | N | H | H | H |
| 131 | N | N | A | A | A |
| 132 | N | N | P | P | P |
| 133 | P | P | H | H | H |
| 134 | P | P | D | D | D |
| 135 | P | P | A | A | A |
| 136 | P | P | N | N | N |
| 137 | H | H | H | H | D |
| 138 | H | H | H | H | A |
| 139 | H | H | H | H | N |
| 140 | H | H | H | H | P |
| 141 | D | D | D | D | H |
| 142 | D | D | D | D | A |
| 143 | D | D | D | D | N |
| 144 | D | D | D | D | P |
| 145 | A | A | A | A | H |
| 146 | A | A | A | A | D |
| 147 | A | A | A | A | N |
| 148 | A | A | A | A | P |
| 149 | N | N | N | N | D |
| 150 | N | N | N | N | H |
| 151 | N | N | N | N | A |
| 152 | N | N | N | N | P |
| 153 | P | P | P | P | H |
| 154 | P | P | P | P | D |
| 155 | P | P | P | P | A |
| 156 | P | P | P | P | N |

As used herein, the term “probe” refers to a molecular framework encompassing association elements suitable for interaction with a macromolecular biological target, such as but not limited to DNA, RNA, peptides, and proteins, said proteins being those such as but not limited to enzymes and receptors.

As used herein, the term “framework” refers to a unique chemical structure endowed with chemical and physical characteristics such that one or more appropriate association elements may be arranged and displayed thereon.

As used herein, the term “input fragment” refers to a generic molecular substitution upon a framework which is accomplished easily with a wide range of related chemical reagents. This substitution is advantageously accomplished at one or more active hydrogen sites on a framework.

As used herein, the terms “binding element” or “association element” refer to a specific point of association between two molecular species. Such points of association are those such as but not limited to hydrogen bond donor, hydrogen bond acceptor, Van der Waals interaction—promoting group, a pi-stacking—promoting group, a positively charged group, or a negatively charged group.

As used herein, the term “association” refers to the binding of one molecule to another in either a noncovalent or reversible covalent manner. Examples of “association” may include the binding of organic molecule and a peptide, an organic molecule and a protein, or an organic molecule and a polynucleotide species such as a RNA oligomer or DNA oligomer.

In a first aspect, the present invention provides a Probe Set containing probes useful for screening against biological targets, said probe comprised of an arbitrary selection of one of more frameworks, wherein said frameworks are modified by one or more input fragments. The probes of the invention may contain at least three pharmacophoric features. The probes of the invention may also contain at least three recognition elements. The one or more probes of the Probe Set of the invention are useful in engendering association or “binding” to macromolecular biological targets, thereby evoking one or more pharmacological consequences. In the above arbitrary selection of frameworks, the choice of said frameworks may be either totally random or may involve some proportion of pre-existing knowledge as to desirable frameworks for a given biological target.

The invention provides a probe comprising one of the following molecular formulae displayed in Chart 1.

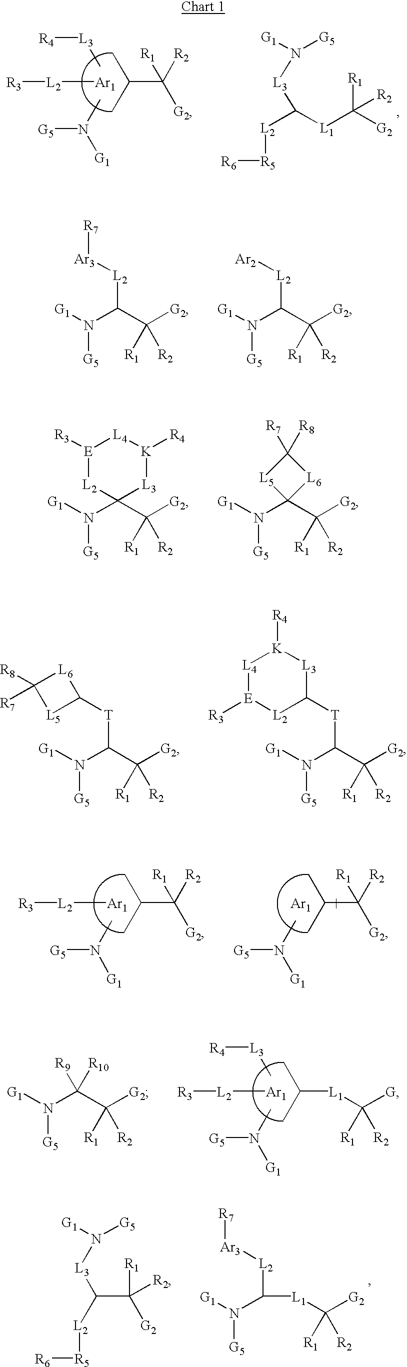

Ar₁ comprises aryl, heteroaryl, fused cycloalkylaryl, fused cycloakylheteroaryl, fused heterocyclylaryl, or fused heterocyclylheteroaryl;

L₁ comprises alkylene;

L₂ and L₃ independently comprise alkylene, alkenylene, alkynylene, or a direct bond;

R₁ and R₂ independently comprise alkyl, alkenyl, alkynyl, cycloalkyl, heterocyclyl, aryl, heteroaryl, or hydrogen;

R₁ and R₂ may be taken together to constitute an oxo group;

R₃ and R₄ independently comprise alkyl, alkenyl, alkynyl, cycloalkyl, heterocyclyl, aryl, heteroaryl, hydrogen, —O-G₃, —O-G₄, -G₃, -G₄, —N(G₆)G₃, or —N(G₆)G₄;

R₃ and R₄ may be taken together to constitute a cycloalkyl or heterocyclyl ring, or, where L₄ is a direct bond, R₃ and R₄ may be taken together to constitute a fused aryl or heteroaryl ring;

R₅ comprises alkylene, alkenylene, alkynylene, cycloalkylene, heterocyclylene, arylene, or heteroarylene;

R₆ comprises alkyl, alkenyl, alkynyl, cycloalkyl, heterocyclyl, aryl, heteroaryl, or hydrogen;

Ar₂ comprises arylene, heteroarylene, fused arylene, or fused heteroarylene;

Ar₃ comprises arylene, heteroarylene, fused arylene, or fused heteroarylene;

T comprises alkylene, alkenylene, alkynylene or a direct bond;

E and K independently comprise N or CH;

L₄ comprises alkylene, —O—, —C(O)—, —S—, —S(O)—, —S(O)₂—, or a direct single or double bond;

L₅ and L₆ are, independently, alkylene or a direct bond, with the proviso that both L₅ and L₆ are not both a direct bond;

R₇ and R₈ indpendently comprise alkyl, cycloalkyl, heterocyclyl, aryl, heteroaryl, alkoxy, alkylaryl, -alkylene-aryl, -alkylene-heteroaryl, —O-aryl, —O-heteroaryl, or hydrogen;

R₇ and R₈ may further be taken together to constitute a cycloalkyl or heterocyclyl ring;

R₉ comprises alkyl, alkenyl, alkynyl, cycloalkyl, heterocyclyl, aryl, heteroaryl, alkylaryl, alkylheteroaryl, or hydrogen;

R₁₀ comprises alkyl, alkenyl, alkynyl, cycloalkyl, heterocyclyl, aryl, heteroaryl, alkylaryl, alkylheteroaryl, or the side chain of a natural or non-natural alpha-amino acid in which any functional groups may be protected;

G₁, G₃, G₄ and G₁₄ independently comprise

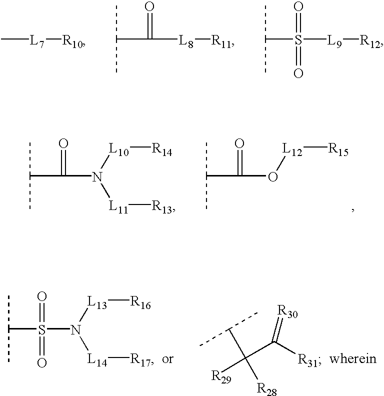

L₇, L₈, L₉, L₁₀, L₁₁, L₁₂, L₁₃, and L₁₄ independently comprise alkylene, alkenylene, alkynylene, cycloalkylene, cycloalkenylene, arylene, heterocyclylene, heteroarylene, fused cycloalkylarylene, fused cycloakylheteroarylene, fused heterocyclylarylene, fused heterocyclylheteroarylene, or a direct bond; and

R₁₁, R₁₂, R₁₃, R₁₄, R₁₅, R₁₆, and R₁₇ independently comprise alkyl, alkenyl, alkynyl, cycloalkyl, cycloalkenyl, heterocyclyl, heteroaryl, aryl, fused cycloalkylaryl, fused cycloakylheteroaryl, fused heterocyclylaryl, fused heterocyclylheteroaryl, NR₁₈R₁₉, OR₁₈, SR₁₈, or hydrogen, where R₁₈ and R₁₉ are as defined below;

R₂₈ comprises alkyl, alkenyl, alkynyl, aryl, heteroaryl, -alkenylene-aryl, or -alkenylene-heteroaryl;

R₂₉ comprises H, alkyl, alkenyl, alkynyl, -alkylene-aryl, or -alkylene-heteroaryl;

R₃₀ comprises 0 or H/OH;

R₃₁ comprises H, alkyl, or aryl;

G₂ comprises

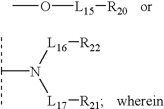

L₁₅, L₁₆, and L₁₇ independently comprise alkylene, alkenylene, alkynylene, cycloalkylene, cycloalkenylene, arylene, heterocyclylene, heteroarylene, fused cycloalkylarylene, fused cycloakylheteroarylene, fused heterocyclylarylene, fused heterocyclylheteroarylene, or a direct bond; and

R₂₀, R₂₁, and R₂₂ independently comprise alkyl, alkenyl, alkynyl, cycloalkyl, cycloalkenyl, heterocyclyl, heteroaryl, aryl, fused cycloalkylaryl, fused cycloakylheteroaryl, fused heterocyclylaryl, fused heterocyclylheteroaryl, NR₂₃R₂₄, OR₂₃, SR₂₃, or hydrogen, wherein R₂₃ and R₂₄ are as defined below;

G₅, G₈, and G₁₃ independently comprise

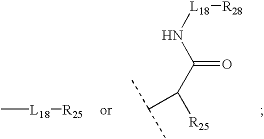

wherein L₁₈ comprises alkylene, alkenylene, alkynylene, cycloalkylene, cycloalkenylene, arylene, heterocyclylene, heteroarylene, fused cycloalkylarylene, fused cycloakylheteroarylene, fused heterocyclylarylene, fused heterocyclylheteroarylene, -alkylene-(aryl)₂, or a direct bond; and

R₂₅ comprises alkyl, alkenyl, alkynyl, cycloalkyl, cycloalkenyl, heterocyclyl, heteroaryl, aryl, fused cycloalkylaryl, fused cycloakylheteroaryl, fused heterocyclylaryl, fused heterocyclylheteroaryl, NR₂₆R₂₇, OR₂₆, SR₂₆, or hydrogen, where R₂₆ and R₂₇ are as defined below;

R₁₈, R₁₉, R₂₃, R₂₄, R₂₆, and R₂₇ independently comprise hydrogen, alkyl, alkynyl, alkenyl, cycloalkyl, cycloalkenyl, aryl, heterocyclyl, or heteroaryl;

optionally, G₁ and G₅ may be taken together in combination to constitute a heterocyclic or heteroaryl ring, wherein said heterocyclic or heteroaryl ring may be optionally substituted by a group

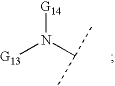

optionally, G₂ and one of G₁ or G₅ may be taken together in combination to constitute a heterocyclic ring;

optionally, G₂ of one probe and one of G₁, G₃, G₄, G₅ or G₆ of another probe may be taken together in combination to constitute a direct bond;

optionally, G₂ of a first probe and G₁ of a second probe may be taken together in combination to constitute a direct bond, where also G₂ of that second probe is taken in combination with G₁ of that first probe to constitute a direct bond;

optionally, one of G₁, G₃, G₄, G₅ or G₆ of one probe and one of G₁, G₃, G₄, G₅ or G₆ of another probe may be taken together in combination to constitute a group comprising;

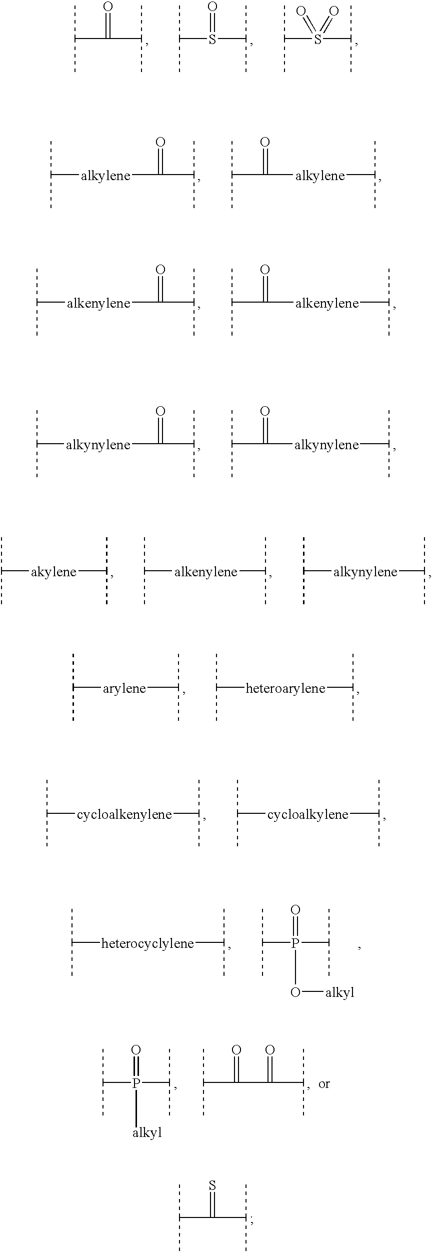

The present invention also provides a Probe Set comprising at least one probe of formulae displayed in Chart 1. The Probe Set will generally comprise a plurality of probes wherein the individual probes comprise molecular structures that are described by the formulae displayed in Chart 1.

The invention also provides probes taken as one or more of the following molecular formulae displayed in Chart 2.

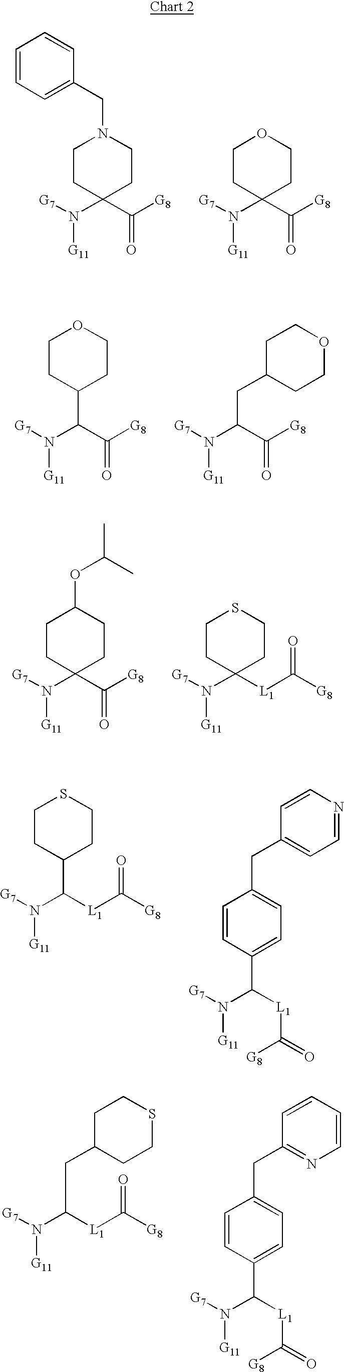

G₇, G₉, and G₁₀ independently comprise

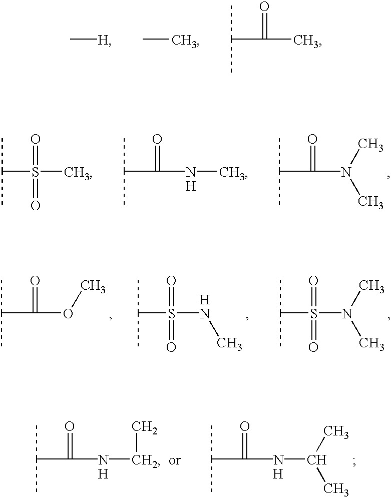

G₈ comprises

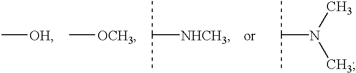

G₁₁ and G₁₂ independently comprise hydrogen or —CH₃;

Optionally, G₈ of one probe and one of G₇, G₉, or G₁₀ of another probe may be taken together in combination to constitute a direct bond.

The present invention also provides a Probe Set comprising at least one probe of formulae displayed in Chart II. The Probe Set will generally comprise a plurality of probes wherein the individual probes comprise molecular structures that are described by the formulae displayed in Chart II.

In probes of the above described probe set, the various functional groups represented should be understood to have a point of attachment at the functional group having the hyphen. In other words, in the case of —C₁₋₆ alkylaryl, it should be understood that the point of attachment is the alkyl group; an example would be benzyl. In the case of a group such as —C(O)—NH—C₁₋₆ alkylaryl, the point of attachment is the carbonyl carbon.

Also included within the scope of the invention are the individual enantiomers of the probes described above as well as any wholly or partially racemic mixtures thereof. The present invention also covers the individual enantiomers of the probes described above as mixtures with diastereoisomers thereof in which one or more stereocenters are inverted.

As used herein, the term “lower” refers to a group having between one and six carbons.

As used herein, the term “alkyl” refers to a straight or branched chain hydrocarbon having from one to ten carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkyl” group may containing one or more O, S, S(O), or S(O)₂ atoms. Examples of “alkyl” as used herein include, but are not limited to, methyl, n-butyl, n-pentyl, isobutyl, and isopropyl, and the like.

As used herein, the term “alkylene” refers to a straight or branched chain divalent hydrocarbon radical having from one to ten carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkylene” group may containing one or more O, S, S(O), or S(O)₂ atoms. Examples of “alkylene” as used herein include, but are not limited to, methylene, ethylene, and the like.

As used herein, the term “alkenyl” refers to a hydrocarbon radical having from two to ten carbons and at least one carbon-carbon double bond, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkenyl” group may containing one or more O, S, S(O), or S(O)₂ atoms.

As used herein, the term “alkenylene” refers to a straight or branched chain divalent hydrocarbon radical having from two to ten carbon atoms and one or more carbon-carbon double bonds, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkenylene” group may containing one or more O, S, S(O), or S(O)₂ atoms. Examples of “alkenylene” as used herein include, but are not limited to, ethene-1,2-diyl, propene-1,3-diyl, methylene-1,1-diyl, and the like.

As used herein, the term “alkynyl” refers to a hydrocarbon radical having from two to ten carbons and at least one carbon-carbon triple bond, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkynyl” group may containing one or more O, S, S(O), or S(O)₂ atoms.

As used herein, the term “alkynylene” refers to a straight or branched chain divalent hydrocarbon radical having from two to ten carbon atoms and one or more carbon-carbon triple bonds, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkynylene” group may containing one or more O, S, S(O), or S(O)₂ atoms. Examples of “alkynylene” as used herein include, but are not limited to, ethyne-1,2-diyl, propyne-1,3-diyl, and the like.

As used herein, “cycloalkyl” refers to a alicyclic hydrocarbon group with one or more degrees of unsaturation, having from three to twelve carton atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. “Cycloalkyl” includes by way of example cyclopropyl, cyclobutyl, cyclopentyl, cyclohexyl, cycloheptyl, or cyclooctyl, and the like.

As used herein, the term “cycloalkylene” refers to an non-aromatic alicyclic divalent hydrocarbon radical having from three to twelve carbon atoms and optionally possessing one or more degrees of unsaturation, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Examples of “cycloalkylene” as used herein include, but are not limited to, cyclopropyl-1,1-diyl, cyclopropyl-1,2-diyl, cyclobutyl-1,2-diyl, cyclopentyl-1,3-diyl, cyclohexyl-1,4-diyl, cycloheptyl-1,4-diyl, or cyclooctyl-1,5-diyl, and the like.

As used herein, the term “heterocyclic” or the term “heterocyclyl” refers to a three to twelve-membered heterocyclic ring having one or more degrees of unsaturation containing one or more heteroatomic substitutions selected from S, SO, SO₂, O, or N, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Such a ring may be optionally fused to one or more of another “heterocyclic” ring(s) or cycloalkyl ring(s). Examples of “heterocyclic” include, but are not limited to, tetrahydrofuran, 1,4-dioxane, 1,3-dioxane, piperidine, pyrrolidine, morpholine, piperazine, and the like.

As used herein, the term “heterocyclylene” refers to a three to twelve-membered heterocyclic ring diradical optionally having one or more degrees of unsaturation containing one or more heteroatoms selected from S, SO, SO₂, O, or N, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Such a ring may be optionally fused to one or more benzene rings or to one or more of another “heterocyclic” rings or cycloalkyl rings. Examples of “heterocyclylene” include, but are not limited to, tetrahydrofuran-2,5-diyl, morpholine-2,3-diyl, pyran-2,4-diyl, 1,4-dioxane-2,3-diyl, 1,3-dioxane-2,4-diyl, piperidine-2,4-diyl, piperidine-1,4-diyl, pyrrolidine-1,3-diyl, morpholine-2,4-diyl, piperazine-1,4-dyil, and the like.

As used herein, the term “aryl” refers to a benzene ring or to an optionally substituted benzene ring system fused to one or more optionally substituted benzene rings, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Examples of aryl include, but are not limited to, phenyl, 2-naphthyl, 1-naphthyl, 1-anthracenyl, and the like.

As used herein, the term “arylene” refers to a benzene ring diradical or to a benzene ring system diradical fused to one or more optionally substituted benzene rings, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Examples of “arylene” include, but are not limited to, benzene-1,4-diyl, naphthalene-1,8-diyl, and the like.

As used herein, the term “heteroaryl” refers to a five- to seven-membered aromatic ring, or to a polycyclic heterocyclic aromatic ring, containing one or more nitrogen, oxygen, or sulfur heteroatoms, where N-oxides and sulfur monoxides and sulfur dioxides are permissible heteroaromatic substitutions, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. For polycyclic aromatic ring systems, one or more of the rings may contain one or more heteroatoms. Examples of “heteroaryl” used herein are furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, and indazole, and the like.

As used herein, the term “heteroarylene” refers to a five- to seven-membered aromatic ring diradical, or to a polycyclic heterocyclic aromatic ring diradical, containing one or more nitrogen, oxygen, or sulfur heteroatoms, where N-oxides and sulfur monoxides and sulfur dioxides are permissible heteroaromatic substitutions, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. For polycyclic aromatic ring system diradicals, one or more of the rings may contain one or more heteroatoms. Examples of “heteroarylene” used herein are furan-2,5-diyl, thiophene-2,4-diyl, 1,3,4-oxadiazole-2,5-diyl, 1,3,4-thiadiazole-2,5-diyl, 1,3-thiazole-2,4-diyl, 1,3-thiazole-2,5-diyl, pyridine-2,4-diyl, pyridine-2,3-diyl, pyridine-2,5-diyl, pyrimidine-2,4-diyl, quinoline-2,3-diyl, and the like.

As used herein, the term “fused cycloalkylaryl” refers to a cycloalkyl group fused to an aryl group, the two having two atoms in common. Examples of “fused cycloalkylaryl” used herein include 1-indanyl, 2-indanyl, 1-(1,2,3,4-tetrahydronaphthyl), and the like.

As used herein, the term “fused cycloakylheteroaryl” refers to a cycloalkyl group fused to an heteroaryl group, the two having two atoms in common. Examples of “fused cycloalkylheteroaryl” used herein include 5-aza-1-indanyl and the like.

As used herein, the term “fused heterocyclylaryl” refers to a heterocyclyl group fused to an aryl group, the two having two atoms in common. Examples of “fused heterocyclylaryl” used herein include 2,3-benzodioxin and the like.

As used herein, the term “fused heterocyclylheteroaryl” refers to a heterocyclyl group fused to an heteroaryl group, the two having two atoms in common. Examples of “fused heterocyclylheteroaryl” used herein include 3,4-methylenedioxypyridine and the like.

As used herein, the term “side chain of a natural or non-natural alpha-amino acid” meand a group R within a natural or non-natural alpha-amino acid of formula H₂N—CH(R)—CO2H. Examples of such side chains are those such as but not limited to the side chains of alanine, arginine, asparagine, cysteine, cystine, aspartic acid, glutamic acid, tert-leucine, histidine, 5-hydroxylysine, 4-hydroxyproline, isoleucine, leucine, lysine, methionine, phenylalanine, proline, serine, threonine, tryptophan, tyrosine, valine, alpha-aminoadipic acid, alpha-aminoburyric acid, homoserine, alpha-methylserine, thyroxine, pipecolic acid, ornithine, and 3,4-dihydroxyphenylalanine. Functional groups in the side chains of a natural or non-natural alpha-amino acid may be protected. Carboxyl groups may be esterified such as but not limited to a alkyl ester, or may be substiruted by an carboxyl protecting group. Amino groups may be substituted by an acyl group, aroyl group, heteroaroyl group, alkoxycarbonyl group, or amino-protecting group. Hydroxyl groups may be converted to esters or ethers or may be substituted by alcohol protecting groups. Thiol groups may be converted to thioethers.

As used herein, the term “direct bond”, where part of a structural variable specification, refers to the direct joining of the substituents flanking (preceding and succeeding) the variable taken as a “direct bond”.

As used herein, the term “alkoxy” refers to the group RₐO—, where Rₐ is alkyl.

As used herein, the term “alkenyloxy” refers to the group RₐO—, where Rₐ is alkenyl.

As used herein, the term “alkynyloxy” refers to the group RₐO—, where Rₐ is alkynyl.

As used herein, the term “alkylsulfanyl” refers to the group RₐS—, where Rₐ is alkyl.

As used herein, the term “alkenylsulfanyl” refers to the group RₐS—, where Rₐ is alkenyl.

As used herein, the term “alkynylsulfanyl” refers to the group RₐS—, where Rₐ is alkynyl.

As used herein, the term “alkylsulfenyl” refers to the group RₐS(O)—, where Rₐ is alkyl.

As used herein, the term “alkenylsulfenyl” refers to the group RₐS(O)—, where Rₐ is alkenyl.

As used herein, the term “alkynylsulfenyl” refers to the group RₐS(O)—, where Rₐ is alkynyl.

As used herein, the term “alkylsulfonyl” refers to the group RₐSO₂—, where Rₐ is alkyl.

As used herein, the term “alkenylsulfonyl” refers to the group RₐSO₂—, where Rₐ is alkenyl.

As used herein, the term “alkynylsulfonyl” refers to the group RₐSO₂—, where Rₐ is alkynyl.

As used herein, the term “acyl” refers to the group RₐC(O)—, where Rₐ is alkyl, alkenyl, alkynyl, cycloalkyl, cycloalkenyl, or heterocyclyl.

As used herein, the term “aroyl” refers to the group RₐC(O)—, where Rₐ is aryl.

As used herein, the term “heteroaroyl” refers to the group RₐC(O)—, where Rₐ is heteroaryl.

As used herein, the term “alkoxycarbonyl” refers to the group RₐOC(O)—, where Rₐ is alkyl.

As used herein, the term “acyloxy” refers to the group RₐC(O)O—, where Rₐ is alkyl, alkenyl, alkynyl, cycloalkyl, cycloalkenyl, or heterocyclyl.

As used herein, the term “aroyloxy” refers to the group RₐC(O)O— where Rₐ is aryl.

As used herein, the term “heteroaroyloxy” refers to the group RₐC(O)O— where Rₐ is heteroaryl.

As used herein, the term “optionally” means that the subsequently described event(s) may or may not occur, and includes both event(s) which occur and events that do not occur.

As used herein, the term “substituted” refers to substitution with the named substituent or substituents, multiple degrees of substitution being allowed unless otherwise stated.

As used herein, the terms “contain” or “containing” can refer to in-line substitutions at any position along the above defined alkyl, alkenyl, alkynyl or cycloalkyl substituents with one or more of any of O, S, SO, SO₂, N, or N-alkyl, including, for example, —CH₂—O—CH₂—, —CH₂—SO₂—CH₂—, —CH₂—NH—CH₃ and so forth.

Whenever the terms “alkyl” or “aryl” or either of their prefix roots appear in a name of a substituent (e.g. arylalkoxyaryloxy) they shall be interpreted as including those limitations given above for “alkyl” and “aryl”. Alkyl or cycloalkyl substituents shall be recognized as being functionally equivalent to those having one or more degrees of unsaturation. Designated numbers of carbon atoms (e.g. C₁₋₁₀) shall refer independently to the number of carbon atoms in an alkyl, alkenyl or alkynyl or cyclic alkyl moiety or to the alkyl portion of a larger substituent in which the term “alkyl” appears as its prefix root.

As used herein, the term “oxo” shall refer to the substituent ═O.

As used herein, the term “halogen” or “halo” shall include iodine, bromine, chlorine and fluorine.

As used herein, the term “mercapto” shall refer to the substituent —SH.

As used herein, the term “carboxy” shall refer to the substituent —COOH.

As used herein, the term “cyano” shall refer to the substituent —CN.

As used herein, the term “aminosulfonyl” shall refer to the substituent —SO₂NH₂.

As used herein, the term “carbamoyl” shall refer to the substituent —C(O)NH₂.

As used herein, the term “sulfanyl” shall refer to the substituent —S—.

As used herein, the term “sulfenyl” shall refer to the substituent —S(O)—.

As used herein, the term “sulfonyl” shall refer to the substituent —S(O)₂—.

The compounds can be prepared readily according to the following reaction Schemes (in which variables are as defined before or are defined) using readily available starting materials, reagents and conventional synthesis procedures. In these reactions, it is also possible to make use of variants which are themselves known to those of ordinary skill in this art, but are not mentioned in greater detail.

Common names and definitions for resin reagents used herein include:

| | |
|---|---|
| Merrifield | p-Hydroxymethyl polystyrene |
| Wang | (4-Hydroxymethyl)phenoxymethyl polystyrene |
| Wang carbonate | 4-(p-nitrophenyl carbonate) phenoxymethyl |
| | polystyrene |
| Rink Resin | 4-(2′,4′-Dimethoxyphenyl-Fmco-aminomethyl)- |
| | phenoxy polystyrene resin |
| Wang Bromo | alpha-Bromo-alpha-methylphenaceyl polystyrene resin |
| Resin | |
| THP Resin | 3,4-Dihydro-2H-pyran-2-ylmethoxymethyl polystyrene |

Aldehyde resin can refer to the following:

Formylpolystyrene,

4-Benzyloxybenzaldehyde polystyrene,

3-Benzyloxybenzaldehyde polystyrene,

4-(4-Formyl-3-methoxyphenoxy)butyryl-aminomethyl polystyrene,

2-(4-Formyl-3-methoxyphenoxy)ethyl polystyrene,

2-(3,5-dimethoxy-4-formylphenoxy)ethoxy-methyl polystyrene,

2-(3,5-dimethoxy-4-formylphenoxy)ethoxy polystyrene,

(3-Formylindolyl)acetamidomethyl polystyrene,

(4-Formyl-3-methoxyphenoxy) grafted (polyethyleneglycol)-polystyrene; or

4-formyl-3-methoxyphenoxy)methylpolystyrene.

Abbreviations used herein are as follows

APCI=atmospheric pressure chemical ionization

BOC=tert-butoxycarbonyl

BOP=(1-benzotriazolyloxy)tris(dimethylamino)phosphonium hexafluorophosphate

BuOH=butyl alcohol

d=day

DBU=1,8-diazabicyclo[5.4.0]undec-7-ene

DCB=1,2-dichlorobenzene

DCC=dicyclohexylcarbodiimide

DCE=1,2 Dichloroethane

DCM=dichloromethane

DIAD=diisopropyl azodicarboxylate

DIEA=diisopropylethylamine

DIPCDI=1,3-diisopropylcarbodiimide

DMAP=4-Dimethylaminopyridine

DME=1,2-dimethoxyethane

DMF=N,N-dimethylformamide

DMS=Dimethyl sulfide

DMPU=1,3-dimethypropylene urea

DMSO=dimethylsulfoxide

EDC=1-ethyl-3-(3-dimethylaminopropyl)-carbodiimide hydrochloride

EDTA=ethylenediamine tetraacetic acid

ELISA=enzyme-linked immunosorbent assay

Eq.or equiv.=equivalents

ESI=electrospray ionization

ether=diethyl ether

EtOAc=ethyl acetate

EtOH=ethyl alcohol

FBS=fetal bovine serum

Fmoc=9-fluorenylmethyloxycarbonyl

g=gram

h=hour

HBTU=O-benzotriazol-1-yl-N,N,N′,N′-tetramethyluronium hexafluorophosphate

HMPA=hexamethylphosphoric triamide

HOBt=1-hydroxybenzotriazole

HOAc=glacial acetic acid

Hz=hertz

i.v.=intravenous

kD=kiloDalton

L=liter

LAH=lithium aluminum hydride

LDA=lithium diisopropylamide

LPS=lipopolysaccharide

M=molar

m/z=mass to charge ratio

mbar=millibar

MeOH=methanol

mg=milligram

min=minute

mL=milliliter

mM=millimolar

mmol=millimole

mol=mole

mp=melting point

MS=mass spectrometry

N=normal

NMM=N-methylmorpholine, 4-methylmorpholine

NMP=1-methyl-2-pyrrolidinone

NMR=nuclear magnetic resonance spectroscopy

p.o.=per oral

PBS=phosphate buffered saline solution

PMA=phorbol myristate acetate

PPh₃=triphenyl phosphine

PS=Polystyrene

ppm=parts per million

psi=pounds per square inch

R_{f}=relative TLC mobility

rt=room temperature

s.c.=subcutaneous

SPA=scintillation proximity assay

TBu=tert-butyl

TEA=triethylamine

TES=triethylsilane

TFA=trifluoroacetic acid

THF=tetrahydrofuran

THP=tetrahydropyranyl

TLC=thin layer chromatography

Tol=toluene

Trityl (Trt)=triphenylmethyl

Tᵣ=retention time

The following Reaction Schemes describe methods of synthesis of the probes. Reaction Scheme 1 describes a method of synthesis of the probes, wherein X is NH, O, —C(R₁)(R₂)—O—, or —C(R₁)(R₂)—NH—. M is a framework with the appropriate valences to display the W, Q, X, and Y motifs; W is N; Q is O, N, or a direct bond, Y is NH, O, or a direct bond, PG₁, PG₂, PG₃, and PG₄ are amino protecting groups, alcohol protecting groups, or carboxyl protecting groups as appropriate, or H; G₁, G₂, G₃, G₄, G₅ and G₆ have the meanings designated above. W, Q, and Y may independently be taken as a) substituents of the M moiety, or b) contained within a ring structure embodied in whole or in part by the M moiety. M can represent any alpha-amino acid fragment excluding —NH₂ and —CO₂H fragments. In other words, M can represent the alpha-carbon and its substituents of an elaborate alpha-amino acid. Where “prime” symbols (′) are used to designate variables, such variables are defined generically as above but may be same or different relative to their “unprime” counterparts, with the proviso that one and only one of PG₁, PG₂, PG₃, PG4, PG₁′, PG₂′, PG₃′, or PG₄′ may be a polymeric substance such as polystyrene or a suitably modified polystyrene adorned with a

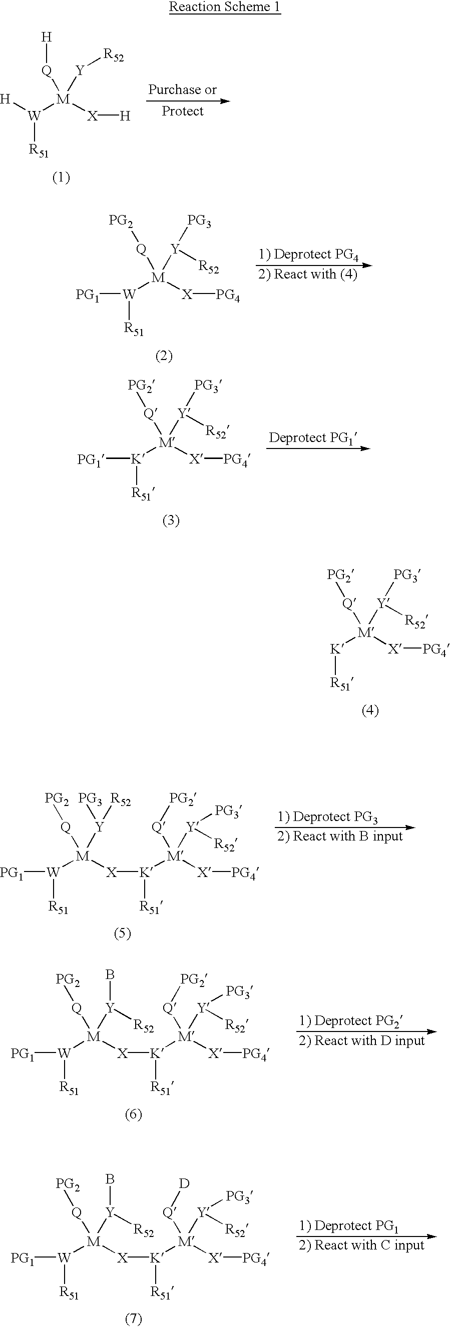

A intermediate (1) may be protected at W, Q, Y, and X with appropriate reagents. Alternately, the desired product (2) may be purchased commercially. G₅ where G₅ is alkyl or substituted alkyl may be introduced at this stage by treatment of (2) where R₂₈ is H with, for example, formaldehyde followed by isolation of the adduct and treatment with NaBH₃CN. (3) may be joined to a polymer by treatment of (3) where PG₄′ is H and X′ is —C(O)— with Merrifield resin and cesium carbonate in DMF, or by treatment of (3) where PG₄′ is H and X′ is —C(O)— with Wang resin and, for example, DIPCDI in DMF in the presence or absence of DMAP and/or HOBt. (3) may be deprotected at K′ and reacted with the acid (2) (where X is —C(O)— and PG₄ is H using, for example, DIC in DMF in the presence or absence of DMAP and/or HOBt to form (5). Successive amine and alcohol protecting groups may be removed and inputs introduced, as described further in Reaction Scheme 1. For example, where PG₃ is a FMOC group, treatment of (4) with piperidine in DCM is followed by introduction of a reagent such as acetic anhydride and pyridine to give (6) where B is —C(O)CH₃. Deprotection of alcohol, carboxyl, and amine protecting groups may be employed according to established art, as in J. W. Barton, “Protective Groups In Organic Chemistry”, J. G. W. McOmie, Ed., Plenum Press, New York, N.Y., 1973; T. W. Greene, “Protective Groups in Organic Synthesis”, John Wiley and Sons, New York, N.Y., 1981; or M. Bodansky, “Principles of Peptide Synthesis”, Springer-Verlag, Berlin Heidelberg, 1993.

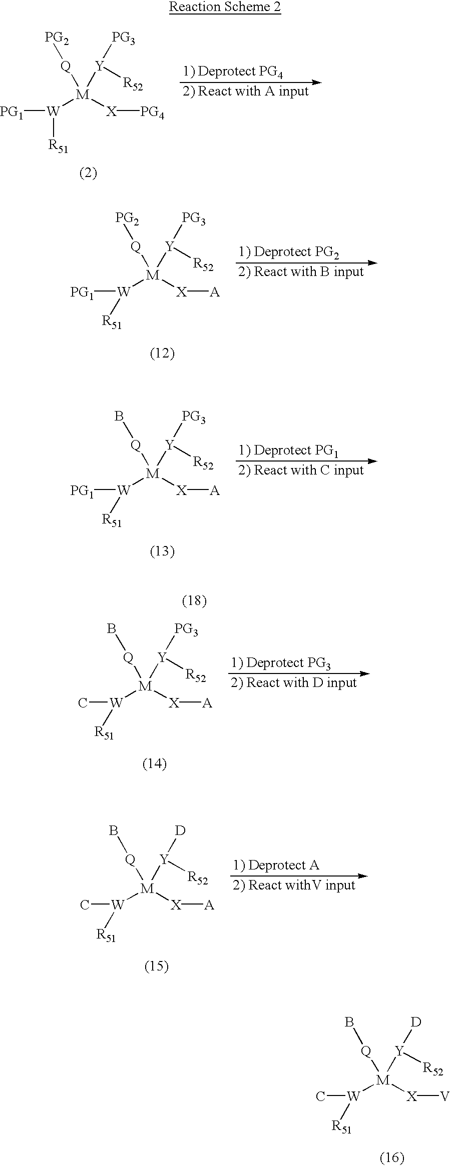

Reaction Scheme 2 describes the synthesis of a probe of formula (1)₆, where a single “M” framework is employed in the synthesis of the probe (16). X, having the same meaning as above, may be attached to a solid support in the same way. The input A may be a linker to a polystyrene solid support, such as the Wang, p-nitrophenoxycarbonyl-Wang, 2-tetrahydropyranyl-5-methoxy-Merrifield, Merrifield, or Rink resin, where X is NH, O, —C(R₁)(R₂)—O—, or —C(R₁)(R₂)—NH— Successive amine and alcohol protecting groups may be removed and inputs introduced, as described further in Reaction Scheme 2.

Introduction of G₁, G₃, and G₄ inputs may be accomplished by the use of;

a) acetic anhydride in pyridine or TEA/DMAP, in the case of —C(O)CH₃;

b) methanesulfonyl chloride in DCM with TEA/DMAP, in the case of —SO₂CH₃;

c) methyl isocyanate, ethyl isocyanate, or isopropyl isocyanate in the presence or absence of pyridine, in the case of —C(O)N(H)CH₃, —C(O)N(H)CH₂CH₃; or —C(O)N(H)CH(CH₃)₂;

d) N,N-dimethylcarbamyl chloride in DCM with TEA/DMAP, in the case of —C(O)N(CH₃)₂;

e) Methyl chloroformate in DCM with TEA/DMAP, for —C(O)OCH₃;

f) CH₃NHSO₂C₁ or CH₃N(PG₅)SO₂Cl in TEA/DMAP, followed by removal of PG₅ with, for example, piperidine in DMF where PG₅ is FMOC, in the case of —SO₂—NHCH₃;

g) (CH₃)₂NSO₂Cl in TEA/DMAP, in the case of —S(O)₂N(CH₃)₂;

Introduction of G₂ inputs may be accomplished by the use of;

a) diazomethane in ethyl acetate, or methyl iodide in DMF in the presence of DIEA, where a carboxylic acid is being modified;

b) methylamine or methylamine hydrochloride and DIC in DMF in the presence or absence of HOBT, where a carboxylic acid is being modified, for —NHCH₃;

c) methylamine in a solvent such as dioxane or isopropanol, where an ester is being modified, for —NHCH₃;

d) dimethylamine or dimethylamine hydrochloride and DIC in DMF in the presence or absence of HOBt, where a carboxylic acid is being modified, for —N(CH₃)₂;

e) dimethylamine in a solvent such as dioxane or isopropanol, where an ester is being modified, for —N(CH₃)₂;

f) Sodium methoxide in methanol, or methanol and diisopropylethylamine in THF, where an ester is being modified, for —OCH₃;

g) Water and diisopropylethylamine in THF, or alkali metal hydroxide in THF-methanol-water or methanol-water, or THF-water, for —OH;

The conversion of (10) to (11), and (15) to (16), may involve a cleavage of (10) and (15) from a polymer support. In the case of (11) and (14) where PG₄ or PG₄′ is a Wang resin linkage, treatment of (11) or (14) with TFA in DCM followed by filtration and concentration affords the carboxylic acid. In the case of (11) and (14) where PG₄ or PG₄′ is a Merrifield resin linkage, treatment of (11) or (14) with aqueous lithium hydroxide or sodium hydroxide, followed by filtration and neutralization with a proton-form ion exchange resin, followed by concentration, affords the carboxylic acid. The carboxylic acid may be processed to the ester or to the amide as above. Alternately, in the case of (11) and (14) where PG₄ or PG₄′ is a Wang resin linkage, or a Merrifield resin linkage, treatment of (11) or (14) with methylamine or dimethylamine in a polar solvent such as DMF, isopropanol, or dioxane, followed by filtration and concentration, affords the methylamide or dimethylamide. In the case of (11) and (14) where PG₄ or PG₄′ is a Rink resin linkage, treatment of (11) or (14) with TFA in DCM followed by filtration and concentration affords the carboxamide. In the case of (11) and (14) where PG₄ or PG₄′ is a carbamate or carbonate linkage to Wang resin, treatment of (11) or (14) with TFA in DCM followed by filtration and concentration affords the alcohol or amine.

Reaction Scheme 3 provides a synthesis of probes of formulae (25) and (26). The protected amino acid (17) is deprotected at the carboxylate oxygen and protected with A to afford (18). A may be taken as an alkyl input or as a linker to a polymer support. In this scheme and ensuing schemes, M represents a probe framework of variable nature, such as but not limited to to 1,1-cycloalkyl or amino-protected 4,4-piperidinyl. L₁₉ represents alkylene or a direct bond. The amino protecting group of (18) is deprotected and the free amine is reductively aminated with (19) employing, for example, sodium triacetoxyborohydride as the reducing agent in a solvent such as THF, to afford (20). R₅₃ and R₅₄ may be groups such as but not limited to, independently, alkyl or alkylene-aryl. The amine in (20) is alkylated with a bromoalkylene carboxylate such as bromoacetic acid, to afford (22). (22) is reacted with an amine (23) to provide (24). (24) may be modified with a G₂ input as decribed previously to afford (25). Alternately, (24) may be, where R₅₆ is H, cyclized by heating at a temperature of from 40° C. to 100° C. in a solvent such as toluene, to afford (26).

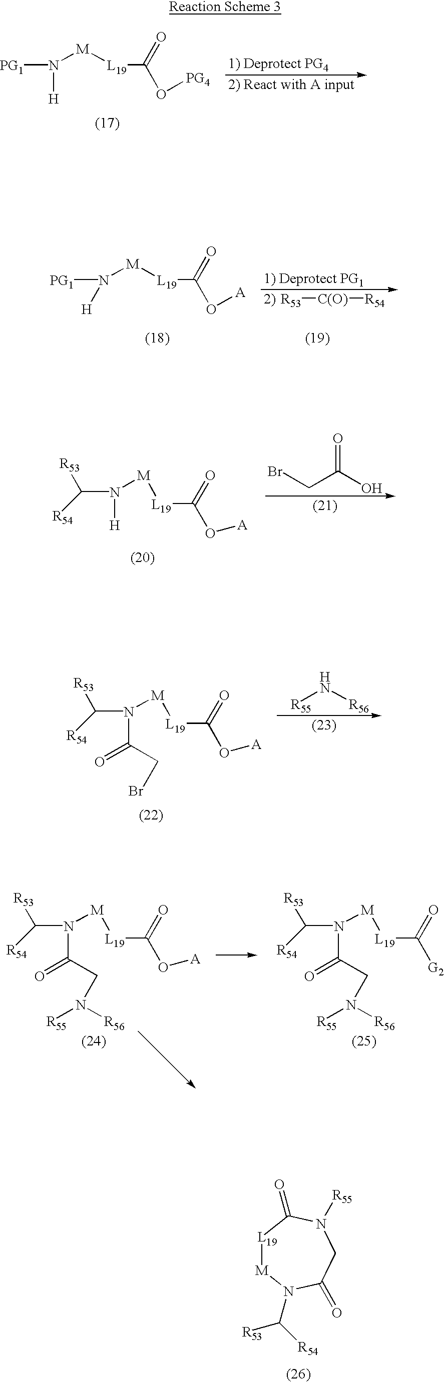

Reaction Scheme 4 describes a synthesis of probes of formulae (33) and (35). An aldehyde resin, such as but not limited to 4-benzyloxybenzaldehyde polystyrene (27) is reductively aminated with an amine (28) to afford (29). R₅₇ in this instance is a group such as but not limited to heteroaryl or -alkylene-aryl. The resin (29) is coupled to (30) employing a reagent such as DIPCDI and HOBt/DMAP to afford (31). The amino protecting group PG₁ is removed and the amino group is employed in reductive amination with the carbonyl compound (19,) where R₅₃ and R₅₄ have the meaning outlined previously. The amine (32) is treated with a reagent such as TFA in DCM to provide the amide (3.) The acid (34), free of amino substitution, may be subjected to the above selected reaction sequences of coupling to resin (29) and cleavage to provide (35).

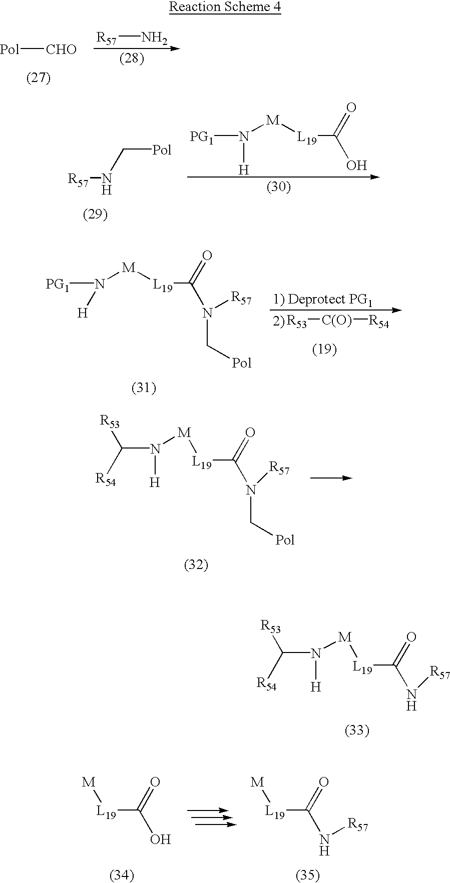

Reaction Scheme 5 describes the synthesis of a probe of formula (40). The protected or solid-supported ester (18), where A may be a solid support such as Wang resin, is deprotected and the free amine is reacted with a bromoacid (36) in the presence of a coupling agent such as DIPCDI or EDC, in the presence of HOBt, to give (37). L₂₀ may be a group such as but not limited to alkylene or alkylene-arylene. The bromide (37) may be reacted with a thiol reagent (38) to afford (39). In this instance, R₅₈ may be a group such as bur not limited to aryl, heteroaryl, or alkyl. The thioether (39) is subjected to introduction of the G₂ input as described previously to afford (40).

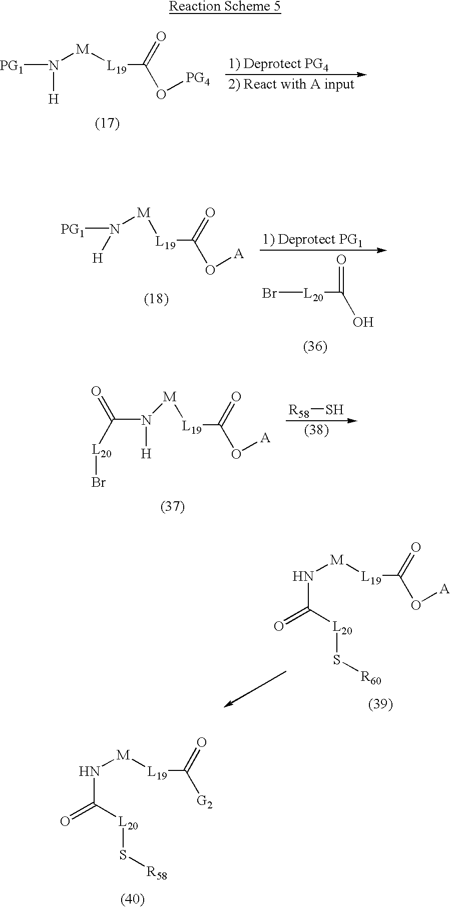

Reaction Scheme 6 describes the synthesis of probes of formulae (44) and (46). The intermediate (41) where R₆₀ is —OH, is coupled to a resin such as Wang carbonate or the chlorocarbonate resin formed by treatment of Wang resin with phosgene, diphosgene, or triphosgene, in the presence of a base such as TEA in a solvent such as DCM or THF, to form (42). Alternately, R₆₀ may be —NH₂ or —NH—R, wherein R is a group such as but not limited to alkyl or cycloalkyl. The amino protecting group PG₁ is removed, and the amine is reductively coupled with the carbonyl compound (19) as described previously. The product (43) may be modified with a substituent R₄₀ in the manner decribed for G₁, G₃, G₄ inputs previously, to afford (45). Alternately, (43) may be cleaved from the resin with, for example TFA in DCM to afford (44). (45) may be cleaved from the resin in like manner to afford (46).

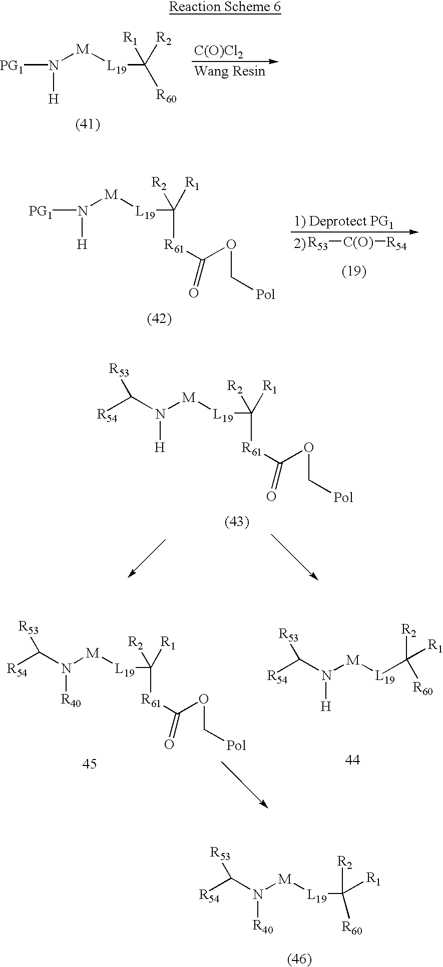

Reaction Scheme 7 describes the preparation of probes of formula (52) and (53). The bromoamide (37) descrived previously may be treated with hydrazine in a solvent such as DMF or THF, to afford (47). The hydrazine adduct may be treated with a 1,3-diketone such as (49) to afford the pyrazole (51). R₆₃, R₆₄, and R₆₅ may be groups such as but not limited to alkyl, alkenyl, -alkylene-aryl, or hydrogen. The intermediate (51) may be deprotected or cleaved from solid support introducing G₂ input to afford (53). The hydrazide (47) may be treated with a keto acid (48) in a solvent such as dichloroethane or THF, at a temperature of from 25° C. to 100° C., to afford the adduct (50). L₂₁ is preferably methylene or ethylene, optionally substituted with groups such as but not limited to alkyl, alkenyl, aryl, alkylene-heteroaryl, and the like. R₆₂ is a group such as but not limited to aryl, alkyl-aryl and the like. Introduction of the G₂ input as described previously affords the probe (52).

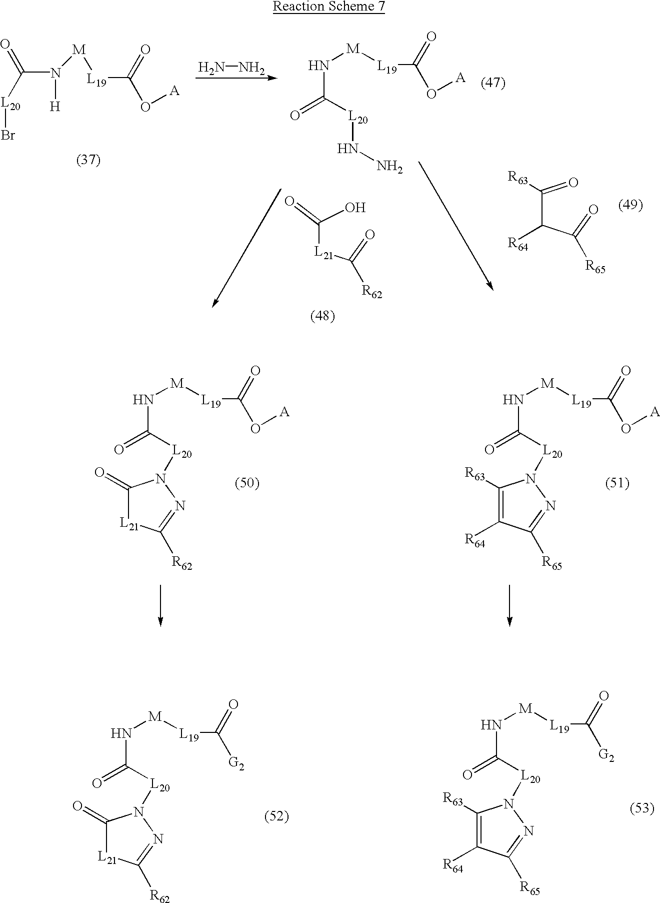

Reaction Scheme 8 describes the synthesis of a probe of formula (61). An aldehyde resin as defined before is reductively aminated with an amine (54) employing a reagent such as sodium cyanoborohydride in a solvent such as THF, to afford (55). R₆₇ and R₆₆ are, independently, groups such as but not limited to alkyl, hydrogen, or are taken together to form a heterocyclyl ring or cycloalkyl ring. The nitrogen of (55) may be protected with a amino protecting group such as Fmoc. The primary alcohol is then oxidized to the aldehyde employing a reagent such as pyridine-sulfur trioxide complex and DMSO, followed by TEA treatment, to afford (56). (56) is then treated with an isocyanide (57) and anthranilic acid (58) in methanol of methanol-THF at a tempoerature of from 25° C. to 100° C., to afford the adduct (59). R₆₈ may be a group selected from, but not limited to, alkyl or aryl. The protecting group PG₁ is removed using methods known in the art. The product is treated in a solvent such as chlorobenzene at a temperature of from 50° C. to 150° C., employing a catalytic amount of a lanthamide triflate such as terbium (III) triflate, to afford the cyclized product (60). Cleavage from the polymeric support is accomplished by treatment of (60) with TFA in DCM, DCM-dimethylsulfide, or water-dimethyl sulfide, to afford (61). In this example, Ar₁ represents an optionally substituted aryl or heteroaryl ring system.

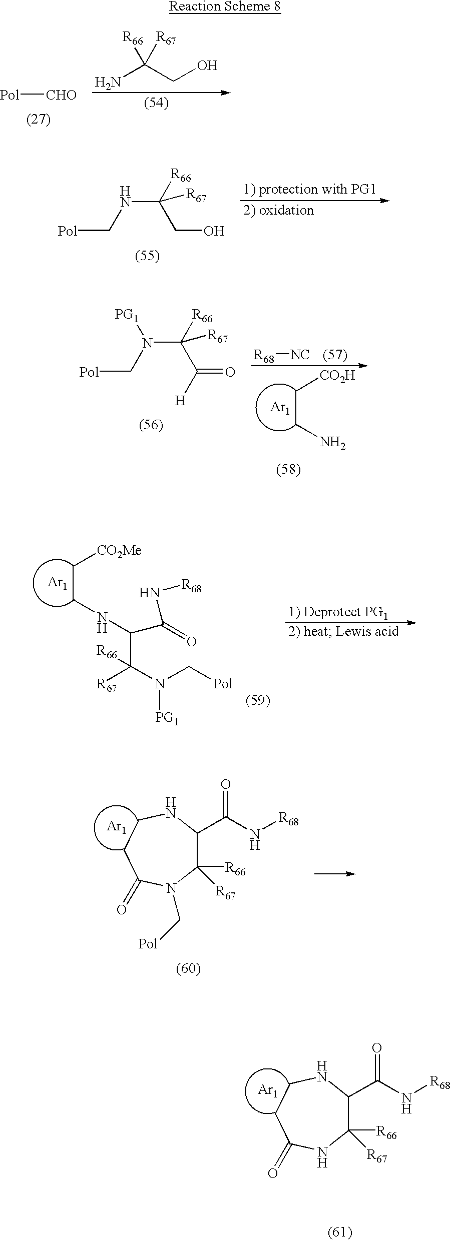

Reaction Scheme 9 describes the synthesis of a probe of formula (68). The protected carboxylic acid (62) is deprotected and reacted with a polymer support such as Wang resin, employing DIPCDI and HOBt/DMAP in DCM, to afford (63). The amino protecting group PG₁ is removed to afford (64), and the resulting amine is reacted with a boronic acid (65) and a keto compound (66) at a temperature of from 25° C. to 80° C., in a solvent such as toluene or THF, to afford the adduct (67). R₆₉ is preferably chosen as but not limited to hydrogen, alkyl, or alkylene-aryl. R₇₀ is alkenyl, aryl, or alkenyl substituted by groups such as but not limited to cycloalkyl, aryl, or alkyl. R₇₂ is a group such as but not limited to alkyl or hydrogen. R₇₁ is a group such as but not limited to alkyl, aryl, or hydrogen. R₇₃ may be 0 or H/OH. The product (67) is then cleaved from the resin with introduction of the G₂ input to afford (68). For example, where G₂ is OH, treatment of (67) where POL is Wang resin with TFA in DCM at a temperature of from 25° C. to 50° C. affords (68).

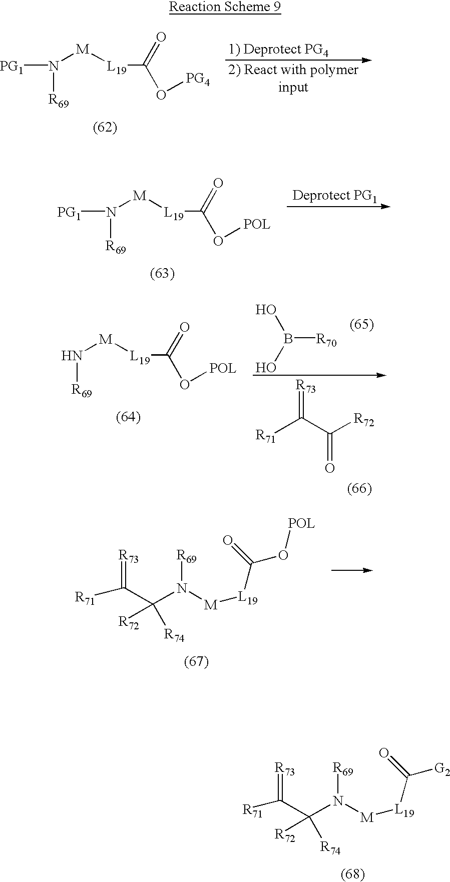

Reaction Scheme 10 provides a synthesis of a probe of formula (70). The protected carboxylic acid (62) is deprotected and reacted with a polymer support such as but not limited to Wang resin, as before. R₆₉ is preferably chosen as but not limited to H, alkyl, or alkylene-aryl. The amino protecting group is removed to afford (64) and the free amine is reacted with an isocyanate R₇₀-NCO to afford (69). R₇₀ is a group such as but not limited to alkyl, alkylene-aryl, or alkylene-cycloalkyl. The compound (69) is heated at a temperature of from 40° C. to 120° C. in the presence or absence of TEA, in a solvent such as THF or toluene, to afford (70). In this example, L₁₉ is preferably a direct bond or a substituted methylene or ethylene group, where substituents are those such as but not limited to alkyl, alkyene-aryl, and the like.

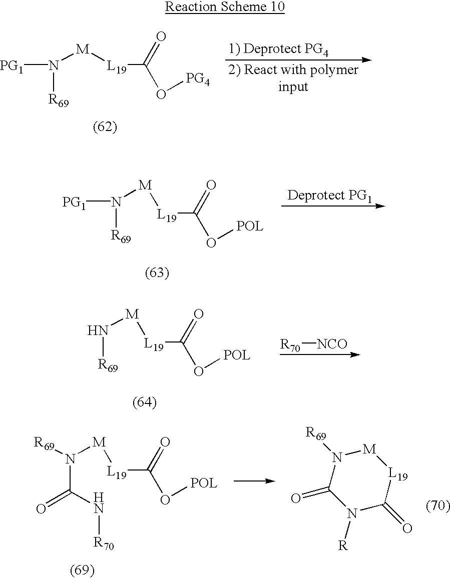

Reaction Scheme 11 describes the synthesis of a probe of formula (76). The protected amino acid (71) is deprotected at the carboxyl group and reacted with a polymeric reagent at the carboxyl group, such as Wang resin, to afford (72). The amino protecting group is removed to provide (73) and the free amine is reacted with an isocyanate R₇₀—NCO in a solvent such as DCM, at a temperature of from 0° C. to 50° C., to afford (74). R₇₀ is a group sych as but not limited to akyl, alkylene-aryl, or alkylene-cycloalkyl. (74) is treated with a ketene reagent such as diketene (where R₇₁ is methyl) at a temperature of from 25° C. to 100° C. in a solvent such as THF, DCM, or DMF, to afford (75). The G₂ input is introduced as detailed before to provide the probe (76).

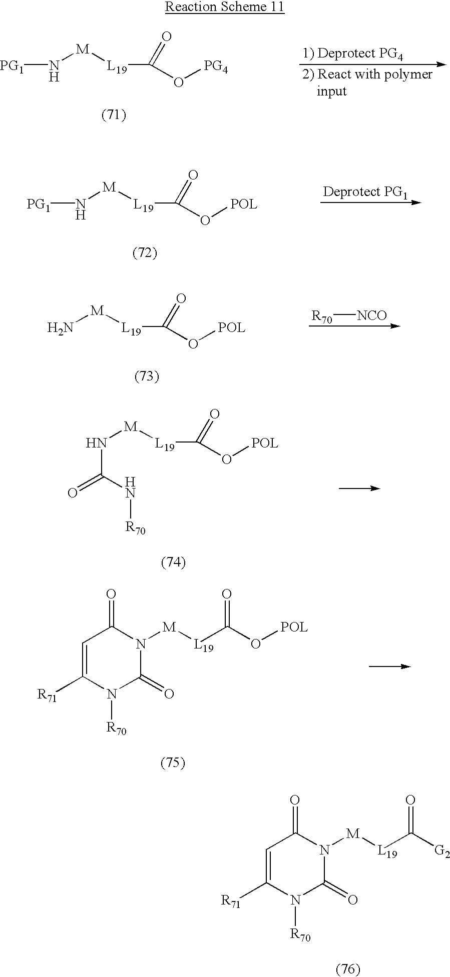

Reaction Scheme 12 provides the synthesis of a probe of formula (82). In this scheme, L₁₉ is preferably a direct bond. The amino acid (73) on polymer support is treated with an isocyanide (77), an aldehyde (78), and a N-protected anthanilic acid (79) in a solvent such as TNF or DCM, at a temperature of from 25° C. to 80° C., to afford the adduct 80. Ar₂ represents an optionally substituted aryl or heteroaryl ring system. The protecting group PG₁ is removed. PG₁ is a group such as Fmoc, and it may be removed by treatment with piperidine in a solvent such as DMF, at a temperature of from 25° C. to 50 ° C. Heating of (81) in a solvent such as toluene at a temperature of from 50° C. to 110° C. provides the probe (82), with cleavage from the solid support.

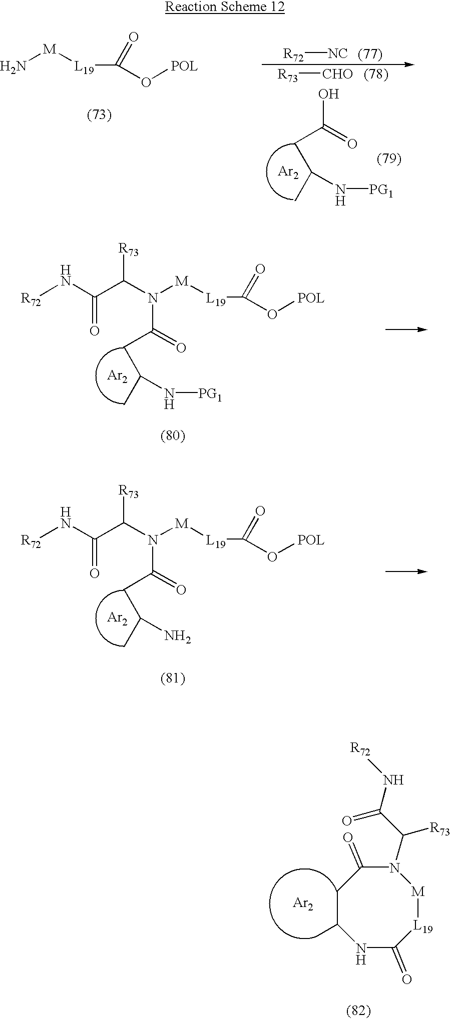

Reaction Scheme 13 describes the synthesis of probes of formulae (87) and (88). The protected amino acid (71) is deprotected at the carboxyl group and reacted with a polymer support, such as but not limited to Wang resin, to afford (72). The amino protecting group PG₁ is removed to afford (73). Where PG₁ is Fmoc, removal may be effected by treatment of (72) with piperidine in a solvent such as DMF, at a temperature of from 25° C. to 50° C. The amine may be treated with a substituted heteroaryl group (83), in a solvent such as DMF or chlorobenzene, at a temperature of from 25° C. to 120 ° C., to afford (85). LG₂ is a leaving group such as fluoro or chloro, and the leaving group LG₂ is preferably located adjacent to a heteroatom in the heteroaryl ring systen hAr. The amine (73) may be treated with an aryl ring system (84) to provide (86). In (84), LG₂ has the same meaning as for (85) and is preferably located vicinally or opposite to an electron withdrawing subsrituent such as but not limited to —NO₂ or —CN. The substitution products (85) and (86) may be transformed to the products (87) and (88) with introduction of the G₂ input as described previously.

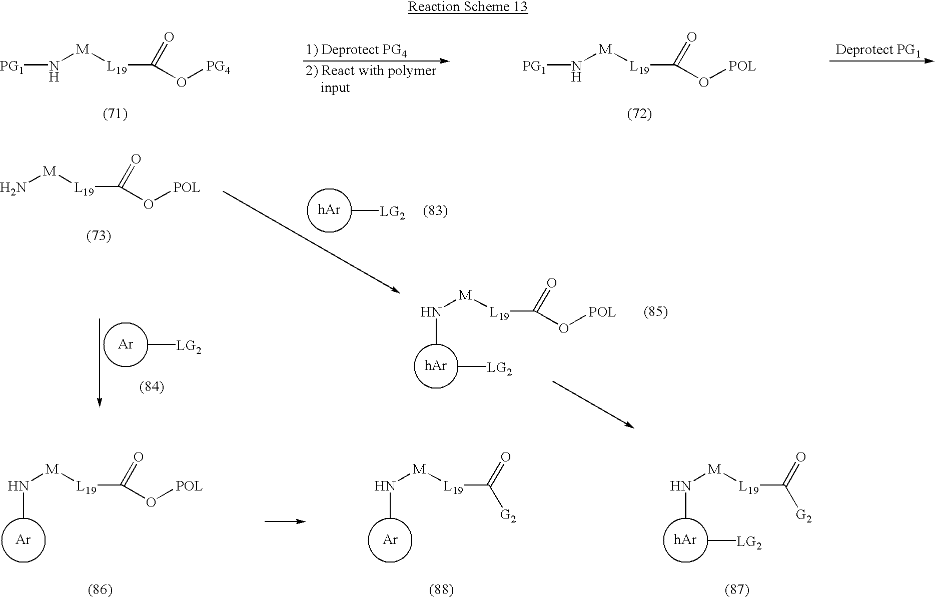

Reaction Scheme 14 describes the synthesis of a probe of formula (91). A protected amino acid is deprotected and reacted with a polymeric support, as described before, such as Wang resin. The amino protecting group PG₁ is removed, where PG₁ is Fmoc, by treatment with piperidine in a solvent such as DMF, at a temperature of from 25° C. to 50° C., to afford (73). Treatment of (73) with the reagents (77), (78), and (89) in a solvent such as THF or DCM, at a temperature of from 25° C. to 80° C., to afford the adduct (90). The variables R₇₂ and R₇₃ in (77) and (78) have the meaning described previously; R₇₄ may be a group such as but not limited to cycloalkyl, aryl, or alkyl. The G₂ input may be introduced into this compound with cleavage from the resin as described before to afford (91).

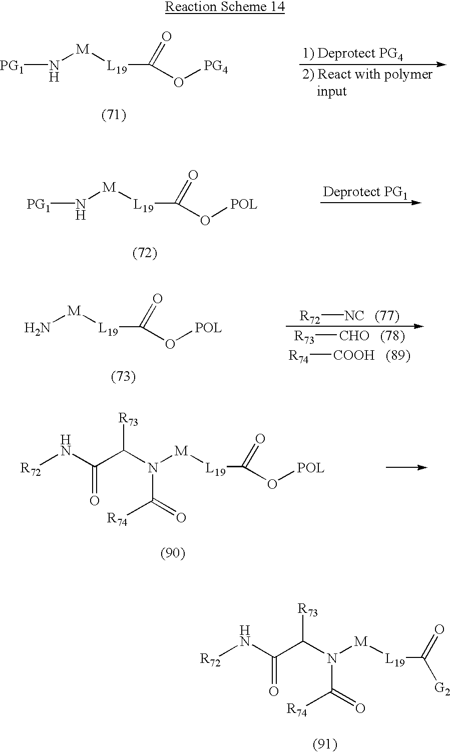

In the above schemes, “PG₁”, “PG₂”, “PG₃”, and “PG₄” may represent amino protecting groups. The term “amino protecting group” as used herein refers to substituents of the amino group commonly employed to block or protect the amino functionality while reacting other functional groups on the compound. Examples of such amino-protecting groups include the formyl group, the trityl group, the phthalimido group, the trichloroacetyl group, the chloroacetyl, bromoacetyl and iodoacetyl groups, urethane-type blocking groups such as benzyloxycarbonyl, 4-phenylbenzyloxycarbonyl, 2-methylbenzyloxycarbonyl, 4-methoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyloxycarbonyl, 4-bromobenzyloxycarbonyl, 3-bromobenzyloxycarbonyl, 4-nitrobenzyloxycarbonyl, 4-cyanobenzyloxy-carbonyl, 2-(4-xenyl)iso-propoxycarbonyl, 1,1-diphenyleth-1-yloxycarbonyl, 1,1-diphenylprop-1-yloxycarbonyl, 2-phenylprop-2-yloxycarbonyl, 2-(p-toluyl)prop-2-yloxycarbonyl, cyclopentanyloxycarbonyl, 1-methylcyclopentanyloxycarbonyl, cyclohexanyloxycarbonyl, 1-methylcyclohexanyloxycarbonyl, 2-methylcyclohexanyloxycarbonyl, 2-(4-toluylsulfonyl)ethoxycarbonyl, 2(methylsulfonyl)ethoxycarbonyl, 2-(triphenylphosphino)ethoxycarbonyl, 9-fluorenylmethoxycarbonyl (“FMOC”), t-butoxycarbonyl (“BOC”), 2-(trimethylsilyl)ethoxycarbonyl, allyloxycarbonyl, 1-(trimethylsilylmethyl)prop-1-enyloxycarbonyl, 5-benzisoxalylmethoxycarbonyl, 4-acetoxybenzyloxycarbonyl, 2,2,2-trichloroethoxycarbonyl, 2-ethynyl-2-propoxycarbonyl, cyclopropylmethoxycarbonyl, 4-(decyloxy)benzyloxycarbonyl, isobornyloxycarbonyl, 1-piperidyloxycarbonyl and the like; the benzoylmethylsulfonyl group, the 2-(nitro)phenylsulfenyl group, the diphenylphosphine oxide group and like amino-protecting groups. The species of amino-protecting group employed is not critical so long as the derivatized amino group is stable to the condition of subsequent reaction(s) on other positions of the compound of Formula (I) and can be removed at the desired point without disrupting the remainder of the molecule. Preferred amino-protecting groups are the allyloxycarbonyl, the t-butoxycarbonyl, 9-fluorenylmethoxycarbonyl, and the trityl groups. Similar amino-protecting groups used in the cephalosporin, penicillin and peptide art are also embraced by the above terms. Further examples of groups referred to by the above terms are described by J. W. Barton, “Protective Groups In Organic Chemistry”, J. G. W. McOmie, Ed., Plenum Press, New York, N.Y., 1973, and T. W. Greene, “Protective Groups in Organic Synthesis”, John Wiley and Sons, New York, N.Y., 1981. The related term “protected amino” defines an amino group substituted with an amino-protecting group discussed above.

In the above schemes, “PG₁”, “PG₂”, “PG₃”, and “PG₄” may represent a hydroxyl protecting group. The term “hydroxyl protecting group” as used herein refers to substituents of the alcohol group commonly employed to block or protect the alcohol functionality while reacting other functional groups on the compound. Examples of such alcohol-protecting groups include the 2-tetrahydropyranyl group, 2-ethoxyethyl group, the trityl group, the trichloroacetyl group, urethane-type blocking groups such as benzyloxycarbonyl, and the trialkylsilyl group, examples of such being trimethylsilyl, tert-butyldimethylsilyl, phenyldimethylsilyl, triiospropylsilyl and thexyldimethylsilyl. The choice of of alcohol-protecting group employed is not critical so long as the derivatized alcohol group is stable to the condition of subsequent reaction(s) on other positions of the compound of the formulae and can be removed at the desired point without disrupting the remainder of the molecule. Further examples of groups referred to by the above terms are described by J. W. Barton, “Protective Groups In Organic Chemistry”, J. G. W. McOmie, Ed., Plenum Press, New York, N.Y., 1973, and T. W. Greene, “Protective Groups in Organic Synthesis”, John Wiley and Sons, New York, N.Y., 1981. The related term “protected hydroxyl” or “protected alcohol” defines a hydroxyl group substituted with a hydroxyl-protecting group as discussed above.

In the above schemes, “PG₁”, “PG₂”, “PG₃”, and “PG₄” may represent a carboxyl protecting group. The term “carboxyl protecting group” as used herein refers to substituents of the carboxyl group commonly employed to block or protect the —OH functionality while reacting other functional groups on the compound. Examples of such alcohol-protecting groups include the 2-tetrahydropyranyl group, 2-ethoxyethyl group, the trityl group, the allyl group, the trimethylsilylethoxymethyl group, the 2,2,2-trichloroethyl group, the benzyl group, and the trialkylsilyl group, examples of such being trimethylsilyl, tert-butyldimethylsilyl, phenyldimethylsilyl, triiospropylsilyl and thexyldimethylsilyl. The choice of carboxyl protecting group employed is not critical so long as the derivatized alcohol group is stable to the condition of subsequent reaction(s) on other positions of the compound of the formulae and can be removed at the desired point without disrupting the remainder of the molecule. Further examples of groups referred to by the above terms are described by J. W. Barton, “Protective Groups In Organic Chemistry”, J. G. W. McOmie, Ed., Plenum Press, New York, N.Y., 1973, and T. W. Greene, “Protective Groups in Organic Synthesis”, John Wiley and Sons, New York, N.Y., 1981. The related term “protected carboxyl” defines a carboxyl group substituted with a carboxyl-protecting group as discussed above.

General Procedures

1.Attachment to Resin

1A. Hydroxymethyl Polystyrene

1.A.1 DIPCDI/DMAP

Hydroxymethyl polystyrene (0.1 mmol) was treated with 1 M solutions (DMF) of: a suitably protected amino acid or carboxylic acid (0.4 mmol, 4 equiv), DIPCDI (0.4 mmol, 4 equiv), and DMAP (0.01 mmol, 0.1 equiv). The slurry was shaken at room temperature for 16 h, filtered, and the resin washed consecutively with DMF (3×), MeOH (3×), and DCM (3×).

1.A.2 HBTU/DIEA

Hydroxymethyl polystyrene (0.1 mmol) was treated with 1 M solutions (DMF) of: a suitably protected amino acid or carboxylic acid (0.4 mmol, 4 equiv), HBTU (0.4 mmol, 4 equiv), and DIEA (0.8 mmol, 8 equiv). The slurry was shaken at room temperature for 16 h, filtered, and the resin washed consecutively with DMF (3×), MeOH (3×), and DCM (3×).

1B. Wang Resin

1.B.1 DIPCDI/DMAP

Wang Resin (0.1 mmol) was treated with 1M solutions (DMF) of: a suitably protected amino acid or carboxylic acid (0.4 mmol, 4 equiv), DIPCDI (0.4 mmol, 4 equiv), and DMAP (0.01 mmol, 0.1 equiv). The slurry was shaken at room temperature for 16 h, filtered, and the washed consecutively with DMF (3×), MeOH (3×), and DCM (3×).

1.B.2 HBTU/DIEA

Wang Resin (0.1 mmol) was treated with 1 M solutions (DMF) of: a suitably protected amino acid or carboxylic acid (0.4 mmol, 4 equiv), HBTU (0.4 mmol, 4 equiv), and DIEA (0.8 mmol, 8 equiv). The slurry was shaken at room temperature for 16 h, filtered, and the resin washed consecutively with DMF (3×), MeOH (3×), and DCM (3×).

IC. Rink Resin

1.C.1 DIPCDI/HOBt

Rink Resin (0.1 mmol) was treated with piperidine according to the general procedure, 2.A. The resulting resin was treated with 1 M solutions (DMF) of: a suitably protected amino acid or carboxylic acid (0.4 mmol, 4 equiv), DIPCDI (0.4 mmol, 4 equiv), and HOBt (0.4 mmol, 0.4 equiv). The slurry was shaken at room temperature for 16 h, filtered, and the resin washed consecutively with DMF (3×), MeOH (3×), and DCM (3×).

1.C.2 HBTU/DIEA

Rink Resin (0.1 mmol) was treated with piperidine according to the general procedure, 2.A. The resulting resin was treated 1 M solutions (DMF) of: a suitably protected amino acid or carboxylic acid (0.4 mmol, 4 equiv), HBTU (0.4 mmol, 4 equiv), and DIEA (0.8 mmol, 8 equiv). The slurry was shaken at room temperature for 16 h, filtered, and the resin washed consecutively with DMF (3×), MeOH (3×), and DCM (3×).

ID. Aldehyde Resin

1.D.1 DIPCDI/HOBt

Aldehyde Resin (0.1 mmol) was reductively aminated with a primary amine according to the general procedure, 5.B. The resulting resin was treated with 1M solutions (DMF) of: a suitably protected amino acid or carboxylic acid (0.4 mmol, 4 equiv), DIPCDI (0.4 mmol, 4 equiv), and HOBt (0.4 mmol, 0.4 equiv). The slurry was shaken at room temperature for 16 h, filtered, and the resin washed consecutively with DMF (3×), MeOH (3×), and DCM (3×).

1.D.2 HBTU/DIEA

Aldehyde Resin (0.1 mmol) was reductively aminated with a primary amine according to the general procedure 5.B. The resulting resin was treated 1 M solutions (DMF) of: a suitably protected amino acid or carboxylic acid (0.4 mmol, 4 equiv), HBTU (0.4 mmol, 4 equiv), and DIEA (0.8 mmol, 8 equiv). The slurry was shaken at room temperature for 16 h, filtered, and the resin washed consecutively with DMF (3×), MeOH (3×), and DCM (3×).

1.D.3 Ugi

Aldehyde Resin (0.1 mmol) was treated with solutions of: suitably protected amino acid or carboxylic acid (1 M, MeOH or MeOH—CHCl₃) (0.3 mmol, 3 equiv), amine (1 M, CHCl₃) (0.3 mmol, 3 equiv), and isocyanide (1 M, MeOH) (0.3 mmol, 3 equiv). The slurry was heated to 60° C. for 16 h, filtered, and the resin washed consecutively with DMF (3×), MeOH (3×), and DCM (3×).

1.D.4. DIPCDI/HOBt, Triple Coupling

Aldehyde Resin (0.1 mmol) was reductively aminated with a primary amine according to the general procedure5.B. The resulting resin was treated with 5 eq. of carboxylic acid (1 M in DMF), 5 eq. of DIPCDI (1M in DMF) and 5 eq. of HOBt (1M in DMF). The reaction was agitated for 24 hours. The resin was then washed using 3× DMF, and 3× DCM. The acylation-washing procedure was then repeated two more times.

1.D.5 Reductive Amination Only

Aldehyde Resin (0.1 mmol) was reductively aminated with a primary amine according to the general procedure, 5.B.

1.D.6 DIPCDI/HOBt (1 h)

Aldehyde Resin (0.1 mmol) was reductively aminated with a primary amine according to the general procedure, 5.B. The resulting resin was treated with 1 M solutions (DMF) of: a suitably protected amino acid or carboxylic acid (0.5 mmol, 5 equiv), DIPCDI (0.5 mmol, 5 equiv), and HOBt (0.5 mmol, 0.5 equiv). The slurry was shaken at room temperature for 1 h, filtered, and the resin washed consecutively with DMF (3×), MeOH (3×), and DCM (3×).

1E. Wang Carbonate Resin

1.E.1 Method 1

Wang Carbonate resin (0.1 mmol) was treated with 1M solutions (DCM) of: an amine (0.5 mmol, 5 equiv) and DIEA (1.0 mmol, 10 equiv). The slurry was shaken at room temperature for 16 h, filtered, and the resin washed consecutively with DMF (3×), MeOH (3×), and DCM (3×).

1.E.2 Method 2

Wang Carbonate resin (0.1 mmol) was treated with 1M solutions (DCM or DMF) of: an amine (0.4 mmol, 4 equiv) and DIEA (8.0 mmol, 8 equiv). The slurry was shaken at room temperature for 16 h, filtered, and the resin washed consecutively with DMF (3×), MeOH (3×), and DCM (3×).

1F. Wang Bromo Resin

Wang Bromo Resin was treated with 1 M solutions (DMF) of: an amine (4.0 mmol, 40 equiv) and DIEA (1.0 mmol, 10 equiv). The resulting mixture was heated at 50° C. for 16 h, filtered and then washed consecutively with DMF (3×), MeOH (3×), and DCM (3×).

1G. THP Resin

THP Resin was treated with 1 M solutions (1,2-dichloroethane) of: an alcohol (0.3 mmol, 3 equiv) and p-toluenesulphonate (1.0 mmol, 10 equiv). The resulting mixture was heated at 80° C. for 16 h, quenched with excess pyridine, filtered and then washed consecutively with DMF (3×), MeOH (3×), and DCM (3×).

2. Deprotection

2.A. Removal of Fmoc Protecting Group

The Fmoc group was removed by treatment with 2 ml of 20% piperdine in DMF for 20-60 minutes. The resin was then washed using 3×DMF, 3×MeOH, and 3×DCM.

2.B. Removal of Boc/t-bu Based Protecting Group

The Boc or t-butyl based protecting group was removed by treatment with 2 ml of 20% TFA in DCM for 20-60 minutes. The resin was then washed using 3×DMF, 3×10% TEA in DCM, 3×MeOH, and 3×DCM.

2.C. Removal of O-Trityl Protecting Group

The trityl group was removed by treatment with 2 ml of a DCM-TFA-triethylsilane (94:1:5) for 1 minute. The resin was drained and the procedure repeated 4 times. The resin was then washed using 3×DMF, 3×MeOH, and 3×DCM.

3. Acylations

3.A. DIPCDI/HOBt

0.1 mmol of resin-bound amine or resin bound aryl hydrazine was treated with 4 eq. of carboxylic acid (1 M in DMF), 4 eq. of DIPCDI (1 M in DMF) and 4 eq. of HOBt (1 M in DMF). The reaction was agitated for 24 hours. The resin was then washed using 3×DMF, 3×MeOH, and 3×DCM.

3.B. HBTU/DIEA

0.1 mmol of resin-bound amine was treated with 4 eq. of carboxylic acid (1 M in DMF), 4 eq. HBTU (1 M in DMF), and 8 eq. of DIEA (neat or 1 M in DMF). The reaction was agitated for 24 hours. The resin was then washed using 3×DMF, 3×MeOH, and 3×DCM.

3.C. Anhydrides

3.C.1. Commercially Available

0.1 mmol of resin-bound amine was treated with 8 eq. of anhydride (1 M in DCM) and 2 eq. of TEA (1 M in DCM). The reaction was agitated for 8 hours. The resin was then washed using 3×DMF, 3×MeOH, and 3×DCM.

3.C.2. Non-Commercially Available

For non-commercially available anhydrides, 8 eq. of the carboxylic acid (1 M in DCM) was treated with 4 eq. of DIPCDI (neat) for 5 minutes followed by addition to the resin-bound amine. The reaction was agitated for 8 hours. The resin was then washed using 3×DMF, and 3×DCM.

3.D. DIPCDI/HOBT/TEA

0.1 mmol of resin-bound amine was treated with 5 eq. of carboxylic acid (1M in DMF), 5 eq. of DIPCDI (1 M in DMF), 10 eq. of TEA (1 M in DMF) and 5 eq. of HOBt (1 M in DMF). The reaction was agitated for 24 hours. The resin was then washed using 3×DMF, 3×MeOH, and 3×DCM.

3.E. Acid Chloride

0.1 mmol of resin-bound amine was treated with 5 eq. of acid chloride (1 M in DCM), and 10 eq. of TEA (1 M in DCM). The reaction was agitated for 24 hours. The resin was then washed using 3×DMF, 3×MeOH, and 3×DCM.

3.F. Method 6

0.1 mmol of resin bound carboxylic acid was treated with 5 eq. of an amine (1 M in DMF), 5 eq. of DIPCDI (1 M in DMF) and 5 eq. of HOBt (1 M in DMF). The reaction was agitated for 16 hours. The resin was washed with 3×DMF, 3×MeOH, and 3×DCM.

3.G. Method 7

0.1 mmol of resin bound carboxylic acid in 0.4 ml of DMF was treated with 2 eq. of an amine equivalent (i.e. ammonium chloride), 1.5 eq. of HBTU, 1.5 eq. of HOBt and 4 eq. of DIEA. The reaction was agitated for 16 hours. The resin was washed with 3×DMF, 3×MeOH, and 3×DCM to give the unsubstituted primary amide.

3.H. DIPCDI/HOBt

0.1 mmol of resin-bound amine or resin bound aryl hydrazine was treated with 4 eq. of carboxylic acid (1 M in DMF), 4 eq. of DIPCDI (1M in DMF) and 4 eq. of HOBt (1 M in DMF). The reaction was agitated for 24 hours. The resin was then washed using 3×DMF, and 3×DCM. The entire procedure was then repeated two more times.

4. Sulfonamide Formation and Sulfonyl Urea Formation

4.A. Method 1 Sulfonamide Formation

0.1 mmol of resin-bound amine was treated with 7 eq. of sulfonyl chloride (1M in DCM) and 2 eq. of TEA (1M in DCM). The reaction was agitated for 16 hours. The resin was then washed using 3×DMF, 3×MeOH, and 3×DCM.

4.B. Sulfonyl Urea Formation

4.B.1 Method 1

0.1 mmol of resin-bound amine was treated with 5 eq. of a sulfamoyl chloride (1M in DCM) and 10 eq. of TEA (1M in DCM). The reaction was heated to 50° C. for 16 hours. The resin was then washed using 3×DMF, 3×MeOH, and 3×DCM.

4.B.2 Method 2

0.1 mmol of a resin-bound amine was treated with 3 eq. of a 1,1′-sulfonyldiimidazole (0.5 M in DCM/DMF, 50:50) and 6 eq. of DIEA (0.5 M in DCM/DMF, 50:50). The mixture was agitated for 4 hours. The resin was washed with 3×DMF, 3×MeOH, and 3×DCM. The resin bound sulfonylimidazole was treated with 3.5 eq. of an amine (1 M in DMF) and 10 eq. of DIEA (1 M in DMF). The mixture was agitated for 16 hours followed by heating for 4 hours at 50° C. The resin was washed with 3×DMF, 3×MeOH, and 3×DCM.

5. Reductive Amination

5.A. Resin-Bound Amine

0.1 mmol of resin-bound amine was treated with 4 eq. of aldehyde or ketone (1 M in DCE) and 2 eq. of HOAc (1 M in DCE) and 7 eq. of NaCNBH₃ (1 M in THF). The reaction was agitated for 16 hours. The resin was then washed using 3×DMF, 3×10% TEA in DCM, 3×MeOH, and 3×DCM.

5.B. Resin-Bound Carbonyl (Aldehyde or Ketone) Treated with Nucleophillic Amine

0.1 mmol of resin-bound carbonyl was treated with 5 eq. of amine (1M in DCE) and 2 eq. of HOAc (1 M in DCE) and 7 eq. of NaCNBH₃ (1 M in THF). The reaction was agitated for 16 hours. The resin was then washed using 3×DMF, 3×10% TEA in DCM, 3×MeOH, and 3×DCM.

5.C. Resin-Bound Carbonyl (Aldehyde or Ketone) Treated with Non-Nucleophillic Amine

0.1 mmol of resin-bound carbonyl was treated with 20 eq. of amine (1M in DCE) and 2 eq. of HOAc (1 M in DCE) and 7 eq. of NaCNBH₃ (1 M in THF). The reaction was agitated for 16 hours. The resin was then washed using 3×DMF, 3×10% TEA in DCM, 3×MeOH, and 3×DCM.

6. Urea Formation

6A. Isocyante

A resin bound amine (0.1 mmol) was treated with a 1 M solution (DCM) of an isocyante (0.7 mmol, 7 equiv). The slurry was shaken at room temperature for 16 h, filtered, and the resin washed consecutively with DMF (3×), MeOH (3×), and DCM (3×).

6B. Triphosgene/Amine

A resin bound amine (0.1 mmol) was treated with 1 M solutions (DCM) of: triphogene (0.3 mmol, 3 equiv) and DIEA (1.0 mmol, 10 equiv). The slurry was shaken at room temperature for 3 h, filtered, and the resin washed consecutively with DMF (3×), and DCM (3×). The resulting resin was treated with 1 M solutions (DMF) of: an amine (0.5 mmol, 5 equiv) and DIEA (1.0 mmol, 10 equiv). The slurry was shaken at room temperature for 16 h, filtered, and the resin washed consecutively with DMF (3×), MeOH (3×), and DCM (3×).

6C. Carbamoyl Chloride

A resin bound amine (0.1 mmol) was treated with 1 M solutions (DCM) of: an N,N-disubstituted carbamoyl chloride (0.5 mmol, 5 equiv) and DIEA (1.0 mmol, 10 equiv). The slurry was shaken at room temperature for 16 h, filtered, and the resin washed consecutively with DMF (3×), MeOH (3×), and DCM (3×).

7. Carbamate Formation

7A. Chloroformate

7.A.1 Method 1

A resin bound amine (0.1 mmol) was treated with 1 M solutions (DCM) of a chloroformate (0.5 mmol, 5 equiv) and DIEA (1.0 mmol, 10 equiv). The slurry was shaken at room temperature for 16 h, filtered, and the resin washed consecutively with DMF (3×), MeOH (3×), and DCM (3×).

7.A.2 Method 2

A resin bound amine (0.1 mmol) was treated with solutions of: a chloroformate (1 M, NMP) (0.11 mmol, 1.1 equiv) and DIEA (1M, NMP) (0.2 mmol, 2 equiv). The slurry was shaken at room temperature for 18 h, filtered, and the resin washed consecutively with DMF (3×), MeOH (3×), and DCM (3×).

7B. Triphosgene/Alcohol

A resin bound amine (0.1 mmol) was treated with 1 M solutions (DCM) of: triphogene (0.3 mmol, 3 equiv) and DIEA (1.0 mmol, 10 equiv). The slurry was shaken at room temperature for 3 h, filtered, and the resin washed consecutively with DMF (3×), and DCM (3×). The resulting resin was treated with a 1 M solution (DCM) of: an alcohol (1.0 mmol, 5 equiv) and DIEA (0.10 mmol, 1 equiv). The slurry was heated to reflux for 16 h, filtered, and the resin washed consecutively with DMF (3×), MeOH (3×), and DCM (3×).

8. Alpha-Halo Carbonyl Substitution

8.A. Amine Substitution

8.A.1. Method 1

To 0.1 mmol of resin bound alpha-halo carbonyl was added 5 eq. of amine (1 M in DMF) and 10 eq. of DIEA (1M in DMF). The reaction was agitated for 16 hours. The resin was washed with 3×DMF, 3×MeOH, and 3×DCM.

8.A.2. Method 2

To 0.1 mmol of resin bound alpha-halo carbonyl was added 5 eq. of amine (1 M in DMF) and 10 eq. of DIEA (1M in DMF). The reaction was heated at 60° C. for 16 hours. The resin was washed with 3×DMF, 3×MeOH, and 3×DCM.

8.B. Thiol Substitution

8.B.1 Method 1

To 0.1 mmol of resin bound alpha-halo carbonyl was added 5 eq. of thiol (1 M in DMF) and 10 eq. of DIEA (1M in DMF). The reaction was agitated for 16 hours. The resin was washed with 3×DMF, 3×MeOH, and 3×DCM.

8.B.2 Method 2

To 0.1 mmol of resin bound alpha-halo carbonyl was added 5 eq. of thiol (1 M in DMF) and 10 eq. of DIEA (1M in DMF). The reaction was heated to 60° C. for 16 hours. The resin was washed with 3×DMF, 3×MeOH, and 3×DCM.

8.C. Hydrazine Substitution

To 0.1 mmol of resin bound alpha-halo carbonyl was added 5 eq. of hydrazine hydrate (15% in Dioxane, V/V). The reaction was agitated for 16 hours. The resin was washed with 3×DMF, and 3×DCM.

8.D. Thiosemicarbazide Addition

8.D.1. Method 1 Thiosemicarbazide Addition

To 0.1 mmol of resin bound alpha-halo carbonyl was added 10 eq. of thiosemicarbazide (1M in DMF). The reaction was agitated for 16 hours. The resin was washed with 3×DMF, 3×MeOH, and 3×DCM.

8.D.2. Method 2 Substituted Thiosemicarbazide Addition

To 0.1 mmol of resin bound alpha-halo carbonyl was added 10 eq. of a substituted thiosemicarbazide (1M in DMF). The reaction was agitated for 16 hours. The resin was washed with 3×DMF, 3×MeOH, and 3×DCM.

8.E. Thiourea Addition

8.E.1 Method 1 Thiourea Addition

To 0.1 mmol of resin bound alpha-halo carbonyl was added 10 eq. of thiourea (1M in DMF). The reaction was agitated for 16 hours. The resin was washed with 3×DMF, 3×MeOH, and 3×DCM.

8.E.2 Method 2 Substituted Thiourea Addition

To 0.1 mmol of resin bound alpha-halo carbonyl was added 10 eq. of a substituted thiourea (1M in DMF). The reaction was agitated for 16 hours. The resin was washed with 3×DMF, 3×MeOH, and 3×DCM.

9. Ugi Reactions

9A. Method 1

A resin bound amine (0.1 mmol) was treated with solutions of: an aldehyde or ketone (1 M, THF or MeOH) (0.5 mmol, 5 equiv), carboxylic acid (0.5M, THF) (0.5 mmol, 5 equiv), and isocyanide (1M, MeOH) (0.5 mmol, 5 equiv). The slurry was shaken at room temperature for 16 h, filtered, and the resin washed consecutively with DMF (3×), MeOH (3×), and DCM (3×).

9B. Method 2

A resin bound amine (0.1 mmol) was treated with solutions of: an aldehyde or ketone (1 M, THF or MeOH) (0.5 mmol, 5 equiv), carboxylic acid (0.5M, THF) (0.5 mmol, 5 equiv), isocyanide (1M, MeOH) (0.5 mmol, 5 equiv), and zinc chloride (0.5M, THF) (0.25 mmol, 2.5 equiv). The slurry was shaken at room temperature for 16 h, filtered, and the resin washed consecutively with DMF (3×), MeOH (3×), and DCM (3×).

9C. Method 3

A resin bound amine (0.1 mmol) was treated with solutions of: an aldehyde or ketone or hemiacetal (1 M, CHCl₃) (1.0 mmol, 10 equiv), carboxylic acid (1 M, MeOH or MeOH—CHCl₃) (1.0 mmol, 10 equiv), and isocyanide (1M, MeOH) (1.0 mmol, 10 equiv). The slurry was heated to 60° C. for 16 h, filtered, and the resin washed consecutively with DMF (3×), MeOH (3×), and DCM (3×).

9D. Method 4

A resin bound aldehyde or ketone (0.1 mmol) was treated with solutions of: an anthranilic acid (1 M, MeOH) (0.5 mmol, 5 equiv), and titanium isopropoxide (1 M, MeOH) (1.0 mmol, 10 equiv). The slurry was shaken at room temperature for 72 h, filtered, and the resin washed DCM (2×). The resulting resin was treated with an isocyanide (1 M, MeOH) (0.5 mmol, 5 equiv), shaken at room temperature for 18 h, filtered, and washed consecutively with DMF (3×), MeOH (3×), and DCM (3×).

9.E. Method 5

0.1 mmol of resin-bound isocyanide was treated with 10 eq. of an amine (1 M in MeOH), 10 eq. of a carboxylic acid (1 M in MeOH) and 10 eq. of an aldehyde (1 M in CHCl₃). The resin was agitated for 16 hours. The resin was washed with 3×DMF, 3×MeOH, and 3×DCM.

9.F. Method 6

0.1 mmol of resin-bound aldehyde was treated with 10 eq. of an amine (1 M in MeOH), 10 eq. of a carboxylic acid (1 M in CHCl₃) and 10 eq. of an isocyanide (1 M in MeOH). The resin was agitated for 16 hours. The resin was washed with 3×DMF, 3×MeOH, and 3×DCM.

9.G. Method 7

0.1 mmol of resin-bound carboxylic acid was treated with 10 eq. of an aldehyde, ketone or hemiacetal (1 M in CHCl₃), 10 eq. of a amine (1 M in MeOH) and 10 eq. of an isocyanide (1 M in MeOH). The resin was agitated for 16 hours. The resin was washed with 3×DMF, 3×MeOH, and 3×DCM.

9H. Method 8

A resin bound, secondary amine (0.1 mmol) was treated with solutions of: an aldehyde or ketone (1M, CHCl₃) (1.0 mmol, 10 equiv), isocyanide (1M, MeOH) (1.0 mmol, 10 equiv) and a catalytic amount of acetic acid. The slurry was heated to 60° C. for 16 h, filtered, and the resin washed consecutively with DMF (3×), MeOH (3×), and DCM (3×).

10. Mitsunobu Reaction

10.A. Resin-Bound Phenol

To 0.1 mmol of resin bound phenol was added 10 eq. of the alcohol (1M in THF), and 10 eq. of triphenylphosphine (1M in THF) followed by agitating the mixture for 30 min. To the mixture was added 10 eq. of DIAD (1M in THF). The reaction was agitated for 16 hours. The resin was washed with 3×DMF, 3×MeOH, and 3×DCM.

10.B. Resin-Bound Alcohol

To 0.1 mmol of resin bound phenol was added 10 eq. of a phenol or thiophenol (1M in THF), and 10 eq. of triphenylphosphine (1M in THF) followed by agitating the mixture for 30 min. To the mixture was added 10 eq. of DIAD (1M in THF). The reaction was agitated for 16 hours. The resin was washed with 3×DMF, 3×MeOH, and 3×DCM.

11. Cleavages

11.A. Wang/Rink Acidolysis

To 0.1 mmol of resin bound product was added 2 ml of 20% TFA in DCM. The reaction was agitated for 30-120 minutes. The cleaved product was collected and the solvent evaporated.

11.B. Alkyl Amine Cleavage

To 0.1 mmol of resin bound product on wang or Merrifield resin was added 2 ml of 1M methylamine in THF. The reaction was agitated for 16 hours. The cleaved product was collected and the solvent evaporated.

11.C. Alkyl Amine Cleavage with Heat

To 0.1 mmol of resin bound product on wang or Merrifield resin was added 2 ml of 1M alkyl amine in THF. The reaction was heated at 60° C. for 16 hours. The cleaved product was collected and the solvent evaporated.

11.D. Basic Cyclitive Cleavage for Hydantoins and 7-Membered Rings

To 0.1 mmol of resin bound product on wang or Merrifield resin was added 2 ml of 1M TEA in THF. The reaction was heated at 60° C. for 16 hours. The cleaved product was collected and the solvent evaporated.

11.E. Acidic Cyclitive Cleavage for 7-Membered Rings

To 0.1 mmol of resin bound product on Merrifield resin was added 2 ml of 10% HOAc in DCE. The reaction was heated at 60° C. for 24 hours. The cleaved product was collected and the solvent evaporated.

11.F. Cleavage of Alcohol from THP Resin

To 0.1 mmol of resin bound product on THP resin was added 2 ml of a solution of acetic acid/THF/water (5/3/1.5, v/v). The reaction was heated at 80° C. for 16 hours. The cleaved product was collected and the solvent evaporated.

11.G. Cyclitive Cleavage to Form Benzodiazapine

11.G.1 Method 1

To 0.1 mmol of resin bound product on Wang or Merrifield resin was added 2 ml of a solution of 2% acetic acid in DCE. The reaction was heated at 100° C. for 16 hours. The cleaved product was collected and the solvent evaporated.

11.G.2. Method 2

To 0.1 mmol of resin bound product on Wang or Merrifield resin was added 2 ml of a solution of 20% acetic acid in isobutanol. The reaction was heated at 100° C. for 16 hours. The cleaved product was collected and the solvent evaporated.

11.H. Hydroxide Cleavage

To 0.1 mmol of resin bound product on Wang and Merrifield resin was added 2 ml of a 50:50 solution of 1.0 M NaOH/THF or 1.0 M NaOH/dioxane. The reaction was agitated for 16 hours. The cleaved product was collected, neutralized and the solvent was evaporated.

11.I. Wang Carbonate Cleavage

11.I.1 Method 1

To 0.1 mmol of resin bound product was added 2 ml of a solution of 20% TFA in DCM. The reaction was agitated for 30-120 minutes. The cleaved product was collected and the solvent evaporated.

11.I.2 Method 2

To 0.1 mmol of resin bound product was added 2 ml of a solution of 2% TFA in toluene. The reaction was heated at 60° C. for 16 hours. The cleaved product was collected and the solvent evaporated.

11.J. Alcoholic Cleavage with Heat

To 0.1 mmol of resin bound product on Wang or Merrifield resin was added 1 ml of 1 M aliphatic alcohol in THF and 1 ml of 1 M TEA in THF. The reaction was heated at 50° C. for 16 hours. The cleaved product was collected and the solvent evaporated.

11.K. Cyclitive Cleavage to form 2-Aminoimidazolones

0.1 mmol of resin-bound N,N,S-trisubstituted thiourea was treated with 1 ml of DMSO at 80° C. for 16 hours. The cleaved product was collected and the solvent evaporated.

11.L. Cleavage from Aldehyde Resin

11.L.1. Method 1

To 0.1 mmol of resin bound product on aldehyde resin was added 2 ml of a solution of TFA/DMS/H₂O (90:5:5). The reaction was agitated for 24 hours. The cleaved product was collected and the solvent evaporated.

11.L.2. Method 2

To 0.1 mmol of resin bound product on aldehyde resin was added 2 ml of a solution of 5% TFA in DCM. The reaction was agitated for 30-120 minutes. The cleaved product was collected and the solvent evaporated.

11.L.3. Method 3

To 0.1 mmol of resin bound product on aldehyde resin was added 2 ml of a solution of 20% TFA in DCM. The reaction was agitated for 30-120 minutes. The cleaved product was collected and the solvent evaporated.

11.M. Cleavage from Trityl Resin

To 0.1 mmol of resin bound product on aldehyde resin was added 2 ml of a solution of TFA/TES/DCM (5:1:94). The reaction was agitated for 30-120 minutes. The cleaved product was collected and the solvent evaporated.

12. Phthalazines/Pyridazinones

12.A. Method 1

A resin bound hydrazine (0.1 mmol) was treated with a solution of a gamma-ketoacid (0.5M, THF-EtOH) (1.0 mmol, 10 equiv). The slurry was heated to 60° C. for 16 h, filtered, and the resin washed consecutively with DMF (3×), MeOH (3×), and DCM (3×).

13. Pyrazoles

13A. Method 1

A resin bound hydrazine (0.1 mmol) was treated with a solution of: a 1,3-diketone (1M, DMF) (1.0 mmol, 10 equiv) and DIEA (1 M, DMF) (1.0 mmol, 10 equiv). The slurry was heated to 100° C. for 16 h, filtered, and the resin washed consecutively with DMF (3×), MeOH (3×), and DCM (3×).

13B. Method 2

A resin bound hydrazine (0.1 mmol) was treated with a solution of: a 1,3-diketone (1M, 1,2-dichloroethane) (1.0 mmol, 10 equiv) and DIEA (1M, 1,2-dichloroethane) (1.0 mmol, 10 equiv). The slurry was heated to 80° C. for 16 h, filtered, and the resin washed consecutively with DMF (3×), MeOH (3×), and DCM (3×).

13.C. Method 3

0.1 mmol of the a resin bound hydrazide was treated with 10 eq. of a 1,3-diketone (1 M in DCE) and 10 eq of TEA (1 M in DCE). The mixture was heated at 80° C. for 16 hours. The resin was washed with 3×DMF, 3×MeOH, and 3×DCM.

14. Pyrazolinones

14A. Method 1

A resin bound hydrazine (0.1 mmol) was treated with solutions of: a beta-ketoester (1M, DMF) (1.0 mmol, 10 equiv) and DIEA (1 M, DMF) (1.0 mmol, 10 equiv). The slurry was heated to 100° C. for 16 h, filtered, and the resin washed consecutively with DMF (3×), MeOH (3×), and DCM (3×).

15. Uracils

15A. Method 1 1,3-Disubstituted Uracils

A resin bound urea (0.1 mmol) was treated with HOAc (2 mL), TEA (60 μL), and diketene (100 μL) The slurry was heated to 100° C. for 3 h, filtered, and the resin washed consecutively with HOAc (3×), DMF (3×), MeOH (3×), and DCM (3×).

15B. Method 2 6-Amino Uracils

A resin bound urea (0.1 mmol) was treated with a solution of cyanoacetic acid (0.5 M, acetic anhydride) (0.5 mmol, 5 equiv. The slurry was heated to 70° C. for 4 h, filtered, and the resin washed consecutively with DMF (3×), MeOH (3×), and DCM (3×).

16. Miscellaneous Cyclizations

16.A. Benzodiazepine

16.A.1 Method 1 Cyclization to Bezodiazepine

0.1 mmol of the resin bound uncyclized Ugi methylester product was treated with 2 ml of 0.002 M Terbium(III)trifluoromethane sulfonate in 1,2-dichlorobenzene. The mixture was heated at 120° C. for 18 hours. The resin was washed with 3×DCB, 3×DMF, 3×MeOH, and 3×DCM.

16.A.2. Method 2 Bezodiazapine Formation

To 0.1 mmol of resin bound product on THP resin was added 2 ml of a solution of acetic acid/THF/water (5/3/1.5, v/v). The reaction was heated at 80° C. for 16 hours.

16.B. Method 2 Diketopiperazine Formation

16.B.2. Method 1

To 0.1 mmol of resin bound product on THP resin was added 2 ml of a solution of acetic acid/THF/water (5/3/1.5, v/v). The reaction was heated at 80° C. for 16 hours.

16.B.2. Method 2

To 0.1 mmol of resin bound product on wang or Merrifield resin was added 2 ml of a solution of 2% TFA in toluene. The reaction was heated at 60° C. for 16 hours.

16.C. 4 Formation of 1,3,4-thiadiazoles

0.1 mmol of the a resin bound 1-carbonyl-thiosemicarbazide was treated with 10 eq. of HOAc (1 M in dioxane). The mixture was agitated for 16 hours. The resin was washed with 3×DMF, 3×MeOH, and 3×DCM.

16.D. Formation of 1,3,4-oxadiazoles

0.1 mmol of the a resin bound 1-carbonyl-semicarbazide was treated with 1 ml of dioxane. The mixture was heated at 80° C. for 16 hours. The resin was washed with 3×DMF, 3×MeOH, and 3×DCM.

16.E. Formation of [1,3]thiazolo[2,3-c][1,2,4]triazoles

0.1 mmol of the a resin bound, substituted N′-1,3-thiazol-2-ylhydrazide was treated with 10 eq. of HOAc (1 M in 1,2-dichloroethane). The mixture was heated to 50° C. for 16 hours. The resin was washed with 3×DMF, 3×MeOH, and 3×DCM.

16.F. Hydantoins

0.1 mmol of a dipeptide amide was treated with 1.5 eq. of phosgene (20% solution in toluene), triethyl amine (1 M in DCM), and 1 mL of DCM. The mixture was agitated for 16 hours and evaporated.

16.G. Intramolecular cyclization of a methylsulfonium iodide

0.1 mmol of resin bound methylsulfonium iodide dipetide is suspended in 1 mL 1 M DBU in DMF/DCM 1:1 (10 mmol; 10 eq) and shaken overnight. The resin is washed with DMF (3×), DCM (3×), and MeOH(3×). The entire procedure was repeated, and subjected to a second cyclization.

17. 9-Fluorenylmethyl Addition to Amine

A resin bound amine (0.1 mmol) was treated with solutions of: 9H-fluoren-9-ylmethyl 3-nitrobenzenesulfonate (1M, DMF) (1.0 mmol, 10 equiv) and DIEA (1M, DMF) (1.0 mmol, 10 equiv. The slurry was shaken at room temperature for 16 h, filtered, and the resin washed consecutively with DMF (3×), MeOH (3×), and DCM (3×).

18. Thiourea Formation

A resin bound amine (0.1 mmol) was treated with a solution of Fmoc-isothiocyante (0.5M, DCM) (0.5 mmol, 5 equiv). The slurry was shaken at room temperature for 16 h, filtered, and the resin washed consecutively with DMF (3×), MeOH (3×), and DCM (3×).

19. Alkylation or Arylation of Amines, Phenols or Thiols

19A. Alkylation of Phenols

A resin bound phenol (0.1 mmol) was treated with solutions of: an alkyl halide (1M, DMF) (0.5 mmol, 5 equiv) and DBU (1M, DMF) (1.0 mmol, 10 equiv). The slurry was heated to 50° C. for 16 h, filtered, and the resin washed consecutively with DMF (3×), MeOH (3×), and DCM (3×).

19B. Alkylation or Arylation of Amines

19.B.1 Alkyl Halides

A resin bound amine (0.1 mmol) was treated with solutions of: an alkyl halide (1 M, DMF) (0.5 mmol, 5 equiv) and DBU (1 M, DMF) (1.0 mmol, 10 equiv). The slurry was heated to 50° C. for 16 h, filtered, and the resin washed consecutively with DMF (3×), MeOH (3×), and DCM (3×).

19.B.2 Substituted Ethylene Oxides

A resin bound amine (0.1 mmol) was treated with a solution of a substituted ethylene oxides (1 M, isopropanol) (0.5 mmol, 5 equiv). The slurry was heated to 50° C. for 48 h, filtered, and the resin washed consecutively with DMF (3×), MeOH (3×), and DCM (3×).

19.B.3 Aryl Halides

A resin bound amine (0.1 mmol) was treated with solutions of: 4-chloroquinazolines, 1-chlorophthalazines, or 5-bromo-1-aryl-1H-tetrazoles (0.5M, DMF-THF) (0.5 mmol, 5 equiv) and TEA (1 M, DMF) (1.0 mmol, 10 equiv). The slurry was heated to 55° C. for 16 h, filtered, and the resin washed consecutively with DMF (3×), MeOH (3×), and DCM (3×).

19.B.4 Alkylation of Amine with a Dichloro Heterocycle

0.1 mmol of a resin bound amine was heated with a dichloroheterocycle (0.2 mmol; 2 eq) and 3 eq of DIEA in 2 mL n-BuOH at 80° C. for 24 hours. The resin was then washed with DMF (3×), DCM (3×), and MeOH(3×).

19.B.5 Amine Substitutution on a Chloroheterocycle

0.1 mmol of a resin bound chloroheterocycle was heated with an amine (0.5 mmol; 5 eq) in 2 mL n-BuOH at 90° C. for 12 hours. The resin was then washed with DMF (3×), DCM (3×), and MeOH (3×).

19.B.6 3-[(Dimethylamino)methylene]-1,3-dihydro-2H-indol-2-ones

A resin bound amine (0.1 mmol) was treated with a solution of: a 3-[(dimethylamino)methylene]-1,3-dihydro-2H-indol-2-one (0.5M, DMF-THF) (0.5 mmol, 5 equiv). The slurry was heated to 55° C. for 16 h, filtered, and the resin washed consecutively with DMF (3×), MeOH (3×), and DCM (3×).

19.B.7. Trazine

0.1 mmol of a resin-bound amine was treated with 3 eq. of a 2-substituted-4,6-dichloro-1,3,5-triazine (0.5 M in DCM/DMF, 50:50) and 6 eq. of DIEA (0.5 M in DCM/DMF, 50:50). The mixture was agitated for 4 hours. The resin was washed with 3×DMF, 3×MeOH, and 3×DCM. The resin bound 2-substituted-4-chloro-1,3,5-triazine was treated with 3.5 eq. of an amine (1 M in DMF) and 10 eq. of DIEA (1 M in DMF). The mixture was agitated for 16 hours followed by heating for 4 hours at 50° C. The resin was washed with 3×DMF, 3×MeOH, and 3×DCM

19.B.8 Alkyl Triflates

A resin bound amine (0.1 mmol) was treated with a solution of: an alkyl triflate (1.0M, DCM) (0.1 mmol, 1 equiv), pyridine (1.0M, DCM) (0.1 mmol, 1 equiv) and DIEA (1.0M, DCM) (0.5 mmol, 5 equiv). The slurry was shaken for 16 h, filtered, and the resin washed consecutively with DMF (3×), MeOH (3×), and DCM (3×).

19.B.9 Formation of a Methylsulfonium Iodide

0.1 mmol of a resin bound thioether is suspended in 2 mL neat methyl iodide and shaken overnight. The resin is then washed with DMF (3×) and DCM (3×).

19.B.10 Nucleophlic Aromatic Substitution

0.1 mmol of resin bound fluoro-nitro benzoic acid was treated with 4eq of an amine and 8 eq of DIEA in 2 mL DMF at room temperature overnight. The resin was then washed with DMF (3×), DCM (3×), and MeOH (3×).

20. Preparation of Amines and Amino Acids with Organoboron Derivatives

0.1 mmol of resin-bound amine was treated with 10 eq. of carbonyl component (i.e. ethyl glyoxylate, pyruvic acid, salisaldehyde, methylpyruvate, glyceraldehyde, glyoxylic acid, 1 M in DCM) and 10 eq. of a boronic acid (1 M in DCM/Tol. 50:50). The reaction was agitated for 16 h. The resin was washed with 3×DMF, 3×MeOH, and 3×DCM.

21. Oxidation of Resin-Bound Alcohol

0.1 mmol of resin-bound alcohol was purged with nitrogen for 1 hour and mixed with anhydrous DMSO (2×volume of DMSO used for Pyr-SO₃). 8.6 eq. of Pyr-SO₃ was purged with nitrogen for 30 min. and anhydrous DMSO (10 ml of DMSO for 1.0 g of Pyr-SO₃) and triethylamine (1:1 mixture with DMSO) were added. This mixture was stirred for 15 min. after which it was added to the resin-DMSO mixture. The mixture was shaken for 4 hours after which the resin was washed with 3×DMSO and 6×THF and dried in vacuo.

22. Preparation of Resin-Bound Thiouronium Salt

0.1 mmol of chloromethylated polystyrene was treated with 5 eq. of a substituted thiourea in (2 M in dioxane/EtOH, 4:1). The mixture was heated at 90° C. for 16 hours. The resin was washed with 3×EtOH (at 70° C.), 3×dioxane and 3×pentane and dried in vacuo.

23. Formylation

A resin bound amine (0.1 mmol) was treated with a solution of formic acetic anhydride (1 M, DCM) (1.0 mmol, 10 equiv). The slurry was shaken for 16 h, filtered, and the resin washed consecutively with DMF (3×), MeOH (3×), and DCM (3×).

24. Isocyanide Formation

A resin bound formamide (0.1 mmol) was treated with solutions of: TEA (1M, DCM) (0.5 mmol, 5 equiv) and POCl₃ (1M, DCM) (0.15 mmol, 1.5 equiv). The slurry was shaken for 16 h, filtered, and the resin washed consecutively with DMF (3×), MeOH (3×), and DCM (3×).

25. Hydrazide Formation

A resin bound ester (0.1 mmol) was treated with 2 mL of a 15% solution of hydrazine hydrate in dioxane. The slurry was shaken for 16 h, filtered, and the resin washed consecutively with DMF (3×), MeOH (3×), and DCM (3×).

26. Indazole Formation

A resin bound hydrazine (0.1 mmol) was treated with solutions of: a substituted 2-fluoro-bezaldehyde or 2-fluoro-arylketone (1 M, DMF) (1.0 mmol, 10 equiv). The slurry was heated to 100° C. for 16 h, filtered, and the resin washed consecutively with DMF (3×), MeOH (3×), and DCM (3×).

27. Beta-Ketoamide Formation

A resin bound amine (0.1 mmol) was treated with a solution of diketene(1 M, DCM) (0.5 mmol, 5 equiv)and 2 mL of DCM. The slurry was shaken for 4 h, filtered, and the resin washed consecutively with DMF (3×), and DCM (3×).

28. Beta-Ketoester Formation

A resin bound alcohol (0.1 mmol) was treated with solutions of: diketene(1 M, DCM) (0.3 mmol, 3 equiv), DMAP (1 M, DCM) (0.01 mmol, 0.1 equiv), and 2 mL of DCM. The slurry was shaken for 4 h, filtered, and the resin washed consecutively with DMF (3×), and DCM (3×).

29. 1-carbonyl-semicarbazides

A resin bound hydrazide (0.1 mmol) was treated with a solution of an isocyanate (1 M, DCM) (0.2 mmol, 2 equiv), and 2 mL of DCM. The slurry was shaken for 16 h, filtered, and the resin washed consecutively with DMF (3×), MeOH (3×), and DCM (3×).

30.1-carbonyl-thiosemicarbazides

A resin bound hydrazide (0.1 mmol) was treated with a solution of an isothiocyanate (1 M, DCM) (0.2 mmol, 2 equiv), and 2 mL of DCM. The slurry was shaken for 16 h, filtered, and the resin washed consecutively with DMF (3×), MeOH (3×), and DCM (3×).

31. 1,3-Thiazolidin-4-ones

A resin bound hydrazide (0.1 mmol) was treated with a solution of an aldehyde (1 M, reagent alcohol) (1.0 mmol, 10 equiv). The slurry was heated to 55° C. for 16 h and filtered. The resulting resin with solutions of: a mercaptoacetic acid (1 M, dioxane) (1.0 mmol, 10 equiv) and TEA (1M, dioxane) (1.0 mmol, 10 equiv). The slurry was heated to 55° C. for 16 h, filtered, and the resin washed consecutively with DMF (3×), MeOH (3×), and DCM (3×).

32. Reduction of Aromatic Nitro

0.1 mmol of resin containing a nitro aromatic was treated with 10 eq. of SnCl₂ in 2 ml of DMF overnight. The resin was then washed with DMF (3×), DCM (3×), and MeOH (3×).

33. Reduction of Esters with Resin-Bound Borohydride Resin

0.1 mmol of of an ester was dissolved in DCM/MeOH (1 M, 50:50) and treated with 5 eq. of (polystyrylmethyl)trimethylammonium borohydride for 16 hours at room temperature. The resin was drained and the solvent was evaporated to give the primary alcohol.

Example Probe Libraries;

Probe Library 1

An Fmoc protected amino acid was attached to Rink resin according to general procedure 1.C.2 and the amino group deprotected according to general procedure 2.A. The amine was acylated with bromoacetic acid or 2-substituted 2-bromoacetic acid according to general procedure 3.C.2. The resin was treated with hydrazine hydrate according to general procedure 8.C. followed by reaction with a gamma-ketoacid according to general procedure 12.A. Cleavage from the resin was done according to general procedure 11.A.

Probe Library 2

An Fmoc protected amino acid was attached to reductively aminated Aldehyde resin according to general procedure 1.D.2 and the amino group deprotected according to general procedure 2.A. The amine was acylated with bromoacetic acid or 2-substituted 2-bromoacetic acid according to general procedure 3.C.2. The resin was treated with hydrazine hydrate according to general procedure 8.C. followed by reaction with a gamma-ketoacid according to general procedure 12.A. Cleavage from the resin was done according to general procedure 11.L.2.

Probe Library 3

Rink resin was deprotected 2.A. and treated with an aldehyde or ketone, carboxylic acid and an isocyanide according to general procedure 9.C. Cleavage from the resin was done according to general procedure 11.A.

Probe Library 4.

A Boc or Fmoc protected alpha-amino acid was attached to hydroxymethyl PS according to general procedure 1.A.1. and the amino group deprotected according to general procedure 2.A for Fmoc and 2.B. for Boc. The amine was reacted with triphosgene followed by an amine according to general procedure 6.B. Cyclization/cleavage from the resin was done according to general procedure 11.D.

Probe Library 5.

A Boc or Fmoc protected alpha-amino acid was attached to hydroxymethyl PS according to general procedure 1.A.1. and the amino group deprotected according to general procedure 2.A for Fmoc and 2.B. for Boc. The amine was reductively aminated with an aldehyde or ketone according to general procedure 5.A. The amine was reacted with triphosgene followed by an amine according to general procedure 6.B. Cyclization/cleavage from the resin was done according to general procedure 11.D.

Probe Library 6

An Fmoc protected alpha-amino acid was attached to Wang Resin according to general procedure 1.B.1. and the amino group deprotected according to general procedure 2.A. The amine was reacted with triphosgene followed by an amine according to general procedure 6.B. Cyclization/cleavage from the resin was done according to general procedure 11.D.

Probe Library 7

A Boc or Fmoc protected beta-amino acid was attached to hydroxymethyl PS according to general procedure 1.A.1. and the amino group deprotected according to general procedure 2.A for Fmoc and 2.B. for Boc. The amine was reductively aminated with an aldehyde or ketone according to general procedure 5.A. The resulting amine was acylated with bromoacetic acid or 2-substituted 2-bromoacetic acid according to general procedure 3.C.2. The resin was treated with a primary amine according to general procedure 8.A.1. Cyclization/cleavage from the resin was done according to general procedure 11.D. or 11.E.

Probe Library 8

Bromo-pyruvic acid was attached to reductively aminated aldehyde resin according to general procedure 1.D.4. The resulting resin was treated with thiosemicarbazide according to general procedure 8.D.1. followed by reaction with a 1,3-diketone according to general procedure 13.B. The final product was cleaved from the resin according to general procedure 11.L.2.

Probe Library 9

An Fmoc protected amino acid was attached to Rink resin according to general procedure 1.C.2 and the amino group deprotected according to general procedure 2.A. The amine was acylated with bromoacetic acid or 2-substituted 2-bromoacetic acid according to general procedure 3.C.2. The resin was treated with hydrazine hydrate according to general procedure 8.C. followed by reaction with a 1,3-diketone according to general procedure 13.A. Cleavage from the resin was done according to general procedure 11.A.

Probe Library 10

An Fmoc protected amino acid was attached to reductively aminated aldehyde resin according to general procedure 1.D.2 and the amino group deprotected according to general procedure 2.A. The amine was acylated with bromoacetic acid or 2-substituted 2-bromoacetic acid according to general procedure 3.C.2. The resin was treated with hydrazine hydrate according to general procedure 8.C. followed by reaction with a 1,3-diketone according to general procedure 13.A. Cleavage from the resin was done according to general procedure 11.L.2.

Probe Library 11

A 2-amino alcohol was reductively aminated onto aldehyde resin according to general procedure 1.D.5. The secondary amine was protected with Fmoc using Fmoc chloroformate according to general procedure 7.A.2. The alcohol was oxidized according to general procedure 21 and the resulting resin used in an Ugi reaction according to general procedure 9.D. The Fmoc group was removed according to general procedure 2.A. and the resulting resin bound molecule cyclized to the benzodiazepine according to general procedure 16.A.1. The final benzodiazepine was liberated from the resin according to general procedure 11.L.1.

Probe Library 12

A carboxy-phenol was attached to reductively aminated aldehyde resin according to general procedure 1.D.6. The resulting resin bound phenol was then subjected to the Mitsunobu reaction according to general procedure 10.A. Cleavage from the resin was done according to general procedure 11.L.2.

Probe Library 13

An Fmoc/Boc protected alpha-amino acid (Fmoc on the alpha-amine and Boc on the side-chain amine) was coupled onto hydroxymethyl polystyrene resin using general procedure 1.A.1. The side-chain amine was deprotected using general procedure 2.B. The side chain amine was then reacted with an anhydride, sulfonyl chloride, carbamoyl chloride, or isocyanate using general procedures 3.C.1, 4.A, 6.C, 6A, respectively or left unreacted. The alpha-amine was deprotected using general procedure 2.A. The alpha-amine was then reacted with an anhydride, sulfonyl chloride, carbamoyl chloride, or isocyanate using general procedures 3.C.1, 4.A, 6.C, 6A, respectively or left unreacted. The product was cleaved from the resin using general procedure 11.B or 11.H.

Probe Library 14

An Fmoc/Boc protected alpha-amino acid (Fmoc on the alpha-amine and Boc on the side-chain amine) was coupled onto hydroxymethyl polystyrene resin using general procedure 1.A.1. The alpha-amine was deprotected using general procedure 2.A. The alpha-amine was then reacted with an anhydride, sulfonyl chloride, carbamoyl chloride, or isocyanate using general procedures 3.C.1, 4.A, 6.C, 6A, respectively or left unreacted. The side-chain amine was deprotected using general procedure 2.B. The side chain amine was then reacted with an anhydride, sulfonyl chloride, carbamoyl chloride, or isocyanate using general procedures 3.C.1, 4.A, 6.C, 6A, respectively or left unreacted. The product was cleaved from the resin using general procedure 11.B or 11.H.

Probe Library 15

A Boc or Fmoc protected amino acid was coupled onto hydroxymethyl polystyrene resin using general procedure 1.A.1. The resin bound protected amino acid was then deprotected using general procedure 2.A for Fmoc or 2.B for Boc protecting groups. The resin bound amine was then reacted using general procedure 9.A. using a substituted or un-substituted Fmoc-protected 2-aminobenzoic acid as the carboxylic acid component. The resin bound Ugi product was deprotected using general procedure 2.A. The resin bound amine was then cyclized and cleaved using general procedure 11.G.1

Probe Library 16

A Boc or Fmoc protected amino acid was coupled onto hydroxymethyl polystyrene resin using general procedure 1.A.1. The resin bound protected amino acid was then deprotected using general procedure 2.A for Fmoc or 2.B for Boc protecting groups. The resin bound amine was then reacted using general procedure 9.A. using a substituted or un-substituted Fmoc-protected 2-aminobenzoic acid as the carboxylic acid component. The resin bound Ugi product was deprotected using general procedure 2.A. The resin bound amine was then cyclized and cleaved using general procedure 11.G.2.

Probe Library 17

An Fmoc protected amino ester alcohol was coupled onto THP resin using general procedure 1.G. The resin bound protected amino ester was then deprotected using general procedure 2.A. The resin bound amine was then reacted using general procedure 9.A Method 1 using a substituted or un-substituted Fmoc-protected 2-aminobenzoic acid as the carboxylic acid component. The resin bound Ugi product was deprotected using general procedure 2.A. The resin bound amine was then cyclized and cleaved using general procedure 11.F. and 16.A.2.

Probe Library 18

A mono Fmoc protected diamino ester was coupled onto Wang carbonate using general procedure 1.E.2. The resin bound protected amino acid was then deprotected using general procedure 2.A. The resin bound amine was then reacted using general procedure 9.B. using an Fmoc-protected amino acid as the carboxylic acid component. The resin bound Ugi product was deprotected using general procedure 2.A. The resin bound amine was then cyclized and cleaved using general procedure 11.1.2. and 16.B.1.

Probe Library 19

An Fmoc protected amino ester alcohol was coupled onto THP resin using general procedure 1.G. The resin bound protected amino ester was then deprotected using general procedure 2.A. The resin bound amine was then reacted using general procedure 9.B. using an Fmoc-protected amino acid as the carboxylic acid component. The resin bound Ugi product was deprotected using general procedure 2.A. The resin bound amine was then cyclized and cleaved using general procedure 11.F. and 16.A.2.

Probe Library 20

A Boc protected amino acid on hydroxymethyl polystyrene resin was deprotected using general procedure 2.B. An Fmoc/Boc protected alpha-amino acid (Fmoc on the alpha-amine and Boc on the side chain amine) was coupled the resin bound amine using general procedure 3A. The side chain amine was deprotected using general procedure 2.B. The side chain amine was then acylated using general procedure 3.A. The alpha-amine was deprotected using general procedure 2.A. The alpha-amine was acylated using general procedure 3.A. The product was cleaved from the resin using general procedure 11.B.

Probe Library 21

A Boc protected amino acid on hydroxymethyl polystyrene resin was deprotected using general procedure 2.B. An Fmoc/Boc protected alpha-amino acid (Fmoc on the alpha-amine and Boc on the side chain amine) was coupled onto the resin bound amine using general procedure 3A. The side chain amine was deprotected using general procedure 2.B. The side chain amine was then acylated using general procedure 3.A. The alpha-amine was deprotected using general procedure 2.A. The alpha-amine was acylated using general procedure 3.A. The product was cleaved from the resin using general procedure 11.B.

Probe Library 22

A primary amine was loaded onto aldehyde resin using general procedure 1.D.5. The amine was then acylated using general procedure 3.C.2. The resin bound alpha-bromo amide was then reacted with a amine using general procedure 8.A.1. The product was then cleaved from the resin using general procedure 11.L.2.

Probe Library 23

A primary amine was loaded onto aldehyde resin using general procedure 1.D.5. The amine was then acylated using general procedure 3.C.2. The resin bound substituted alpha-bromo amide was then reacted with an amine using general procedure 8.A.2. The product was then cleaved from the resin using general procedure 11.L.2.

Probe Library 24

A primary amine was loaded onto aldehyde resin using general procedure 1.D.5. The amine was then acylated using general procedure 3.C.2. The resin bound alpha-bromo amide was then reacted with a thiol using general procedure 8.B.1. The product was then cleaved from the resin using general procedure 11.L.2.

Probe Library 25

A primary amine was loaded onto aldehyde resin using general procedure 1.D.5. The amine was then acylated using general procedure 3.C.2. The resin bound substituted alpha-bromo amide was then reacted with a thiol using general procedure 8.B.2. The product was then cleaved from the resin using general procedure 11.L.2.

Probe Library 26

An Fmoc or Boc protected amino acid was coupled onto hydroxymethyl polystyrene resin using either general procedure 1.A.1. or 1.A.2. The amine was deprotected using general procedure 2.A. for Fmoc removal or 2.B. for Boc removal. The resin-bound amine was then acylated using general procedure 3.C.2. The resin bound alpha-bromo amide was then reacted with an amine using general procedure 8.A.1. The product was then cleaved from the resin using general procedure 11.B, 11.H., or 11.J.

Probe Library 27

An Fmoc or Boc protected amino acid was coupled onto hydroxymethyl polystyrene resin using either general procedure 1.A.1. or 1.A.2. The amine was deprotected using general procedure 2.A. for Fmoc removal or 2.B. for Boc removal. The resin-bound amine was then acylated using general procedure 3.C.2. The resin bound substituted alpha-bromo amide was then reacted with an amine using general procedure 8.A.2. The product was then cleaved from the resin using general procedure 11.B, 11.H., or 11.J.

Probe Library 28

An Fmoc or Boc protected amino acid was coupled onto hydroxymethyl polystyrene resin using either general procedure 1.A.1. or 1.A.2. The amine was deprotected using general procedure 2.A. for Fmoc removal or 2.B. for Boc removal. The resin-bound amine was then acylated using general procedure 3.C.2. The resin bound alpha-bromo amide was then reacted with a thiol using general procedure 8.B.1. The product was then cleaved from the resin using general procedure 11.B, 11.H., or 11.J.

Probe Library 29

An Fmoc or Boc protected alpha-amino acid was coupled onto hydroxymethyl polystyrene resin using either general procedure 1.A.1. or 1.A.2. The amine was deprotected using general procedure 2.A. for Fmoc removal or 2.B. for Boc removal. The resin-bound amine was then acylated using general procedure 3.C.2. The resin bound substituted alpha-bromo amide was then reacted with a thiol using general procedure 8.B.2. The product was then cleaved from the resin using general procedure 11.B, 11.H., or 11.J.

Probe Library 30

An Fmoc alpha-amino acid was coupled onto Rink resin using either general procedure 1.C.1. or 1.C.2. The amine was deprotected using general procedure 2.A. The resin-bound amine was then acylated using general procedure 3.C.2. The resin bound alpha-bromo amide was then reacted with an amine using general procedure 8.A.1. The product was then cleaved from the resin using general procedure 11.A.

Probe Library 31

An Fmoc alpha-amino acid was coupled onto Rink resin using either general procedure 1.C.1. or 1.C.2. The amine was deprotected using general procedure 2.A. The resin-bound amine was then acylated using general procedure 3.C.2. The resin bound substituted alpha-bromo amide was then reacted with an amine using general procedure 8.A.2. The product was then cleaved from the resin using general procedure 11.A.

Probe Library 32

An Fmoc alpha-amino acid was coupled onto Rink resin using either general procedure 1.C.1. or 1.C.2. The amine was deprotected using general procedure 2.A. The resin-bound amine was then acylated using general procedure 3.C.2. The resin bound alpha-bromo amide was then reacted with a thiol using general procedure 8.B.1. The product was then cleaved from the resin using general procedure 11.A.

Probe Library 33

An Fmoc alpha-amino acid was coupled onto Rink resin using either general procedure 1.C.1. or 1.C.2. The amine was deprotected using general procedure 2.A. The resin-bound amine was then acylated using general procedure 3.C.2. The resin bound substituted alpha-bromo amide was then reacted with a thiol using general procedure 8.B.2. The product was then cleaved from the resin using general procedure 11.A.

Probe Library 34

An Fmoc alpha-amino acid was coupled onto Wang resin using either general procedure 1.B.1. or 1.B.2. The amine was deprotected using general procedure 2.A. The resin-bound amine was then acylated using general procedure 3.C.2. The resin bound alpha-bromo amide was then reacted with an amine using general procedure 8.A.1. The product was then cleaved from the resin using general procedure 11.A.

Probe Library 35

An Fmoc alpha-amino acid was coupled onto Wang resin using either general procedure 1.B.1. or 1.B.2. The amine was deprotected using general procedure 2.A. The resin-bound amine was then acylated using general procedure 3.C.2. The resin bound substituted alpha-bromo amide was then reacted with an amine using general procedure 8.A.2. The product was then cleaved from the resin using general procedure 11.A.

Probe Library 36

An Fmoc alpha-amino acid was coupled onto Wang resin using either general procedure 1.B.1. or 1.B.2. The amine was deprotected using general procedure 2.A. The resin-bound amine was then acylated using general procedure 3.C.2. The resin bound alpha-bromo amide was then reacted with a thiol using general procedure 8.B.1. The product was then cleaved from the resin using general procedure 11.A.

Probe Library 37

An Fmoc alpha-amino acid was coupled onto Wang resin using either general procedure 1.B.1. or 1.B.2. The resin bound amine was deprotected using general procedure 2.A. The resin-bound amine was then acylated using general procedure 3.C.2. The resin bound substituted alpha-bromo amide was then reacted with a thiol using general procedure 8.B.2. The product was then cleaved from the resin using general procedure 11.A.

Probe Library 38

An Fmoc protected amino acid was attached to an amine on aldehyde resin using general procedure 1.D.1. The resin bound amino acid was deprotected using general procedure 2.A. The resin-bound amine was then acylated using general procedure 3.C.2. The resin bound alpha-bromo amide was then reacted with an amine using general procedure 8.A.1. The product was then cleaved from the resin using general procedure 11.L.2.

Probe Library 39

An Fmoc protected amino acid was attached to an amine on aldehyde resin using general procedure 1.D.1. The resin bound amino acid was deprotected using general procedure 2.A. The resin bound amine was then acylated using general procedure 3.C.2. The resin bound substituted alpha-bromo amide was then reacted with an amine using general procedure 8.A.2. The product was then cleaved from the resin using general procedure 11.L.2.

Probe Library 40

An Fmoc protected amino acid was attached to an amine on aldehyde resin using general procedure 1.D.1. The resin bound amino acid was deprotected using general procedure 2.A. The resin bound amine was then acylated using general procedure 3.C.2. The resin bound alpha-bromo amide was then reacted with a thiol using general procedure 8.B.1. The product was then cleaved from the resin using general procedure 11.L.2.

Probe Library 41

An Fmoc protected amino acid was attached to an amine on aldehyde resin using general procedure 1.D.1. The resin bound amino acid was deprotected using general procedure 2.A. The resin bound amine was then acylated using general procedure 3.C.2. The resin bound substituted alpha-bromo amide was then reacted with a thiol using general procedure 8.B.2. The product was then cleaved from the resin using general procedure 11.L.2.

Probe Library 42

An Fmoc protected amino acid was attached to an amine on aldehyde resin using general procedure 1.D.2. The resin bound amino acid was deprotected using general procedure 2.A. The resin-bound amine was then acylated using general procedure 3.C.2. The resin bound alpha-bromo amide was then reacted with an amine using general procedure 8.A.1. The product was then cleaved from the resin using general procedure 11.L.2.

Probe Library 43

An Fmoc protected amino acid was attached to an amine on aldehyde resin using general procedure 1.D.2. The resin bound amino acid was deprotected using general procedure 2.A. The resin bound amine was then acylated using general procedure 3.C.2. The resin bound substituted alpha-bromo amide was then reacted with an amine using general procedure 8.A.2. The product was then cleaved from the resin using general procedure 11.L.2.

Probe Library 44

An Fmoc protected amino acid was attached to an amine on aldehyde resin using general procedure 1.D.2. The resin bound amino acid was deprotected using general procedure 2.A. The resin bound amine was then acylated using general procedure 3.C.2. The resin bound alpha-bromo amide was then reacted with a thiol using general procedure 8.B.1. The product was then cleaved from the resin using general procedure 11.L.2.

Probe Library 45

An Fmoc protected amino acid was attached to an amine on aldehyde resin using general procedure 1.D.2. The resin bound amino acid was deprotected using general procedure 2.A. The resin bound amine was then acylated using general procedure 3.C.2. The resin bound substituted alpha-bromo amide was then reacted with a thiol using general procedure 8.B.2. The product was then cleaved from the resin using general procedure 11.L.2.

Probe Library 46

An Fmoc protected amino acid was attached to an amine on aldehyde resin using general procedure 1.D.2. The resin bound amino acid was deprotected using general procedure 2.A. The resin bound amine was then reacted with a carbonyl component and either a vinyl or aryl boronic acid using general procedure 20. The free acid is acylated using general procedure 3.F. or left un-reacted. The product was then cleaved and collected using general procedure 11.L.2.

Probe Library 47

An Fmoc protected amino acid was attached to Wang resin using either general procedure 1.B.1 or 1.B.2. The resin bound amino acid was deprotected using general procedure 2.A. The resin bound amine was then reacted with carbonyl component and either a vinyl or aryl boronic acid using general procedure 20. The free acid is acylated using general procedure 3.F. or left un-reacted. The product was then cleaved and collected using general procedure 11.A.

Probe Library 48

An Fmoc or Boc protected amino acid was attached to Merrifield resin using either general procedure 1.A.1 or 1.A.2. The resin Fmoc or Boc protected bound amino acid was deprotected using either general procedure 2.A or 2.B. The resin bound amine was then reacted with a carbonyl component and either a vinyl or aryl boronic acid using general procedure 20. The free acid is acylated using general procedure 3.F. or left un-reacted. The product was then cleaved and collected using general procedure 11.B.

Probe Library 49

An Fmoc/Boc protected alpha-amino acid (Fmoc on the alpha-amine and Boc on the side chain amine) was coupled onto hydroxymethyl polystyrene resin using general procedure 1.A.1. The side chain Boc protected amine was deprotected using general procedure 2.B. The resin bound side chain amine was reacted with an anhydride, a sulfonyl chloride, a carbamoyl chloride, or an isocyanate using general procedures 3.C.1, 4.A., 6.C. or 6.A., respectively. The Fmoc protected resin bound alpha-amine was deprotected using general procedure 2.A. An Fmoc/Boc protected alpha-amino acid (Fmoc on the alpha-amine and Boc on the side chain amine) was coupled onto the resin bound alpha-amine using general procedure 3.A. The side chain Boc protected amine was deprotected using general procedure 2.B. The resin bound side chain amine was reacted with an anhydride, a sulfonyl chloride, a carbamoyl chloride, or an isocyanate using general procedures 3.C.1, 4.A., 6.C. or 6.A., respectively or left un-reacted. The Fmoc protected resin bound alpha-amine was deprotected using general procedure 2.A. The resin bound alpha-amine was reacted with an anhydride, a sulfonyl chloride, a carbamoyl chloride, or an isocyanate using general procedures 3.C.1, 4.A., 6.C. or 6.A., respectively or left un-reacted. The product was cleaved from the resin using general procedure 11.B., 11.C., 11.H., or 11.J.

Probe Library 50

An Fmoc/Boc protected alpha-amino acid (Fmoc on the alpha-amine and Boc on the side chain amine) was coupled onto hydroxymethyl polystyrene resin using general procedure 1.A.1. The side chain Boc protected amine was deprotected using general procedure 2.B. The resin bound side chain amine was reacted with an anhydride, a sulfonyl chloride, a carbamoyl chloride, or an isocyanate using general procedures 3.C.1, 4.A., 6.C. or 6.A., respectively. The Fmoc protected resin bound alpha-amine was deprotected using general procedure 2.A. An Fmoc/Boc protected alpha-amino acid (Fmoc on the alpha-amine and Boc on the side chain amine) was coupled onto the resin bound alpha-amine using general procedure 3.A. The side chain Boc protected amine was deprotected using general procedure 2.B. The resin bound side chain amine was reacted with an anhydride, a sulfonyl chloride, a carbamoyl chloride, or an isocyanate using general procedures 3.C.1, 4.A., 6.C. or 6.A., respectively or left un-reacted. The Fmoc protected resin bound alpha-amine was deprotected using general procedure 2.A. The product was cleaved from the resin using general procedure 11.B., 11.C., 11. H., or 11.J.

Probe Library 51

An Fmoc/Boc protected alpha-amino acid (Fmoc on the alpha-amine and Boc on the side chain amine) was coupled onto hydroxymethyl polystyrene resin using general procedure 1.A.1. The side chain Boc protected amine was deprotected using general procedure 2.B. The resin bound side chain amine was reacted with an anhydride, a sulfonyl chloride, a carbamoyl chloride, or an isocyanate using general procedures 3.C.1, 4.A., 6.C. or 6.A., respectively. The Fmoc protected resin bound alpha-amine was deprotected using general procedure 2.A. An Fmoc/Boc protected alpha-amino acid (Fmoc on the alpha-amine and Boc on the side chain amine) was coupled onto the resin bound alpha-amine using general procedure 3.A. The Fmoc protected resin bound alpha-amine was deprotected using general procedure 2.A. The resin bound alpha-amine was reacted with an anhydride, a sulfonyl chloride, a carbamoyl chloride, or an isocyanate using general procedures 3.C.1, 4.A., 6.C. or 6.A., respectively or left un-reacted. The side chain Boc protected amine was deprotected using general procedure 2.B. The product was cleaved from the resin using general procedure 11.B. or 11.H.

Probe Library 52

An Fmoc or Boc protected alpha-amino acid was coupled onto hydroxymethyl polystyrene resin using general procedure 1.A.1. The resin bound protected alpha-amine was deprotected using general procedure 2.A. or 2.B. An Fmoc/Boc protected alpha-amino acid (Fmoc on the alpha-amine and Boc on the side chain amine) was coupled onto the resin bound alpha-amine using general procedure 3.A. The Fmoc protected resin bound alpha-amine was deprotected using general procedure 2.A. The resin bound alpha-amine was reacted with a carboxylic acid, an aldehyde or ketone, an anhydride, a sulfonyl chloride, a sulfamoyl chloride, a carbamoyl chloride, or an isocyanate using general procedures 3.A., 5.A., 3.C.1, 4.A., 4.B.1, 6.C. or 6.A., respectively or left un-reacted. The side chain Boc protected amine was deprotected using general procedure 2.B. The resin bound side chain amine was reacted with a carboxylic acid, an aldehyde or ketone, an anhydride, a sulfonyl chloride, a sulfamoyl chloride, a carbamoyl chloride, or an isocyanate using general procedures 3.A., 5.A., 3.C.1, 4.A., 4.B.1, 6.C. or 6.A., respectively or left un-reacted. The product was cleaved from the resin using general procedure 11.B., 11.C., 11.H., or 11.J.

Probe Library 53

An Fmoc or Boc protected alpha-amino acid was coupled onto hydroxymethyl polystyrene resin using general procedure 1.A.1. The resin bound protected alpha-amine was deprotected using general procedure 2.A. or 2.B. An Fmoc/Boc protected alpha-amino acid (Fmoc on the alpha-amine and Boc on the side chain amine) was coupled onto the resin bound alpha-amine using general procedure 3.A. The side chain Boc protected amine was deprotected using general procedure 2.B. The resin bound side chain amine was reacted with a carboxylic acid, an aldehyde or ketone, an anhydride, a sulfonyl chloride, a sulfamoyl chloride, a carbamoyl chloride, or an isocyanate using general procedures 3.A., 5.A., 3.C.1, 4.A., 4.B.1, 6.C. or 6.A., respectively or left un-reacted. The Fmoc protected resin bound alpha-amine was deprotected using general procedure 2.A. The resin bound alpha-amine was reacted with a carboxylic acid, an aldehyde or ketone, an anhydride, a sulfonyl chloride, a sulfamoyl chloride, a carbamoyl chloride, or an isocyanate using general procedures 3.A., 5.A., 3.C.1, 4.A., 4.B.1, 6.C. or 6.A., respectively or left un-reacted. The product was cleaved from the resin using general procedure 11.B., 11.C., 11.H., or 11.J.

Probe Library 54

An Fmoc/Boc protected alpha-amino acid (Fmoc on the alpha-amine and Boc on the side chain amine) was coupled onto hydroxymethyl polystyrene resin using general procedure 1.A.1. The side chain Boc protected amine was deprotected using general procedure 2.B. The resin bound side chain amine was reacted with a carboxylic acid, an aldehyde or ketone, an anhydride, a sulfonyl chloride, a sulfamoyl chloride, a carbamoyl chloride, or an isocyanate using general procedures 3.A., 5.A., 3.C.1, 4.A., 4.B.1, 6.C. or 6.A. The resin bound protected alpha-amine was deprotected using general procedure 2.A. An Fmoc protected alpha-amino acid was coupled onto the resin bound alpha-amine using general procedure 3.A. The Fmoc protected resin bound alpha-amine was deprotected using general procedure 2.A. The resin bound alpha-amine was reacted with a carboxylic acid, an aldehyde or ketone, an anhydride, a sulfonyl chloride, a sulfamoyl chloride, a carbamoyl chloride, or an isocyanate using general procedures 3.A., 5.A., 3.C.1, 4.A., 4.B.1, 6.C. or 6.A., respectively or left un-reacted. The product was cleaved from the resin using general procedure 11.B., 11.C., 11.H., or 11.J.

Probe Library 55

An Fmoc/Boc protected alpha-amino acid (Fmoc on the alpha-amine and Boc on the side chain amine) was coupled onto hydroxymethyl polystyrene resin using general procedure 1.A.1. The resin bound protected alpha-amine was deprotected using general procedure 2.A. An Fmoc protected alpha-amino acid was coupled onto the resin bound alpha-amine using general procedure 3.A. The Fmoc protected resin bound □-amine was deprotected using general procedure 2.A. The resin bound alpha-amine was reacted with a carboxylic acid, an aldehyde or ketone, an anhydride, a sulfonyl chloride, a sulfamoyl chloride, a carbamoyl chloride, or an isocyanate using general procedures 3.A., 5.A., 3.C.1, 4.A., 4.B.1, 6.C. or 6.A., respectively or left un-reacted. The side chain Boc protected amine was deprotected using general procedure 2.B. The product was cleaved from the resin using general procedure 11.B., 11.C., 11.H., or 11.J.

Probe Library 56

An Fmoc/Boc protected alpha-amino acid (Fmoc on the alpha-amine and Boc on the side chain amine) was coupled onto hydroxymethyl polystyrene resin using general procedure 1.A.1. The side chain Boc protected amine was deprotected using general procedure 2.B. The resin bound side chain amine was reacted with a carboxylic acid, an aldehyde or ketone, an anhydride, a sulfonyl chloride, a sulfamoyl chloride, a carbamoyl chloride, or an isocyanate using general procedures 3.A., 5.A., 3.C.1, 4.A., 4.B.1, 6.C. or 6.A. The resin bound protected alpha-amine was deprotected using general procedure 2.A. A Boc protected alpha-amino acid was coupled onto the resin bound alpha-amine using general procedure 3.A. The Boc protected resin bound amine was deprotected using general procedure 2.B. The resin bound amine was reacted with a carboxylic acid, an aldehyde or ketone, an anhydride, a sulfonyl chloride, a sulfamoyl chloride, a carbamoyl chloride, or an isocyanate using general procedures 3.A., 5.A., 3.C.1, 4.A., 4.B.1, 6.C. or 6.A., respectively or left un-reacted. The product was cleaved from the resin using general procedure 11.B., 11.C., 11.H., or 11.J.

Probe Library 57

An Fmoc/Boc protected alpha-amino acid (Fmoc on the alpha-amine and Boc on the side chain amine) was coupled onto hydroxymethyl polystyrene resin using general procedure 1.A.1. The resin bound protected alpha-amine was deprotected using general procedure 2.A. A Boc protected amino acid was coupled onto the resin bound alpha-amine using general procedure 3.A. The Boc protecting groups are removed using general procedure 2.B. The product was cleaved from the resin using general procedure 11.B., 11.C., 11.H., or 11.J.

Probe Library 58

Either a Boc or Fmoc protected amino acid was attached to Merrifield resin according to general procedure 1.A.1. The amino acid was deprotected according to general procedure 2.B for Fmoc amino acids or 2.A for Boc amino acids and the product was removed from the resin according to general procedure 11.C.

Probe Library 59

Either a Boc or Fmoc protected amino acid was attached to Merrifield resin according to general procedure 1.A.1. The amino acid was deprotected according to general procedure 2.B for Fmoc amino acids or 2.A for Boc amino acids and the product was removed from the resin according to general procedure 11.B.

Probe Library 60

Either a Boc or Fmoc protected amino acid was attached to Merrifield resin according to general procedure 1.A.1. The amino acid was deprotected according to general procedure 2.B for Fmoc amino acids or 2.A for Boc amino acids and the product was removed from the resin according to general procedure 11.J.

Probe Library 61

Either a Boc or Fmoc protected amino acid was attached to Merrifield resin according to general procedure 1.A.1. The amino acid was deprotected according to general procedure 2.B for Fmoc amino acids or 2.A for Boc amino acids and the product was removed from the resin according to general procedure 11.H.

Probe Library 62

Either a Boc or Fmoc protected amino acid was attached to Merrifield resin according to general procedure 1.A.1. The amino acid was deprotected according to general procedure 2.B for Fmoc amino acids or 2.A for Boc amino acids and the carbamate formed according to general procedure 7.B. The product was removed from the resin according to general procedure 11.B.

Probe Library 63

Either a Boc or Fmoc protected amino acid was attached to Merrifield resin according to general procedure 1.A.1. The amino acid was deprotected according to general procedure 2.B for Fmoc amino acids or 2.A for Boc amino acids and the carbamate formed according to general procedure 7.B. The product was removed from the resin according to general procedure 11.J.

Probe Library 64

Either a Boc or Fmoc protected amino acid was attached to Merrifield resin according to general procedure 1.A.1. The amino acid was deprotected according to general procedure 2.B for Fmoc amino acids or 2.A for Boc amino acids and the carbamate formed according to general procedure 7.B. The product was removed from the resin according to general procedure 11H.

Probe Library 65

Either a Boc or Fmoc protected amino acid was attached to Merrifield resin according to general procedure 1.A.1. The amino acid was deprotected according to general procedure 2.B for Fmoc amino acids or 2.A for Boc amino acids and the carbamate formed according to general procedure 7.B. The product was removed from the resin using general procedure 11.C.

Probe Library 66

Either a Boc or Fmoc protected amino acid was attached to Merrifield resin according to general procedure 1.A.1. The amino acid was deprotected according to general procedure 2.B for Fmoc amino acids or 2.A for Boc amino acids and the carbamate formed according to general procedure 7.A.1. The product was removed from the resin according to general procedure 11.B.

Probe Library 67

Either a Boc or Fmoc protected amino acid was attached to Merrifield resin according to general procedure 1.A.1. The amino acid was deprotected according to general procedure 2.B for Fmoc amino acids or 2.A for Boc amino acids and the carbamate formed according to general procedure 7.A.1. The product was removed from the resin according to general procedure 11.C.

Probe Library 68

Either a Boc or Fmoc protected amino acid was attached to Merrifield resin according to general procedure 1.A.1. The amino acid was deprotected according to general procedure 2.B for Fmoc amino acids or 2.A for Boc amino acids and the carbamate formed according to general procedure 7.A.1. The product was removed from the resin according to general procedure 11.H.

Probe Library 69

Either a Boc or Fmoc protected amino acid was attached to Merrifield resin according to general procedure 1.A.1. The amino acid was deprotected according to general procedure 2.B for Fmoc amino acids or 2.A for Boc amino acids and the carbamate formed according to general procedure 7.A.1. The product was removed from the resin according to general procedure 11.J.

Probe Library 70

Either a Boc or Fmoc protected amino acid was attached to Merrifield resin according to general procedure 1.A.1. The amino acid was deprotected according to general procedure 2.B for Fmoc amino acids or 2.A for Boc amino acids and reductively aminated according to general procedure 5.A. The product was removed from the resin according to general procedure 11.B.

Probe Library 71

Either a Boc or Fmoc protected amino acid was attached to Merrifield resin according to general procedure 1.A.1. The amino acid was deprotected according to general procedure 2.B for Fmoc amino acids or 2.A for Boc amino acids and reductively aminated according to general procedure 5.A. The product was removed from the resin according to general procedure 11.H.

Probe Library 72

Either a Boc or Fmoc protected amino acid was attached to Merrifield resin according to general procedure 1.A.1. The amino acid was deprotected according to general procedure 2.B for Fmoc amino acids or 2.A for Boc amino acids and reductively aminated according to general procedure 5.A. The product was removed from the resin according to general procedure 11.J.

Probe Library 73

Either a Boc or Fmoc protected amino acid was attached to Merrifield resin according to general procedure 1.A.1. The amino acid was deprotected according to general procedure 2.B for Fmoc amino acids or 2.A for Boc amino acids and reductively aminated according to general procedure 5.A. The product was removed from the resin according to general procedure 11.C.

Probe Library 74

Either a Boc or Fmoc protected amino acid was attached to Merrifield resin according to general procedure 1.A.1. The amino acid was deprotected according to general procedure 2.B for Fmoc amino acids or 2.A for Boc amino acids and the sulfonamide formed according to general procedure 4.A. The product was removed from the resin according to general procedure 11.J.

Probe Library 75

Either a Boc or Fmoc protected amino acid was attached to Merrifield resin according to general procedure 1.A.1. The amino acid was deprotected according to general procedure 2.B for Fmoc amino acids or 2.A for Boc amino acids and the sulfonamide formed according to general procedure 4.A. The product was removed from the resin according to general procedure 11.B.

Probe Library 76

Either a Boc or Fmoc protected amino acid was attached to Merrifield resin according to general procedure 1.A.1. The amino acid was deprotected according to general procedure 2.B for Fmoc amino acids or 2.A for Boc amino acids and the sulfonamide formed according to general procedure 4.A. The product was removed from the resin according to general procedure 11.H

Probe Library 77

Either a Boc or Fmoc protected amino acid was attached to Merrifield resin according to general procedure 1.A.1. The amino acid was deprotected according to general procedure 2.B for Fmoc amino acids or 2.A for Boc amino acids and the sulfonamide formed according to general procedure 4.A. The product was removed from the resin using dimethylamine according to general procedure 11.C.

Probe Library 78

Either a Boc or Fmoc protected amino acid was attached to Merrifield resin according to general procedure 1.A.1. The amino acid was deprotected according to general procedure 2.B for Fmoc amino acids or 2.A for Boc amino acids and the sulfonyl urea formed according to general procedure 4.B.1. The product was removed from the resin according to general procedure 11.B.

Probe Library 79

Either a Boc or Fmoc protected amino acid was attached to Merrifield resin according to general procedure 1.A.1. The amino acid was deprotected according to general procedure 2.B for Fmoc amino acids or 2.A for Boc amino acids and the sulfonyl urea formed according to general procedure 4.B.1. The product was removed from the resin according to general procedure 11.C.

Probe Library 80

Either a Boc or Fmoc protected amino acid was attached to Merrifield resin according to general procedure 1.A.1. The amino acid was deprotected according to general procedure 2.B for Fmoc amino acids or 2.A for Boc amino acids and the sulfonyl urea formed according to general procedure 4.B.1. The product was removed from the resin according to general procedure 11H.

Probe Library 81

Either a Boc or Fmoc protected amino acid was attached to Merrifield resin according to general procedure 1.A.1. The amino acid was deprotected according to general procedure 2.B for Fmoc amino acids or 2.A for Boc amino acids and the sulfonyl urea formed according to general procedure 4.B.1. The product was removed from the resin according to general procedure 11.J.

Probe Library 82

Either a Boc or Fmoc protected amino acid was attached to Merrifield resin according to general procedure 1.A.1. The amino acid was deprotected according to general procedure 2.B for Fmoc amino acids or 2.A for Boc amino acids and the urea formed according to general procedure 6.B. The product was removed from the resin according to general procedure 11.B.

Probe Library 83

Either a Boc or Fmoc protected amino acid was attached to Merrifield resin according to general procedure 1.A.1. The amino acid was deprotected according to general procedure 2.B for Fmoc amino acids or 2.A for Boc amino acids and the urea formed according to general procedure 6.B. The product was removed from the resin according to general procedure 11.C.

Probe Library 84

Either a Boc or Fmoc protected amino acid was attached to Merrifield resin according to general procedure 1.A.1. The amino acid was deprotected according to general procedure 2.B for Fmoc amino acids or 2.A for Boc amino acids and the urea formed according to general procedure 6.B. The product was removed from the resin according to general procedure 11.H.

Probe Library 85

Either a Boc or Fmoc protected amino acid was attached to Merrifield resin according to general procedure 1.A.1. The amino acid was deprotected according to general procedure 2.B for Fmoc amino acids or 2.A for Boc amino acids and the urea formed according to general procedure 6.B. The product was removed from the resin according to general procedure 11.J.

Probe Library 86

Either a Boc or Fmoc protected amino acid was attached to Merrifield resin according to general procedure 1.A.1. The amino acid was deprotected according to general procedure 2.B for Fmoc amino acids or 2.A for Boc amino acids and the urea formed according to general procedure 6.A. The product was removed from the resin according to general procedure 11.B.

Probe Library 87

Either a Boc or Fmoc protected amino acid was attached to Merrifield resin according to general procedure 1.A.1. The amino acid was deprotected according to general procedure 2.B for Fmoc amino acids or 2.A for Boc amino acids and the urea formed according to general procedure 6.A. The product was removed from the resin according to general procedure 11.C.

Probe Library 88

Either a Boc or Fmoc protected amino acid was attached to Merrifield resin according to general procedure 1.A.1. The amino acid was deprotected according to general procedure 2.B for Fmoc amino acids or 2.A for Boc amino acids and the urea formed according to general procedure 6.A. The product was removed from the resin according to general procedure 11.H.

Probe Library 89

Either a Boc or Fmoc protected amino acid was attached to Merrifield resin according to general procedure 1.A.1. The amino acid was deprotected according to general procedure 2.B for Fmoc amino acids or 2.A for Boc amino acids and the urea formed according to general procedure 6.A. The product was removed from the resin according to general procedure 11.J.

Probe Library 90

Either a Boc or Fmoc protected amino acid was attached to Merrifield resin according to general procedure 1.A.1. The amino acid was deprotected according to general procedure 2.B for Fmoc amino acids or 2.A for Boc amino acids and the urea formed according to general procedure 6.C. The product was removed from the resin according to general procedure 11.B.

Probe Library 91

Either a Boc or Fmoc protected amino acid was attached to Merrifield resin according to general procedure 1.A.1. The amino acid was deprotected according to general procedure 2.B for Fmoc amino acids or 2.A for Boc amino acids and the urea formed according to general procedure 6.C. The product was removed from the resin according to general procedure 11.C.

Probe Library 92

Either a Boc or Fmoc protected amino acid was attached to Merrifield resin according to general procedure 1.A.1. The amino acid was deprotected according to general procedure 2.B for Fmoc amino acids or 2.A for Boc amino acids and the urea formed according to general procedure 6.C. The product was removed from the resin according to general procedure 11.H.

Probe Library 93

Either a Boc or Fmoc protected amino acid was attached to Merrifield resin according to general procedure 1.A.1. The amino acid was deprotected according to general procedure 2.B for Fmoc amino acids or 2.A for Boc amino acids and the urea formed according to general procedure 6.C. The product was removed from the resin according to general procedure 11.J.

Probe Library 94

Either a Boc or Fmoc protected amino acid was attached to Merrifield resin according to general procedure 1.A.1. The amino acid was deprotected according to general procedure 2.B for Fmoc amino acids or 2.A for Boc amino acids and acylated according to general procedure 3.A. The product was removed from the resin according to general procedure 11.B.

Probe Library 95

Either a Boc or Fmoc protected amino acid was attached to Merrifield resin according to general procedure 1 A.1. The amino acid was deprotected according to general procedure 2.B for Fmoc amino acids or 2.A for Boc amino acids and acylated according to general procedure 3.A. The product was removed from the resin according to general procedure 11.J.

Probe Library 96

Either a Boc or Fmoc protected amino acid was attached to Merrifield resin according to general procedure 1.A.1. The amino acid was deprotected according to general procedure 2.B for Fmoc amino acids or 2.A for Boc amino acids and the acylated according to general procedure 3.A. The product was removed from the resin according to general procedure 11.H.

Probe Library 97

Either a Boc or Fmoc protected amino acid was attached to Merrifield resin according to general procedure 1.A.1. The amino acid was deprotected according to general procedure 2.B for Fmoc amino acids or 2.A for Boc amino acids and then acylated according to general procedure 3.A. The product was removed from the resin according to general procedure 11.C.

Probe Library 98

Either a Boc or Fmoc protected amino acid was attached to Merrifield resin according to general procedure 1.A.1. The amino acid was deprotected according to general procedure 2.B for Fmoc amino acids or 2.A for Boc amino acids and acylated according to general procedure 3.A. The product was removed from the resin according to general procedure 11.B.

Probe Library 99

Either a Boc or Fmoc protected amino acid was attached to Merrifield resin according to general procedure 1.A.1. The amino acid was deprotected according to general procedure 2.B for Fmoc amino acids or 2.A for Boc amino acids and acylated according to general procedure 3.A. The product was removed from the resin according to general procedure 11.J.

Probe Library 100

Either a Boc or Fmoc protected amino acid was attached to Merrifield resin according to general procedure 1.A.1. The amino acid was deprotected according to general procedure 2.B for Fmoc amino acids or 2.A for Boc amino acids and the acylated according to general procedure 3.A. The product was removed from the resin according to general procedure 11.H.

Probe Library 101

Either a Boc or Fmoc protected amino acid was attached to Merrifield resin according to general procedure 1.A.1. The amino acid was deprotected according to general procedure 2.B for Fmoc amino acids or 2.A for Boc amino acids and then acylated according to general procedure 3.A. The product was removed from the resin according to general procedure 11.C.

Probe Library 102

An Fmoc-protected amino acid was attached to Rink resin according to general procedure 1.C.1. The amino acid was deprotected according to general procedure 2.B. The free amine was then acylated according to general procedure 3.A. The product was removed from the resin according to general procedure 11.A.

Probe Library 103

An Fmoc-protected amino acid was attached to Rink resin according to general procedure 1.C.1. The amino acid was deprotected according to general procedure 2.B. The free amine was then reductively aminated according to general procedure 5.A. The product was removed from the resin according to general procedure 11.A.

Probe Library 104

An Fmoc-protected amino acid was attached to Rink resin according to general procedure 1.C.1. The amino acid was deprotected according to general procedure 2.B. The sulfonamide was then formed according to general procedure 4.A. The product was removed from the resin according to general procedure 11.A.

Probe Library 105

An Fmoc-protected amino acid was attached to Wang resin according to general procedure 1.B.1. The amino acid was deprotected according to general procedure 2.A. The free amine was then acylated according to general procedure 3.A and the product released from the resin according to general procedure 11.A.

Probe Library 106

An Fmoc-protected amino acid was attached to Wang resin according to general procedure 1.B.1. The free amine was then reductively aminated according to general procedure 5.A. The product was removed from the resin according to general procedure 11.A.

Probe Library 107

An Fmoc-protected amino acid was attached to Wang resin according to general procedure 1.B.1. The sulfonamide was formed according to general procedure 4.A. The product was removed from the resin according to general procedure 11.A

Probe Library 108

An Fmoc protected amino acid was attached to Wang resin according to general procedure 1.B.1. The amino acid was deprotected according to general procedure 2.A and acylated according to general procedure 3.C.1. The product was removed from the resin using general procedure 11.A.

Probe Library 109

An Fmoc protected amino acid was attached to Wang resin according to general procedure 1.B.1. The amino acid was deprotected according to general procedure 2.A and the urea formed according to general procedure 6.C. The product was removed from the resin using general procedure 11.A

Probe Library 110

An Fmoc protected amino acid was attached to Wang resin according to general procedure 1.B.1. The amino acid was deprotected according to general procedure 2.A and the urea formed according to general procedure 6.A. The product was removed from the resin using general procedure 11.A

Probe Library 111

An Fmoc protected amino acid was attached to Wang resin according to general procedure 1.B.1. The amino acid was deprotected according to general procedure 2.A and the urea formed according to general procedure 6.B. The product was removed from the resin using general procedure 11.A

Probe Library 112

An Fmoc protected amino acid was attached to Wang resin according to general procedure 1.B.1. The amino acid was deprotected according to general procedure 2.A and the sulfonyl urea formed according to general procedure 4.B.1. The product was removed from the resin using general procedure 11.A

Probe Library 113

An Fmoc protected amino acid was attached to Wang resin according to general procedure 1.B.1. The amino acid was deprotected according to general procedure 2.A and the carbamate formed according to general procedure 7.A.1. The product was removed from the resin using general procedure 11.A

Probe Library 114

An Fmoc protected amino acid was attached to Wang resin according to general procedure 1.B.1. The amino acid was deprotected according to general procedure 2.A and the urea formed according to general procedure 7.B. The product was removed from the resin using general procedure 11.A

Probe Library 115

Aldehyde resin was reductively aminated and acylated with an Fmoc amino acid according to general procedure 1.D.1. The product was cleaved from the resin using general procedure 11.L.2.

Probe Library 116

Aldehyde resin was reductively aminated and acylated with an Fmoc amino acid according to general procedure 1.D.1. The amino acid was deprotected according to general procedure 2.A and the product was cleaved from the resin using general procedure 11.L.2.

Probe Library 117

Aldehyde resin was reductively aminated and acylated with a Boc amino acid according to general procedure 1.D.1. The product was cleaved from the resin using general procedure 11.L.2.

Probe Library 118

Aldehyde resin was reductively aminated according to general procedure 1.D.5. The amine was then acylated according to procedure 3.A. The product was cleaved from the resin using general procedure 11.L.2.

Probe Library 119

Aldehyde resin is prepared according to general procedure 1.D.5. The sulfonamide is then formed according to general procedure 4.A. The product is cleaved from the resin according to general procedure 11.L.2.

Probe Library 120

Aldehyde resin was reductively aminated and acylated with an Fmoc protected amino acid to general procedure 1.D.1. The amino acid was deprotected according to general procedure 2.A. The free amine was then reductively aminated according to general procedure 5.A. The product was cleaved from the resin using general procedure 11.L.2.

Probe Library 121

Aldehyde resin was reductively aminated and acylated with an Fmoc protected amino acid to general procedure 1.D.1. The amino acid was deprotected according to general procedure 2.A. and the urea formed according to general procedure 6.A. The product was cleaved from the resin using general procedure 11.L.2.

Probe Library 122

Aldehyde resin was reductively aminated and acylated with an Fmoc protected amino acid to general procedure 1.D.1. The amino acid was then deprotected according to general procedure 2.A. and followed by acylation of the free amine according to procedure 3.A. The product was cleaved from the resin using general procedure 11.L.2.

Probe Library 123

Aldehyde resin was reductively aminated and acylated with an Fmoc protected amino acid to general procedure 1.D.1. The amino acid was then deprotected according to general procedure 2.A. and followed by acylation of the free amine according to procedure 3.C.1. The product was cleaved from the resin using general procedure 11.L.2.

Probe Library 124

Aldehyde resin was reductively aminated and acylated with an Fmoc protected amino acid to general procedure 1.D.1. The amino acid was then deprotected according to general procedure 2.A. followed by sulfonyl urea formation according to procedure 4.B.1.. The product was cleaved from the resin using general procedure 11.L.2.

Probe Library 125

Aldehyde resin was reductively aminated and acylated with an Fmoc protected amino acid to general procedure 1.D.1. The amino acid was then deprotected according to general procedure 2.A. followed by urea formation according to procedure 6.C. The product was cleaved from the resin using general procedure 11.L.2

Probe Library 126

Aldehyde resin was reductively aminated and acylated with an Fmoc protected amino acid to general procedure 1.D.1. The amino acid was then deprotected according to general procedure 2.A. and followed by the formation of the sulfonamide according to procedure 4.A. The product was cleaved from the resin using general procedure 11.L.2.

Probe Library 127

Aldehyde resin was reductively aminated and acylated with an Fmoc protected amino acid to general procedure 1.D.1. The amino acid was then deprotected according to general procedure 2.A. and followed by carbamate formation according to procedure 7.B. The product was cleaved from the resin using general procedure 11.L.2.

Probe Library 128

Aldehyde resin was reductively aminated and acylated with an Fmoc protected amino acid to general procedure 1.D.1. The amino acid was then deprotected according to general procedure 2.A. and followed by urea formation according to procedure 6.B. The product was cleaved from the resin using general procedure 11.L.2.

Probe Library 129

Aldehyde resin was reductively aminated and acylated with an Fmoc protected amino acid to general procedure 1.D.1. The amino acid was then deprotected according to general procedure 2.A. and followed by carbamate formation according to procedure 7.A.1. The product was cleaved from the resin using general procedure 11.L.2.

Probe Library 130

Aldehyde resin is prepared according to general procedure 1.D.5. The amine is then reductively aminated according to general procedure 5.A. The product is cleaved from the resin according to general procedure 11.L.2.

Probe Library 131

Aldehyde resin is prepared according to general procedure 1.D.5. The urea is then formed according to general procedure 6.A. The product is cleaved from the resin according to general procedure 11.L.2.

Probe Library 132

Aldehyde resin is prepared according to general procedure 1.D.5. The urea is then formed according to general procedure 6.B. The product is cleaved from the resin according to general procedure 11.L.2.

Probe Library 133

Aldehyde resin is prepared according to general procedure 1.D.5. The urea is then formed according to general procedure 6.C. The product is cleaved from the resin according to general procedure 11.L.2.

Probe Library 134

Aldehyde resin is prepared according to general procedure 1.D.5. The sulfonyl urea is then formed according to general procedure 4.B.1. The product is cleaved from the resin according to general procedure 11.L.2.

Probe Library 135

Aldehyde resin is prepared according to general procedure 1.D.5. The carbamate is then formed according to general procedure 7.A.1. The product is cleaved from the resin according to general procedure 11.L.2.

Probe Library 136

Aldehyde resin is prepared according to general procedure 1.D.5. The carbamate is then formed according to general procedure 7.B. The product is cleaved from the resin according to general procedure 11.L.2.

Probe Library 137

Either a Boc or Fmoc protected amino acid was attached to Merrifield resin according to general procedure 1.A.1. The amino acid was deprotected according to general procedure 2.B for Fmoc amino acids or 2.A for Boc amino acids. The amine was acylated with a second Fmoc or Boc protected amino acid according to procedure 3.A and the protecting groups removed according to general procedure 2B for Fmoc amino acids or 2A for Boc amino acids and the product was removed from the resin according to general procedure 11.C.

Probe Library 138

Either a Boc or Fmoc protected amino acid was attached to Merrifield resin according to general procedure 1.A.1. The amino acid was deprotected according to general procedure 2.B for Fmoc amino acids or 2.A for Boc amino acids. The amine was acylated with a second Fmoc or Boc protected amino acid according to procedure 3.A and the protecting groups removed according to general procedure 2B for Fmoc amino acids or 2A for Boc amino acids and the product was removed from the resin according to general procedure 11.B.

Probe Library 139

Either a Boc or Fmoc protected amino acid was attached to Merrifield resin according to general procedure 1.A.1. The amino acid was deprotected according to general procedure 2.B for Fmoc amino acids or 2.A for Boc amino acids. The amine was acylated with a second Fmoc or Boc protected amino acid according to procedure 3.A and the protecting groups removed according to general procedure 2B for Fmoc amino acids or 2A for Boc amino acids and the product was removed from the resin according to general procedure 11.J.

Probe Library 140

Either a Boc or Fmoc protected amino acid was attached to Merrifield resin according to general procedure 1.A.1. The amino acid was deprotected according to general procedure 2.B for Fmoc amino acids or 2.A for Boc amino acids. The amine was acylated with a second Fmoc or Boc protected amino acid according to procedure 3.A and the protecting groups removed according to general procedure 2B for Fmoc amino acids or 2A for Boc amino acids and the product was removed from the resin according to general procedure 11.H.

Probe Library 141

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The carbamate was then formed according to general procedure 7.B. The product was removed from the resin according to general procedure 11.B.

Probe Library 142

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The carbamate was then formed according to general procedure 7.B. The product was removed from the resin according to general procedure 11.C

Probe Library 143

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The carbamate was then formed according to general procedure 7.B. The product was removed from the resin according to general procedure 11.H.

Probe Library 144

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The carbamate was then formed according to general procedure 7.B. The product was removed from the resin according to general procedure 11.J

Probe Library 145

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The carbamate was then formed according to general procedure 7.A.1. The product was removed from the resin according to general procedure 11.B.

Probe Library 146

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The carbamate was then formed according to general procedure 7.A.1. The product was removed from the resin according to general procedure 11.C.

Probe Library 147

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The carbamate was then formed according to general procedure 7.A.1. The product was removed from the resin according to general procedure 11.H.

Probe Library 148

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The carbamate was then formed according to general procedure 7.A.1. The product was removed from the resin according to general procedure 11.J.

Probe Library 149

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The free amine was then reductively aminated according to procedure 5.A. The product was removed from the resin according to general procedure 11.B.

Probe Library 150

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The free amine was then reductively aminated according to procedure 5.A. The product was removed from the resin according to general procedure 11.C.

Probe Library 151

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The free amine was then reductively aminated according to procedure 5.A. The product was removed from the resin according to general procedure 11.H.

Probe Library 152

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The free amine was then reductively aminated according to procedure 5.A. The product was removed from the resin according to general procedure 11.J.

Probe Library 153

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The sulfonamide was then formed according to procedure 4.A. The product was removed from the resin according to general procedure 11.B.

Probe Library 154

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The sulfonamide was then formed according to procedure 4.A. The product was removed from the resin according to general procedure 11.C.

Probe Library 155

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The sulfonamide was then formed according to procedure 4.A. The product was removed from the resin according to general procedure 11.H.

Probe Library 156

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The sulfonamide was then formed according to procedure 4.A. The product was removed from the resin according to general procedure 11.J.

Probe Library 157

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The sulfonyl urea was then formed according to procedure 4.B.1. The product was removed from the resin according to general procedure 11.B.

Probe Library 158

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The sulfonyl urea was then formed according to procedure 4.B.1. The product was removed from the resin according to general procedure 11.C.

Probe Library 159

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The sulfonyl urea was then formed according to procedure 4.B.1. The product was removed from the resin according to general procedure 11.H.

Probe Library 160

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The sulfonyl urea was then formed according to procedure 4.B.1. The product was removed from the resin according to general procedure 11.H.

Probe Library 161

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The urea was then formed according to procedure 6.B. The product was removed from the resin according to general procedure 11.B.

Probe Library 162

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The urea was then formed according to procedure 6.B. The product was removed from the resin according to general procedure 11.C.

Probe Library 163

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The urea was then formed according to procedure 6.B. The product was removed from the resin according to general procedure 11.H.

Probe Library 164

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The urea was then formed according to procedure 6.B. The product was removed from the resin according to general procedure 11.J.

Probe Library 165

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The urea was then formed according to procedure 6.A. The product was removed from the resin according to general procedure 11.B.

Probe Library 166

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The urea was then formed according to procedure 6.A. The product was removed from the resin according to general procedure 11.C.

Probe Library 167

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The urea was then formed according to procedure 6.A. The product was removed from the resin according to general procedure 11.H.

Probe Library 168

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The urea was then formed according to procedure 6.A. The product was removed from the resin according to general procedure 11.J

Probe Library 169

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The urea was then formed according to procedure 6.C. The product was removed from the resin according to general procedure 11.B.

Probe Library 170

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The urea was then formed according to procedure 6.C. The product was removed from the resin according to general procedure 11.C.

Probe Library 171

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The urea was then formed according to procedure 6.C. The product was removed from the resin according to general procedure 11.H.

Probe Library 172

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids. The urea was then formed according to procedure 6.C. The product was removed from the resin according to general procedure 11.J

Probe Library 173

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids and then acylated according to general procedure 3.A. The product was removed from the resin according to general procedure 11.B.

Probe Library 174

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids and then acylated according to general procedure 3.A. The product was removed from the resin according to general procedure 11.C.

Probe Library 175

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids and then acylated according to general procedure 3.A. The product was removed from the resin according to general procedure 11.H.

Probe Library 176

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids and then acylated according to general procedure 3.A. The product was removed from the resin according to general procedure 11.J

Probe Library 177

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids and then acylated according to general procedure 3.C.1. The product was removed from the resin according to general procedure 11.B.

Probe Library 178

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids and then acylated according to general procedure 3.C.1. The product was removed from the resin according to general procedure 11.C.

Probe Library 179

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids and then acylated according to general procedure 3.C.1. The product was removed from the resin according to general procedure 11.H.

Probe Library 180

Either a Boc or Fmoc protected amino acid was attached to Merrifield resin according to general procedure 1.A.1. The amino acid was deprotected according to general procedure 2.B for Fmoc amino acids or 2.A for Boc amino acids. The resin was then acylated with a second Fmoc or Boc protected amino acid according to procedure 3.A and the protecting groups removed according to general procedure 2B for Fmoc amino acids or 2A for Boc amino acids and then acylated according to general procedure 3.C.1. The product was removed from the resin according to general procedure 11.J

Probe Library 181

An Fmoc-protected amino acid was attached to Wang resin according to general procedure 1.B.1. The amino acid was deprotected according to general procedure 2.A. The free amine was acylated with an Fmoc amino acid according to general procedure 3.A and the Fmoc group removed according to general procedure 2.A. The product released from the resin according to general procedure 11.A.

Probe Library 182

An Fmoc-protected amino acid was attached to Wang resin according to general procedure 1.B.1. The amino acid was deprotected according to general procedure 2.A. The free amine was acylated with an Fmoc amino acid according to general procedure 3.A and the Fmoc group removed according to general procedure 2.A. The free amine was then acylated according to general procedure 3.A and the product released from the resin according to general procedure 11.A.

Probe Library 183

An Fmoc-protected amino acid was attached to Wang resin according to general procedure 1.B.1. The amino acid was deprotected according to general procedure 2.A. The free amine was acylated with an Fmoc amino acid according to general procedure 3.A and the Fmoc group removed according to general procedure 2.A. The free amine was then reductively aminated according to general procedure 5.A. The product was removed from the resin according to general procedure 11.A.

Probe Library 184

An Fmoc-protected amino acid was attached to Wang resin according to general procedure 1.B.1. The amino acid was deprotected according to general procedure 2.A. The free amine was acylated with an Fmoc amino acid according to general procedure 3.A and the Fmoc group removed according to general procedure 2.A. The sulfonamide was formed according to general procedure 4.A. The product was removed from the resin according to general procedure 11.A

Probe Library 185

An Fmoc protected amino acid was attached to Wang resin according to general procedure 1.B.1. The amino acid was deprotected according to general procedure 2.A. The free amine was acylated with an Fmoc amino acid according to general procedure 3.A and the Fmoc group removed according to general procedure 2.A. The free amine was then acylated according to general procedure 3.C.1. The product was removed from the resin using general procedure 11.A.

Probe Library 186

An Fmoc protected amino acid was attached to Wang resin according to general procedure 1.B.1 The amino acid was deprotected according to general procedure 2.A. The free amine was acylated with an Fmoc amino acid according to general procedure 3.A and the Fmoc group removed according to general procedure 2.A. The urea was then formed according to general procedure 6.C. The product was removed from the resin using general procedure 11.A

Probe Library 187

An Fmoc protected amino acid was attached to Wang resin according to general procedure 1.B.1. The amino acid was deprotected according to general procedure 2.A. The free amine was acylated with an Fmoc amino acid according to general procedure 3.A and the Fmoc group removed according to general procedure 2.A. The urea was then formed according to general procedure 6.A. The product was removed from the resin using general procedure 11.A

Probe Library 188

An Fmoc protected amino acid was attached to Wang resin according to general procedure 1.B.1. The amino acid was deprotected according to general procedure 2.A. The free amine was acylated with an Fmoc amino acid according to general procedure 3.A and the Fmoc group removed according to general procedure 2.A. The urea was then formed according to general procedure 6.B. The product was removed from the resin using general procedure 11.A

Probe Library 189

An Fmoc protected amino acid was attached to Wang resin according to general procedure 1.B.1. The amino acid was deprotected according to general procedure 2.A. The free amine was acylated with an Fmoc amino acid according to general procedure 3.A and the Fmoc group removed according to general procedure 2.A. The sulfonyl urea formed according to general procedure 4.B.1. The product was removed from the resin using general procedure 11.A

Probe Library 190

An Fmoc protected amino acid was attached to Wang resin according to general procedure 1.B.1. The amino acid was deprotected according to general procedure 2.A. The free amine was acylated with an Fmoc amino acid according to general procedure 3.A and the Fmoc group removed according to general procedure 2.A. The carbamate formed according to general procedure 7.A.1. The product was removed from the resin using general procedure 11.A

Probe Library 191

An Fmoc protected amino acid was attached to Wang resin according to general procedure 1.B.1. The amino acid was deprotected according to general procedure 2.A. The free amine was acylated with an Fmoc amino acid according to general procedure 3.A and the Fmoc group removed according to general procedure 2.A. The urea formed according to general procedure 7.B. The product was removed from the resin using general procedure 11.A

Probe Library 192

Aldehyde resin was reductively aminated and acylated with an Fmoc amino acid according to general procedure 1.D.1. The amino acid was deprotected according to general procedure 2.A. The free amine was then acylated with an Fmoc amino acid according to general procedure 3.A and the Fmoc group removed according to general procedure 2.A. The amino acid was deprotected according to general procedure 2.A and the product was cleaved from the resin using general procedure 11.L.2.

Probe Library 193

Aldehyde resin was reductively aminated and acylated with an Fmoc protected amino acid to general procedure 1.D.1. The amino acid was deprotected according to general procedure 2.A. The free amine was then acylated with an Fmoc amino acid according to general procedure 3.A and the Fmoc group removed according to general procedure 2.A. The free amine was then reductively aminated according to general procedure 5.A. The product was cleaved from the resin using general procedure 11.L.2.

Probe Library 194

Aldehyde resin was reductively aminated and acylated with an Fmoc protected amino acid to general procedure 1.D.1. The amino acid was deprotected according to general procedure 2.A. The free amine was then acylated with an Fmoc amino acid according to general procedure 3.A and the Fmoc group removed according to general procedure 2.A. The urea was then formed according to general procedure 6.A. The product was cleaved from the resin using general procedure 11.L.2.

Probe Library 195

Aldehyde resin was reductively aminated and acylated with an Fmoc protected amino acid to general procedure 1.D.1. The amino acid was deprotected according to general procedure 2.A. The free amine was then acylated with an Fmoc amino acid according to general procedure 3.A and the Fmoc group removed according to general procedure 2.A. The free amine was then acylated according to procedure 3.A. The product was cleaved from the resin using general procedure 11.L.2.

Probe Library 196

Aldehyde resin was reductively aminated and acylated with an Fmoc protected amino acid to general procedure 1.D.1. The amino acid was deprotected according to general procedure 2.A. The free amine was then acylated with an Fmoc amino acid according to general procedure 3.A and the Fmoc group removed according to general procedure 2.A, followed by acylation of the free amine according to procedure 3.C.1. The product was cleaved from the resin using general procedure 11.L.2.

Probe Library 197

Aldehyde resin was reductively aminated and acylated with an Fmoc protected amino acid to general procedure 1.D.1. The amino acid was deprotected according to general procedure 2.A. The free amine was then acylated with an Fmoc amino acid according to general procedure 3.A and the Fmoc group removed according to general procedure 2.A., followed by sulfonyl urea formation according to procedure 4.B.1.. The product was cleaved from the resin using general procedure 11.L.2.

Probe Library 198

Aldehyde resin was reductively aminated and acylated with an Fmoc protected amino acid to general procedure 1.D.1. The amino acid was deprotected according to general procedure 2.A. The free amine was then acylated with an Fmoc amino acid according to general procedure 3.A and the Fmoc group removed according to general procedure 2.A, followed by urea formation according to procedure 6.C. The product was cleaved from the resin using general procedure 11.L.2

Probe Library 199

Aldehyde resin was reductively aminated and acylated with an Fmoc protected amino acid to general procedure 1.D.1 The amino acid was deprotected according to general procedure 2.A. The free amine was then acylated with an Fmoc amino acid according to general procedure 3.A and the Fmoc group removed according to general procedure 2.A, followed by the formation of the sulfonamide according to procedure 4.A. The product was cleaved from the resin using general procedure 11.L.2.

Probe Library 200

Aldehyde resin was reductively aminated and acylated with an Fmoc protected amino acid to general procedure 1.D.1. The amino acid was deprotected according to general procedure 2.A. The free amine was then acylated with an Fmoc amino acid according to general procedure 3.A and the Fmoc group removed according to general procedure 2.A., followed by carbamate formation according to procedure 7.B. The product was cleaved from the resin using general procedure 11.L.2.

Probe Library 201

Aldehyde resin was reductively aminated and acylated with an Fmoc protected amino acid to general procedure 1.D.1. The amino acid was deprotected according to general procedure 2.A. The free amine was then acylated with an Fmoc amino acid according to general procedure 3.A and the Fmoc group removed according to general procedure 2.A., followed by urea formation according to procedure 6.B. The product was cleaved from the resin using general procedure 11.L.2.

Probe Library 202

Aldehyde resin was reductively aminated and acylated with an Fmoc protected amino acid to general procedure 1.D.1. The amino acid was deprotected according to general procedure 2.A. The free amine was then acylated with an Fmoc amino acid according to general procedure 3.A and the Fmoc group removed according to general procedure 2.A., followed by carbamate formation according to procedure 7.A.1. The product was cleaved from the resin using general procedure 11.L.2.

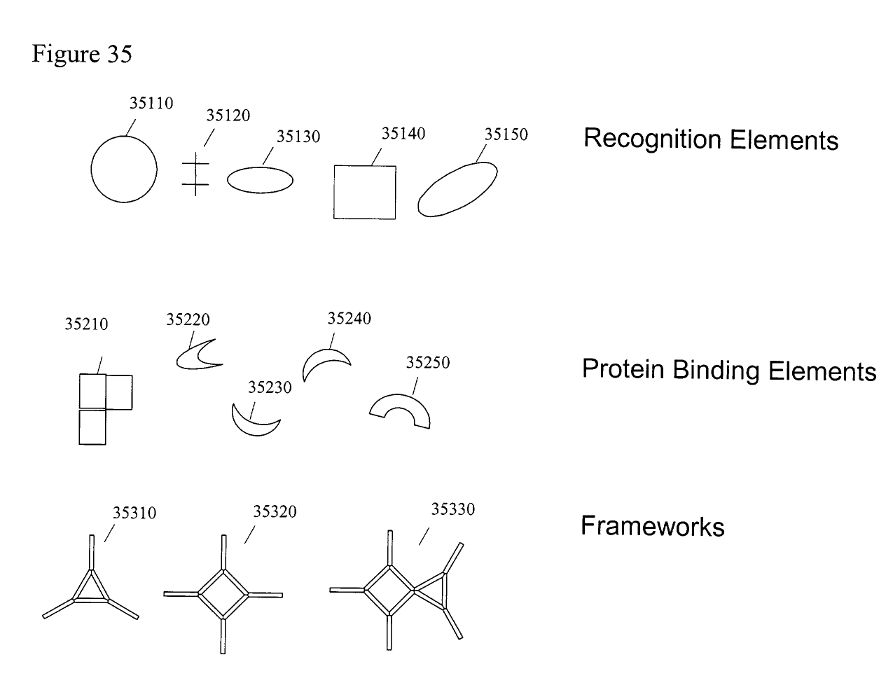

The conceptual framework for the present invention as discussed herein is represented pictorily in FIGS. 35 through 42. FIG. 35 graphically depicts representations of recognition elements, protein binding elements, and frameworks. The depictions are not intended to refer to specific chemical structures.

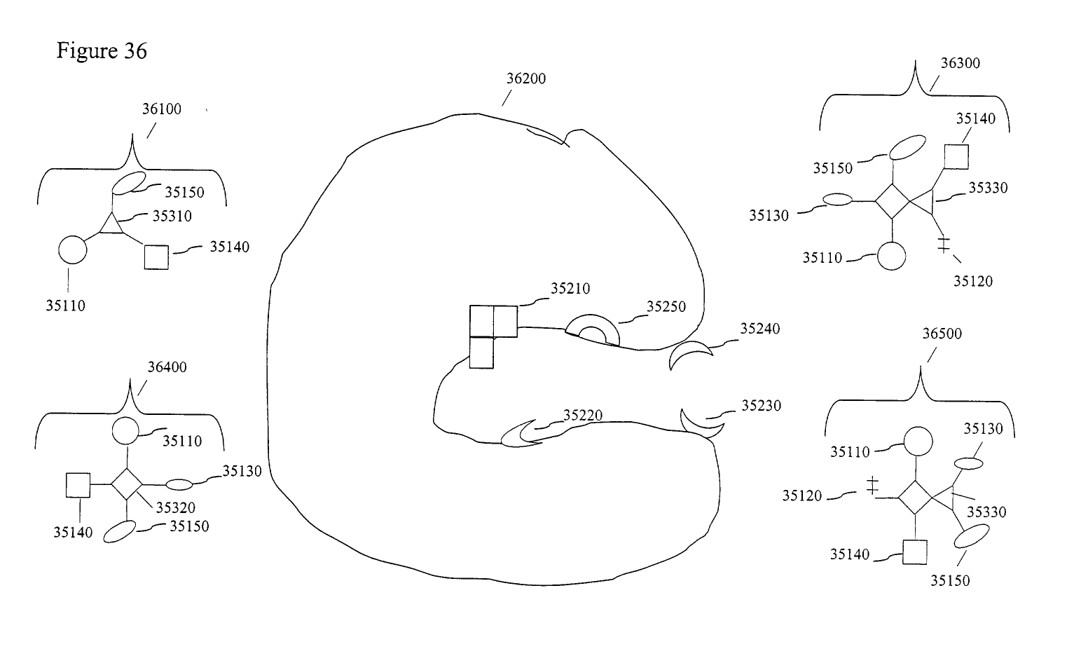

FIG. 36 depicts protein binding elements as displayed on an active site on a target protein (36200).

FIG. 36 also depicts probes 36100, 36300, 36400, 36500 comprising frameworks and recognition elements.

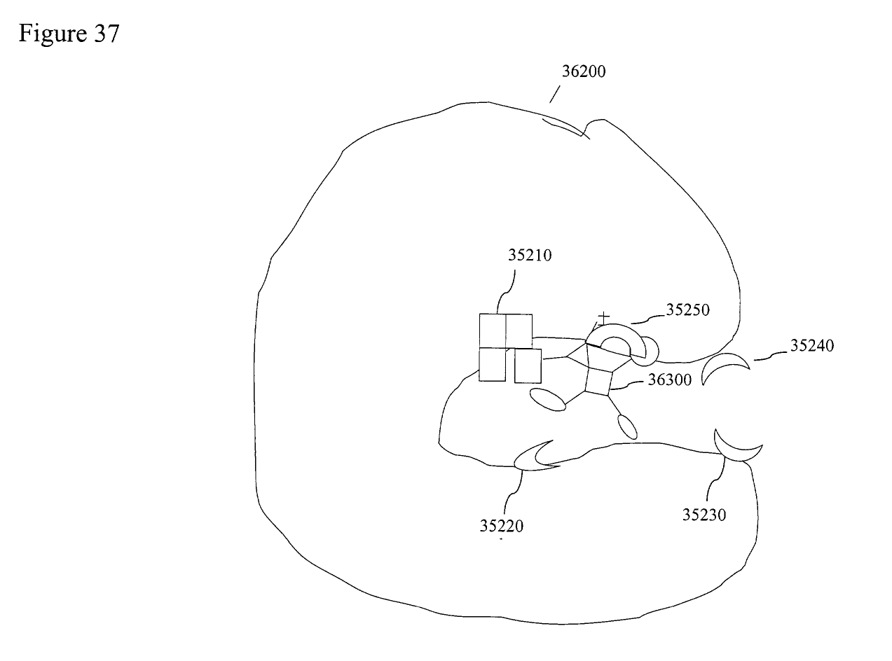

FIG. 37 depicts a probe 36300 associating with protein binding elements.

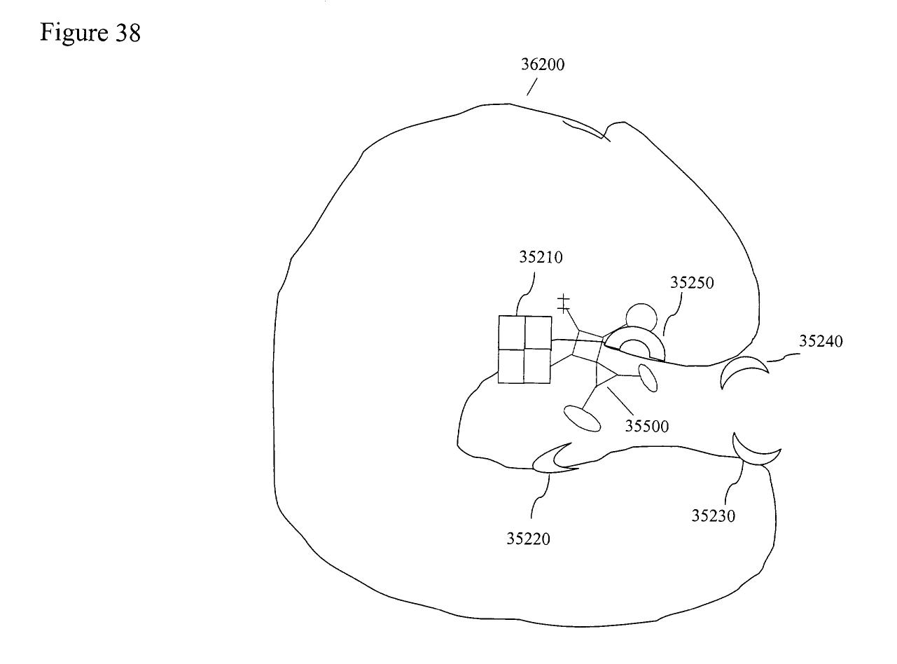

FIG. 38 depicts a probe associating with protein binding elements.

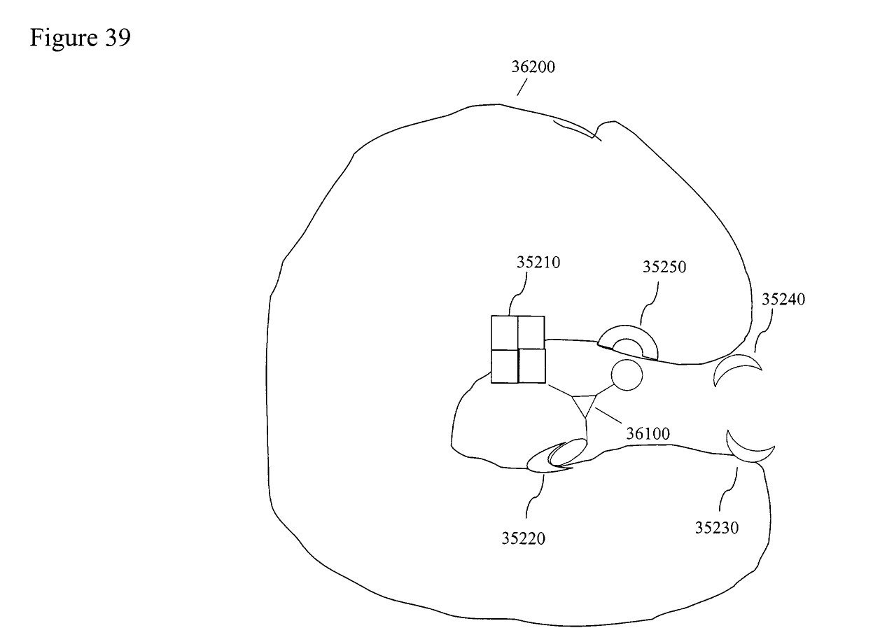

FIG. 39 depicts a probe associating with protein binding elements.

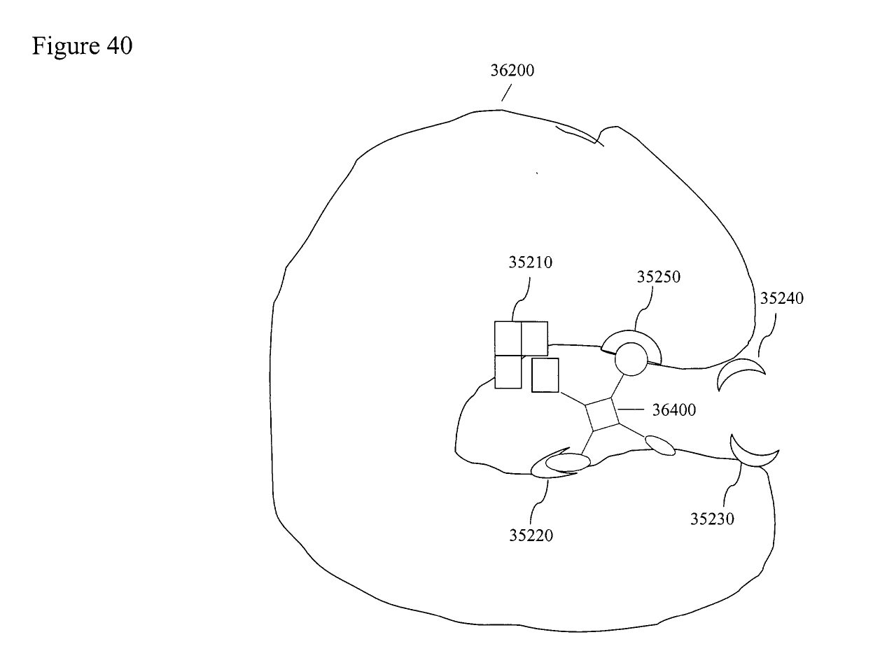

FIG. 40 depicts a probe associating with protein binding elements.

FIGS. 37 through 40 depict attempted association of a set of probes with a protein target.

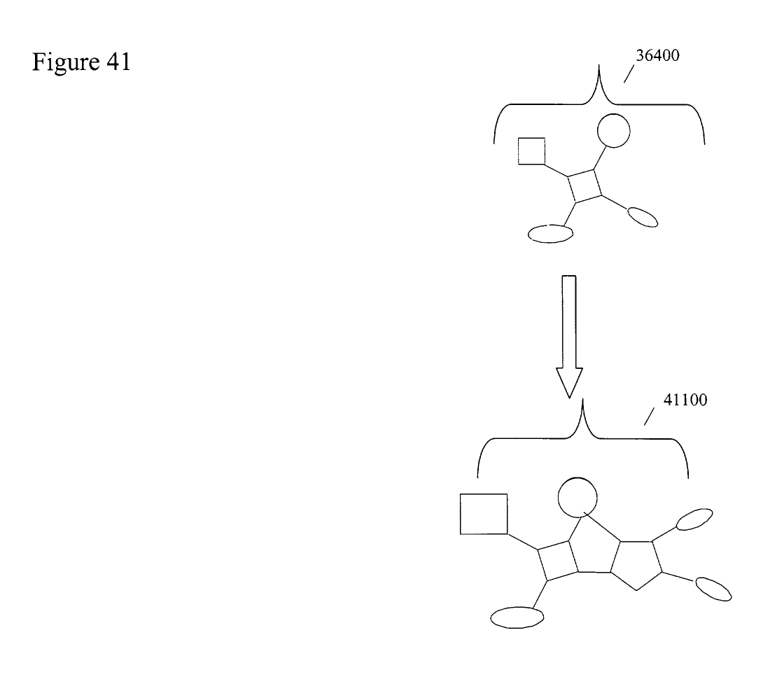

FIG. 41 depicts the creation of a second generation probe or drug candidate comprising a hit probe, addition frameworks, and recognition elements.

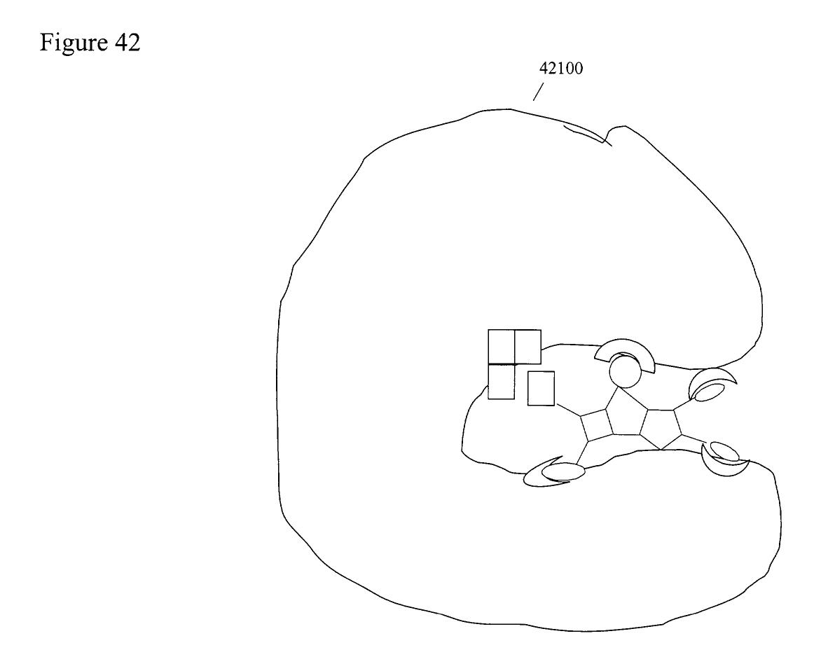

FIG. 42 depicts the association of the second generation probe or drug candidate with the protein binding target.

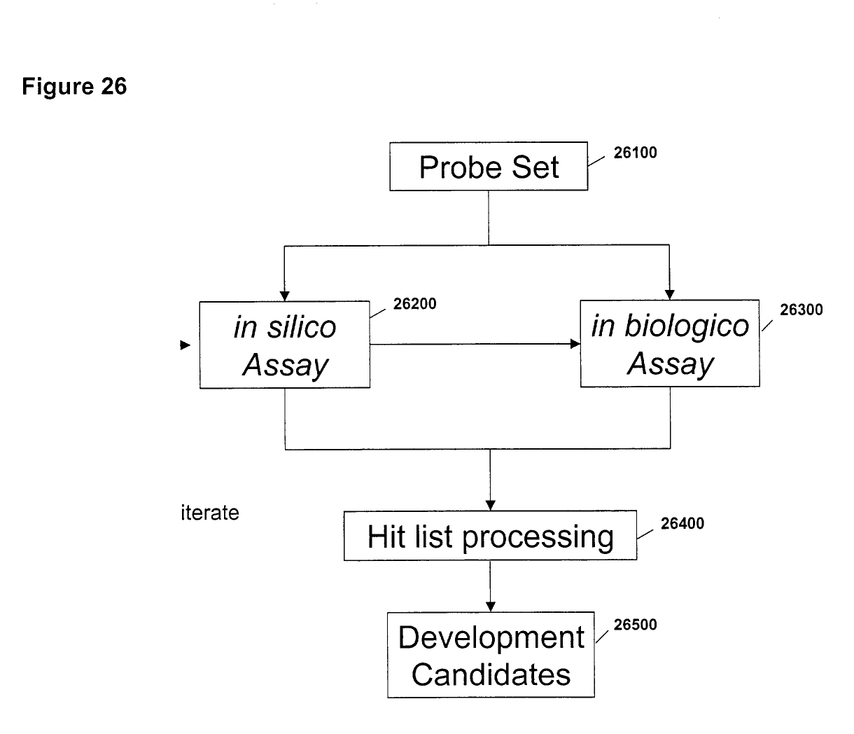

The present invention provides a drug discovery method using a Probe Set of the present invention. The drug discovery method of the present invention can use in silico and in biologico screening of probes separately, in parallel, or in combination, to identify drug development candidates. As shown in FIG. 26, a Probe Set (26100) of the present invention may be used in the in silico (26200) and in biologico (26300) screening of biological target(s).

To obtain the Probe Set (261000), the appropriate input fragments and frameworks for a Candidate Probe Set (302000), or for a suitable subset thereof, are defined. The appropriate for the reagents for connecting the input fragments and frameworks are assigned computationally. FIG. 30 contains a block diagram of the steps followed to create a Probe Set for used in the drug discovery method. The Candidate Probe Set is enumerated in silico (30510). As used herein, “enumeration” is defined as the computational rendering or listing of the individual members of a set of probes formed by the modification of a set of frameworks with input fragments. Several computational programs including, but not limited to Cerius²® (Accelrys Incorporated, San Diego, Calif.), Project Library (MDL Information Systems, San Leandro, Calif.) or Molecular Operating Environment (MOE, Chemical Computing Group, Montreal, Canada), CombiLibMaker (Tripos, St. Louis, Mo.) can be used for computer enumeration of the probe sets.

Physicochemical descriptors are then calculated for the probes or a suitable subset (30515). A non-exhaustive listing of descriptors which may be used for the description of the probes are given in Table 6. The values of the calculated descriptors define the “positions” of the probes of the Candidate Probe Set, or a suitable subset thereof, in a multi-dimensional space, which is herein refered to as “Chemistry Space” (30520). While the physical world is in three dimensions, the dimensionality of the above defined “Chemistry Space” is chosen to best suit the requirements of the drug discovery method and typically has dimensions greater than than three. Although, it is possible to have a defined “Chemistry Space” of one, two, or three dimensions.

Principal Components Analysis (PCA) is an efficient data-reduction technique. PCA involves a mathematical procedure that transforms a number of (potentially) correlated descriptors into a (smaller) number of uncorrelated descriptors called principal components. The first principal component accounts for most of the variability in the data (if possible), and each succeeding component accounts for the remaining variability.

The “reduced” dimensionality may permit visualization of the “Chemistry Space.”The “diversity” or “similarity” of compounds positioned in “Chemistry Space” is intuitively related to the inter-compound distance as measured in that space. In “Chemistry Space,” an axis may correspond to a structure-related property such as the presence or absence of a chlorine substituent, or the presence or absence of an aromatic ring, or the atomic charge, or polarizability. The Principal Components calculated from a Principal Component Analysis (PCA) may be used as axes of the “Chemistry Space,” as correlations between equivalent (orthogonal) descriptors are removed during this analysis. Computer programs, either developed in-house or commercially available, such as but not limited to “C².Diversity” from Accelrys, Inc. (San Diego, Calif.) or “Diverse Subset” in MOE (Chemical Computing Group Inc., Montreal, Canada), or “DiverseSolutions” or “Selector” (Tripos, Inc., St. Louis, Mo.) can identify probes that are diverse or similar by calculating their inter-compound distances in “Chemistry Space”.

In the present embodiment, a PCA was performed on a subset of the descriptors listed in Table 6, in order to position the Candidate Probe Set in “Chemistry Space”, and to reduce the dimensionality of the descriptor space to allow a graphical representation of “Chemistry Space” and visual analysis of the diversity or similarity of the probes with respect to one another.

Other statistical methods of data analysis and data reduction may be used in lieu of PCA. These other methods are known to those skilled in the art such as Chi² statistics, partial least squares (PLS), neural networks, and others.

The Candidate Probe Set or a subset may then be synthesized (30525) according to the methods described above and illustrated in schemes 1-9. Each synthesized probe is assigned a registration ID. The synthesized probes are then stored in plates or other suitable containers and labeled using bar coding or other means to associate an ID with the plate or other container. The location of the probe in the plate or other container is recorded. The probe structure, composition, quality assurance data including, but not limited to, spectroscopic data, chemical analysis data, purity information, and concentration, registration ID, location of the probe on the plate (e.g. row/column information), the physical location of the plate, and other relevant compound, plate, and inventory related attributes may be recorde in a database (30535) and associated with the probe registration ID using methods known to one skilled in the art. Data determined in silico for each probe such as, but not limited to, descriptors, ADME data, drug-like characteristics (Lipinski et al., Adv. Drug Delivery Rev., 23, 3-25, 1997), and other calculated data may also be recorde in a database and associated with the probe registration ID at this time. The above described procedure permits one to locate any probe that has been synthesized including the plate or other container in which it is stored.

Following the optional synthesis of the each of the probes of the Candidate Probe Set, or a suitable subset thereof, a Probe Set is defined (261000) and can be screend either in silico or in biologico against a particular therapeutic agent. Further, the data from in silico or in biologico screens of the Probes Set can be used to modify or narrow additional in silico or in biologico screens.

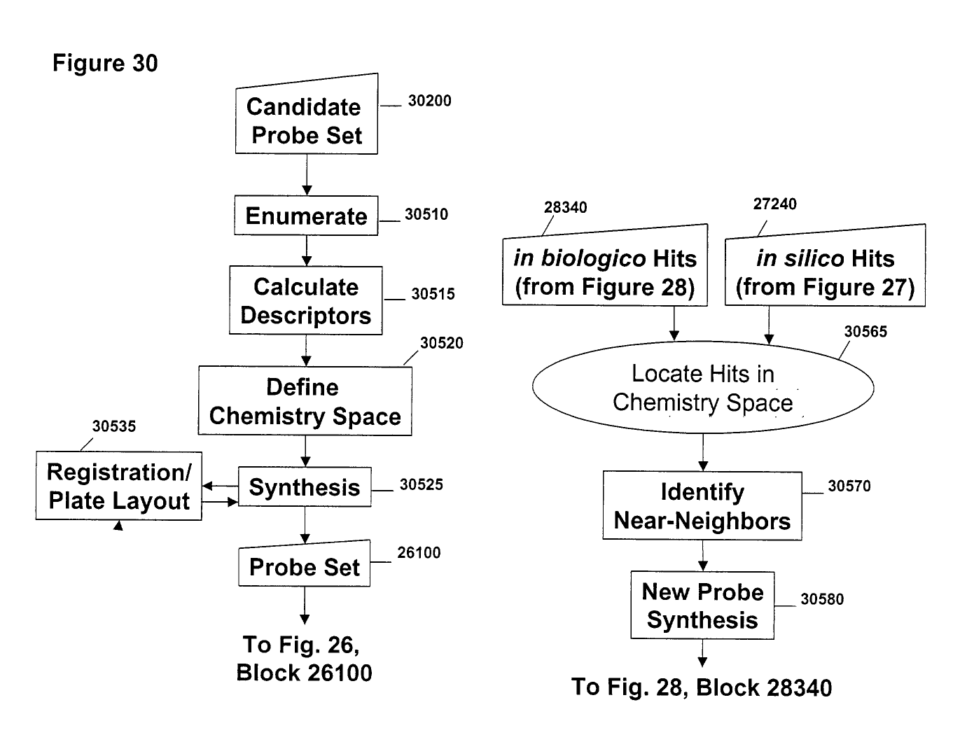

FIG. 28 is a more detailed block diagram of the in biologico screening method referred to in FIG. 26 as block 26300. In FIG. 28, the Probe Set (261000) synthesized in FIG. 30 or a suitable subset of the Probe Set (28310) is screened (28330) against one or more biological targets. Binding constants, association constants, IC₅₀ values, or other appropriate measurements of biological activity are obtained and recorded in a database wherein the data is associated with the probe registration ID. The in biologico probe hits, defined as having a specific biological activity above a threshold, are selected (28340) and advanced as Development Candidates (265000). In addition, the in biologico probe hit list may be further processed according to either or both of the methods described in block diagrams in FIGS. 29 and 30.

In FIG. 30, the most active compound(s) is (are) examined for “closeness” to neighbors in “Chemistry Space” which may not yet have been screened in biologico. The in biologico probe hits are located in “Chemistry Space” (30565), and the nearest neighbors to the in biologico probe are identified (30570). Probes “close” in “Chemistry Space” (or other property space) to the in biologico probe hits are selected for subsequent testing (28310). The positions of compounds in the “Chemistry Space” define their similarity: compounds that are close in “Chemistry Space” to a hit are similar, and therefore are more likely to show biological activity than compounds that are remotely located in “Chemistry Space.” In the event that a “neighbor” probe has not been synthesized, the probe may synthesized and registered (30580).

Another approach to describe the degree of diversity (and therefore of similarity) between two probes, is to calculate the pairwise Tanimoto coefficients between “fingerprints” of the probes. Fingerprints are bit-strings (sequences of 1's and 0's) representing the presence or absence of various substructural features within the molecular structure of a probe. Each bit represents an axis in a multi-dimensional chemistry space. Fingerprints typically consist of hundreds or even thousands of bits. Thus, a 1000-bit fingerprint represents a point in a 1000-dimensional chemistry space. Similar compounds are expected to be located near each other in this space; dissimilar or “diverse” compounds are expected to be further apart from each other.

The fingerprints of the probes can be calculated using computer programs available from vendors such as but not limited to MDL Information Systems (San Leandro, Calif.) (ISIS fingerprints) or Daylight Chemical Information Systems Inc. (Mission Viejo, Calif.) (Daylight fingerprints). Other fingerprint definitions have also been described in the literature and may be utilized in a similar manner.

The Tanimoto coefficient between two fingerprints is calculated as Tc=[Nab]/[Na+Nb−Nab], where Na is the number of bits set “on” in molecule a; Nb the number of bits set “on” in molecule b, and Nab the number of bits set “on” in common to both molecules. Two completely identical molecules will have a Tc of 1. Two compounds will be described as similar if they have a Tanimoto coefficient greater than a cutoff value. This value depends on the fingerprints used, but is usually 0.8 or above. Computer programs developed described herein allow the selection of probes within a set of probes (261000 or 302000) that have a Tc above a user-defined cutoff with respect to in silico (27240) or in biologico (28340) screening hits.

An alternate method for identifying near neighbors of the hits obtained in silico or in biologico involves the use of the Tanimoto coefficient (Tc) to locate probes near to a “hit” in a chemistry space. This allows one to select the probes within a user selected cutoff distance from a probe hit in a chemistry space.

TABLE 6
Nonexhaustive List of Molecular Descriptors Calculated for Probes

Multigraph information content indices:
Information-content descriptors:
Bonding Information Content.
Structural Information Content.
Information Content.
Complementary Information Content.
Information of atomic composition index.
Information indices based on distance and edge matrices:
Vertex distance/magnitude.
Vertex adjacency/magnitude.
Edge adjacency/magnitude.
Edge distance/magnitude.
Structural and thermodynamic descriptors:
Molecular weight.
Number of rotatable bonds (Ignoring all terminal hydrogen atoms).
Number of hydrogen-bond acceptors.
Number of hydrogen-bond donors.
log of the octanol/water partition coefficient
Topological descriptors:
Balaban indices.
Kappa indices.
Wiener index
Zagreb index
Kier & Hall subgraph count index
Zeroeth order.
First order.
Second order.
Third order (path, cluster and ring).
Kier & Hall molecular connectivity index
Zeroeth order.
First order.
Second order.
Third order (path, cluster and ring).
Kier & Hall valence-modified connectivity index.
Zeroeth order.
First order.
Second order.
Third order (path, cluster and ring).
Kier and Hall E-state descriptors:
Forty-two Kier and Hall electrotopological descriptors (“E-state
fingerprints”) are
included in the calculations.
Pearlman “BCUT” descriptors:
Descriptors related to hydrogen bonding, charge distribution,
polarizability, accounting
for atomic accessibility and three-dimensional structure

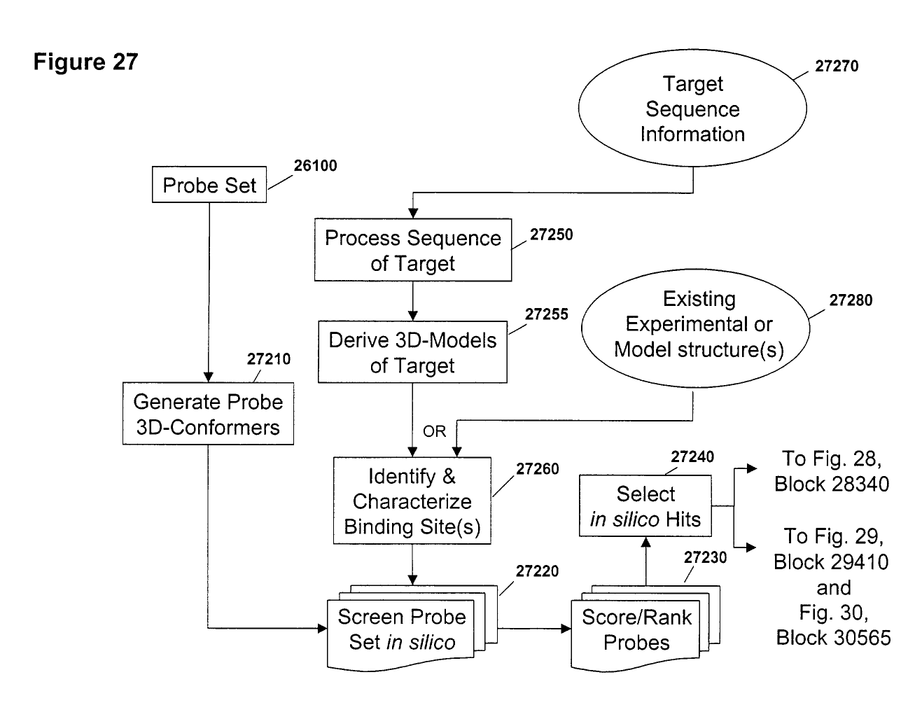

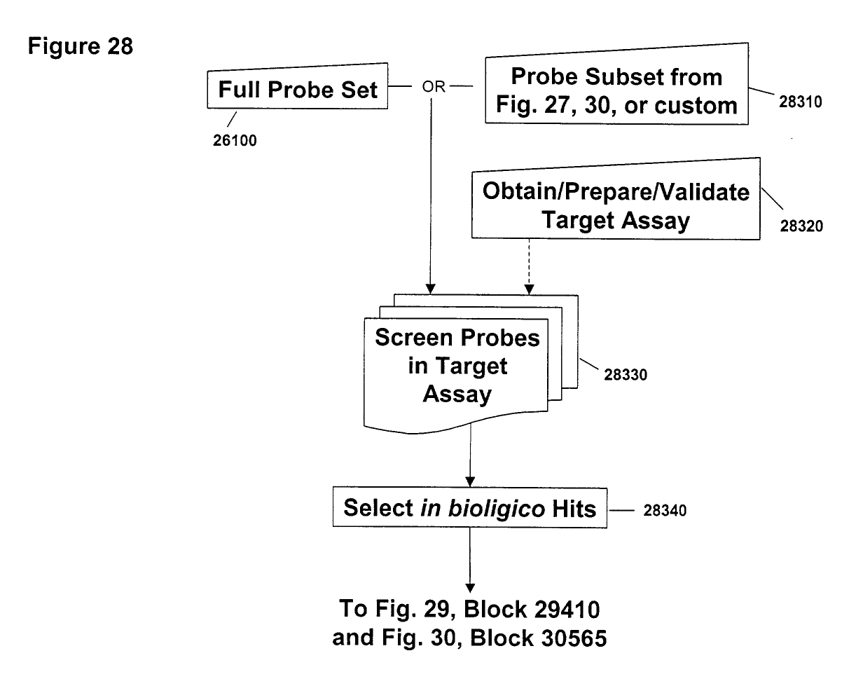

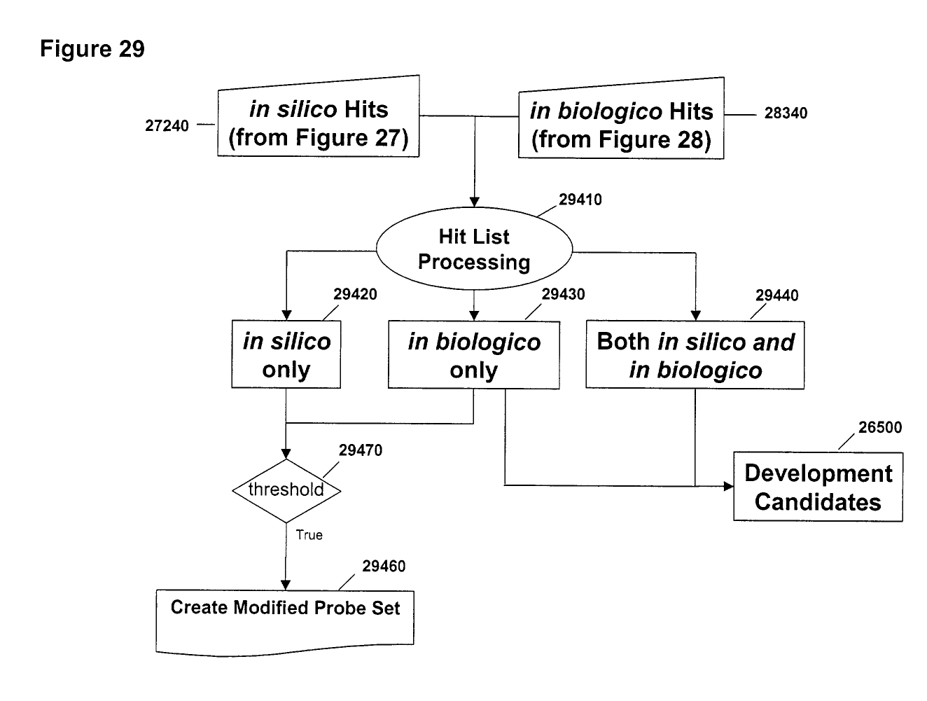

Referring again to FIG. 26, an embodiment of the second aspect provides a computer-based (in silico) screening method (26200) for using the Probe Set (261000) in the discovery of Development Candidates (265000) against one or more therapeutic targets in drug discovery. The in silico screening method is detailed in the block diagram in FIG. 27. Additional detailed aspects of the this in silico screening method are detailed below.

If the molecular target is a protein, the target's sequence (27270) is compared to sequences of proteins of known three-dimensional structures. Multiple sequence alignment (27250) may be performed using sequence threading algorithms, other methods and algorithms known by those skilled in the art, or using methods such as those described below. Sequence alignment attempts to align several protein sequences such that regions of structural and/or functional similarity are identified and highlighted. Different matrices are used to perform such alignment, such as but not limited to the freely available engines ClustalW (Jeanmougin, F., Thompson, J. D., Gouy, M., Higgins, D. G. and Gibson, T. J. (1998) Trends Biochem Sci, 23, 403-5) or MatchBox (Depiereux, E., Baudoux, G., Briffeuil, P., Reginster, I., De Bolle, X., Vinals, C., Feytmans, E.(1997) Comput. Appl. Biosci. 13(3) 249-256). Databases of protein sequences can be used to identify protein sequences that possess some (user defined) degree of similarity with the protein target of unknown structure, such as but not limited to the freely available internet-based programs FASTA or BLAST. Commercially available computer programs, such as but not limited to MOE (Chemical Computing Group Inc, Montreal, Canada), or Modeler© (Andrej Sali, Rockefeller University, New York, N.Y., http://guitar.rockefeller.edu/modeller/modeller.html) can perform database searches and sequence alignments as an integrated process. Emphasis can be put on finding similarity among sequences that are known to be associated to certain biological functions, in order to predict not only the structure but also the possible function of the target protein.

Once a protein of known three-dimensional structure (template) has been identified as homologous to the target protein sequence, one or more three-dimensional structures of the target protein may be built (27255) based on the three-dimensional structure of the template using homology modeling techniques known to one skilled in the art.

In homology modeling, one attempts to develop models of an unknown protein from homologous proteins. These proteins will have some measure of sequence similarity and a conservation of folds among the homologues. It is hypothesized that for a set of proteins to be homologous, their three-dimensional structures are conserved to a greater extent than their sequences. This observation has been used to generate models of proteins from homologues with very low sequence similarities.

The steps to creating a homology model may be summarized as follows:

a. Identifying homologous proteins and determine the extent of their sequence similarity with one another and the unknown;

b. aligning the sequences

c. identifying structurally conserved and structurally variable regions

d. generating coordinates for core (structurally conserved) residues of the unknown structure from those of the known structure(s)

e. generating conformations for the loops (structurally variable) in the unknown structure

f. building the side-chain conformations

g. refining and evaluate the unknown structure

Several commercially available computer programs, such as but not limited to MOE (Chemical Computing Group Inc, Montreal, Canada), Insight-II® (Accelrys, Inc., San Diego, Calif.), Homology (Accelrys, San Diego, Calif.), and Composer™ (Tripos, Inc., St. Louis, Mo.) can be used to perform homology modeling. Threading algorithms are described in Godzik A, Skolnick J, Kolinski A. 1992, J Mol Biol 227:227-238 and in other literature. Commercially available threading software includes MatchMaker™ (Tripos, Inc., St. Louis, Mo.).

Several templates can be identified and used to derive one or more three-dimensional structures for the target protein. These different three-dimensional structures for the target protein may be used in a parallel fashion in the in silico screening process (27220) described below. Once three-dimensional structure(s) of the target protein(s) is (are) obtained (27255), computer programs are used to predict possible drug association sites (27260) in these three-dimensional structures.

Several computer programs can be used to identify possible association site(s) (27260), such as but not limited to the shape-based approach from “Cerius²® LigandFit” (Accelrys Inc, San Diego, Calif.), or the mixed size/properties approach from “MOE Site Finder” (Chemical Computing Group Inc., Montreal, Canada).

In the case of shape-based methods, the sites are defined based on the shape of the target protein. Within the volume of the target protein, a flood-filling algorithm is employed to search unoccupied, connected grid points, which form the cavities (sites). All sites detected can be browsed according to their size, and a user defined size cutoff eliminates sites smaller than the specified size. Mixed shape/properties sites are defined as connections of hydrophobic and hydrophilic spheres in contact with mainly hydrophobic regions of the target protein. The sites are ranked according to the number of hydrophobic contacts made with the receptor, therefore including information about the chemistry of the receptor in addition to its geometry.

Possible association sites, once identified using the one or more of the methods described above, are used to perform in silico screening (27220) of the probes (261000) or a suitable subset. The screening may be separated into two parts: (i) the docking and (ii) the scoring/ranking (27230) of probes. Both processes may be performed in parallel.

The probe set (261000) is treated sequentially and can be processed in parallel. For each probe, a user-defined number of three-dimensional conformers (27210) are generated by rotating the bonds of the probe. Typically, one thousand conformers are generated for each probe through a Monte-Carlo procedure. Other conformational search procedures such as but not limited to simulated annealing, knowledge-based search, systematic conformational search, and others known to one skilled in the art may be employed.

Each of these conformers is docked in the association site (27220) using computational methods such as, but not limited to, those described below. One such method employs the alignment of the non mass-weighted three-dimensional principal moments of inertia of the probes with that of the association site. The conformer is shifted in its best alignment orientation in the association site to improve the docking. The orientation of the conformer that optimizes the fit between the principal moments of inertia of the probe and the association site is saved to disk, the docking score is calculated (27230) as described below for that conformer and the docking process repeats with a new conformer of the same probe. Computer programs such as but not limited to “Cerius²® Ligand Fit” from Accelrys Inc. (San Diego, Calif.), DOCK, (University of California at San Francisco, UCSF), F.R.E.D. (OpenEye Scientific Software, Santa Fe, N.M.) and others can be used for the docking procedure.

After docking of the conformers as described above, a score is calculated (27230) for each of the probe's conformers in the association site. Several scoring functions can be used for that purpose. One such scoring function is described below.

In this approach, ΔE, the non-bonded interactions between the probe and the target protein, is calculated from the coulombic and van der Waals terms of an empirical potential energy function. ΔE is defined theoretically as: ΔE=E(complex)−[E(Probe)+E(protein)], where E(complex) is the potential energy of the (protein+docked probe) complex, E(probe) is the internal potential energy of the probe in its docked conformation, and E(protein) is the potential energy of the protein alone, i.e., with no probe docked. The protein may be kept fixed during the docking procedure and therefore E(protein) would need to be estimated only once. E(complex) can be calculated either from an explicit description of all the atoms of the protein, or from a grid representation of the association site, the latter being faster in the case where a large number of compounds is to be screened. This approach includes explicitly the calculation of van der Waals interactions between atoms using a Lennard-Jones function. This scoring function favors probes that are small (minimizing van der Waals clashes) and that have large charge-charge interactions between the probe and the receptor (maximizing the electrostatic interactions). The scoring function also disfavors probes and/or conformers that exhibit large van der Waals clashes between the probes and the receptor.

Other scoring functions may be used. These include, but are not limited to LUDI (Böhm, H. J. J. Comp. Aided Molec. Design, 8, 243-256 (1994)); PLP (piecewise linear potential, Gehlhaar et al, Chem. Bio., 2, 317-324 (1995); DOCK (Meng, E. C., Shoichet, B. K., and Kuntz, I. D. J. Comp. Chem. 1992 13: 505-524); and Poisson-Boltzman (Honig, B. et al, Science, 268, 1144-9 (1995).

Some of the above scoring functions, are implemented in several commercially available software packages such as but not limited to Cerius²® from Accelrys, Inc. (San Diego, Calif.) and MOE (Chemical Computing Group Inc., Montreal, Canada)

This docking (27220)/scoring (27230) process is done independently for each probe. The score calculated for one probe's conformers does not depend on the calculations for other probes or conformers. Therefore, this process is highly scalable, and can be distributed among any number of computers that have the required programs. For two computers for instance, the probes can be divided in two groups that will be docked and scored in parallel. Ultimately, each probe could be docked and scored individually on one processor. Massively parallel computer architecture could then be used to linearly improve the efficiency of the process. The docking (27220)/scoring (27230) approaches described above can be used to perform massive throughput in silico screening (27220) of compounds.

Each combination of protein structure and probe conformer may be rank ordered based on the scores calculated as described above. In the present embodiment, the two highest-ranking protein structure-probe conformer complexes (based on their scores) are saved for each probe. Optionally, several scoring functions (as described above) may also be utilized yielding a set of scores for each protein structure-probe conformer complex and a consensus score and rank order determined from the set of scores and utilized for the final ranking. Other methods for rank ordering, known to one skilled in the art may also be employed.

The above rank ordered probe list is used to select a subset of probes from the entire probe set to be considered for in biologico screening. This subset may be determined using one or more of the following protocols or other protocols known to one skilled in the art.

a. A user specified percentage of the rank ordered probe list

b. The first “N” members of the rank ordered probe list, where “N” is the number of probes requested by the user

c. The sample plates containing the probes selected in either protocol a or b

d. The first “M” sample plates containing the probes selected in either protocol a or b where “M” is user specified

e. Optionally, the nearest neighbors of the probes selected in either protocol a or b, where the neighbor selection criteria is user specified (the nearest neighbors of the probes are themselves probes)

f. The sample plates containing the probes selected in protocol e.

g. The first “M” sample plates containing the probes selected in protocol f, where “M” is user specified.

h. A diverse subset of the high ranking probes

The corresponding sample plates containing the probe subset from protocol h

In the above protocols, the user specified percentage may typically range from 10 to 60 percent. More preferably between 10 and 50 percent. The number of samples or plates designated as “N” or “M” is dependent on the specific in biologico assay, but typically ranges from 1,000 to 100,000 compounds or 10 to 1,000 plates respectively.

The rank ordered probe list (27240 or 28310) obtained as described above is subjected to in biologico screening (28330) against the target(s). Optionally, the entire probe set (261000), or a diverse subset (selected using methods known to one skilled in the art) of the entire probe set, or other means of selection (known to one skilled in the art) of a custom subset may be subjected to in biologico screening (28330) against the target(s). The biological activity measured in this screening (described above) is used in the selection of a subset of probes based on a user-selected level of biological activity measured in the in biologico screening. This subset of probes is defined as the list of in biologico hits (28340).

Optionally, the nearest neighbors of the in biologico hits selected above may be determined (30570) using methods for neighbor list selection as described above and subjected to further in biologico screening (28330). In the case where one or more near neighbor probe(s) have not been synthesized, they may be synthesized (30580).

As illustrated in FIG. 29, the lists of in silico and in biologico hits are divided into three categories (29410): hits found only in silico (29420), hits found only in biologico (29430), and hits found both in silico and in biologico (29440). The members of category 29420 are in silico hits that are not identified as hits in biologico. Conversely, members of category 29430 are in biologico hits that are not identified as in silico hits. The members of category 29440 are in silico hits that are also identified as in biologico hits. A population of category 29440 serves to validate the entire process and especially the in silico protocols. In practice, a population of 10 percent or more of the selected in silico hits (27240) is considered to be a strong validation.

The hits populating categories 29440 and 29430 are considered Development Candidates (265000) and may optionally utilized in the generation of more complex probes and included in a Candidate Probe Set (302000).

Optionally, the relative populations of categories 29420, 29430, and 29440 may be reviewed to determine if there is a need to refine (460) the in silico protocols described FIG. 27. In practice, if category 29420 contains more than 50 to 60 percent of the in silico hits (27240) (the threshold level, 29470), refinement is recommended. Likewise, if category 29430 is populated (the threshold level, 29470), refinement is also recommended.

In the case where neighbors of the in silico hits and/or the plates containing the in silico hits are subjected to in biologico screening, the potential arises wherein some of the in biologico hits (28340) may not have been selected in the in silico screening (27240). In this case, category 29430 may be populated.

Description of Prediction Method

As set forth above, methods of the present invention may utilize computer software to perform in one or more of the steps in silico. A detailed description of embodiments of computer systems and software suitable for use in the present invention is set forth in U.S. provisional patent application Serial No. ______, Attorney Docket Number 41305.272624 (TTP2002-03), filed on Apr. 10, 2002, the disclosure of which is herein incorporated by reference. Details relating to embodiment of the software are also set forth below.

Embodiments of this system provide a system and method for integrated computer-aided molecular discovery. In an embodiment of this system, the user is provided with an integrated user interface that provides the user with the capabilities of a broad array of components, such as calculation engines, from a variety of commercial and custom applications. The calculations are model independent. Therefore, implementation of new calculation methods is very simple. An embodiment of this system is capable of utilizing many different computer platforms, including UNIX and LINUX, and allows load balancing for heterogeneous clusters.

Since the system is able to utilize a variety of applications and components, the system is extremely flexible. The user and/or system administrator chooses the components to use for performing each task or sub-task.

Also, an embodiment of this system provides enormous benefits in terms of scalability. Each of the processes of the system may be executed in a parallel manner utilizing a heterogeneous cluster of networked computers. These computers may be different in terms of both hardware and operating system from one another. The system determines which nodes of the cluster are available and offloads a portion of the processing for any step to the underutilized node.

The flexibility of an embodiment of this system provides advantages to many different members of the computer-aided molecular discovery market. For example, a laboratory or other organization can increase the efficiency of its scientists, decrease the underutilization of its computing resources, and easily integrate the variety of applications necessary to perform discovery. Also, by utilizing an embodiment of this system, software developers are able to create custom or additional commercial components that can be easily integrated with highly popular commercial applications. An embodiment of this system also provides great flexibility to software sellers. The sellers can tout the benefit of multiple commercial applications, which can be integrated under a single easy-to-use interface. System integrators also benefit from utilizing an embodiment of this system. The process of integration becomes much simpler because the integrator is not forced to write various separate applications to integrate each of the various components a molecular discovery lab utilizes.

Further details and advantages of the present system are set forth below.

Embodiments of this system provide systems and method for performing computer-aided molecular discovery within an integrated user interface, utilizing a variety of third-party and custom components from a variety of applications. One embodiment provides horizontal integration, utilizing various application components to perform a step in a molecular discovery process, such as structure alignment. Another embodiment utilizes various application components to perform multiple steps in a molecular discovery process, such as the steps of detecting a set of potential binding sites and then eliminating obviously wrong sites from the set. Yet another embodiment incorporates both horizontal and vertical integration. An embodiment of this system may utilize application components that execute on any hardware/operating system platform and may provide the ability to execute components in a parallel manner. In addition, an embodiment of this system may execute any portion of the discovery process in an iterative manner in order to attempt to enhance the results and/or simplify the process for the user.

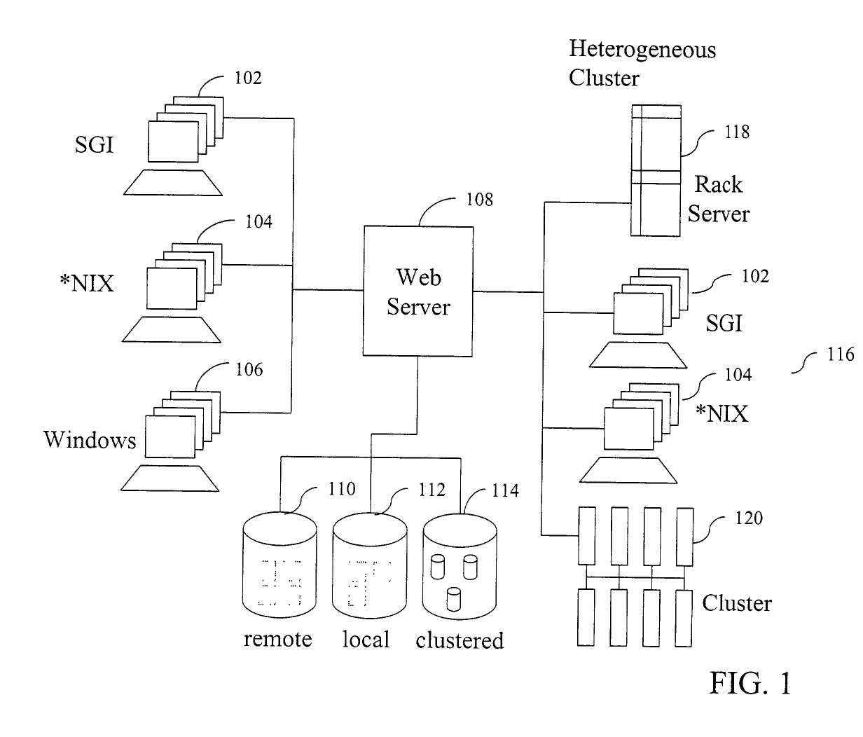

FIG. 1 illustrates an exemplary environment for an embodiment of this system utilizing both horizontal and vertical integration as well as parallel execution. In the embodiment shown, user workstation displays user interface. The workstation may provide a command line interface, a graphical user interface, or any other interface with which a user may interact. A variety of hardware and operating system combinations may support the interface, including Silicon Graphics (SGI) workstations 102, Unix and Linux (*NIX) workstations 104, and workstations capable of supporting one of the many flavors of Microsoft Windows 106.

In the embodiment shown, the user workstation 102-106 accesses a web server 108. The web server generates the user interface, accepts parameters from the user interface, and inserts those parameters into a database to, among other purposes, initiate program flow in the application as is discussed in detail below. In order to present the user interface and provide various other features, the web server 108 accesses a variety of databases, including remote databases 110 and local databases 112, such as control or administrative databases. These databases may include corporate or commercial databases. These databases may be stand-alone databases on a single database server, such as those exemplified by databases 102 and 104, or these databases may include clustered databases 114.

In one embodiment of this system, the web server 108 uses CGI (Common Gateway Interface), XML, and standard data access modules to provide the user interface and process user requests. To initiate jobs, the web server 108 also accesses a computer that executes an application component, such as a server or other member of heterogeneous cluster 116.

An application component is a program or portion of a program that can be executed in some manner by the user interface. The component may be an entire commercial application, a single module from a commercial application, a custom component, or some other executable code.

By utilizing variety of application components to perform calculations, an embodiment of this system operates independently from the constraints of any one commercial application. In addition, it is relatively simple to implement new calculation methods. In addition, an embodiment of this system is not limited to operation on a single hardware and software platform. The components may be executed from any platform on which they are designed to function, including *NIX, Microsoft Windows, and other platforms. Not only does this platform independence increase the flexibility of a system according to this system, it also increases the scalability. An embodiment of this system is capable of balancing the processing load for performing calculations across heterogeneous clusters, such as heterogeneous cluster 116.

It is important to note that some commercial applications are only capable of running on a limited number of different hardware and operating system environments. An embodiment of this system does not seek to provide a means for the application to run on hardware or operating systems on which it is not designed to run, but rather to allow the user to control the execution of a component or components of the commercial application from an integrated user interface.

In the embodiment shown in FIG. 1, rather than accessing a single server, the web server 108 access a heterogeneous cluster 116 of computers that execute the application component specified by the web server 108. The heterogeneous cluster may include any type and number of computers, both workstations and servers. In the embodiment shown, the heterogeneous cluster includes a rack server 118, the SGI 102 and *NIX 104 workstations, which also may display the user interface, and a server cluster 120. An example of the manner in which the web server 108 utilizes the heterogeneous cluster 116 is presented in detail below.

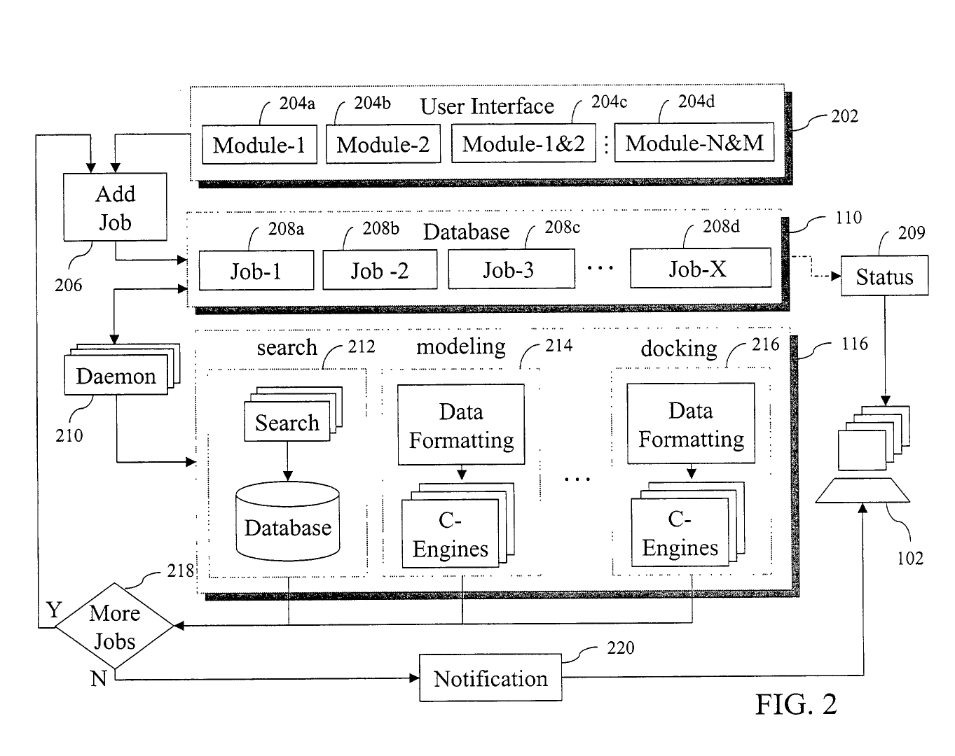

To provide maximum flexibility and scalability, one embodiment of this system utilizes the multi-layer application framework illustrated in FIG. 2 to process requests from the user interface. FIG. 2 will now be described with reference to the exemplary environment shown in FIG. 1. However, the environment shown in FIG. 1 is merely exemplary; the application framework shown in FIG. 2 is in no way limited to operating within the environment shown in FIG. 1.

The application framework shown in FIG. 2 includes a user interface 202 executing on a user workstation, such as an SGI workstation 102. The user interface includes modules 204a-d. The modules 204a-d may be presented individually in the user interface 202, such as with module-1 204a and module 2 204b, or be presented in combination 204c,d. When the user specifies a request in the user interface 102, the embodiment shown in FIG. 2 executes an “Add Job” process 206. The “Add Job” process 206 creates database records in a table in a database, such as local database 110. For each module 204a-d, multiple “Add Job” processes 206 may execute, creating multiple jobs 208. In addition, in a multi-user environment, each user interface creates independent jobs 208. As jobs 208 are created, a “Status” process 209 alerts the user via user workstation 102 or via other means when changes in status of the particular job 208 occur.

In the embodiment shown in FIG. 2, a background process or daemon 210 is activated when jobs 208 are created in the database 110. The daemon 210 executes the code necessary to create processes within the heterogeneous network 116 corresponding the job 208. The daemon 210 may be a background process in a *nix or other environment or may exist as a screen saver in a Microsoft Windows environment.

A hypothetical search provides an example of how the process shown in FIG. 2 might work. A user wishes to search for a protein or nucleic acid structure, so the user enters search criteria in a module 204 in the user interface 202. The search request causes the “Add Job” process 206 to add a job 208 to database 110. The job 208 includes various parameters, including, for example, the sequence, user name, search engines to utilize, and others. The daemon 210 evaluates these parameters and submits the job 208 to one or more application components, search 212 in FIG. 2, for processing. The search component 212 performs the necessary processing and then determines whether additional jobs must be performed 218. If so, the “Add Job” process 206 is again executed. If not, a “Notification” process 220 notifies the user that the process is complete 102. In the example, notification occurs via user workstation 102. However, notification may occur using a variety of methods, including fax, instant messaging, automated phone messaging, or any other means capable of providing notification to a user. As is shown in FIG. 2, an embodiment of this system may utilize various application components, including modeling 214 and docking 216 components.

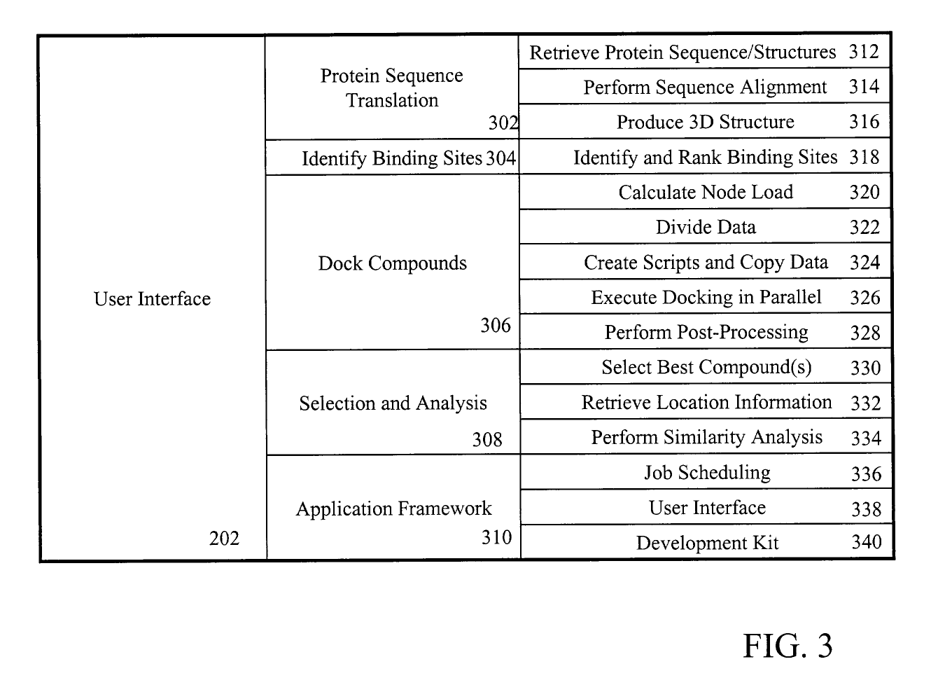

FIG. 3 illustrates an embodiment of this system as a 3-level structure of interrelated modules. The embodiment shown utilizes both horizontal and vertical integration of various application components as well as the capability of executing various components in a parallel manner. The embodiment shown integrates visualization, simulation and application development under the control of a comprehensive user interface 202. The user interface 202 may be a command-line interface, a browser-based interface, or other GUI. The scientific aspects of the embodiment shown include four broad high-level modules 302-308, which include twelve lower-level modules 312-334. In addition, the embodiment shown also includes an application framework module 310, which includes three lower-level modules 336-340. It is important to note that an embodiment of this system need not include all of the modules shown in FIG. 3. The structure shown is merely illustrative of one embodiment of this system.

An embodiment of this system delivers high throughput computer-aided molecular discovery by coupling computational chemistry with high throughput screening. Custom methodology modules can be developed by utilizing tools currently available in the software industry or created independently for data analysis, mining, and visualization. The system may utilize commands, macros, and scripts, allowing applications to be customized by end-users throughout an organization.

For example, one embodiment of this system utilizes the following commercially available software packages: Cerius² (C2) (Accelrys Inc, San Diego, Calif.) and MOE (Chemical Computing Group Inc., Montreal, Canada) as calculation engines in some of its modules. However, an embodiment of this system is not limited to those or other commercially-available applications. The modular structure of an embodiment allows the implementation of other calculation engines.

The five first-level modules include: (1) a Protein Sequence Translation module 302, which automates the translation of a protein sequence to three-dimensional structure(s) in an efficient manner (Protein is used only as an example in this specification; any target may be sequenced and ranked in an embodiment of this system); (2) an Identify Binding Sites module 304, which automates the detection of the desired binding sites, calculates their physico-chemical properties and may perform other functions specified by a user, such as eliminates incorrect sites based; (3) a Dock Compounds module 306, which automates the docking of a large number of compounds in an efficient fashion utilizing parallel approaches to split the process among different processors based on protein structures and protein sites and ranks them utilizing a number of scoring functions; (4) a Selection and Analysis module 308, which selects high ranking probes or compounds (Probe and compound are used interchangeably throughout this specification as examples.) and submit queries to the Oracle and corporate databases to identify the plates they reside in, analyze them, perform identity, similarity and clustering checks, and rank them for in biologico screening by generating structure and site specific reports containing plate numbers, location, and the chemical structure of all their constituents; and (5) an Applications Framework module 310, which provides the user interface, job control, and parallel execution management in the embodiment shown in FIG. 3.

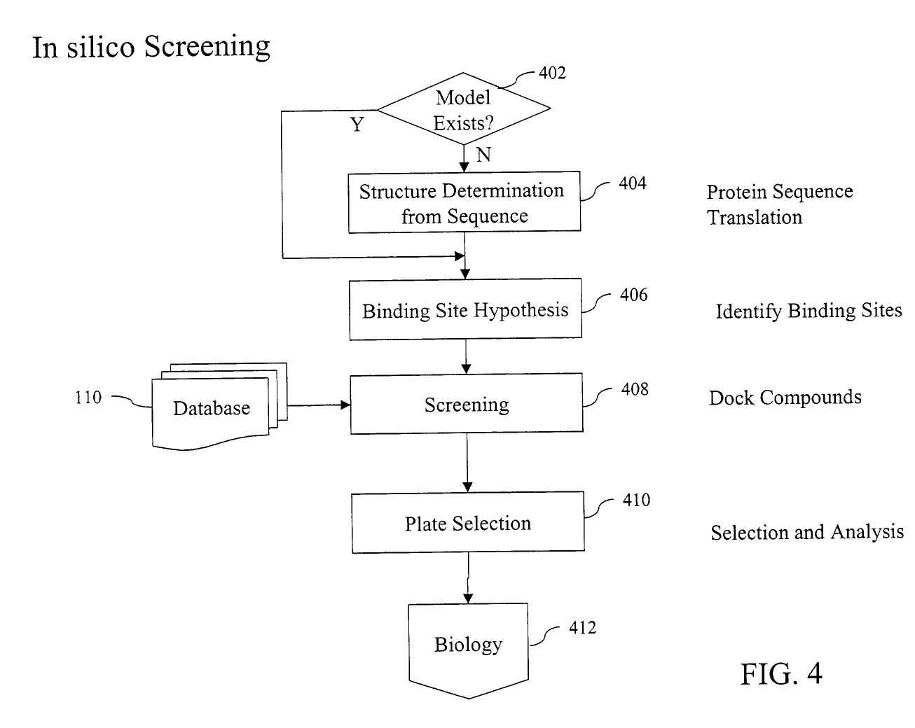

FIG. 4 illustrates the general process utilized by one embodiment of this system in reference to the high-level modules of FIG. 3. Also illustrated on FIG. 4 are exemplary calculation engines that may be applied to each step in the process. The Protein Sequence Translation module 302 first determines if the submitted sequence corresponds to an existing crystal structure or other experimentally determined three-dimensional structures 402. If not, the three-dimensional structure is determined from the sequence 404. The experimental structure(s) may be retrieved from a protein data bank (www.rcsb.org) or determined using a commercial product, such as but not limited to MOE or Insight II. Once the three-dimensional structure is determined, or if the crystal structure already exists, the process proceeds to the next step, the binding site hypothesis 406, which is performed by the Identify Binding Sites module 304. A commercial application, such as MOE, Dock, or Cerius2, may perform the binding site hypothesis step.

The next step in the general process is screening 408, a step performed by the Dock Compounds module 306. Commercial products, which may be used for this step in the process, include but are not limited to MOE, C², and Schrödinger. This step in the process also retrieves data from a database, such as local database 110. The final step in the in silico process is plate selection 410, which is accomplished by the Selection and Analysis module 308. In one embodiment of this system, plate selection is accomplished via custom code. Once the in silico process steps are complete, the compound(s) proceed to in biologico screening 412.

Each of the modules of an embodiment of this system will now be described in detail with reference to FIG. 3. The first high-level module is the Protein Sequence Translation module 302. The goal of this module 302 is to automate the creation of a three-dimensional protein model from a protein sequence. Several databases may be used in a concerted fashion to optimize the structural diversity and relevance of the final three-dimensional model that may be used for in silico screening, including commercial, public, and proprietary databases. This process is not aimed at substituting the scientist, but at performing rapid and automated tasks in a way that may not require user's intervention. In one embodiment of this system, the module 302 generates a series of log files. The scientist has the ability to examine the log files to perform quality control checks and to identify any potential issues and to re-run specific job or jobs with modifications when desired.

The embodiment illustrated in FIG. 3 is merely exemplary. Other embodiments of this system include subsets of the modules shown or additional components. For example, one embodiment of this system provides links to an integrated data analysis solution. In such an embodiment, information from in silico and in biologico screening is combined in an integrated user interface. Such an embodiment is described in Attorney Docket # 41305-272623, which was filed herewith and is hereby incorporated by reference.

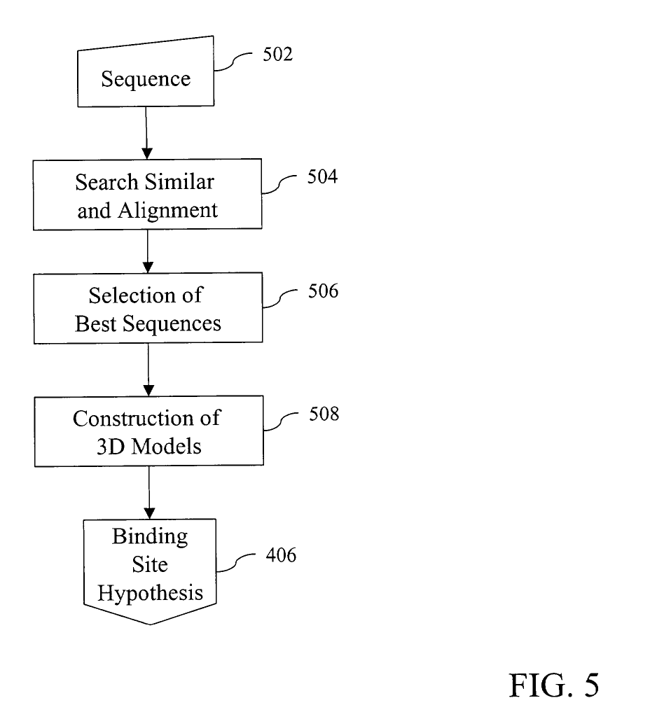

FIG. 5 illustrates the process implemented by the Protein Sequence Translation module 302. The module 302 first accepts the sequence as an input 502. The module 302 searches for similar sequences commercial and/or proprietary databases and performs multi-sequence alignment 504.

Sequence alignment attempts to align several protein sequences such that regions of structural and/or functional similarity are identified and highlighted. Different matrices are used to perform such alignment, such as but not limited to the freely available engines ClustalW (Jeanmougin, F., Thompson, J. D., Gouy, M., Higgins, D. G. and Gibson, T. J., Trends Biochem Sci, 23, 403-5 (1998)) or MatchBox (Depiereux, E., Baudoux, G., Briffeuil, P., Reginster, I., De Bolle, X., Vinals, C., Feytmans, E., Comput Appl. Biosci. 13(3) 249-256 (1997)). Databases of protein sequences can be used to identify protein sequences that possess some (user defined) degree of similarity with the protein target of unknown structure, such as but not limited to the freely available internet-based programs FASTA (http://www.ebi.ac.uk/fasta3/) or BLAST (http://www.ncbi.nlm.nih.gov/BLAST/).

Also, commercially available computer programs, such as but not limited to MOE (Chemical Computing Group Inc, Montreal, Canada), Homology (Accelrys Inc., San Diego, Calif.), and Composer™ (Tripos, Inc., St. Louis, Mo.) can perform database searches of the application's proprietary database and sequence alignments as an integrated process. Emphasis can be put on finding similarity among sequences that are known to be associated to certain biological functions, in order to predict not only the structure but also the possible function of the target protein.

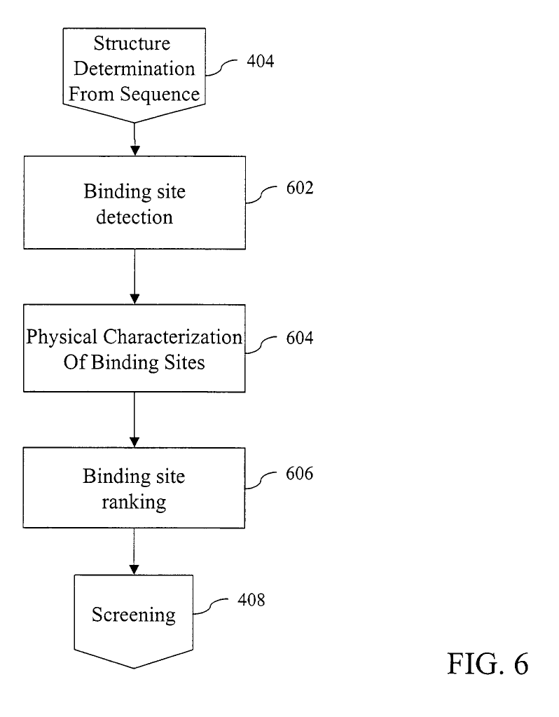

The module 302 next selects the highly homologous sequences 506 with known three-dimensional structures and constructs three-dimensional models 508 (homology models). Once construction of the three-dimensional models is complete, the process proceeds to the binding site hypothesis process 406 described in FIG. 6.

The process illustrated in FIG. 6 begins with the three-dimensional structures output by the Structure Determination from Sequence process 404. These three-dimensional structures are used for binding and/or association site(s) detection 602 (referred to herein as “binding sites”). Once the binding site detection is complete, the binding sites are characterized physically 604. Then the binding sites are ranked 606 and a user-specified number of sites are used for subsequent in silico screening. The process then proceeds to screening 408.

Referring again to FIG. 3, the Protein Sequence Translation module 302 includes three lower-level modules: Retrieve Protein Sequence/Structures 312, Perform Sequence Alignment 314, and Produce 3D Structure 316. In the Retrieve Protein Sequence/Structures module 312, an embodiment of this system starts from a target sequence and retrieves protein structures that have structural/biological similarity with the target sequence. The module processes the target sequence through a search engine, such as BLAST or NCBI, to search for known protein(s) with similar sequence(s). This module 312 may utilize public sequence and three-dimensional structure databases. In one embodiment, the module 312 performs a search in a database, such as a protein data bank (PDB). In another embodiment of this system, the user may perform a keyword search. The keywords describe the biological nature of the protein. For example, kinases, GPCR are keywords that the user may specify. Other modules use the retrieved three-dimensional structures during processing. For example, in the embodiment shown, these three-dimensional protein structures are used to construct a homology model for the target.

Several commercially available computer programs, such as but not limited to MOE (Chemical Computing Group Inc, Montreal, Canada), Insight-II® (Accelrys, Inc., San Diego, Calif.), Modeler© (Andrej Sali, Rockefeller University, New York, N.Y. , http://guitar.rockefeller.edu/modeller/modeller.html) can be used to perform homology modeling. Threading algorithms are described in Godzik A, Skolnick J, Kolinski A., J. Mol. Biol., 227,227-238 (1992) and in other literature. Commercially available threading software includes MatchMaker™ (Tripos, Inc., St. Louis, Mo.).

The next module in the embodiment shown in FIG. 3 is the Perform Sequence Alignment module 314. This module accepts a sequence in a standard format, such as the FASTA format, and searches for proteins of similar sequence in the commercial and corporate databases (e.g. MOE). The module retrieves these three-dimensional protein structures as well as the three-dimensional protein structures from the previous module 312 and performs a sequence alignment on all of them. The aligned chains, including alignment scores, are passed to the subsequent module.

The Produce 3D Structure module 316 runs a homology model engine for the chain with the highest alignment score, and produces a three-dimensional model for the target sequence in PDB format. The user may modify the default values of the homology modeling process via user interface 202. The user may also perform quality control checks and other processes.

In the embodiment shown in FIG. 4, the Produce 3D Structure module 316 is the final lower-level module of the Protein Sequence Translation module 302. The next high-level module is the Identify Binding Sites module 304.

The Identify Binding Sites module 304 includes one lower-level module, the Identify and Rank Binding Sites module 318. This module 318 accepts the three-dimensional model for the target protein and processes it through one of the custom or commercial calculation engines, e.g., C². The module 318 uses the calculation engine to identify possible binding sites for the protein and ranks the binding sites by size, saving the first n binding sites (n specified by the user). These sites are then passed to a specified calculation engine or engines together with the protein information. The module 318 may utilize additional or other algorithms aimed at identifying possible sites as well.

In the case of shape-based methods, the sites are defined based on the shape of the target protein. Within the volume of the target protein, a flood-filling algorithm is employed to search unoccupied, connected grid points, which form the cavities (sites). All sites detected can be browsed according to their size, and a user defined size cutoff eliminates sites smaller than the specified size. Mixed shape/properties sites are defined as connections of hydrophobic and hydrophilic spheres in contact with complementary interacting regions of the target protein. The sites are ranked according to the number of hydrophobic contacts made with the receptor, thereby including information about the chemistry of the protein in addition to its geometry.

Once three-dimensional structure(s) of the target protein(s) is (are) obtained, computer programs are used to predict possible drug association sites in these three-dimensional structures. These results are used in the subsequent in silico screening process. The Dock Compounds module 306 performs this function and is the next high-level module illustrated in FIG. 4. In the embodiment shown, this module 306 uses docking engines in a parallel fashion to screen a library of compounds or a probe set and so on against protein models to predict compounds that have a higher binding affinity with the protein. Various scoring functions and combinations of scoring functions may then be utilized based on user preferences for scoring the docked protein . . . compound complex.

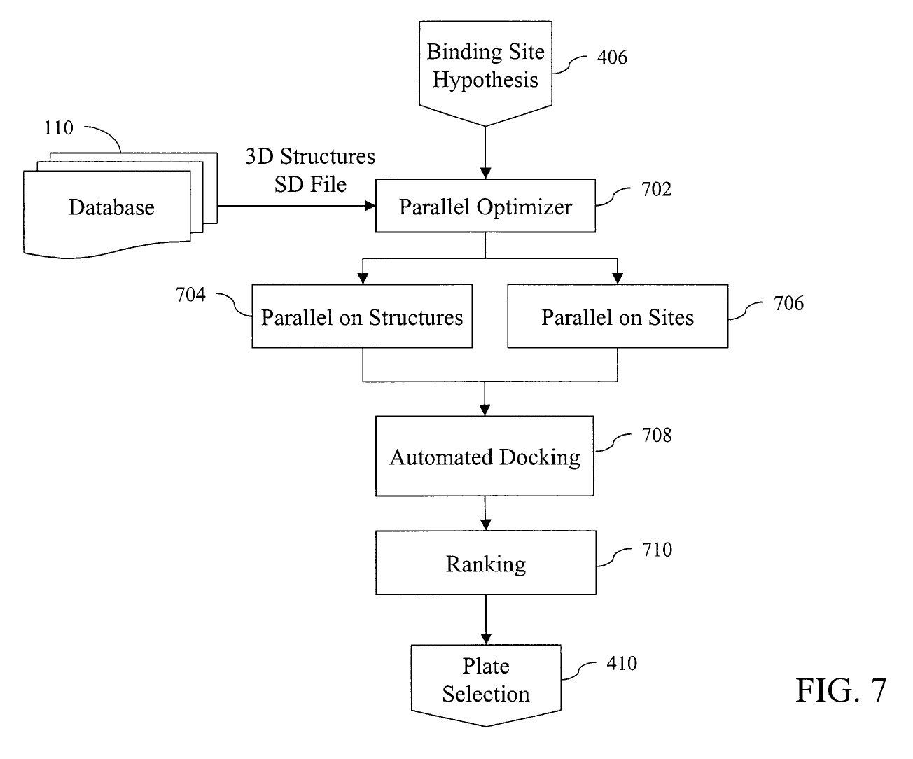

FIG. 7 illustrates the docking or screening process. The process begins with output from the binding site hypothesis process 406. The parallel optimizer extracts three-dimensional structures of the compounds or probes from a database, such as the local database 110, and prepares the data for parallel processing 702. In the embodiment shown, the data is processed in parallel for both compound structures 704 and identified binding sites 706. Next, automated docking is performed 708. Once the docking is complete, the compounds are ranked according to the scoring function value 710. The docking and ranking information is then output to the plate selection process 410.

As used herein, the term “probe” refers to a molecular framework encompassing association elements suitable for interaction with a macromolecular biological target, such as but not limited to DNA, RNA, peptides, and proteins, said proteins being those such as but not limited to enzymes and receptors.

As an example of the process shown in FIG. 7, in one embodiment, a probe set is treated sequentially and docking can be performed in parallel. For each probe, a user-defined number of conformers are generated by rotating the bonds of the probe. Typically, one thousand (1000) conformers are generated for each probe through a Monte-Carlo procedure. Other conformational search procedures such as but not limited to simulated annealing, knowledge-based search, systematic conformational search, and others known to one skilled in the art may be employed.

Each of these conformers is docked in an association site using computational methods such as but not limited to those described below. One such method employs the alignment of the non mass-weighted three-dimensional principal moments of inertia of the probes with that of the association site. The conformer is shifted in its best alignment orientation in the association site to improve the docking. The orientation of the conformer that optimizes the fit between the principal moments of inertia of the probe and the association site is saved to disk, the docking score is calculated as described below for that conformer and the docking process repeats with a new conformer of the same probe. Computer programs such as but not limited to “Cerius²® LigandFit” (Accelrys Inc., San Diego), DOCK (University of California at San Francisco), F.R.E.D. (OpenEye Scientific Software, Santa Fe, N.M.) and others may be used for the docking procedure.

After docking of the conformers, a score is calculated for each of the probe's conformers in the association site. Several scoring functions can be used for that purpose. One such scoring function is described below.

Non-bonded electrostatic interactions and volume exclusion calculations can be performed. In this approach, ΔE, the non-bonded interactions between the probe and the target protein, is calculated from the coulombic and van der Waals terms of an empirical potential energy function. ΔE is defined theoretically as: ΔE=E(complex)−[E(Probe)+E(protein)], where E(complex) is the potential energy of the (protein+docked probe) complex, E(probe) is the internal potential energy of the probe in its docked conformation, and E(protein) is the potential energy of the protein alone, i.e., with no probe docked. The protein may be kept fixed during the docking procedure and therefore E(protein) would need to be estimated only once. E(complex) can be calculated either from an explicit description of all the atoms of the protein, or from a grid representation of the association site, the latter being faster in the case where a large number of compounds is to be screened. This approach includes explicitly the calculation of van der Waals interactions between atoms using a Lennard-Jones function. This scoring function favors probes that are small (minimizing van der Waals clashes) and that have large charge-charge interactions between the probe and the protein (maximizing the electrostatic interactions). The scoring function also disfavors probes and/or conformers that exhibit large van der Waals clashes between the probes and the protein.

Other scoring functions may be used. These include, but are not limited to LUDI (Böhm, H. J. J. Comp. Aided Molec. Design, 8, 243-256 (1994)); PLP (piecewise linear potential, Gehlhaar et al, Chem. Bio., 2, 317-324 (1995); DOCK (Meng, E. C., Shoichet, B. K., and Kuntz, I. D., J. Comp. Chem. 13: 505-524 (1992)); and Poisson-Boltzman (Honig, B. et al, Science, 268, 1144-9 (1995)).

Some of the above scoring functions are implemented in some commercially available software packages such as but not limited to Cerius²® from Accelrys, Inc. (San Diego, Calif.) and MOE (Chemical Computing Group Inc., Montreal, Canada)

This docking/scoring process is done independently for each probe. The score calculated for one probe's conformers does not depend on the calculations for other probes. Therefore, this process is highly scalable, and can be distributed among any number of computers that have the required programs. For two computers for instance, the probes can be divided into two groups that will be docked and scored in parallel. Ultimately, each probe could be docked and scored individually on one processor. Massively parallel computer architecture could then be used to linearly improve the efficiency of the process. The docking/scoring approaches described above can be used to perform massive throughput in silico screening of compounds.

Referring again to FIG. 3, the Dock Compounds module 306 includes various lower-level or sub-modules. The first lower-level module is the Calculate Node Load module 320. This module 320 calculates the load for each node on a given heterogeneous cluster. The Divide Data module 322 then divides the data into several pieces to be processed independently on each node in a parallel fashion. For example, in the case of a large structure database (SD) file of chemical structures, the data is divided so that one member of the heterogeneous cluster 116 processes only a portion of the entire data set. Both of these modules 320 & 322 are pre-processing modules; they initiate and launch the tasks necessary to prepare data for docking.

The Create Scripts and Copy Data module 324 is also a pre-processing module. This module 324 (1) executes programs to create per node docking engine scripts and per node shell scripts that ensure data management and proper data allocation and (2) copies the data to the individual nodes. For example, the module 324 creates scripts that are used by later modules to process each portion of the SD file as divided in the preceding module. Once the file is divided into smaller files, each of the smaller files may be copied, such as by FTP (File Transfer Protocol) to the nodes in the heterogeneous cluster 116.

Once pre-processing is complete, the Execute Docking in Parallel module 326 executes. This module 326 executes the docking programs in parallel, i.e., at the same time on different members of the heterogeneous cluster 116. The module 326 may run on any member of the cluster 116, e.g., on the leading node. In particular, the module 326 executes and manages the execution of all the processes created by preceding modules 322-324 until they have all successfully completed.

In the embodiment shown in FIG. 3, once pre-processing and docking are complete 320-324, the Perform Post-Processing module 328 executes. This module 328 executes programs for post-processing, including programs that (1) combine the individual SD files after calculation of the screening score into one large final SD file, (2) clean up the data on the individual nodes, removing unused files, and (3) perform any additional per node calculation that might be necessary at this point. These modules 322-324 may utilize various formats. For example, to minimize the volume of network traffic utilized by the modules 322-324, the files may be transferred and processed in a compressed format, such as gzip.

The next high-level module in the embodiment shown is the Selection and Analysis module 308. This module includes three lower-level modules: a Select Best Compound(s) module 330, a Retrieve Location Information module 332, and a Perform Similarity Analysis module 334.

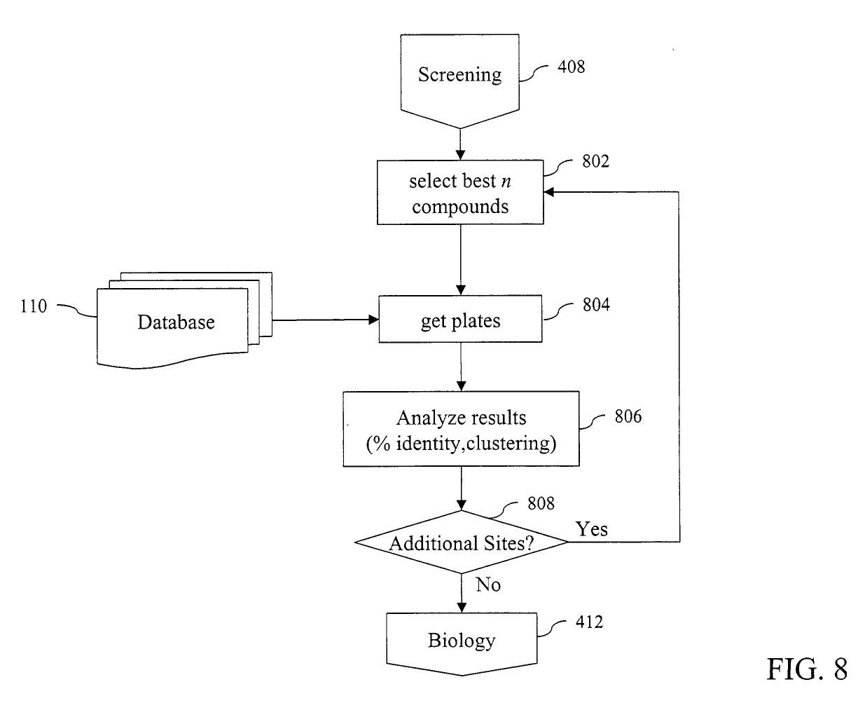

FIG. 8 illustrates the process implemented by the Selection and Analysis module 308. The process shown in FIG. 8 receives output from the screening process 408. Based on the ranking process, the best n compounds are selected (wherein n is specified by the user or otherwise) 802. Using identifying information, such as the compound or ID number, plate information is extracted from the database (110) 804. The plates are analyzed 806. For example, in one embodiment, additional wells from each plate that are not selected in the in silico ranking process, are analyzed to determine if similarities exist with the in silico ranked and selected compounds identified in the screening process. These compounds are optionally considered based on their similarity and closeness with the in silico ranked compounds. The process iterates for each site 808.

Instead of performing in bioligico screening on all of the in silico probe hits obtained, only high-ranking probes are used for subsequent screening activities. Although it may be more relevant to screen only those probes that are identified as in silico probe hits in these plates, various similarity measurements, such as the Tanimoto Coefficient (Tc), may reveal that the other probes in each of the plates containing in silico probe hits to be near neighbors. Hence, all the probes contained in all the plates containing an in silico hit may be subjected to in biologico screening. Once the plate selection process is complete, the results are used for the in biologico screening of the identified and selected compounds 412.

The Selection and Analysis module 308 provides automated selection of chemistry scaffolds. The module 308 also provides automated queries against commercial, public, and proprietary database to select suggested chemistry to be pursued further. In addition, the module 308 provides plate analysis and clustering, providing an indication of confidence in site specificity and identification of scaffolds. The module 308 may also provide automated generation of final reports.

The Select Best Compound(s) module 330 selects the best-ranked conformation for each selected compound. The module 330 next selects the best n compounds or the best m % of all the compounds in their best conformation. The values of n and m may be specified by a system administrator or specified by the user. The module 330 outputs various compound identifiers, such as the compound ID number, so that related information, such as the plate ID number, well ID number, and structure, can be retrieved for each compound.

The Retrieve Location Information module 332 uses the related information to search additional database tables for information, such as the location of the plate identified by the plate ID number. Once a plate has been identified, the information is passed to the next module, the Perform Similarity Analysis module 334. This module 334 may receive information for one or many plates.

The Perform Similarity Analysis module 334 performs similarity analysis between the suggested lists of plates to identify any potentially redundant lists, and provides additional information, such as information to assist in prioritizing list submission for in biologico screening. The module 334 also allows for filtering the lists to remove any plate or compound from the list. This feature allows a user to remove a compound from the screening list for any number of reasons, including, for example, the compounds nature or presence in another project. Various other analysis functionality may also be implemented as part of this module.

In the embodiment of this system illustrated in FIG. 3, the modules 302-308 and sub-modules 312-334 described above execute within the application framework described in relation to FIG. 2. The application framework is illustrated in FIG. 3 as the Application Framework module 310.

The Application Framework module includes three lower-level modules: the Job Scheduling module 336, the User Interface module 338, and the Development Kit module 340.

The Job Scheduling module 336 allows a database such as MySQL or Oracle to be used as a job queuing system for any and all modules of the embodiment shown in FIG. 3. The module 336 includes the Add Job 206 and Daemon 210 shown in FIG. 2 and may also include wrappers for each module as necessary.

The User Interface module 338 provides the user interface 202. In one embodiment, the module 338 provides a web interface for job submissions, job administration, and viewing of job results. The module 338 may allow cross-platform independence, remote access to job information, and other useful functionality.

The Development Kit module 340 provides the capability to add custom modules to the embodiment illustrated in FIG. 3. These modules execute under the application framework as illustrated in FIG. 2. They may be written in any of a number of languages, including, for example Perl and C++.

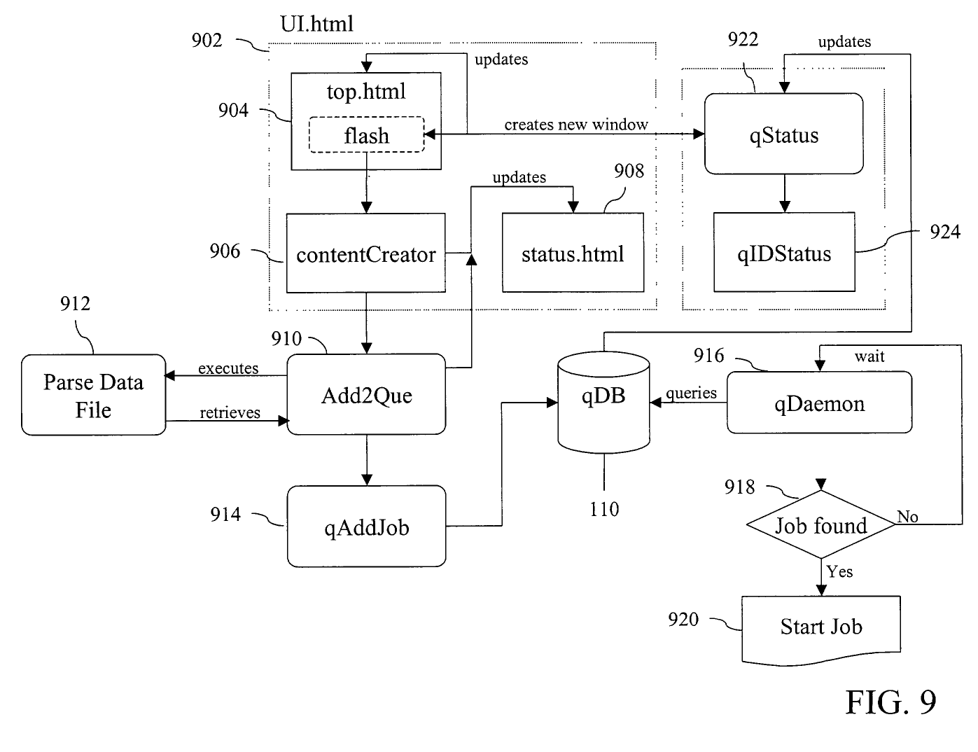

FIG. 9 illustrates the general process of presenting and updating the user interface and scheduling and executing jobs in an embodiment of this system. In the embodiment shown, the interface is an html page named Ul.html 902. UI.html includes top.html 904, which includes a dynamic flash component, contentCreator 906, which generates web page content based on values passed to the script by a flash movie or other user interface element. This script creates all the form elements allowing users to enter information and upload multiple files into the application. Status.html 908, which presents status to a user, is updated by the Add2Queue component 910.

The contentCreator 906 accesses the Add2Que component 910 to create jobs. The Add2Que component 910 reads information about the sequence, for example, from a FASTA or other formatted file 912, checks for errors, and utilizes the data along with user parameters supplied from the contentCreator 906 to execute the qAddJob query 914. The qAddJob query 914 inserts records into the local database qDB 110.

qDB 110 in the embodiment shown is a series of database tables that store information on requested job calculations, what type of calculation types are available for a user's site, how to handle each calculation type, and qDaemon 916 parameters for specific computers, including default parameters. qDB 110 is independent of the computer or user requesting a calculation and the computer that will handle the calculation. One function qDB 110 may implement is to store calculation requests, calculation parameters, input and output data, calculation status, and other information related to requested calculations. Some examples of other information related to a requested calculation include, but is not limited to, who requested the calculation, when the calculation was requested, priority level of the calculation, and searchable user supplied comments related to the requested calculation. The qDB 110 may also stores information input and output data file information, such as name pattern of the files and how many files, for each calculation type.

qDaemon 916 represents a query executing in a background process waiting for jobs to be inserted into the qDB 110. When a new job is found, qDaemon 916 starts a job 920. Changes to the job table in the database 110 are reflected in UI.html 902 via the qStatus 922 and qIDStatus 924 queries.

qDaemon 916 is a precompiled executable daemon that manages calculations running on the computer the daemon was started. The qDaemon 916 determines when to start a calculation based on a number of variables including but not limited to time of day and current CPU usage. qDaemon 916 requests information from the qDB 110 for the next calculation job that the daemon can run; the qDB 110 than returns information for the next available valid requested calculation based on a listed of valid calculation types given by a qDaemon 916 instance, currently waiting requests, and a priority algorithm. If the calculation type requires input data files from the qDB 110, the qDaemon 916 creates any input data files stored in the qDB 110 in a working directory that is also associated with the calculation that is about to run. The qDaemon 916 then calls a calculation specific wrapper script, based on the calculation type, with the requested calculation parameters. If the calculation type requires data files to be uploaded, the qDaemon 916 uploads the output data files to the qDB 110; log files and error log files can be treated as output data files.

Valid calculation types that can be done by a particular instance of a qDaemon 916 are determined at initial startup of the daemon via command line parameters. Multiple instances of QDaemon 916 are allowed on a single computer; this allows multiprocessor computers to run multiple non-parallel calculations simultaneously.

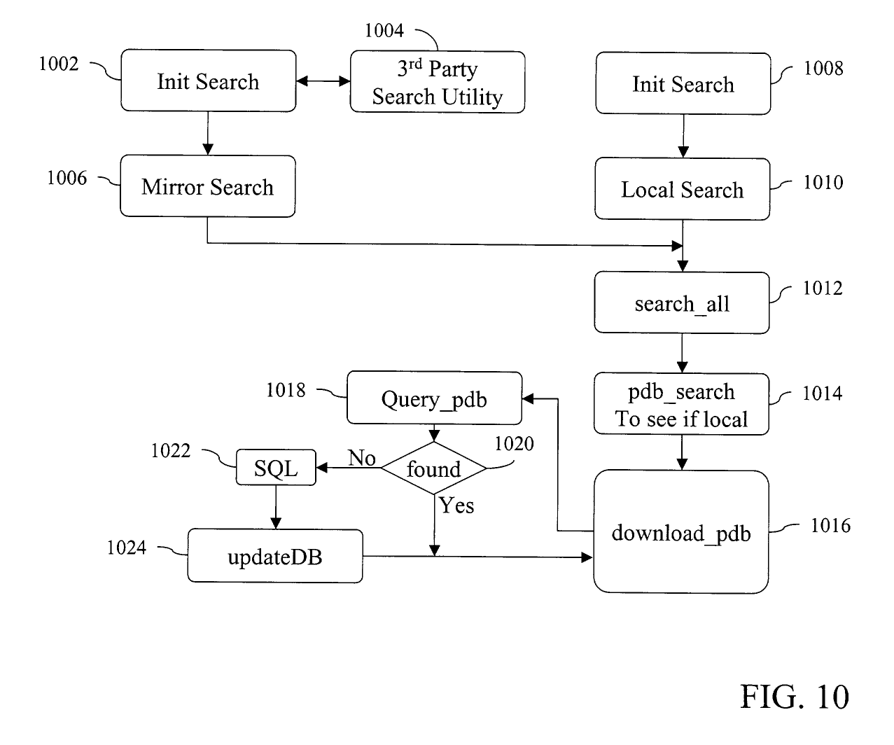

FIG. 10 illustrates the search process in an embodiment of this system. The user begins the process shown by starting a search, such as a BLAST search, of a remote or local database (Init Search). Init Search initiates the BLAST search, pdb file search, or other search programs. This component executes for both remote and local searches. If the search is local, Local Search is executed. Otherwise, Mirror Search is executed.

If the user begins a search of a remote database 1002, the user accesses a third-party search utility 1004. Mirror Search is called for remote public database queries. This component mirrors result files to the local server for searching 1006. In contrast, if the user initializes a local search 1008, the Local Search component parses a local file for searching 1010.

In either a remote or local search, the user can specify what is to be searched. In the embodiment shown, the user specifies “Search All,” triggering execution of the corresponding search_all component 1012. Pdb_search accepts a keyword and queries remote public domain databases for related pdb files. It then mirrors the results locally and parses the result file(s), resulting in a list of pdb file names 1014. Then download_pdb is called 1016.

Download_pdb accepts a list of pdb file names and uses the query_PDB component 1018 to query the local pdb database to see if the pdb files exist locally. If the files exist locally the script reports the results to the log file and ends 1020. If the files are not found locally, download_pdb generates requests necessary to download 1022 the files and then calls updateDB 1024. updateDB 1024 updates the internal database with the names and locations of the downloaded files.

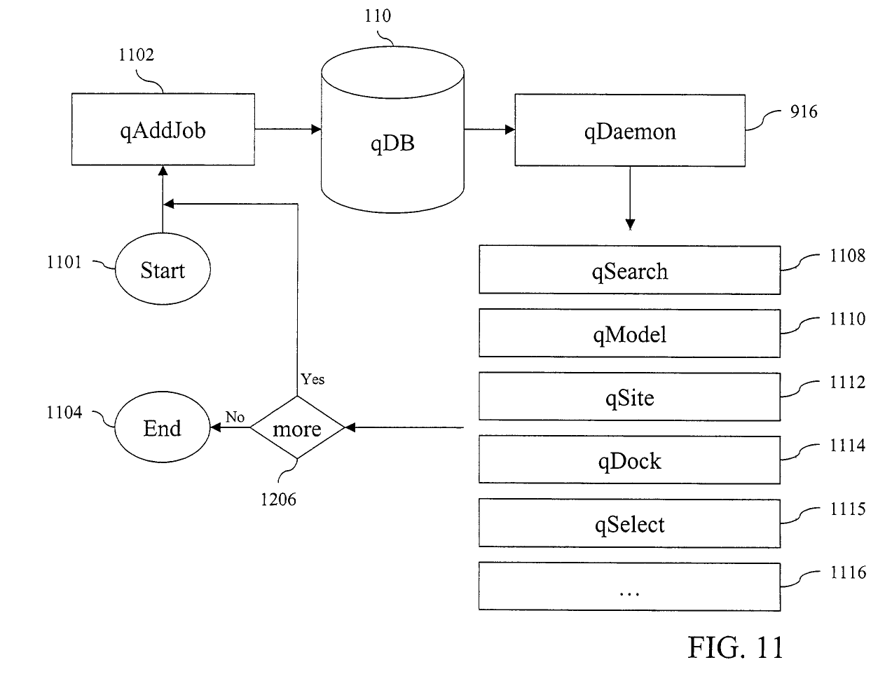

FIG. 11 illustrates the general process of creating and executing jobs in an embodiment of this system. The first step in the process after Start 1101 is the qAddJob process 1102. This process 1102 may execute as a result of a command from a user, an automated system event, or any other process or event that results in the creation and execution of a job. The qAddJob process 1102 simply adds records to the qDB database 110. qDaemon 916 is a background process that waits for jobs to be added to the database 110. When jobs are added to the database 110, the qDaemon process 916 evaluates the records and starts the corresponding process.

In the embodiment shown in FIG. 11, this process may be one of qSearch 1108, qModel 1110, qSite 1112, qDock 1114, or qSelect 1115. It is important to note that this process is not limited to the five jobs shown. Any other process, such as other 1116, may be executed in this manner with little or no change to the integrated user interface. Thus, an embodiment of this system provides great flexibility in the implementation and customization of a computer-aided molecular discovery system.

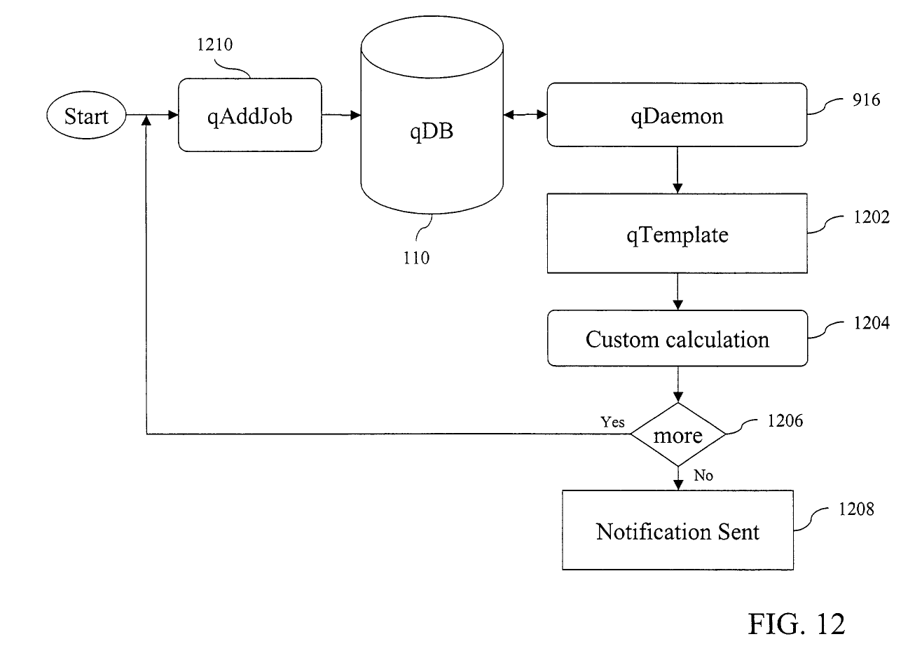

FIG. 12 illustrates utilizing templates and customized jobs in an embodiment of this system. In the embodiment shown, the first process after Start 1201 is the qAddJob 1210 process 1210, which adds a job record to the database, qDB 110. qDaemon 916 again waits for jobs to be added to the database 110. When a job is added, an application template, qTemplate 1202, is executed, which in turn, executes a customized calculation 1204. If additional jobs are spawned from the calculation 1206, another job is simply added to the database, qDB 110, by qAddJob 1210. If not, a notification is sent by some means, such as instant messaging, email, or by another method 1208.

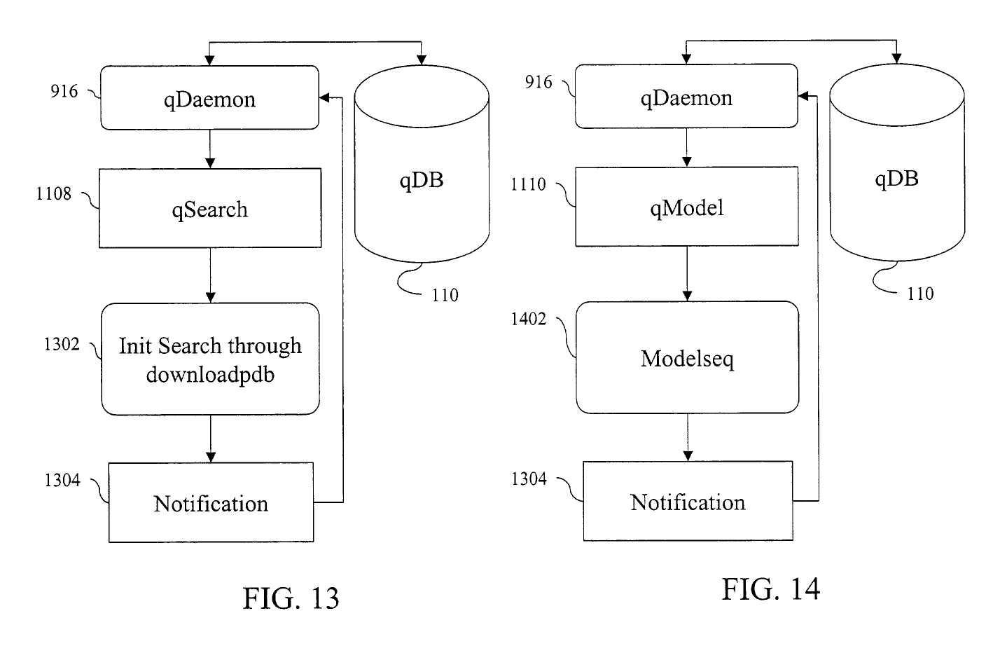

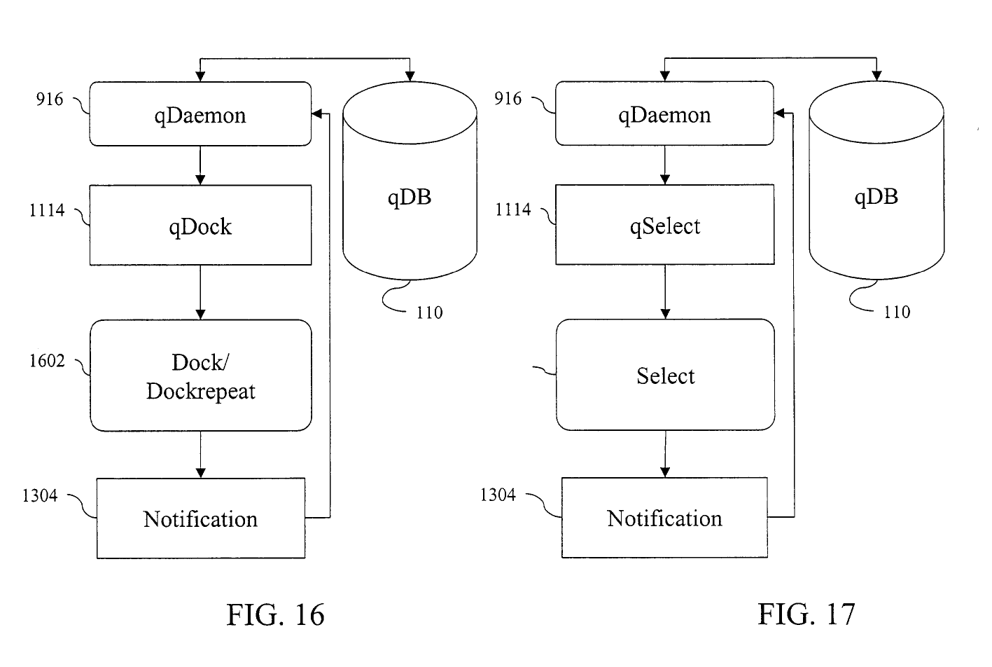

FIGS. 13-17 illustrate the process of providing notification, such as by email or other method, of the completion of a job in an embodiment of this system. As in other aspects of this system, the qDaemon process 916 waits for jobs to be added to the database, qDB 110. When a job is added, qDaemon 916 begins the appropriate job. In the embodiments shown, the job is one of qSearch 1108, qModel 1110, qSite 1112, qDock 1114, qSelect 1115, or other component process 1116. Each of these jobs executes a corresponding process or series of processes, shown as Init Search through download_PDB 1302, Modelseq 1402, Site 1501, and Dock/Dockrepeat 1504, respectively, in the Figures. Once the process is complete, the notification component 1304 provides notification to a user, such as by email, fax, instant messaging, or other suitable communication method.

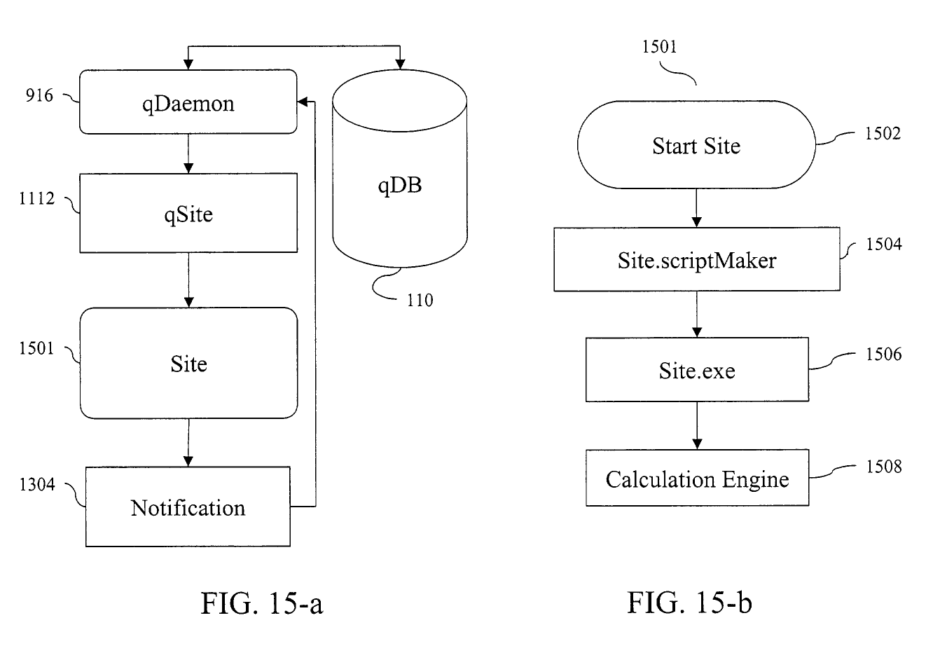

FIG. 15a illustrates the creation and execution of a custom script for a commercial application component in an embodiment of this system. In the embodiment shown, the Site process is started '502 by adding a job to the job database as described above. The execution of the Site process results in the creation of a script, which controls the execution of a third-party commercial, public, or custom application. In FIG. 17, this step is illustrated by the Site.scriptMaker step 1504. This script is then executed in the Site.exe 1506, which executes the calculation engine 1506 necessary to perform calculations for the Site process.

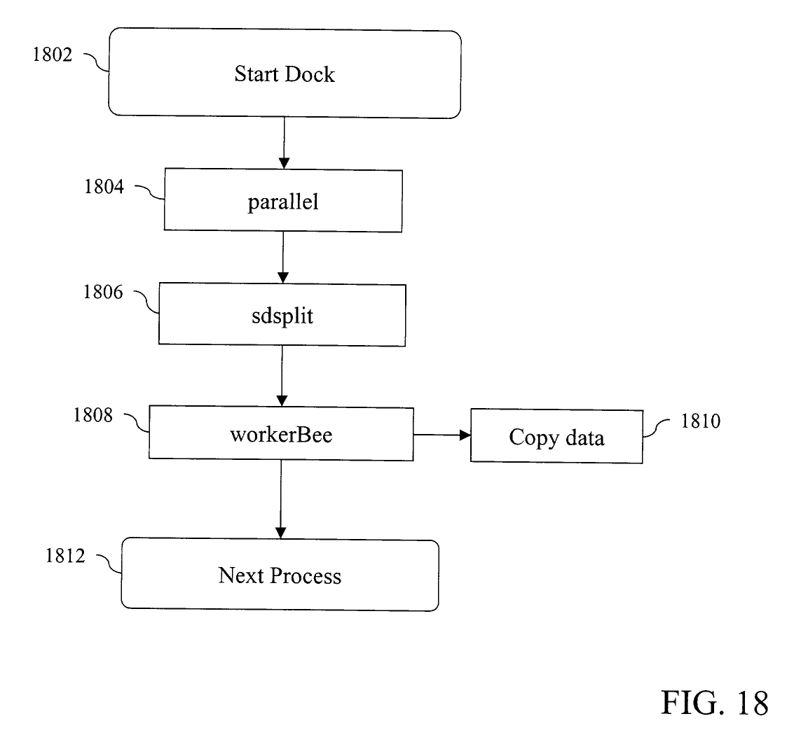

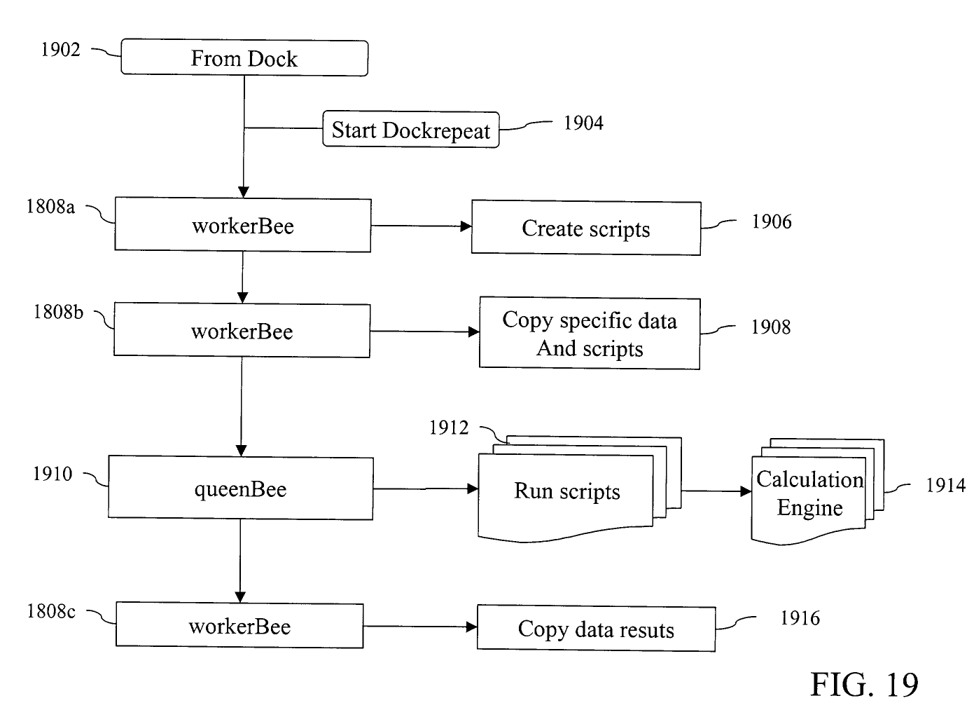

Embodiments of this system provide many benefits over conventional computer-aided molecular discovery systems and processes. One advantage is the ability to parallelize processes across heterogeneous clusters. FIG. 18 illustrates the pre-paralellization process in an embodiment of this system. The docking process is shown in FIG. 18 for purposes of illustration. However, any of the processes of this system may be parallelized in the same manner. In the embodiment shown, the docking process is started 1802. The start of the process triggers the parallel process 1804. In order to process the information in parallel, the data file, which is an SD file in the embodiment shown, must be split into multiple smaller files 1806. The process of splitting is performed by a WorkerBee 1808, which is described in detail below. The WorkerBee 1808 next copies the smaller data files to the appropriate node in the heterogeneous cluster 1810. The next process then begins 1812, which is illustrated in FIG. 19.

FIG. 19 illustrates the paralellization of a process in one embodiment of this system. The efficient parallelization of the process is achieved through a combination of processes called WorkerBees (WBs) that pre-process and post-process the tasks required for parallel runs. A global process, QueenBee (QB) manages the actual run of the docking engine on several nodes. The security of the process is insured by appropriate firewall implementations.

WB is a dynamic process that manages the parallelization of all the tasks involved in in silico screening process. There are usually several WBs handling the pre-processing and the post-processing of the various computational stages in a coherent fashion. As an example, one WB could be creating input files for the docking engine; another WB could manage the distribution of all the chemical structures on all the nodes; another WB could post-process the collection of data.

To perform its function, WB needs to know about the configuration of the computer cluster (input: cluster.conf fille). This file contains information about the server name, common directory for that particular machine, calibration data that are used for heterogeneous cluster load balancing.

The parallelization process can be used on a heterogeneous Unix/Linux cluster, including SGI machines or SUN or IBM or Linux boxes with different CPU mixes.

QB takes in a file describing what programs to run in parallel and run them all at the same time. QB can be located on any member of the cluster but preferably on the leading node of the cluster. Pre-processing WBs create and distribute programs to be run on each node. When it is done, QB runs and manages the execution of all these processes until they have all successfully completed. After completion, Post-processing WBs post-process the data.

The Dock process as illustrated in FIG. 9 provides an illustrative example of the WorkerBees and QueenBee in an embodiment of this system. The process shown in FIG. 19 begins where the process in FIG. 18 stops. The data has been divided; in this case a large SD file of chemical structures to be screened, into several pieces to be processed independently on each node in a parallel fashion. Pre-processing WBs 1808a,b initiate and launch tasks and prepare data.

One WB 1808a creates per node docking engine scripts 1906. Another WB (not shown) creates per node shell scripts that ensure data management and proper data allocation. One WB 1808b copies the data to the individual nodes 1908, e.g. in this case the pieces of the original large SD file. WB 1808b also creates the file that will be used by QB 1910. Queen-Bee 1910 is then run. After completion, post processing WB 1808c is run. Post-processing WB 1808c combines data and copies the data results 1916.

WB 1808c may actually be multiple WBs. For example, in one embodiment, one WB combines the individual SD file after calculation of the in silico screening score into one large final SD file. One WB cleans up the data on the individual nodes, removing unused files. One WB performs any additional per node calculation that might be necessary at this point.

An embodiment of the present system uses a variety of software languages to integrate various components. For example, in one embodiment of the present system, Perl is used to perform integration within the user interface; SVL is used for protein modeling; and C² and other proprietary and public scripts are used to implement procedures within commercial software packages. Also, shell scripts are implemented where necessary, for example, for parallelization of the process. HTML, XML, Java, and JavaScript provide the necessary functionality for presentation with the user interface.

Embodiments of this system may support a variety of functions related to molecular discovery beyond the processes described above. For example, embodiments may support: (1) Large scale (millions) enumeration of library compounds; (2) Parallelized conformation generation; (3) Large scale physico-chemical descriptor and molecular fingerprint calculation; (4) same ligand set, variable protein model analysis; (5) cross-site same protein/variable ligand set analysis; and (5) in silico high-throughput screening of compounds.

In addition to the functionality described in detail above, an embodiment of this system may include a variety of other functions and processes. For example, an embodiment may include administration functions. Various user types are defined, such as administrator, advanced user, and casual or novice user, and the interface and functioning of the system is varied based on the user type.

It is quite likely that some organizations utilizing an embodiment of this system will require that security measures be implemented to ensure that the data generated and consumed by the system will not become known outside the organization. One embodiment of this system operates only within a firewall and utilized secured sockets layer to provide security.

An embodiment of this system may be implemented on a single client site or across multiple client sites, utilizing standard protocols, such as TCP/IP. Therefore, a variety of billing and licensing strategies may be utilized. For example, an organization may purchase an unlimited license, or an organization may simply purchase one or more per-seat licenses. In addition, an embodiment of this system may be implemented as an application or web service to which organizations subscribe.

Description of Sreening Method

Embodiments of this system provide systems and methods for data analysis, including data retrieval, dynamic scripting and execution, mining, storing, and visualization. One embodiment of this system provides an integrated software solution for managing high volumes of numerical data quickly and efficiently. Another embodiment provides a complete and flexible solution data acquisition, management, and manipulation.

The types of data that a system according to this system is capable of managing includes but is not limited to primary and secondary in vivo and vitro screening. An embodiment of this system stores and integrates numerical data, such as biological and chemical data, in a database. The system uses an object-oriented approach for data analysis, programming, mining, storing, and visualization of the data.

Embodiments of this system provide multiple advantages over conventional data analysis tools. A system according to this system provides an integrated user interface in which to view and modify data. When changes are made to either tabular or graphical data, the user interface automatically changes the corresponding data in the other view(s). By automatically changing the data, the user avoids the problem of switching between views, which is common in conventional systems.

An embodiment of this system also allows a user to manage diverse types information, including, for example, information related to molecular discovery that ranges from large amounts of data generated from high-throughput screening programs, through multiple IC50 determinations and profiling, to complex experimental protocols and kinetics studies.

An embodiment of this system also provides a highly flexible user interface. The user interface provides a layout feature. The layout feature of the system enables biologists to vary experiment parameters interactively. For example, using this feature, researchers can easily perform dose response titrations across several assay plates rather than having to create dose responses on single plates.

The user interface in an embodiment of this system provides interactive curve-fitting capabilities combined with powerful graphic and charting tools for statistical analysis, a powerful query and reporting tool for creating structure-activity relationship reports, sample lists and profiles. To provide a richer and more intuitive user interface, each session's information is stored and easily retrieved through the ‘DB Search’ option, which is both fast and efficient.

An embodiment of this system also allows the user to create customized templates for compound screening or other types of analysis. Controls, compounds, and concentrations can all be varied across a plate to allow for optimal placement. Due to this flexibility, an embodiment of this system allows the user to make changes based on the user's expertise in the area.

An embodiment of this system preserves the integrity of raw data. The application is fast and dynamic while maintaining the original data. The system can handle single or multiple plate analysis. Once the information is uploaded, it is stored in a centralized database. Any combination of templates can be defined; redefining controls as well as data locations as needed. The session is stored and readily available, for all future references. Thresholds are definable at a keystroke and can be adjusted for each experiment.

Embodiments of this system provide systems and methods for data analysis, including data retrieval, dynamic scripting and execution, mining, storing, and visualization. One embodiment of this system provides an integrated software solution for managing high volumes of numerical data quickly and efficiently. Another embodiment provides a complete and flexible solution data acquisition, management, and manipulation. The types of data that a system according to this system is capable of managing includes but is not limited to primary and secondary in vivo and vitro screening. An embodiment of this system stores and integrates numerical data, such as biological and chemical data, in a database. The system uses an object-oriented approach for data analysis, programming, mining, storing, and visualization of the data.

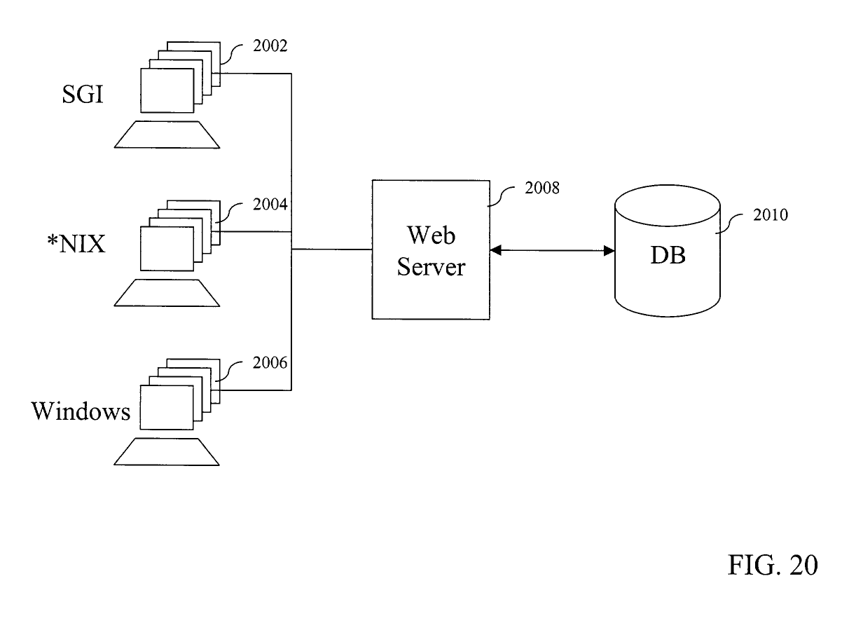

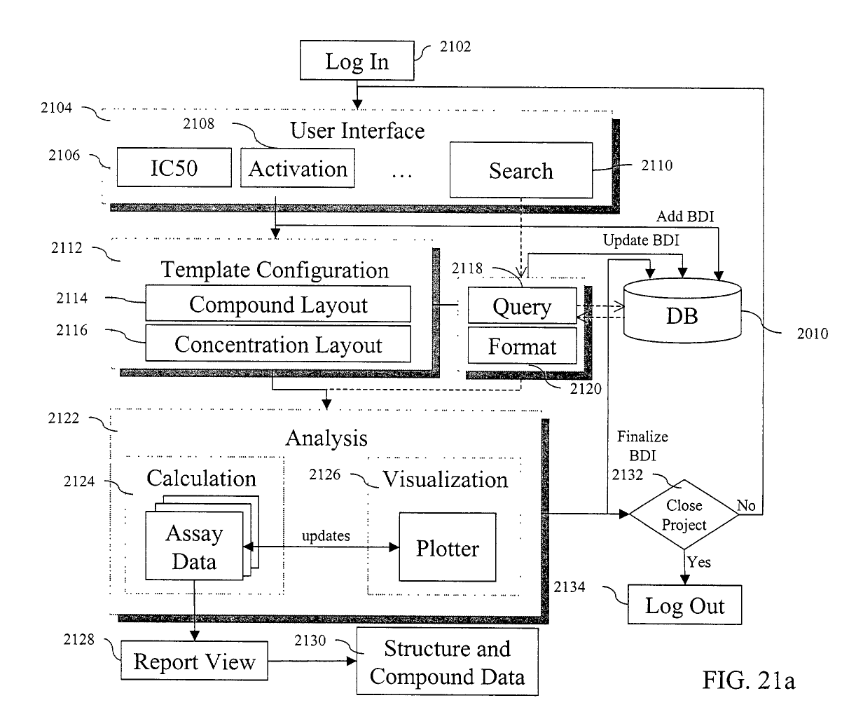

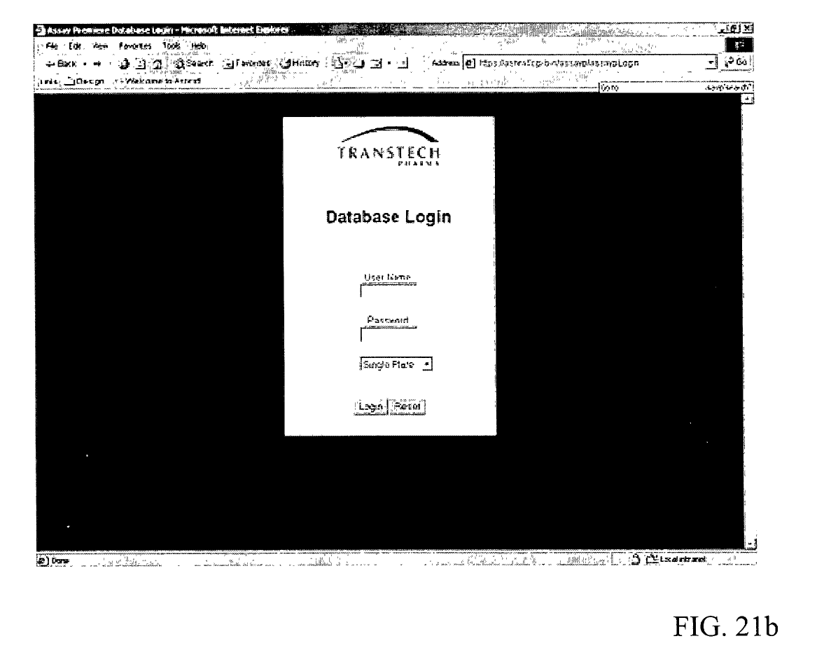

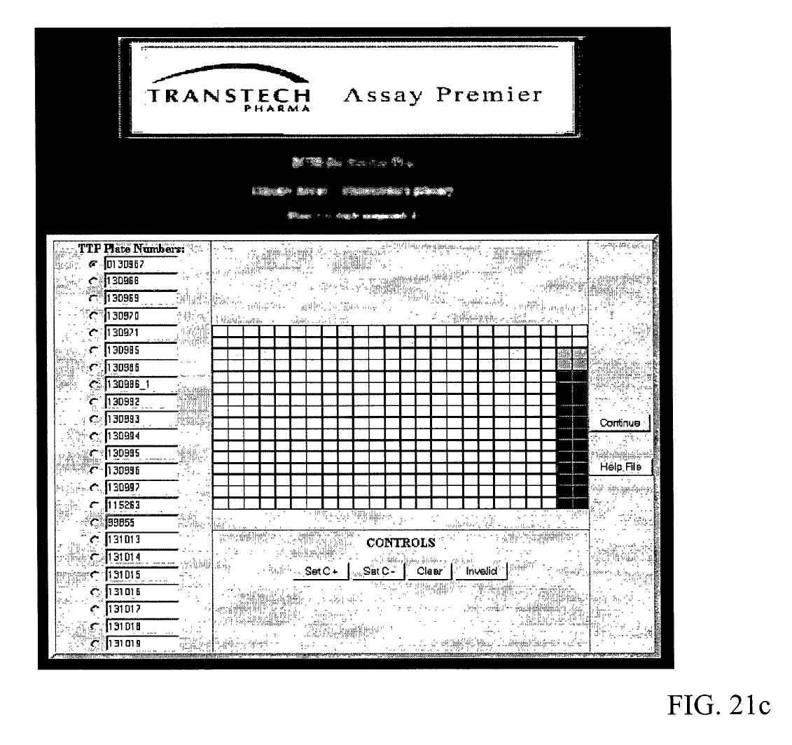

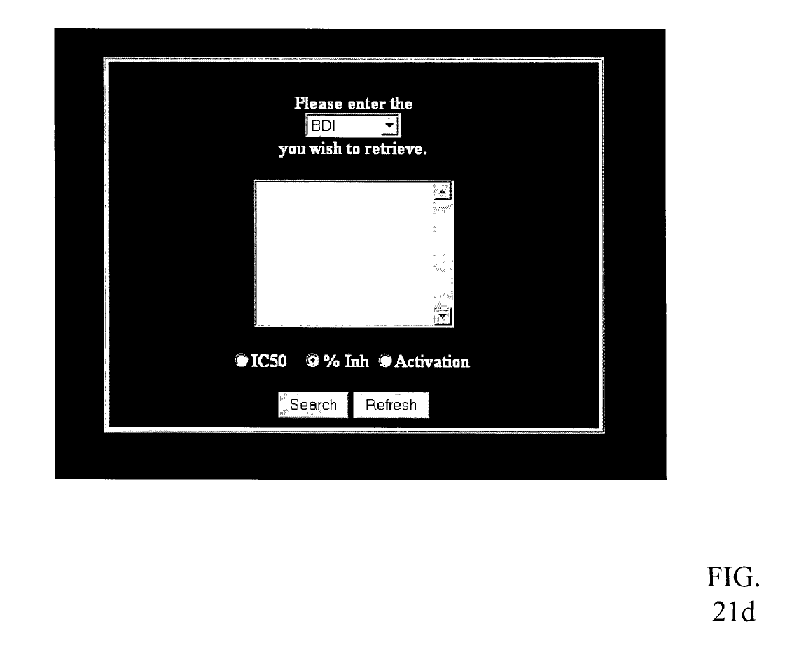

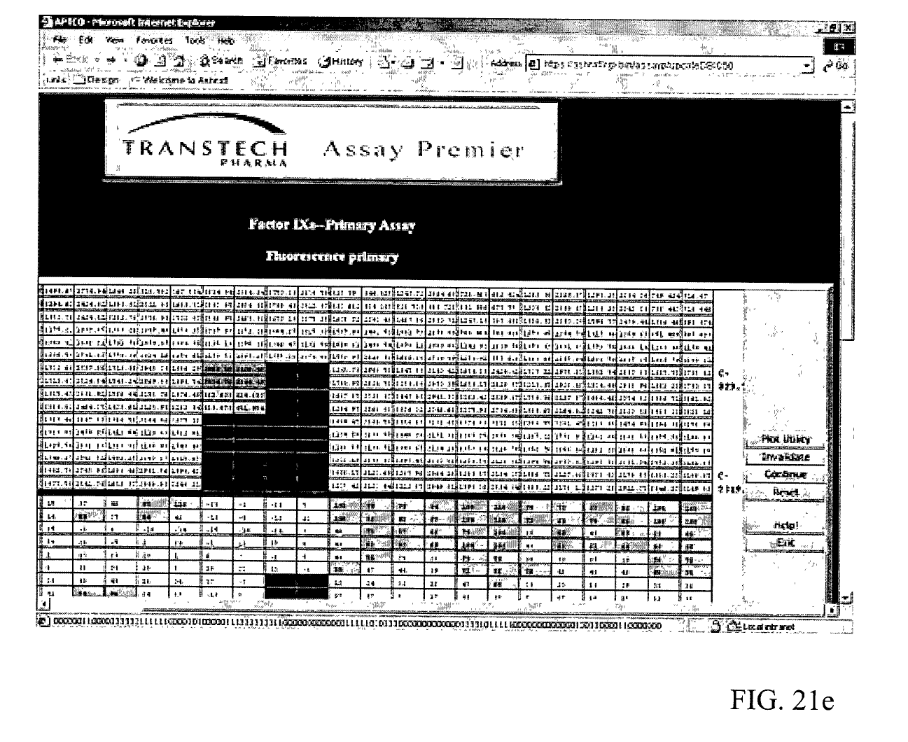

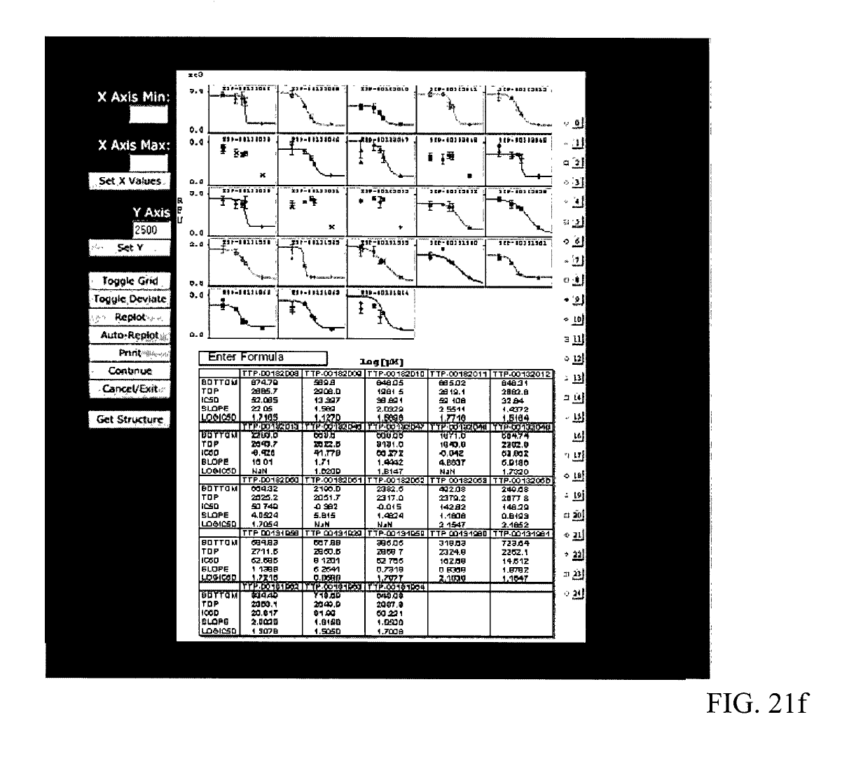

FIG. 20 illustrates an exemplary embodiment of this system. A user accesses the system via a users interface. In the embodiment shown, the user interface is a web-browser-based interface, which can execute on any number of platforms, including Silicon Graphics (SGI) 2002, Unix and LINUX (*NIX) 2004, and Microsoft Windows 2006. A web server 2008 generates the user interface. The web server 2008 also receives parameters and requests from the user interface. To generate the user interface and to respond to user requests, the web server 2008 accesses a database (DB) 2010, such as like MySQL, Oracle, ISIS and others. By utilizing a web-based approach, the embodiment shown in FIG. 21 is platform-independent, both in terms of the server and workstation; any web platform capable of supporting programming languages and features, such as C, C++, cookies, DHTML, Java, JavaScripts, PERL, servlets and others, is capable of supporting the system.

An embodiment of this system manages a wide variety of information. For example, in one embodiment, the system manages information related to molecular discovery that ranges from large amounts of data generated from high-throughput screening programs, through multiple IC50 determinations and profiling, to complex experimental protocols and kinetics studies.

An embodiment of this system provides a highly flexible user interface. The user interface provides a layout feature. The layout feature of the system enables biologists to vary experiment parameters interactively. For example, using this feature, researchers can easily perform dose response titrations across several assay plates rather than having to create dose responses on single plates.

An embodiment of this system provides a security layer to ensure that sensitive data is not compromised. A web-based embodiment easily allows multiple sessions to be run simultaneously from anywhere within a network; a browser is all the client requires to execute the application.

The user interface in an embodiment of this system provides interactive curve-fitting capabilities combined with powerful graphic and charting tools for statistical analysis, a powerful query and reporting tool for creating structure-activity relationship reports, sample lists and profiles. To provide a richer and more intuitive user interface, each session's information is stored and easily retrieved through the ‘DB Search’ option, which is both fast and efficient.

An embodiment of this system preserves the integrity of raw data. The application is fast and dynamic while maintaining the original data. The system can handle single or multiple plate analysis. Once the information is uploaded, it is stored in a centralized database. Any combination of templates can be defined; redefining controls as well as data locations as needed. The session is stored and readily available, for all future references. Thresholds are definable at a keystroke and can be adjusted for each experiment.

In one embodiment of this system, the user interface is a graphical java-based application that is highly customizable for each IC50 analysis. Using the GUI and keyboard routines, the graphical component of the interface, the IC plotter, can be quickly suited for each user. The IC plotter directly accesses the database for it's plotting information and updates the modified data after each analysis. The IC plotter is an extremely powerful component of an embodiment because of its features and flexibility.

The system is an easy to use analysis application that is dynamic, fast and efficient and can be used on any platform. It contains user-friendly features including custom templates, direct data access, centralized databases, flexible project creation and multi-plate projects. It is very advanced; it allows multiple users to simultaneously start new projects, return to previously completed projects and is easily expandable for future experiment types and methods. Reports are dynamically generated within the system at the click of the button. The shading quickly of each well allows the user to interpret the results and is versatile for both color and black-and-white printing. The web-reports are specially formatted for standard page layouts.

FIG. 21a illustrates a view of various aspects of an embodiment of this system as a scientific data analysis application. Initially, the user logs in 2102. FIG. 21b is a screen shot of a login screen in one embodiment of this system. The system provides the user with a user interface 2104. In the embodiment shown, the user interface includes various sections, including IC50 2106, Activation 2108, and Search 2110. Because of the flexibility of the user interface, many other potential sections may be included in the interface.

In the embodiment shown, the user selects either to view (Search) or create (IC50, Activation) a template configuration 2112. The template configuration 2112 refers to a representation of a plate, which will be used to perform an assay. FIG. 21c illustrates such a representation in one embodiment of this system. The template configuration 2112 includes a compound layout 2114 and a compound concentration 2116 option with corresponding user interface attributes. The user uses these views to specify or view where a compound is to be placed on a plate and what the concentration of each of the plate wells will be.

When the user searches for a template configuration, using a form such as the screen shot shown in FIG. 21d, one embodiment of this system utilizes a query component 2118 to access a database (DB) 2010. Results from the database are then formatted by a format component 2120 and provided to some portion of the user interface 2104, template configuration 2112, or analysis components 2122.

When the user has completed the template configuration 2112, the embodiment shown provides an analysis interface 2122. The analysis interface provides various views of the data including a calculation view 2124 and a visualization view 2126. Importantly, these views are not mutually exclusive. Also, data changes in one view are automatically and immediately made to the other corresponding view. Because it is critical in some applications that the integrity of raw data be maintained, one embodiment of this system make a copy of the raw data, and all changes to data occur on the copy of the data, leaving the raw data in its original state, neither altered nor deleted.

In the embodiment shown, assay data is displayed in the calculation or Assay Analysis view 2124 and corresponding plots of the data are displayed in the visualization or IC Plotter view 2126. One embodiment of this system uses the Assay Analysis view 2124 shown in FIG. 21e and the IC Plotter view 2126 shown in FIG. 21f.

In an embodiment of this system, the Assay Analysis view 2124 may be implemented as a java or other modular component (herein referred to as techlet). The Assay Analysis techlet 2124 combines the information gathered from the previous two views and information from a file that may be imported and parsed to display the raw data on the top half and the calculated values on the bottom half. An embodiment may utilize color-coding to enhance the usability of the techlet. For example, for a user to quickly identify which data set they are looking at, the currently selected compound is tinted blue. The user can change which compound they want to be selected by clicking on a numbered button in the user interface.

Additional features may be implemented to enhance the flexibility of the techlet as well. For example, from the Assay Analysis view 2124, the user may highlight data points that are above preferred threshold by clicking and/or dragging over any number of wells. Highlighted wells are shaded with a dark-green and regular wells are shaded with a light-green. The user may also invalidate data points that are too extreme when compared to others in the same data set. Invalidated data will be displayed with a fine red X across the well. For applications in which the integrity of the raw data is necessary, invalidation of the data in the user interface does not affect the raw data; invalidation affects only the copy of the data.

When the user has completed analysis, manipulation, and visualization of the data, the user selects a control, such as a command button labeled ‘Plot’ to access the IC Plotter view or techlet 2126 and visibly interact with the data. An embodiment may include additional features as well. For example, a well that is invalidated within the Assay Analysis view 2124 will be invalidated before the curve-fit and plot is calculated in the IC Plotter 2126. Also, any points that are invalidated during the plot configuration will also be invalidated on the Assay Analysis view 2124.

As noted above, in an embodiment of this system, the IC Plotter 2126 receives the data from Assay Analysis 2124 and creates a plot, or multiple plots—one for each compound on the plate, and displays the first on the main window. To change between compounds to select and display, the user may click on any of the embedded java buttons to change selection or may press <1>˜<0> for the first ten compounds, <Shift>+[<1>˜<0>] for 11 through 20, and <Ctrl>+<Shift>+[<1>˜<5>] for the remaining 21 through 25. Because of constraints on the size of a computer display, the maximum number of compounds displayed at any one time may need to be limited. For example, in one embodiment, the maximum number of compounds, which may be displayed at on time for IC Plotter 2126, is 25 compounds. If a user is analyzing more than 25 compounds, a user interface according to this system may present the additional compounds on additional “pages” within the user interface while maintaining 25 or less compounds per page.

In an embodiment, IC plotter 2126 includes two views: a single plot and a mutiplot view. The single-plot allows for an enlarged and more detailed view of a single compound. If the user presses <ctrl>+[<2>˜<5>] or <M>, then IC Plotter 2126 will change multi-plot mode and anywhere from a 2×2 to 5×5 grid and will display as many compounds as alloted space on the grid. Pressing <M> before any other grid size will display the maximum grid size of 5×5 by default; all future <M>s will toggle between last used grid-size and single-plot. Pressing <Ctrl>+<1> or <M> will return the display to the single-plot with the enlarged, detailed view of the currently selected compound.

The user may set the minimum and maximum ranges of the X and Y axis to best display their data by either entering limits on the HTML or by using the arrow keys to scale and shift the plot as needed. The values of the axis ticks and labels are dynamically recalculated and relabeled on each change. The <Shift> is used to accelerate the scaling and moving of the axis while the <Ctrl> is held or released to toggle between scaling and moving-default is to scale. The named labels for

On the currently selected compound, the user may invalidate any number of data points by clicking and dragging over them. When the user releases the mouse-button, the curve fit is recalculated and plotted if the curve succeeded in fitting to the data. If the curve is not able to fit the data points, then only the data points are displayed—no curve will be drawn. If a fit to the curve is made, but is unacceptable to the user, the user can press <Ctrl>+<Shift>+‘click’ on the compound either in the table or in the plotting region. When a compound is not plotted, the table changes all cell element values of the compound to dashes to indicate that the values are unacceptable.

The lower section of IC Plotter 2126 contains a table with each cell containing each compound. The elements of each cell refer to information displayed on the plot. On the single-plot view, if the user clicks on any cell, then that plot is now displayed in the main window and the cell is highlighted for quick reference. On the multi-plot view, if the newly selected compound is not displayed it will shuffle the currently displayed compounds in and out until the selected compound becomes visible and the table cell will highlight for the selected compound. If the newly selected compound is already displayed, only the table cell will highlight and nothing will be done with the main window.

When the user has completed their analysis of the plots created from their data points, the user may print the currently displayed plot(s) and clicks ‘Done’ to return to Assay Analysis 2126 with their revised data now displayed on the plate layout.

An embodiment of this system may include various keyboard controls to perform functions within the Assay Analysis 2124 and IC Plotter 2126 views, both graphical and non-graphical, within the user interface. The following list of commands is utilized by one embodiement:

Keyboard Select:

| | |
|---|---|
| 1-0 | Selects Compounds 1 through 10 |
| Shift + 1-0 | Selects Compounds 10 through 20 |
| Ctrl + Shft + 1-5 | Selects Compounds 21 through 25 |

Basic Keyboard Control:

| | |
|---|---|
| ‘Left’ | Moves the data left |
| ‘Right’ | Moves the data right |
| ‘Up’ | Inceases the Y-axis Scale |
| ‘Down’ | Decreases the Y-axis Scale |
| Ctrl + ‘Left’ | Decrease the X-axis Scale |
| Ctrl + ‘Right’ | Increase the X-axis Scale |
| Shift + <dir> | Multiple action by 5 |
| ‘G’ | Toggles Grid View on or off |
| ‘D’ | Toggles Stadard Deviation Mode |
| ‘M’ | Toggles between Multi-Plot and Single Plot |

Advanced Keyboard Control:

| | |
|---|---|
| ‘A’ | Toggles Autoplotting on for dynamic plotting or |
| | off to speed up complex calculations |
| ‘P’ or ‘R’ | Forces a replot of the data. |
| ‘I’ | Reinitialize IC-Plotter (soft restart of the |
| | application) |
| ‘[’ | Decrease overall Plot Screen |
| ‘]’ | Increase overall Plot Screen |
| ‘O’ | Toggles Overlay Mode (future release) |
| ‘C’ | Toggles IC50 axis reference lines (future release) |

Additional views may also be provided in an embodiment of this system. For example, the embodiment shown in FIG. 21a includes a report view 2128. From the report view, a user specifies a particular compound about which the user wishes to see additional details. The system then provides the user with a structure and compound data view 2130, which provides details about the compound of interest.

In the embodiment shown in FIG. 21a, once the user is satisfied with changes to the copy of the data that the user is manipulating and viewing, the changes are saved to the DB 110. The user is asked whether or not to close the project currently displayed 2132, and if the user responds affirmatively, the user is logged out 2134.

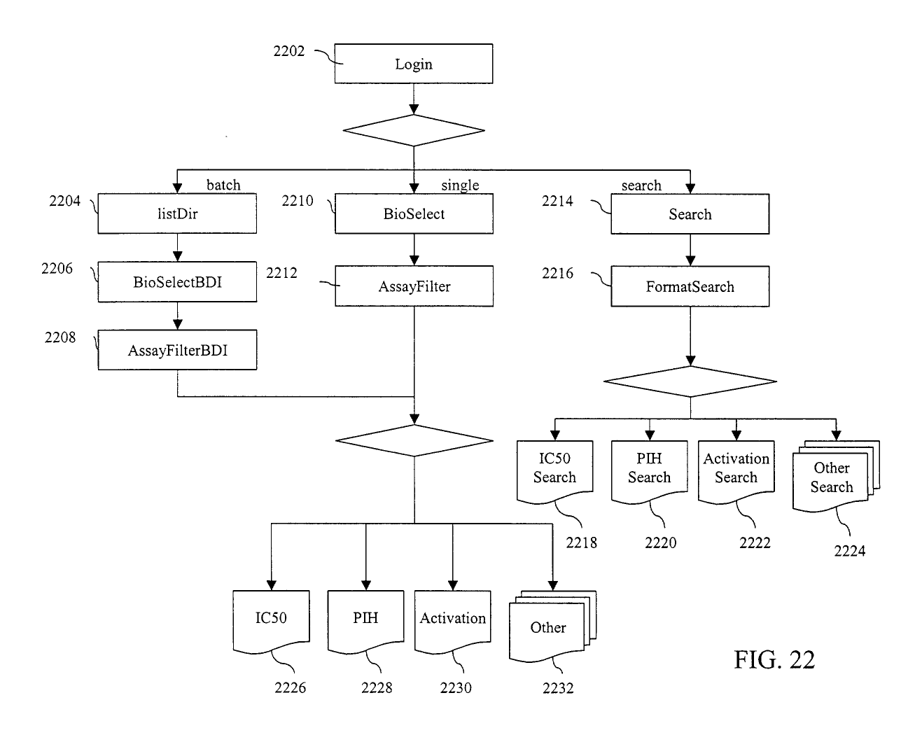

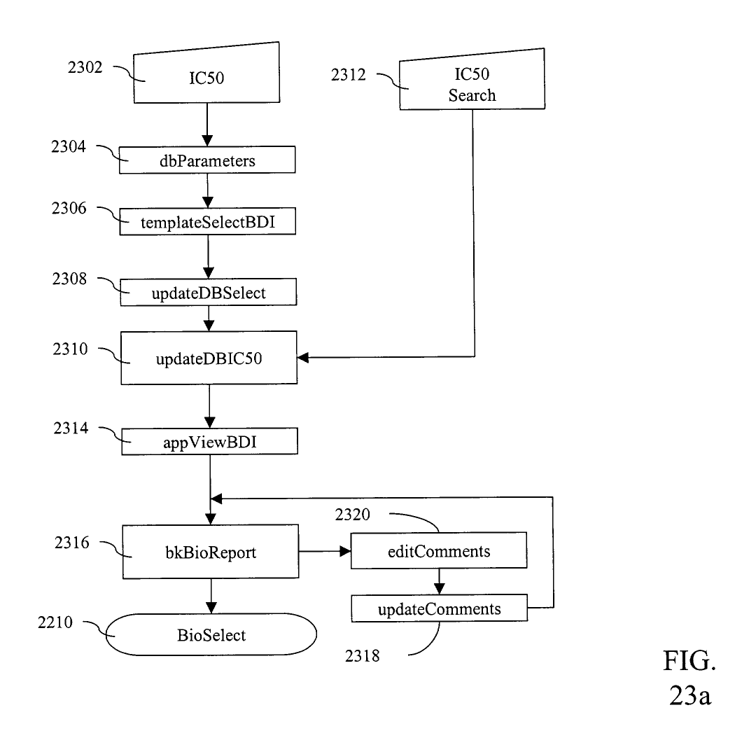

FIG. 22 illustrates the process utilized by an embodiment of this system in presenting the user interface and responding to user requests. In the embodiment shown, when the user accesses the system, the user must login 2202. The system accepts username and password and allows selection of analysis or search options. Analysis includes Single or Batch analysis. In one embodiment as a web browser based application, the submit button on the page is clicked, and a cookie is set with the username and password. The application determines the next page to present based on the analysis type or search option selection.

If batch analysis is selected, they are directed to ListDir304. If the user selects single analysis they are directed to BioSelect 2210. If ‘Search’ is selected, the user is directed to Search 2214. In one embodiment, the next script is executed when the user clicks a command button labeled, ‘Login’. The modules used to create the user interface, responds to user inputs, and perform program control may be one or a combination of any programming language, including but not limited to Perl, Java, C, C++, JavaScript, and HTML.

ListDir 2204

In one embodiment of this system, the ListDir component 2204 uses a default network directory for file uploads. For a multiple plate analysis, the files to be used for this analysis are placed in a new folder within the default network directory. ListDir 2204 reads the contents of the top default directory and lists them within the page with a checkbox next to each listing.

A ‘Select All’ command button causes all check boxes on the user interface page to be selected. ‘Deselect All’ causes all the checkboxes to be deselected. ‘Invert Selection’ reverses the checkbox selection. Clicking the command button labeled ‘Submit’ causes the program to call the BioSelectBDI module 2206.

BioSelectBDI 2206

In an embodiment of this system, the BioSelectBDI component 2206 provides the capability for a user to define the analysis session by target and experiment type for multiple files already uploaded into the user interface. Selection can be made between different calculation types and input parameters change according to the user's selection. In an embodiment implemented as a web-based user interface, HTML form elements are set dynamically as the user interacts with the page.

In one embodiment, a hyperlink is located at the top of the page that allows a user to redirect the project into a search mode. The hyperlink calls the script search.

A command button labeled ‘Submit’ causes a cookie to be set, which contains the selections. As described above, form elements are set based on user selections and the AssayFilterBDI component 2208 is executed.

AssayFilterBDI 2208

In one embodiment of this system, the AssayFilterBDI 2208 component uploads the files previously selected in ListDir 2202, parses the files, and then inserts the data into the database. The user may be presented with additional options. Based on the selections made by the user or on a predefined logic flow in the BioSelectBDI component, the display component is executed. AssayFilterBDI 2208 also determines the plate layout for the project.

To display a potable calculation type, the APTIC component (described below) is executed. If the calculation type is not potable, the appViewBDI component (described below) is executed next.

If any information is missing from previous submissions, the cookie is read. If the information needed is still not available, the system provides the user with a dynamically created submission display to supply the missing information, utilizing either the BioSelect 2210 or BioSelectBDI 2206 components.

Once the AssayFilterBDI component 2208 is complete, output is created by an embodiment of this system, including but not limited to IC50 2226, PIH 2228, Activation 2230, and Other 2232 output. Output may be displayed in the Assay Data 2124 and IC Plotter 2126 views described above.

BioSelect 2210

The BioSelect component 2210 in an embodiment of this system allows the user to define the analysis session by target and experiment type. The user uploads the experiment's data file into User interface. Selection can be made between different calculation types and input parameters change according to the user's selection. Form elements are set dynamically as the user interacts with the page.

The user interface may include a hyperlink on the page that allows a user to perform a search. The hyperlink calls the search component 2214.

In one embodiment, when the user clicks a command button lageled ‘Submit,’ a cookie is set saving the selections, form elements are set based on user selections and form elements are submitted to the AssayFilter component 2212.

AssayFilter 2212

The AssayFilter component 2212 uploades the file previously selected in the BioSelect component 2210 to an archive directory and parses the data file, inserting the data into the database. Based on the selections made in the user interface under control of the BioSelect component 2210, the next component is executed. The AssayFilter component 2212 also determines the plate layout for the project.

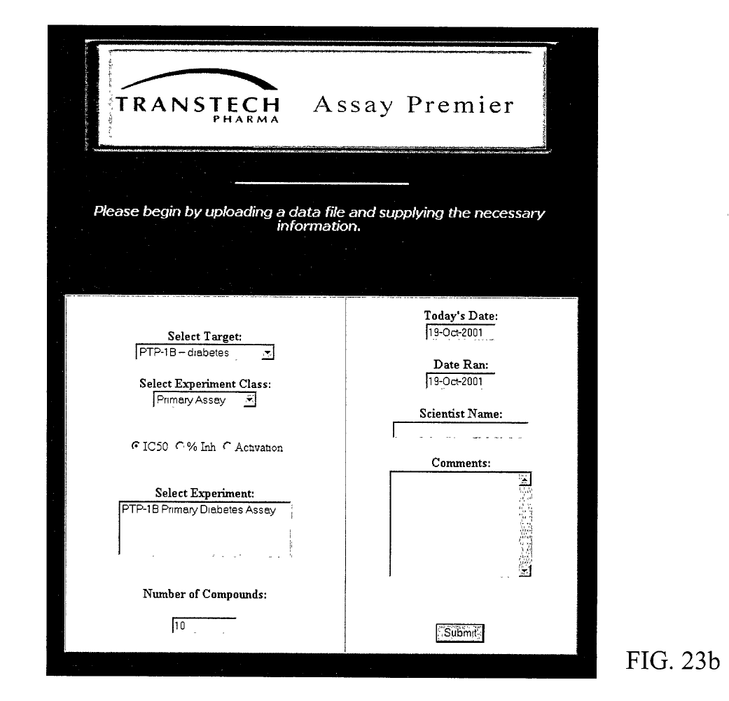

In one embodiment, as with the AssayFilterBDI component 2208, the AssayFilter component 2212 executes the APTIC component (described below) to display a plottable calculation type. If the calculation type is not plottable, the AssayFilter component executes the dbParameters 2304 component (described below in relation to FIG. 23).

If any information is missing from previous submissions, the cookie is read. If the information needed is still not available, the system provides the user with a dynamically created submission display to supply the missing information, utilizing either the BioSelect 2210 or BioSelectBDI 2206 components.

Once the AssayFilter component is complete, output is created by an embodiment of this system, including but not limited to IC50 2226, PIH 2228, Activation 2230, and Other 2232 output.

Search 2214

In an embodiment of this system, to perform a search, the search component 2214 first reads the username and password of the user from a cookie. The application next presents the user with a list of search parameters from which to choose, including but not limited to compound ID number, plate number or BDI number. The user enters the correct information for searching and selectes the type of calculation to be used for each item searched for. The calculation may be a predefined calculation, such as IC50, Activation, or Inhibition, or a custom calculation provided by the user. When a user clicks ‘Search’, the validity of input is checked, the cookie is updated and the form elements are submitted to the format_search component 2216.

Format Search 2216

The Format_Search component 2216 formats the search criteria on the basis of the search type entered by the user. For example, in one embodiment, if the user selects IC50 or Activation, the format_search component 2216 calls the updateDBIC50 component 2310 (described below); otherwise the format_search component calls the appViewBDI2 component 2412 (described below). Comparisons are made between the information in the database and the user defined selections. If an error occurs, or an improper selection has been made the component 2216 detects the error and presents the user interface for Search to the user. If any information is missing, the cookie is checked for missing values. If the information is correct the page continues to the next script.

An embodiment of the present system is capable of performing various types of searches, including but not limited to IC50 2218, PIH 2220, Activation 2222, and Other 2224 searches.

FIG. 23 illustrates the process for analyzing and manipulating IC50 data in an embodiment of this system. Many of the components utilized by an embodiment in performing an IC50 analysis, data manipulation, and search are also used for other types of searches. In such cases, the components are numbered identically in FIGS. 23-25.

Dbparameters 2304

In an embodiment of this system, the dbparameters component 2304 is a dynamic user interface, such as a web page, that is used to provide additional information useful for identifying submitted plates. In one embodiment, the interface includes controls in which a user enters numbers that identify the plate(s). These numbers are used to reference a corporate, proprietary, or other database structure for information relating to these plates.

In some instances, the layout of the plate is derived from previously submitted information within the database structure. In such a situation, the dbparameters component 2304 uses this stored information to fill in at least some of the elements of the user interface, thereby limiting the demands on the user.

In one embodiment, if plate layout information is available, a template representing the plate is dynamically created from that information and displayed on the user interface within the project. The template may be modified by the user within the analysis portion of the user interface, alleviating the need for the user to move between user interface screens to make the modifications.

In an embodiment performing IC50 analysis, manipulation, and/or visualization, the dbparameters component 2304 calls the templateSelectBDI component 2306, passing the user-supplied or database-derived parameters. In other embodiments, such as for analyzing Activation and PIH, the updateBDI_info component 2406 is called.

templateSelectBDI 2306

In an embodiment of this system, the templateSelectBDI component 2306 is a user interface component, such as a web page, that allows users to define a template for use in analysis. In a multiple plate analysis, this template is used for the batch of plates as well. This dynamic interface uses the information from the dbparameters component 2304, either user or database-derived, and additional information from the database(s) to dynamically define a basic template.

In one embodiment, as illustrated by the screen shot of FIG. 23a, plate wells that do not contain compound are colored black. C+ and C− control wells are colored light-grey and dark grey, respectively. Compound wells are a default white.

The user interface provides a means to make changes to the templates. For example, in the embodiment shown in FIG. 23a, command buttons exist within the interface allowing the user to define the mouse interaction with the component or techlet. If the user clicks ‘C+’, mouse drags over the techlet will define C+ control wells. Likewise, if the user clicks ‘C−’, mouse drags over the techlet will define C− control wells. If the user clicks ‘Invalid’, the mouse defines empty wells, and if the user clicks ‘Data’ the mouse defines data wells.

Clicking ‘Reset’ in the embodiment shown, resets the techlet to the default calculated template. Clicking ‘Submit’ sets a cookie and page elements and submits the page elements to the updateDBselect component 2310.

updateDBselect 2310

In the embodiment shown, the updateDBselect component 2310 receives data elements from the templateSelectBDI 2308 component and updates the database with new values created via the template user interface, such as that shown in FIG. 23a. The component 2310 then retrieves values from the database and calls the updateDBIC50 2310 or appViewBDI 2314 component.

updateDBIC50 2310

In one embodiment, as shown in FIG. 23, the updateDBIC50 component 2310 creates a connection to the database and retrieves the necessary data for the APTCO component (described below). The updateDBIC50 component 2310 may also update the database with calculated values from an analysis session and may be executed several times within the session. It may use various other components to perform functions. For example, in one embodiment, the updateDBIC50 component calls the updateDBICflag, which updates the database with calculated values and any changes made relating to the analysis or compounds. In a further embodiment, the component 2310 calls the APTCO component (described below).

appViewBDI 2314

In one embodiment of this system, the appViewBDI component 2314 is a user interface generation script, such as a perl script that generates an html document. The user interface includes the Assay Analaysis View component 2124 described in relation to FIG. 21 above.

The user interface provides the user with a control, such as a text box, for specifying the screening threshold. Changes to the value are reflected in the view 2124 either automatically or in response to a user action, such as clicking a command button.

In one embodiment, elements of the user interface are created dynamically. For example, in one embodiment, buttons are dynamically created for each compound. As each button is selected, the related compound is highlighted in the techlet 2124. Clicking ‘Continue’ updates the cookie, sets form elements and calls both the bkBioReport 2314 and updateDBcalc 2416, updating the database and generating a printable report through the script bkBioReport. The button ‘Help’, displays help.

If multiple plates have been submitted for the current session, buttons appear at the bottom of the techlet 2124, allowing navigation through the array of plates. The buttons indicate usage by arrows. The button first allows a user to go to the first plate. The next button allows navigation to the previous plate display. The third button navigates to the next page and the last button navigates to the last plate in the plate array.

updateBDI info 2406

The updateBDI_info component 2406 is a background component used for database updates. It accepts the information gathered by the dbparameters component 2304 and updates the database. In one embodiment, if information is missing from dbparameters 2304, the updateBDI_Info component recalls the dbparameters user interface. If successful, it calls the templateSelectBDI component 2306.

updateDBcalc 2416

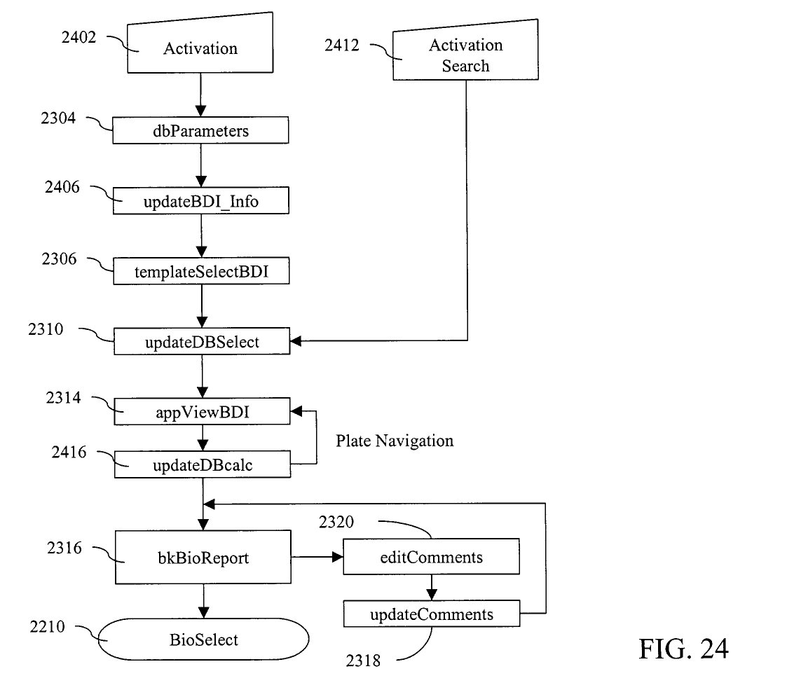

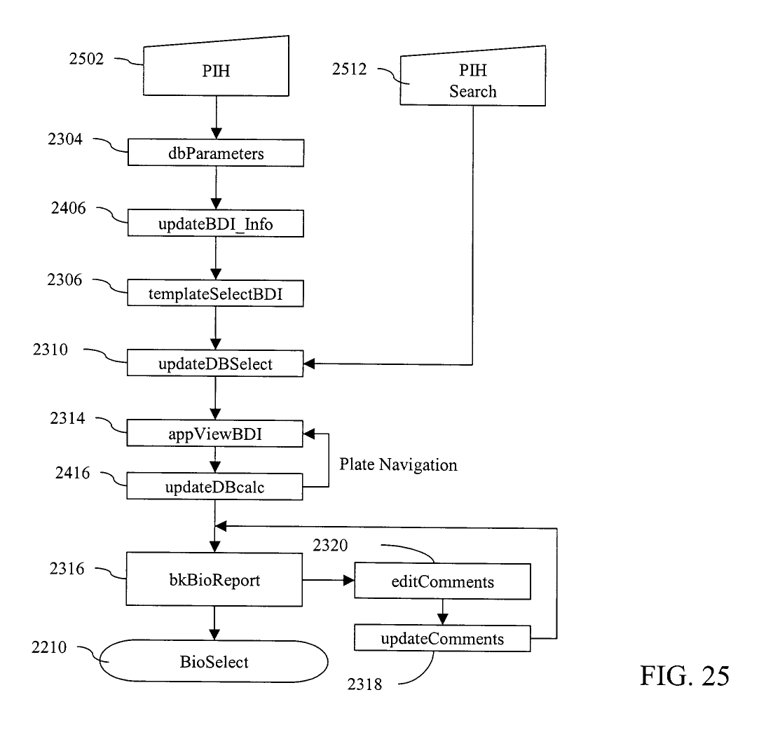

In the embodiments of this system shown in FIGS. 24 and 25, the updateDBcalc component 2416 accepts the updated form elements from appViewBDI 2314 and updates the database. This component 2416 to subsequent components based on user input; if ‘Continue’ is selected by a user, the component 2416 calls the bkBioReport component 2316. If the user is analyzing multiple plates and has selected ‘Next’, ‘Previous’, ‘First’, or ‘Last’, the appViewBDI component 2314 is executed, passing the appropriate parameters to complete the user's request.

APTIC

The APTIC component (not shown) is a component that creates a user interface, such as an HTML page housing a techlet. The user interface allows the user to define the location of compounds within a plate layout. APTIC calls the APTIC2 component (described below).

APTIC2

The APTIC component (not shown) is a component that creates a user interface, such as an HTML page housing a techlet. The user interface allows the user to define the location of concentrations within a plate layout. APTIC calls the APTCO component (described below).

APTCO

The APTCO component creates a user interface that displays the relationships between compound and concentration definitions defined in the previous two components (APTIC and APTIC2). The techlet formulates calculated values dynamically based on the calculation type and the raw data from the data file. If any elements are not present from the database query done by updateDBIC50 2310, they are retrieved from the cookie.

The user interface includes a Screening Threshold control as described above.

Additional user controls, such as buttons, are dynamically created for each compound. As each button is selected, the related compound is highlighted in the techlet. The compounds can be plotted by clicking the ‘Plot’ button. This calls updateDBIC50 2310. By clicking ‘Invalidate’, wells within the plate layout can be removed from the calculation. Clicking ‘Continue’ updates the cookie*, sets form elements and calls both bkBioReport (described above) and updateDBICflag (described above in relation to the udpateDBIC50 component 2310), updating the database and generating a printable report through the script bkBioReport2.

IC Plotter

ICplotBDI (not shown) is executed by APTCO. In one embodiment, the component is a Perl script that generates a HTML document housing a techlet. This techlet dynamically plots the compounds. The techlet also incorporates keyboard and mouse interaction to change aspects of the plotting application.

Buttons are located on the page for interaction with the techlet as well. By entering values within appropriate text boxes and clicking ‘Set Y Axis’ or ‘Set X Axis’ the axis value within the techlet are changed. By clicking ‘Grid’, a visual grid toggles within the techlet display. Clicking ‘Deviate’ causes the display to show a deviated calculation display. For example, the average and standard deviation of a data point may be plotted instead of individual data points at the same concentration, i.e., an experiment may be run multiple times so that a user can show all data points or take an average and a standard deviation of these points.

In one embodiment, the button ‘Replot’ causes a manual recalculation of the plot(s). ‘AutoPlot’ is a button that, when clicked, toggles the techlet's plotting status. In the ‘on’ state, the techlet automatically replots after any change is detected however, in the ‘off’ state the techlet does not automatically redraw itself after a change and must be manually replotted using the ‘Replot’ button. ‘Print’, when clicked, prints the techlet. ‘Get Structure’ is another button that when clicked calls a script called QueryChem.

In one embodiment, when ‘Continue’ is clicked, updateDBIC50 and updateDBICflag are called. These two scripts update the database with the changes made within the techlet and APTCO is refreshed incorporating the changes made while plotting.

If the user clicks ‘Close’, the plotter is closed and no changes are recorded.

QueryChem

In an embodiment of this system, QueryChem (not shown) is a component, such as a script, that generates a HTML form that automatically submits itself to infosearch.html on a separate server.

bkBioReport2

In one embodiment of this system, the bkBioReport2 component (not shown) is a dynamic perl script that generates a printable report with three tables. The first is a table displaying raw data in a relative plate format. The second displays calculated percent inhibition values in a relative plate format. The third displays the percent inhibitions sorted by compound ID and concentration, including an average and standard deviation for each concentration per compound.

The tables are color-coded based on values defined in APTCO and the ICplotter. Green indicates compounds that showed inhibition based on the user defined threshold value. Red indicates an invalid point, not used in calculation. Light Grey indicates C+ and a darker grey indicates a C− value.

Located at the bottom of the page is a legend describing the color codes and three buttons. The first button is ‘Print’, which prints the report. The second button is executed ‘Return to Upload’. When clicked, ‘Return to Upload’ causes the current project to close and returns the user to BioSelect. The third button is executed ‘Edit Comments’.

When ‘Edit Comments’ is clicked, a script called editComments is executed that allows a user to edit the comments stored in the database relating to the analysis session.

bkBioReport 2316

In an embodiment of this system, the blkBioReport component 2316 generates a printable report containing data tables. For example, in one embodiment, the component 2316 creates three tables. The first is a table displaying raw data in a relative plate format. The second displays calculated percent inhibition values in a relative plate format. The third displays the compounds that showed inhibition based on the user-defined threshold in a list format, sorted by inhibition value. The list identifies the compound by ID as well as plate and well location. The compound ID's are hyperlinks that, when clicked, call QueryChem which displays the information from the corporate database for the compound identified by the specific ID number.

The tables are color-coded based on values defined in APTCO and the ICplotter. Green indicates compounds that showed inhibition based on the user defined threshold value. Red indicates an invalid point, not used in calculation. Light Grey indicates C+ and a darker grey indicates a C− value.

Located at the bottom of the page is a legend describing the color codes and three buttons. The first button is ‘Print’, which prints the report. The second button is executed ‘Return to Upload’. When clicked, ‘Return to Upload’ causes the current project to close and returns the user to BioSelect. The third button is executed ‘Edit Comments’.

When ‘Edit Comments’ is clicked, a script called editComments is executed that allows a user to edit the comments stored in the database relating to the analysis session.

editComments 2310

The editComments component 2310 is a script called by both bkBioReport 2316 and bkBioReport2 (described above). The component 2310 retrieves comments from the database that were defined in BioSelect 2210 or BioSelectBDI 2206 and displays the comments in a text area for editing.

When a user clicks ‘Reset’ in this window, the comments are refreshed from the database. When a user clicks ‘Update’, the contents of the text are submitted to updateComments 2318.

updateComments 2318

The updateComments component in an embodiment of this system receives the comments and any changes made in the display of editComments 2320 and these changes are updated to the database and the previous report page (bkBioReport 2316 or bkBioReport2 (not shown)) is refreshed. It may also display a momentary ‘success’ message upon updating and automatically closes itself.

Compound Selection Template

The Compound Selection Template (not shown) allows the user to select areas of the plate that are to be related to an individual compound. The user selects which label they want to relate first, then the user clicks and drags over any number and combination of wells on the plate. These will be highlighted in dark-blue for the current label. When the user selects the next compound label, if there is more than one compound on the plate, then the selected areas of other labels will fade to a light-blue to designate that they have been used.

Once all compounds have been designated on the plate, the user selects the wells to be used for the “controls” of the assay. Light-grey to designate the control-plus, usually the maximum, and dark-grey to designate the control-minus, usually the background. Once the controls have been defined, the user may define the remaining area, if any, as invalid. The invalid regions will be colored black to easily display which areas will not be used.

When all regions have been designated, the user selects ‘Next’ to continue to the Concentration Selection Template.

Concentration Selection Template

In an embodiment of this system, the Concentration Selection Template component is similar to the Compound Selection component or techlet, but it maintains the previous techlet's settings of invalid areas and control point areas, leaving the unused areas as white or cleared. The user again selects the concentrion they wish to relate and then clicks and drags over any number and combination of wells on the plate. These will be high-lighted in dark-blue for the current concentration. When the user selects the next concentration, if there is more than one concentration on the plate, then the selected areas of the other concentrations will fade to light-blue to designate that they have been used.

When all white regions have been designated, the user selects ‘Next’ to continue to the Assay Analysis.

An embodiment of the present system may be used to perform numerical analysis in a variety of situations. For example, embodiments of the present system may be used to perform molecular discovery, pharmaceutical data analysis, chemical efficacy result studies, statistical analysis, and other scientific and mathematical functions.

As is known to one skilled in the art, an embodiment of the present system includes administrative components and data structures. Because data analyzed within the user interface according to the present system may be considered confidential and/or proprietary, and embodiment of the present system will also include various security features. Also, since embodiments of the present system may be used to analyze, manipulate, and visualize various types of data, billing and licensing of the software may take many forms. For example, a developer of software according to the present system may create each of the various components as a stand alone product for licensing purposes. Another developer may create a single integrated application that includes all of the above-described components.

### EXAMPLE PROBES

Mass spectra were acquired on a Micromass ZMD 4000 with an ESI continuous flow probe equipped with a CTC Analytics PAL autosampler and a Waters 600 pump. Samples were dissolved in methanol/tetrahydrofuran at a concentration of 1 mg/mL and transferred to 96 well microtiter plates and data was collected over 30 seconds.

### Example Probe 1

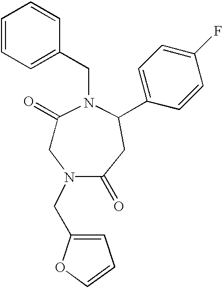

The compound above was prepared with the protocol for Library 7 using: 3-N-Boc-amino-3-(4-fluorophenyl)propionic acid as the amino acid, benzaldehyde for reductive amination, bromoacetic acid, and furfuryl amine. MS (m/z) 463.9 (M+H).

### Example Probe 2

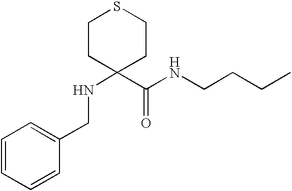

The compound above was prepared with the protocol for Library 120 with n-butyl amine used in reductive amination of resin, 4-N-Fmoc-amino-4-carboxy-tetrahydrothiopyran as the Fmoc amino acid and benzaldehyde as the aldehyde. MS (M/Z) 307.8 (M+H).

### Example Probe 3

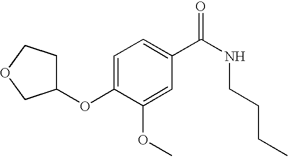

The compound above was prepared with the protocol for Library 12 with n-butyl amine used in reductive amination of resin, 4-hydroxy-3-methoxy-benzoic acid, and tetrahydrofuran-3-ol. MS (M/Z) 294.8 (M+H).

### Example Probe 4

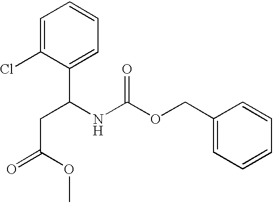

The compound above was prepared with the protocol for Library 63 using: 3-N-Boc-amino-3-(2-chlorophenyl)propionic acid as the amino acid, benzyl alcohol and methanol for cleavage. MS (M/Z) 348.7 (M+H).

### Example Probe 5

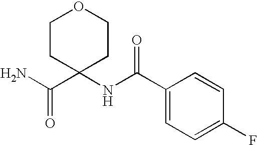

The compound above was prepared with the protocol for Library 102 using 4-N-Fmoc-amino-4-carboxy-tetrahydropyran as the Fmoc amino acid and 4-fluorobenzoic acid. MS (M/Z) 268.7 (M+H).

### Example Probe 6

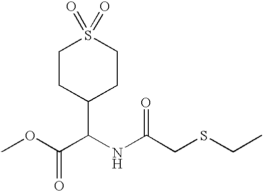

The compound above was prepared with the protocol for Library 95 using: N-Fmoc-amino-4-(1,1-dioxo-tetrahydrothiopyranyl)acetic acid as the amino acid, (ethylthio)acetic acid and methanol for cleavage. MS (M/Z) 324.8 (M+H).

### Example Probe 7

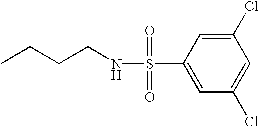

The compound above was prepared with the protocol for Library 119 using: n-butyl amine for reductive amination onto the resin and 3,5-dichlorobenzenesulfonyl chloride. MS (M/Z) 284.7 (M+H).

### Example Probe 8

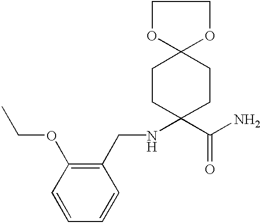

The compound above was prepared with the protocol for Library 103 using N-Fmoc-amino-4-(ethylene ketal)cyclohexanecarboxylic acid as the amino acid and 2-ethoxybenzaldehyde. MS (M/Z) 335.9 (M+H).

### Example Probe 9

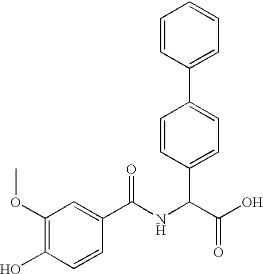

The compound above was prepared with the protocol for Library 105 using 4-N-Fmoc-amino-biphenyl acetic acid as the Fmoc amino acid and 4-hydroxy-3-methoxybenzoic acid. MS (M/Z) 378.8 (M+H).

### Example Probe 10

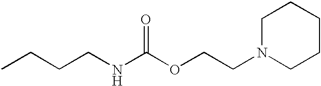

The compound above was prepared with the protocol for Library 136 using: n-butyl amine for reductive amination onto the resin and 2-piperidin-1-ylethanol. MS (M/Z) 229.7 (M+H).

### Example Probe 11

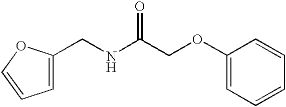

The compound above was prepared with the protocol for Library 118 using: furfuryl amine for reductive amination onto the resin and phenoxy acetic acid. MS (M/Z) 232.7 (M+H).

### Example Probe 12

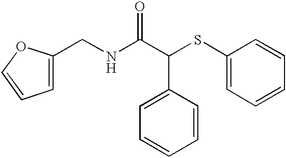

The compound above was prepared with the protocol for Library 24 using: furfuryl amine for reductive amination onto the resin, □-bromo phenyl acetic acid and thiophenol. MS (M/Z) 324.8 (M+H).

### Example Probe 13

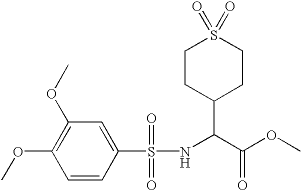

The compound above was prepared with the protocol for Library 74 using: N-Fmoc-amino-4-(1,1-dioxo-tetrahydrothiopyranyl)acetic acid as the amino acid, 3,4-dimethoxybenzenesulfonyl chloride and methanol for cleavage. MS (M/Z) 422.8 (M+H).

### Example Probe 14

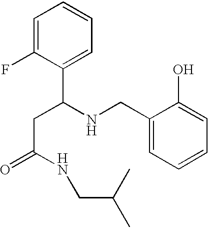

The compound above was prepared with the protocol for Library 73 using: 3-N-Boc-amino-3-(2-fluorophenyl)propionic acid as the amino acid, 2-hydroxybenzaldehyde and isobutylamine for cleavage. MS (M/Z) 345.9 (M+H).

### Example Probe 15

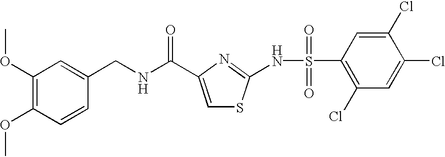

The compound above was prepared with the protocol for Library 126 using: 3,4-dimethoxybenzyl amine for reductive amination onto the resin Fmoc-2-amino-1,3-thiazole-4-carboxylic acid as the amino acid and 2,4,5-trichlorobenzenesulfonyl chloride. MS (M/Z) 538.5 (M+H).

### Example Probe 16

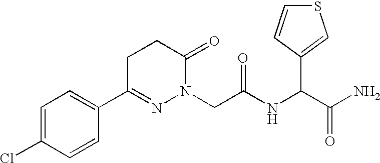

The compound above was prepared with the protocol for Library 1 using: Fmoc-amino-(3-thienyl)acetic acid as the Fmoc amino acid, bromoacetic acid, and 3-(4-chlorobenzoyl) propionic acid. MS (M/Z) 405.71 (M+H).

### Example Probe 17

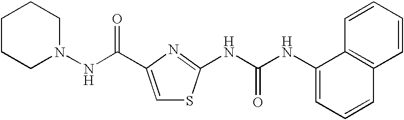

The compound above was prepared with the protocol for Library 121 using: 1-amino-piperidine for reductive amination onto the resin, Fmoc-2-amino-1,3-thiazole-4-carboxylic acid as the amino acid and 1-naphthyl isocyanate. MS (M/Z) 397.8 (M+H).

### Example Probe 18

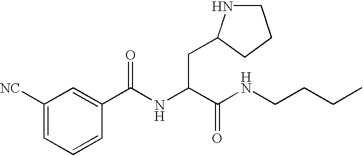

The compound above was prepared with the protocol for Library 122 using: n-butyl amine for reductive amination onto the resin, 2-N-Fmoc-amino-3-(2-N-Boc-amino-pyrrolidinyl)propionic acid as the amino acid and 3-cyanobenzoic acid. MS (M/Z) 343.9 (M+H).

### Example Probe 19

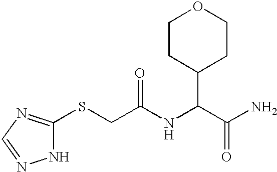

The compound above was prepared with the protocol for Library 32 using N-Fmoc-amino-(4-tetrahydropyranyl)acetic acid as the amino acid, bromoacetic acid, and 4H-1,2,4-triazole-3-thiol. MS (M/Z) 300.7 (M+H).

### Example Probe 20

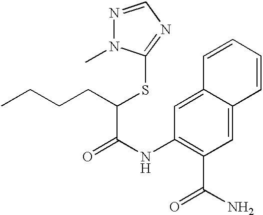

The compound above was prepared with the protocol for Library 33 using N-Fmoc-3-amino-2-naphthoic acid as the amino acid, 2-bromohexanoic acid, and 4-methyl-4H-1,2,4-triazole-3-thiol. MS (M/Z) 398.8 (M+H).

### Example Probe 21

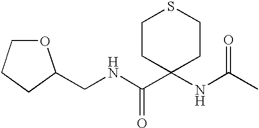

The compound above was prepared with the protocol for Library 123 using tetrahydrofurfuryl amine for reductive amination onto the resin, 4-N-Fmoc-amino-4-carboxy-tetrahydrothiopyran as the amino acid, and acetic anhydride. MS (M/Z) 287.7 (M+H).

### Example Probe 22

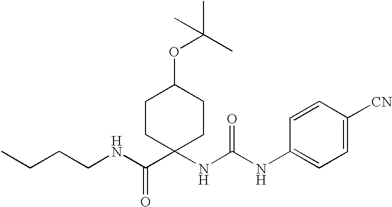

The compound above was prepared with the protocol for Library 128 using n-butyl amine for reductive amination onto the resin, 4-N-Fmoc-amino-(4-t-butoxycyclohexyl)carboxylic acid as the amino acid, and 4-aminobenzonitrile. MS (M/Z) 415.9 (M+H).

### Example Probe 23

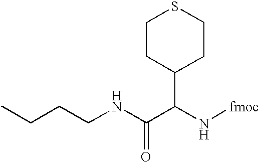

The compound above was prepared with the protocol for Library 115 using n-butyl amine for reductive amination onto the resin, N-Fmoc-amino-(4-tetrahydrothiopyranyl)acetic acid as the amino acid. MS (M/Z) 453.9 (M+H).

### Example Probe 24

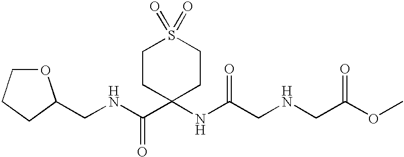

The compound above was prepared with the protocol for Library 38 using tetrahydrofurfurly amine for reductive amination onto the resin, 4-N-Fmoc-amino-4-carboxy-1,1-dioxo-tetrahydrothiopyran as the amino acid, bromoacetic acid, and glycine methyl ester. MS (M/Z) 406.8 (M+H).

### Example Probe 25

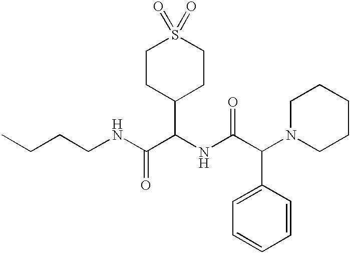

The compound above was prepared with the protocol for Library 42 using n-butyl amine for reductive amination onto the resin, N-Fmoc-amino-4(1,1-dioxo-tetrahydrothiopyranyl)acetic acid as the amino acid, □-bromo phenyl acetic acid, and piperidine. MS (M/Z) 464.9 (M+H).

### Example Probe 26

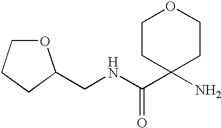

The compound above was prepared with the protocol for Library 116 using tetrahydrofurfurly amine for reductive amination onto the resin, and 4-N-Fmoc-amino-4-carboxy-tetrahydropyran as the amino acid. MS (M/Z) 228.7 (M+H).

### Example Probe 27

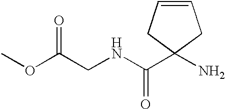

The compound above was prepared with the protocol for Library 117 using glycine methylester for reductive amination onto the resin, and N-Boc-amino-cyclopent-3-ene-carboxylic acid as the amino acid. MS (M/Z) 200.6 (M+H).

### Example Probe 28

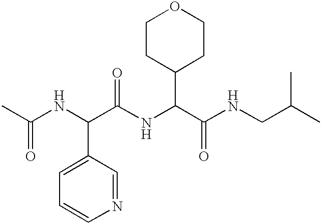

The compound above was prepared with the protocol for Library 178 using N-Fmoc-amino-(4-tetrahydropyranyl)acetic acid as the first amino acid, 3-pyridyl-N-Fmoc-aminoacetic acid as the second amino acid, acetic anhydride and isobutyl amine for cleavage MS (M/Z) 391.9 (M+H).

### Example Probe 29

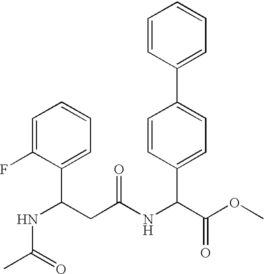

The compound above was prepared with the protocol for Library 180 using N-Fmoc-amino-biphenyl acetic acid as the first amino acid-3-N-Boc-amino-3-(2-fluorophenyl)propionic acid as the second amino acid, acetic anhydride and methanol for cleavage MS (M/Z) 449.9 (M+H).

### Example Probe 30

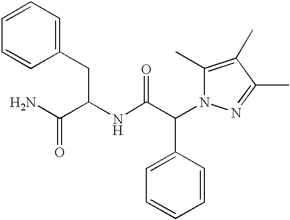

The compound above was prepared with the protocol for Library 9 using, Fmoc-phenylalanine as the Fmoc amino acid, □-bromo phenyl acetic acid, and 3-methyl-2,4-pentanedione. MS (M/Z) 392.0 (M+H).

### Example Probe 31

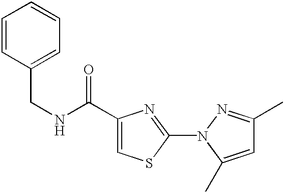

The compound above was prepared with the protocol for Library 8 using benzyl amine used in reductive amination of resin and 2,4-pentanedione as the 1,3-diketone. MS (M/Z) 314.0 (M+H).

### Example Probe 32

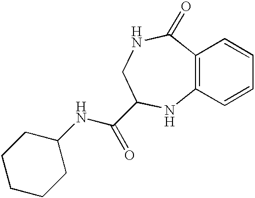

The compound above was prepared with the protocol for Library 11 using ethanolamine used in reductive amination of resin and Fmoc-anthranilic acid and cyclohexyl isocyanide used in the Ugi reaction. MS (M/Z) 389.0 (M+H).

### Example Probe 33

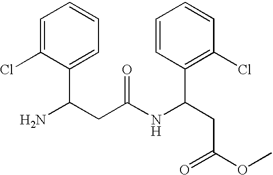

The compound above was prepared with the protocol for library 139 using 3-N-Boc-amino-3-(2-chlorophenyl)propionic acid and methanol for cleavage. MS: M/Z 397.8 (M+2H)⁺.

### Example Probe 34

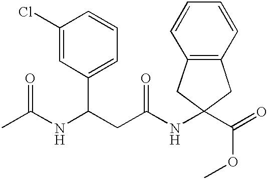

The compound above was prepared with the protocol for library 176 using Fmoc-2-aminoindane-2-carboxylic acid, 3-N-Boc-amino-3-(3-chlorophenyl)propionic acid and acetic anhydride and methanol for cleavage. MS: M/Z 399.9 (M+H)⁺.

### Example Probe 35

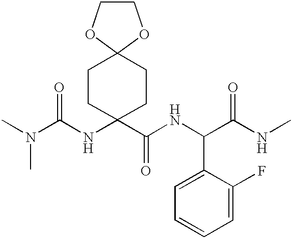

The compound above was prepared with the protocol for library 169 using 3-N-Boc-amino-3-(2-fluorophenyl)propionic acid, N-Fmoc amino-4-(ethylene ketal)cyclohexylcarboxylic acid, dimethylcarbamoyl chloride and methyl amine. MS: M/Z 452.0 (M+H)⁺.

### Example Probe 36

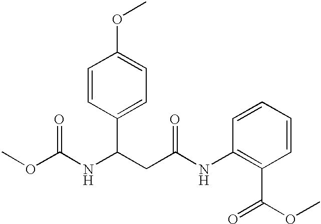

The synthesis of the above molecule was performed using the protocol of library 148 using Fmoc-2-aminobenzoic acid, 3-N-Boc-amino-3-(4-methoxyphenyl)propionic acid methylchloroformate and methanol. MS: M/Z 387.8 (M+H)⁺.

### Example Probe 37

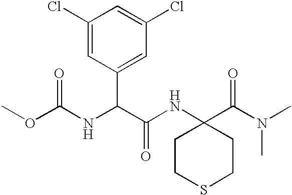

The synthesis of the above molecule was performed using the protocol of library 146 using 4-N-Fmoc-amino-4-carboxytetrahydrothiopyran, N-Fmoc-amino-(3,5-dichlorophenyl)acetic acid, methylchloroformate and dimethylamine. MS: M/Z 450.0 (M+2H)⁺.

### Example Probe 38

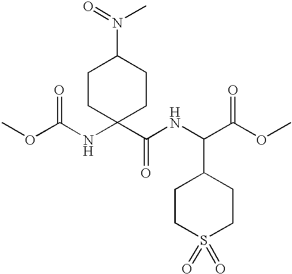

The synthesis of the above molecule was performed using the protocol of library 50 using N-Fmoc-amino-4-(1,1-dioxotetrahydrothiopyranyl)acetic acid, N-Fmoc-amino-(4-N-Boc-piperidinyl)carboxylic acid, methylchloroformate, acetic anhydride, and methanol. MS: M/Z 450.8 (M+2H)⁺.

### Example Probe 39

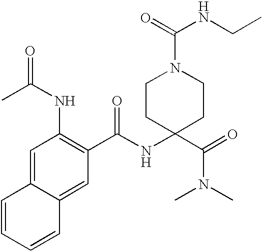

The synthesis of the above molecule was performed using the protocol of library 54 using N-Fmoc-amino-(4-N-Boc-piperidinyl)carboxylic acid, ethyl isocyanate, 3-N-Fmoc-amino-2-naphthoic acid, acetic anhydride and dimethylamine. MS: M/Z 454.9 (M+H)⁺.

### Example Probe 40

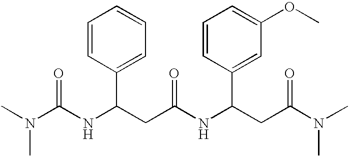

The synthesis of the above molecule was performed using the protocol of library 170 using 3-N-Boc-amino-3-(3-methoxyphenyl)propionic acid, 3-N-Boc-amino-3-phenylpropionic acid, dimethylcarbamoyl chloride and dimethylamine. MS: M/Z 442.0 (M+H)⁺.

### Example Probe 41

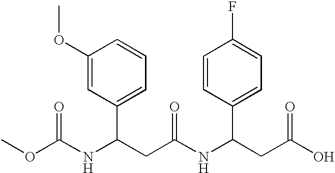

The synthesis of the above molecule was performed using the protocol of library 147 using 3-N-Boc-amino-3-(4-fluorophenyl)propionic acid, 3-N-Boc-amino-3-(3-methoxyphenyl)propionic acid, methylchloroformate and sodium hydroxide. MS: M/Z 419.9 (M+H)⁺.

### Example Probe 42

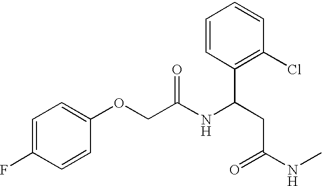

The synthesis of the above molecule was performed using the protocol of library 94 using 3-N-Boc-amino-3-(2-chlorophenyl)propionic acid, (4-fluorophenoxy)acetic acid and methyl amine. MS: M/Z 365.8 (M+H)⁺.

### Example Probe 43

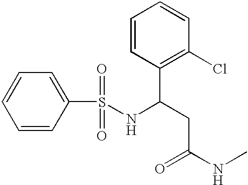

The synthesis of the above molecule was performed using the protocol of library 75 using 3-N-Boc-amino-3-(2-chlorophenyl)propionic acid, benzenesulfonyl chloride and methyl amine. MS: M/Z 353.8 (M+H)⁺.

### Example Probe 44

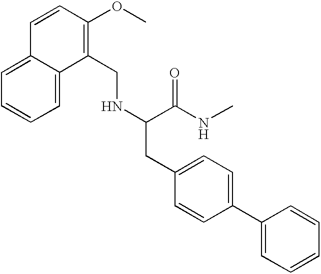

The synthesis of the above molecule was performed using the protocol of library 70 using 2-N-Fmoc-amino-3-biphenylpropionic acid, 2-methoxynaphthaldehyde and methyl amine. MS: M/Z 426.0 (M+H)⁺.

### Example Probe 45

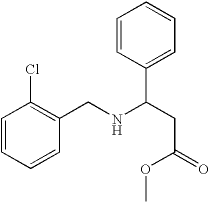

The synthesis of the above molecule was performed using the protocol of library 72 using 3-N-Boc-amino-3-phenylpropionic acid, 2-chlorobenzaldehyde and methanol. MS: M/Z 304.79 (M+H)⁺.

### Example Probe 46

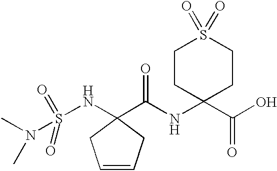

The synthesis of the above molecule was performed using the protocol of library 160 using 4-N-Fmoc-amino-4-carboxy-1,1-dioxotetrahydrothiopyran, N-Boc-amino-cyclopent-3-ene-carboxylic acid, dimethylsulfamoyl chloride and sodium hydroxide. MS: M/Z 410.8 (M+H)⁺.

### Example Probe 47

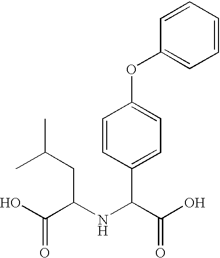

The synthesis of the above molecule was performed using the protocol of library 47 using N-Fmoc-Leucine, glyoxylic acid, and 4-phenoxyphenylboronic acid. MS: M/Z 358.7 (M+H)⁺.

### Example Probe 48

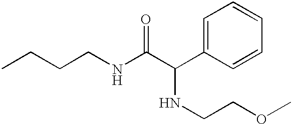

The synthesis of the above molecule was performed using the protocol of library 22 using butylamine, □-phenylbromoacetic acid, and 2-methoxyethylamine. MS: M/Z 265.8 (M+H)⁺.

### Example Probe 49

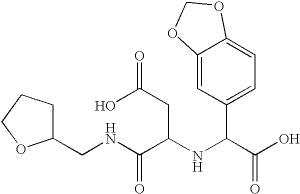

The synthesis of the above molecule was performed using the protocol of library 46 using N-□-Fmoc-L-aspartic acid-□-t-butyl ester, glyoxylic acid, and 3,4-methylenedioxyphenylboronic acid. MS: M/Z 395.7 (M+H)⁺.

### Example Probe 50

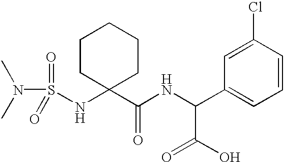

The synthesis of the above molecule was performed using the protocol of library 159 using 3-N-Boc-3-(3-chlorophenyl)propionic acid, N-Fmoc-aminocyclohexylcarboxylic acid, and dimethylsulfamoyl chloride. MS: M/Z 431.6 (M+H)⁺.

### Example Probe 51

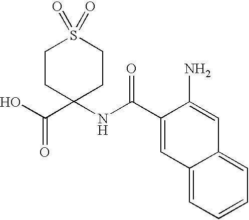

The synthesis of the above molecule was performed using the protocol of library 181 using 4-N-Fmoc-amino-4-carboxy-111-dioxo-tetrahydrothiopyran, and 3-N-Fmoc-2-naphthoic acid. MS: M/Z 363.8 (M+H)⁺.

### Example Probe 52

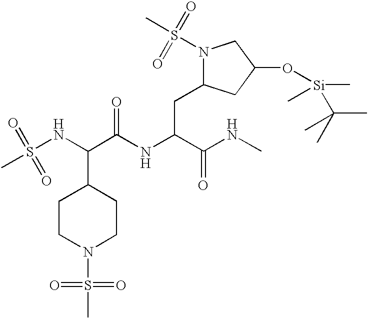

The synthesis of the above molecule was performed using the protocol of library 49 using 2-Fmoc-amino-3-[2-N-Boc-4-(tert-butyldimethylsilyloxy)pyrrolidinyl]propionic acid, and N-Fmoc-amino-(4-N-Boc-piperdinyl)acetic acid, methanesulfonyl chloride, and methylamine. MS: M/Z 563.0 (M+H)⁺.

### Example Probe 53

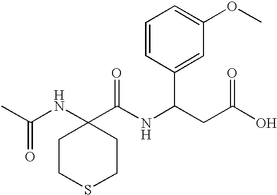

The synthesis of the above molecule was performed using the protocol of library 179 using 3-N-Boc-3-(3-methoxyphenyl)propionic acid, and 4-N-Fmoc-amino-4-carboxy-tetrathiopyran, and acetic anhydride. MS: M/Z 381.8 (M+H)⁺.

### Example Probe 54

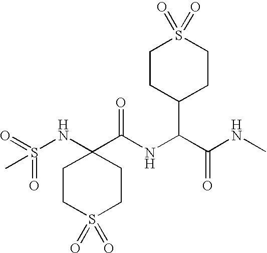

The synthesis of the above molecule was performed using the protocol of library 153 using N-Fmoc-amino-4(1,1-dioxotetrathiopyranyl)acetic acid, and 4-N-Fmoc-amino-4-carboxy-1,1-dioxy-tetrathiopyran, methanesulfonyl chloride, and methylamine. MS: M/Z 474.8 (M+H)⁺.

### Example Probe 55

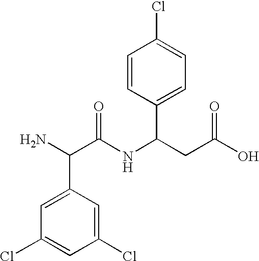

The synthesis of the above molecule was performed using the protocol of library 140 using 3-N-Boc-amino-3-(4-chlorophenyl)propionic acid, and N-Fmoc-amino-(3,5-dichlorophenyl)acetic acid. MS: M/Z 403.6 (M+H)⁺.

### Example Probe 56

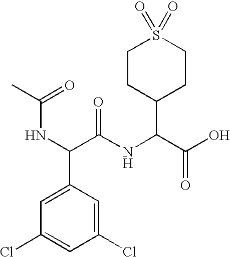

The synthesis of the above molecule was performed using the protocol of library 185 using N-Fmoc-amino-4-(1,1-dioxotetrahydrothiopyranyl)acetic acid, N-Fmoc-amino-(3,5-dichlorophenyl)acetic acid, and acetic anhydride. MS: M/Z 453.8 (M+H)⁺.

### Example Probe 57

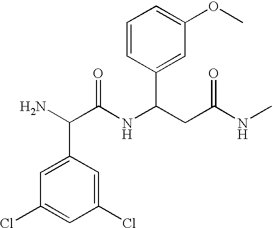

The synthesis of the above molecule was performed using the protocol of library 138 using 3-N-Boc-3-(3-methoxyphenyl)propionic acid, N-Fmoc-amino-(3,5-dichlorophenyl)acetic acid, and methylamine. MS: M/Z 411.8 (M+H)⁺.

### Example Probe 58

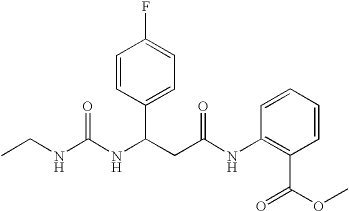

The synthesis of the above molecule was performed using the protocol of library 168 using 2-N-Fmoc-aminobenzoic acid, 3-N-Boc-amino-3-(4-fluorophenyl)propionic acid, ethylisocyanate and methanol. MS: M/Z 388.9 (M+H)⁺.

### Example Probe 59

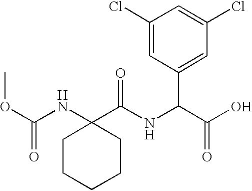

The synthesis of the above molecule was performed using the protocol of library 147 using N-Fmoc-amino-(3,5-dichlorophenyl)acetic acid, N-Fmoc-aminocyclohexylcarboxylic acid, and methylchloroformate. MS: M/Z 405.8 (M+H)⁺.

### Example Probe 60

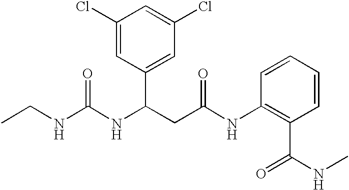

The synthesis of the above molecule was performed using the protocol of library 165 using 2-N-Fmoc-aminobenzoic acid, 3-N-Boc-amino-3-(3,5-dichlorophenyl)acetic acid, ethylisocyanate, and methylamine. MS: M/Z 425.8 (M+H)⁺.

### Example Probe 61

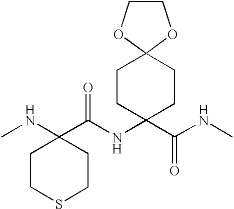

The synthesis of the above molecule was performed using the protocol of library 149 using N-Fmoc-amino-4-(ethyleneketal)cyclohexylcarboxylic acid, 4-N-Fmoc-amino-4-carboxytetrahydrothiopyran, formaldehyde, and methylamine. MS: M/Z 371.9 (M)⁺.

### Example Probe 62

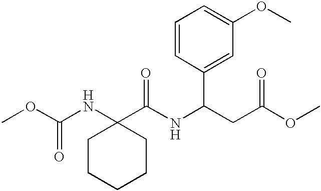

The synthesis of the above molecule was performed using the protocol of library 148 using 3-N-Boc-amino-3-(3-methoxyphenyl)propionic acid, N-Fmoc-aminocyclohexylcarboxylic acid, methylchloroformate, and methanol. MS: M/Z 394.8 (M+H)⁺.

### Example Probe 63

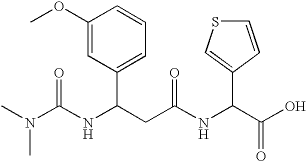

The synthesis of the above molecule was performed using the protocol of library 171 using N-Fmoc-amino-(3-thienyl)acetic acid, 3-N-Boc-amino-3-(3-methoxyphenyl)propionic acid dimethylcarbamoyl chloride, and sodium hydroxide. MS: M/Z 406.9 (M+H)⁺.

### Example Probe 64

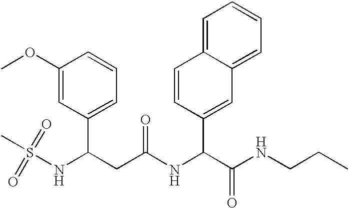

The synthesis of the above molecule was performed using the protocol of library 154 using N-Fmoc-amino-(2-naphthyl)acetic acid, 3-N-Boc-amino-3-(3-methoxyphenyl)propionic acid methanesulfanyl chloride, and propylamine. MS: M/Z 498.95 (M+H)⁺.

### Example Probe 65

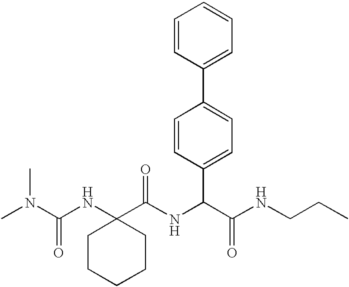

The synthesis of the above molecule was performed using the protocol of library 170 using N-Fmoc-amino-biphenylacetic acid, N-Fmoc-aminocyclohexylcarboxylic acid, dimethylcarbamoyl chloride, and propylamine. MS: M/Z 466.0 (M+H)⁺.

### Example Probe 66

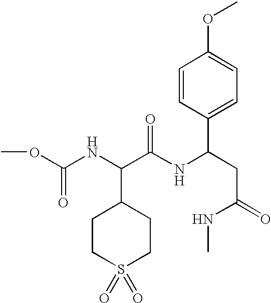

The synthesis of the above molecule was performed using the protocol of library 145 using 3-N-Boc-amino-3-(4-methoxyphenyl)-propionic acid, N-Fmoc-amino-4-(1,1-dioxo-tetrahydrothiopyranyl)acetic acid, methyl chloroformate, and methyl amine. MS: m/z 456.9 (M+H)⁺

### Example Probe 67

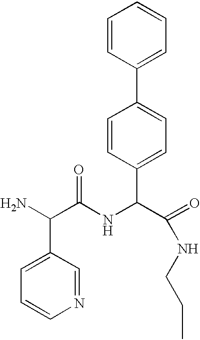

The synthesis of the above molecule was performed using the protocol of library 137 using N-Boc-amino-biphenyl acetic acid, 3-Pyridyl-N-Fmoc-amino acetic acid, and propyl amine. MS: m/z 403.9 (M+H)⁺

### Example Probe 68

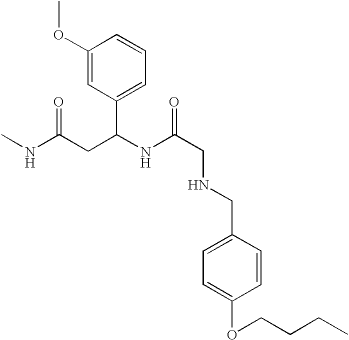

The synthesis of the above molecule was performed using the protocol of library 26 using 3-N-Boc-amino-3-(3-methoxyphenyl)-propionic acid, 4-butoxy benzylamine and methylamine. MS: m/z 428.9 (M+H)⁺

### Example Probe 69

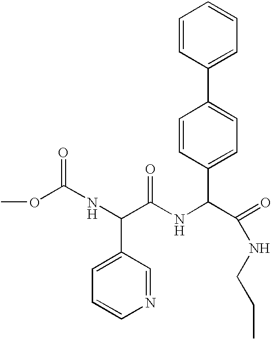

The synthesis of the above molecule was performed using the protocol of library 146 using N-Boc-amino-biphenyl acetic acid, 3-Pyridyl-N-Fmoc-amino acetic acid, methyl chloroformate, and propyl amine. MS: m/z 462.0 (M+H)⁺

### Example Probe 70

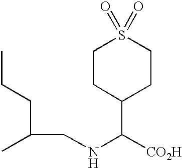

The synthesis of the above molecule was performed using the protocol of library 106 using N-Fmoc-amino-4-(1,1-dioxo-tetrahydrothiopyranyl)acetic acid and 2-methylpentanal. MS: m/z 292.8 (M+H)⁺

### Example Probe 71

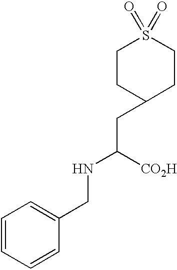

The synthesis of the above molecule was performed using the protocol of library 71 using 2-N-Fmoc-amino-3-[4(1,1-dioxo-tetrahydrothiopyranyl)]propionic acid, benzaldehyde and hydroxide. MS: m/z 312.8 (M+H)⁺

### Example Probe 72

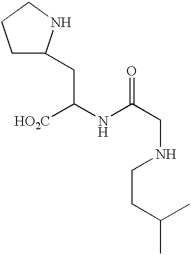

The synthesis of the above molecule was performed using the protocol of library 34 using 2-N-Fmoc-amino-3-(2-N-Boc-amino-pyrrolidinyl)propionic and isovaleraldehyde. MS: m/z 286.9 (M+H)⁺

### Example Probe 73

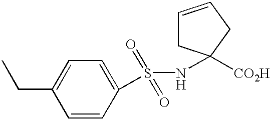

The synthesis of the above molecule was performed using the protocol of library 76 using N-Boc-amino-cyclopent-3-ene-carboxylic acid, 4-ethylbenzenesulfonyl chloride and hydroxide. MS: m/z 296.8 (M+H)⁺

### Example Probe 74

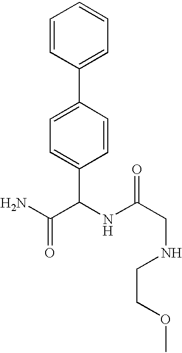

The synthesis of the above molecule was performed using the protocol of library 30 using N-Fmoc-amino-biphenyl acetic acid, bromoacetic acid, and 2-methoxy-ethylamine. MS: m/z 342.9 (M+H)⁺

### Example Probe 75

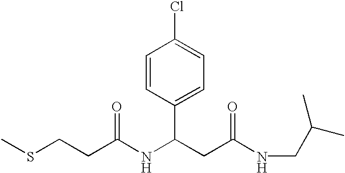

The synthesis of the above molecule was performed using the protocol of library 97 using 3-N-Boc-amino-3-(4-chlorophenyl)-propionic acid, 3-methylmercaptopropionic acid, and isobutylamine. MS: m/z 357.9 (M+H)⁺

### Example Probe 76

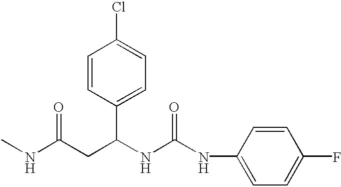

The synthesis of the above molecule was performed using the protocol of library 82 using 3-N-Boc-amino-3-(4-chlorophenyl)-propionic acid, 4-fluoroaniline, and methylamine. MS: m/z 350.8 (M+H)⁺

### Example Probe 77

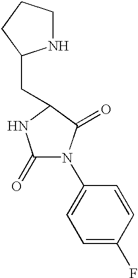

The synthesis of the above molecule was performed using the protocol of library 6 using 2-N-Fmoc-amino-3-(2-N-Boc-amino-pyrrolidinyl)propionic acid and 4-fluoroaniline. MS: m/z 278.8 (M+H)⁺

### Example Probe 78

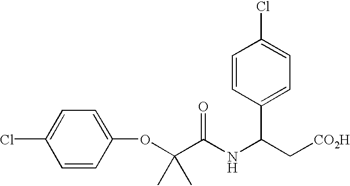

The synthesis of the above molecule was performed using the protocol of library 100 using 3-N-Boc-amino-3-(4-chlorophenyl)-propionic acid, clofibric acid, and hydroxide. MS: m/z 420.7 (M+Na)⁺

### Example Probe 79

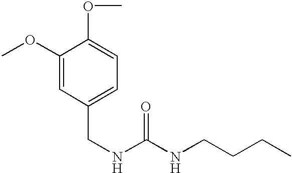

The synthesis of the above molecule was performed using the protocol of library 132 using N-butylamine and 3,4-dimethoxybenzylamine. MS: m/z 267.9 (M+H)⁺

### Example Probe 80

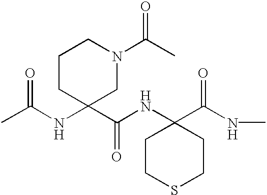

The synthesis of the above molecule was performed using the protocol of library 53 using 4-N-Fmoc-amino-4-carboxytetrahydrothiopyran, N-Fmoc-amino-(3-N-Boc-piperidinyl) carboxylic acid, acetic anhydride, and methyl amine. MS: m/z 385.9 (M+H)⁺

### Example Probe 81

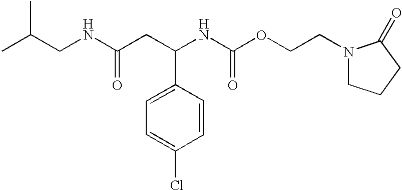

The synthesis of the above molecule was performed using the protocol of library 65 using 3-N-Boc-amino-3-(4-chlorophenyl)propionic acid, 1-(2-hydroxyethyl)-pyrrolidinone, and isobutylamine. MS: M/Z 410.8 (M+H)⁺.

### Example Probe 82

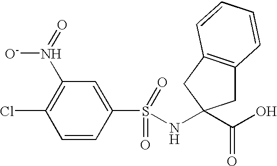

The synthesis of the above molecule was performed using the protocol of library 107 using Fmoc-2-aminoindane-2-carboxylic acid, and 4-chloro-3-nitrobenzenesulfonyl chloride. MS: M/Z 399.3 (M+H)⁺.

### Example Probe 83

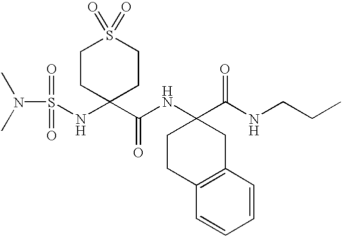

The synthesis of the above molecule was performed using the protocol of library 158 using 2-N-Fmoc-amino-tetrahydro-2-naphthoic acid, 4-N-Fmoc-amino-4-carboxy-1,1-dioxotetrahydrothiopyran, dimethylsulfamoyl chloride and propylamine. MS: M/Z 516.1 (M+H)⁺.

### Example Probe 84

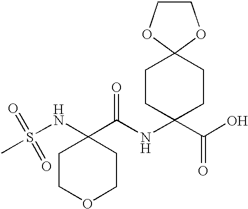

The synthesis of the above molecule was performed using the protocol of library 184 using N-Fmoc-amino-4-(ethyleneketal)cyclohexylcarboxylic acid, 4-N-Fmoc-amino-carboxytetrahydropyran, and methanesulfonyl chloride. MS: M/Z 407.0 (M+H)⁺.

### Example Probe 85

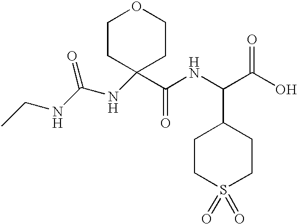

The synthesis of the above molecule was performed using the protocol of library 187 using 2-N-Fmoc-aminobenzoic acid, 4-N-Fmoc-amino-carboxytetrahydropyran, and ethylisocyanate. MS: M/Z 407.3 (M+H)⁺.

### Example Probe 86

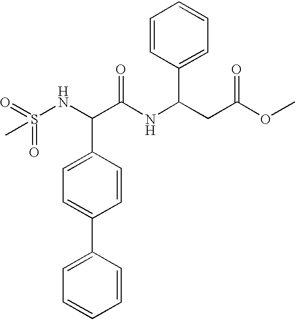

The synthesis of the above molecule was performed using the protocol of library 156 using 3-N-Boc-amino-3-phenylpropionic acid, 2-N-Fmoc-amino-biphenylacetic acid, methanesulfonyl chloride, and methanol. MS: M/Z 467.8 (M+H)⁺.

### Example Probe 87

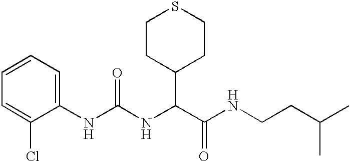

The synthesis of the above molecule was performed using the protocol of library 121 using isoamylamine, 2-N-Fmoc-amino-2-tetrahydrothiopyranacetic acid, 2-chlorophenylisocyanate. MS: M/Z 398.7 (M+H)⁺.

### Example Probe 88

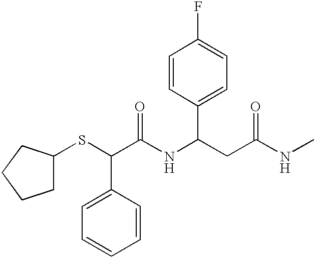

The synthesis of the above molecule was performed using the protocol of library 26 using 3-N-Boc-amino-3-(4-fluorophenyl)propionic acid, alpha-phenylbromoacetic acid, cyclopenylmercaptan, and methylamine. MS: M/Z 415.8 (M+H)⁺.

### Example probe 89

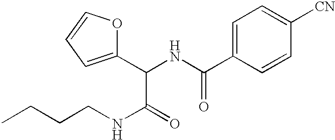

The synthesis of the above molecule was performed using the protocol of library 3 using 4-cyanobenzoic acid, 2-furaldehyde, and n-butylisocyanide. MS: M/Z 326.8 (M+H)⁺.

### Example 90

Thrombin is a suitable target for drug discovery using this method. Thrombin lies in the final common pathway of coagulation and cleaves fibrinogen to fibrin thereby generating the biological polymer which constitutes part of a blood clot in mammals. Therefore, inhibition of thrombin would be expected to exert an antithrombotic effect. In the present embodiment, the X-ray structure of human thrombin (PDB code: 1EB1) retrieved from the protein data bank as used (27280) as the target structure instead of the homology model. In preparing for in silico screening efforts, the inhibitor, and solvent molecules were stripped off the target structures. Alongside, any unfilled valencies in the target structure were occupied with hydrogen atoms and the Gasteiger atomic charges for the target structure was assigned. The association site was characterized (260) by employing the “Cerius²® LigandFit” (Accelrys Inc, San Diego, Calif.) and using the inhibitor three-dimensional structure bound to the target. Since one of the aims of the present embodiment was to discover inhibitor probes for thrombin, as an illustration of the methods involved in the drug discovery process, other association sites identified for the target were not pursued.

In a parallel process, approximately 55,000 of the probe set (261000) compounds representing a subset of the candidate probe set (302000) and encompassing a subset of the framework structures illustrated in schemes 1 through 14, libraries 1 through 202, and examples 1 through 89, were retrieved from the database. The two-dimensional structures of the probes stored in the database were initially cleaned to remove the salts (if present) and subjected to an energy minimization in order to generate the three-dimensional conformation of the probes.

In the next step, in silico screening was performed using the probe set (261000) against the target association site (27260). For each probe, a maximum of one thousand three-dimensional conformations were generated “on the fly” using the Monte Carlo procedure implemented in “Cerius²®” (Accelrys Inc, San Diego, Calif.). Each of these probes conformations was aligned/docked in the target association site (27220). A score value was assigned for each of the target/probe conformer complex using the LigScore_Dreiding scoring function (27230). However, only the top two ranked target/probe conformers for each probe were saved. Subsequently, four more scoring functions (PLP1, PLP2, PMF, and DOCK) were employed to score the two saved target/probe conformer complexes for each probe. A correlation matrix obtained for the five scoring functions showed over 80% correlation between PLP1 and PLP2. Consequently, the results of PLP2 were not used or considered further.

The approximately 110,000 target/probe complexes with the five scoring function values were then imported to the database viewer in MOE (Chemical Computing Group, Montreal, Canada) for rank ordering of the probe set (261000) according to their score values. Two thousand of the top ranked unique probes for each scoring of the four functions were identified, labeled as in silico probe hits (27240) and saved separately. Thus, generating 8,000 in silico probe hits. Subsequently, the plate identification number containing the in silico probe hits along with the number of in silico probe hits in each of these plates were obtained.

Instead of performing in biologico screening on the 8,000 in silico probe hits obtained by filtering the top two thousand best ranked unique probes using each of the four scoring functions, a subset of the 8,000 in silico probe hits were obtained for subsequent screening activities. A subset of the 8,00 in silico probe hits was achieved by selecting the top five ranked plates that contained the maximum number of in silico probe hits for each of the scoring functions resulting in twenty plates used towards in biologico screening against thrombin. Although it was more relevant to screen only those probes that were identified as in silico probe hits in these plates, the computed Tc revealed that the other probes in each of the plates containing in silico probe hits to be near neighbors (30570). Hence, all the probes contained in all the twenty plates were subjected to in biologico screeing against thrombin.

Based on the dose-response nature of the in biologico screened probes, the success of the in silico protocols in discovering probes for any given target is exemplified using one of the in silico probe hits that was also identified as an in biologico hit, too (29440).

Multiple x-ray crystal structures (27280) of thrombin are freely available via the Protein Data Bank (PDB), enabling the selection in silico of a thrombin—associating probe molecule according to this disclosure.

The biological assay (28320) for thrombin inhibitory activity is detailed below. To Nunc 96-well black fluorescence plate wells is added 70 microliters of assay buffer, followed by 10 microliters of 1 millimolar substrate solution. Test probe (10 microliters in 30% DMSO) is then added to wells according to the desired concentrations for the assay. The mixture is incubated at 37° C. for 5 minutes, followed by addition of 10 microliters of thrombin (100 micrograms/mL in assay buffer), to make a final assay volume of 100 microliters. The plate is mixed gently and incubated 15 minutes at 37° C. Stop buffer (100 microliters) is added, and the plate is read by detecting emission at 460 nM. Percent inhibition of test compound is calculated by comparison with control wells. “Assay buffer” is composed of 100 mM KH₂PO₄, 100 mM Na₂HPO₄, 1 mM EDTA, 0.01% BRIJ-35, and 1 mM dithiothreitol (added fresh on the day assay is preformed). “Stop buffer” is composed of 100 mM Na—O(O)CCH₂Cl and 30 mM sodium acetate which is brought to pH 2.5 with glacial acetic acid. Thrombin was purchased from Sigma (cat #T-3399). Thrombin substrate III fluorogenic was purchased from ICN (cat #195915). Sodium acetate, dithiothreitol, and Brij-35 were purchased from Sigma. Sodium monochloroacetate was purchased from Lancaster 223-498-3. Glacial acetic acid was purchased from Alfa Aesar (cat # 33252). Thrombin was stored at −20° C. Thrombin substrate fluorogenic was stored at −20° C. (5 mM in DMSO).

Results are expressed as percentage inhibition at a given test probe concentration in the Table below;

| | | % inhibition | % inhibition |
|---|---|---|---|
| Example | MOLSTRUCTURE | @ 100 μM | @ 50 μM |
| B1 | 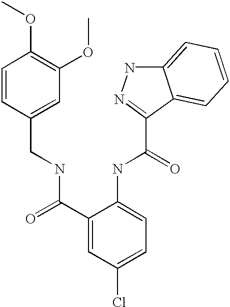 | +++ | ++ |
| B2 | 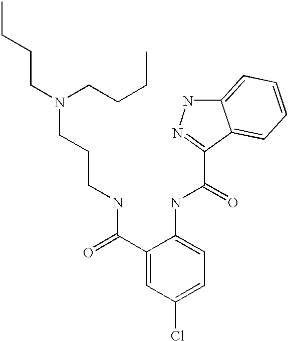 | +++ | ++ |
| B3 | 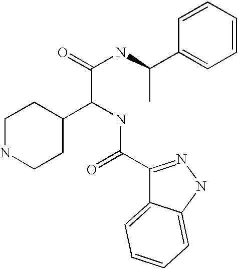 | +++ | ++ |

Key
++++ 75-100%
+++ 40-74%
++ 10-39%
+ 0-10%

Synthesis of Thrombin Inhibitory Library

General Procedure:

Aldehyde resin was reductively aminated with an amine input as described in general procedure 1.D.5. To this was coupled either N-Fmoc-amino-(4-N-Boc-piperidinyl) acetic acid (B-AA1)or 2-N-Fmoc-amino-5-chlorobenzoic acid (B-AA2) as described in general procedure 1.D.1. The Fmoc group was removed with 20% piperidine in DMF as described in general procedure 2A. The resulting free amine was acylated with a carboxylic acid input as described in general procedure 3.A. The resulting diamide was removed from the resin and the Boc groups removed as described in general procedure 11.L.2 to yield either I or II as shown below:

| | |
|---|---|
| | I |
| 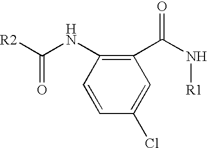 | |
| | II |
| 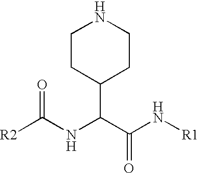 | |

| | | | | Mass | |
|---|---|---|---|---|---|
| | Amino | R1 | R2 | Spectrum | |
| Eg | Acid Input | Amine Input | Acid Input | M/Z | Structure |
| B1 | 2-N- Fmoc- amino-5- chlorobenzoic acid | 3,4- dimethoxybenzyl amine | Indazole-3- carboxylic acid | 465.9 (M + H)⁺ | 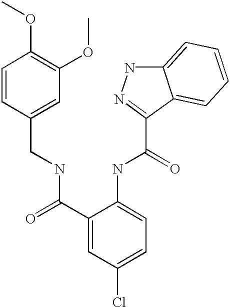 |
| B2 | 2-N- Fmoc- amino-5- chlorobenzoic acid | 3-(Di-N- butylamino)propyl amine | Indazole-3- carboxylic acid | 485.9 (M + H)⁺ | 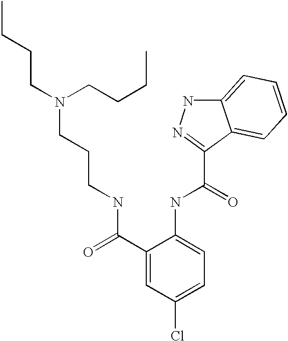 |
| B3 | B-AA1 | Methyl benzylamine | Indazole-3- carboxylic acid | 406.8 (M + H)⁺ | 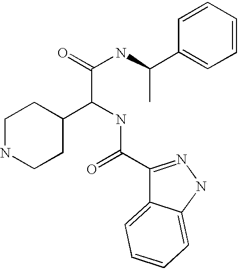 |
| B4 | B-AA1 | Methyl benzylamine | 2- Tetrahydrofuroic acid | 360.8 (M + H)⁺ | 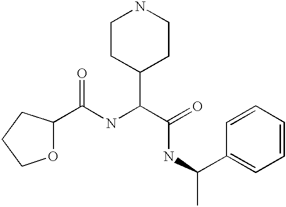 |
| B5 | B-AA1 | Methyl benzylamine | 1- methylindole- 3-carboxylic acid | 420.8 (M + H)⁺ | 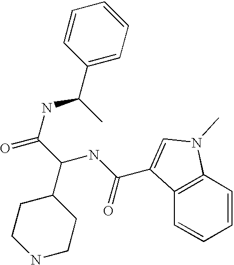 |
| B6 | B-AA1 | 2-aminoindane | 1- methylindole- 3-carboxylic acid | 434.8 (M + H)⁺ | 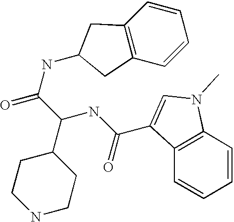 |
| B7 | B-AA1 | isoamylamine | 5- methylpyrazine- 2-carboxylic acid | 348.8 (M + H)⁺ | 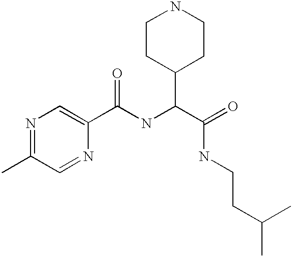 |
| B8 | B-AA1 | Methyl benzylamine | 5- methylpyrazine- 2-carboxylic acid | 382.8 (M + H)⁺ | 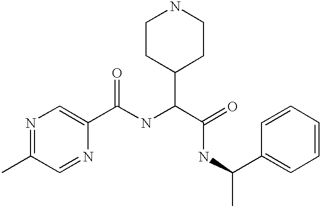 |
| B9 | B-AA1 | 2-aminoindane | 5- methylpyrazine- 2-carboxylic acid | 394.8 (M + H)⁺ | 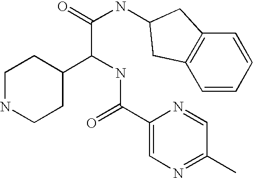 |
| B10 | B-AA1 | isoamylamine | Indazole-3- carboxylic acid | 372.8 (M + H)⁺ | 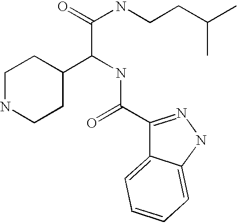 |
| B11 | B-AA1 | 2-aminoindane | Indazole-3- carboxylic acid | 418.7 (M + H)⁺ | 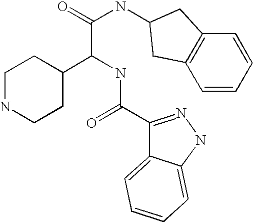 |
| B12 | B-AA1 | Methyl benzylamine | Picolinic Acid | 367.8 (M + H)⁺ | 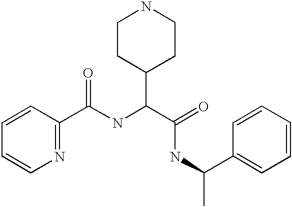 |
| B13 | B-AA1 | 2-aminoindane | Picolinic Acid | 379.8 (M + H)⁺ | 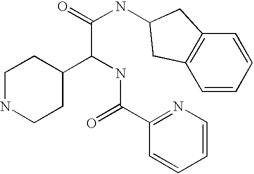 |
| B14 | B-AA2 | 3-(Di-N- butylamino) propylamine | Hydantoin-5- acetic acid | 481.0 (M + H)⁺ | 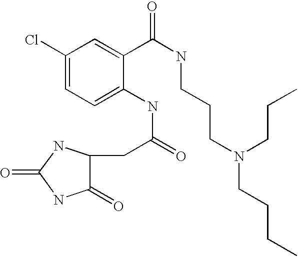 |
| B15 | B-AA2 | 3-(Di-N- butylamino) propylamine | 2- Tetrahydrofuroic acid | 438.8 (M + H)⁺ | 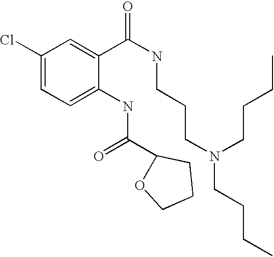 |
| B16 | B-AA2 | isoamylamine | 1- methylindole- 3-carboxylic acid | 398.9 (M + H)⁺ | 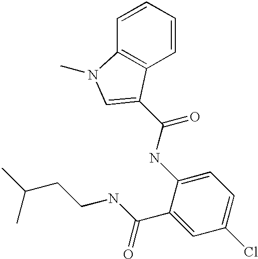 |
| B17 | B-AA2 | Methyl benzylamine | 1- methylindole- 3-carboxylic acid | 432.6 (M + H)⁺ | 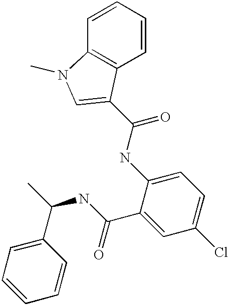 |
| B18 | B-AA2 | 2-aminoindane | 1- methylindole- 3-carboxylic acid | 445.1 (M + H)⁺ | 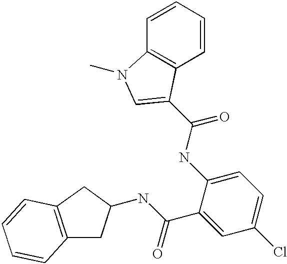 |
| B19 | B-AA2 | Furfurylamine | 1- methylindole- 3-carboxylic acid | 408.8 (M + H)⁺ | 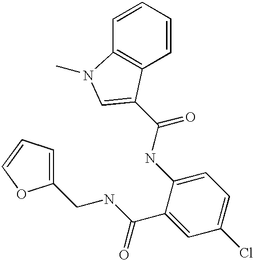 |
| B20 | B-AA2 | 3-(Di-N- butylamino) propylamine | 1- methylindole- 3-carboxylic acid | 498.9 (M + H)⁺ | 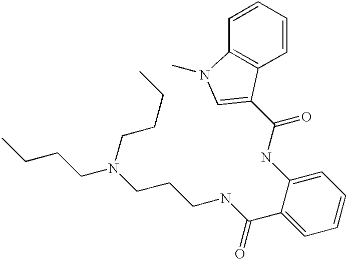 |
| B21 | B-AA2 | 3-(Di-N- butylamino) propylamine | 5- methylpyrazine- 2-carboxylic acid | 461.9 (M + H)⁺ | 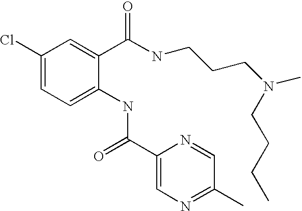 |
| B22 | B-AA2 | Methyl benzylamine | Indazole-3- carboxylic acid | 419.8 (M + H)⁺ | 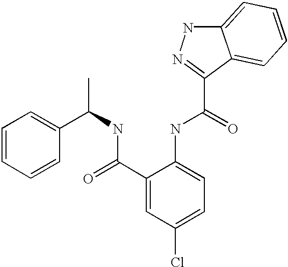 |
| B23 | 2-N- Fmoc- amino-5- chlorobenzoic acid | 2-aminoindane | Indazole-3- carboxylic acid | 432.7 (M + H)⁺ | 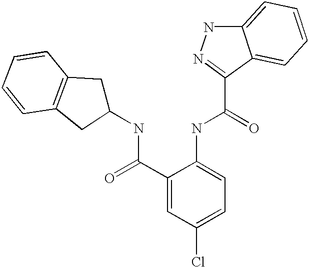 |
| B24 | 2-N- Fmoc- amino-5- chlorobenzoic acid | Furfurylamine | Indazole-3- carboxylic acid | 395.9 (M + H)⁺ | 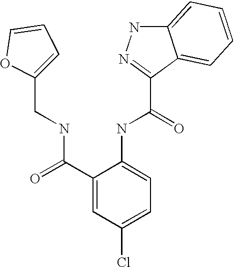 |
| B25 | 2-N- Fmoc- amino-5- chlorobenzoic acid | 3-(Di-N- butylamino)propyl amine | 5- methylpyrazine- 2-carboxylic acid | 493.9 (M + H)⁺ | 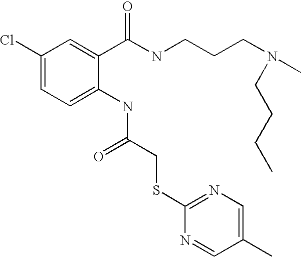 |
| B26 | 2-N- Fmoc- amino-5- chlorobenzoic acid | 3,4- dimethoxybenzylamine | 1- Benzofuran- 2-carboxylic acid | 465.9 (M + H)⁺ | 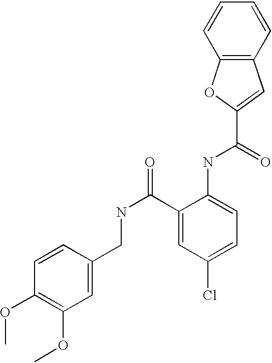 |
| B27 | 2-N- Fmoc- amino-5- chlorobenzoic acid | 3-(Di-N- butylamino)propyl amine | Coumarilic acid | 485.7 (M + H)⁺ | 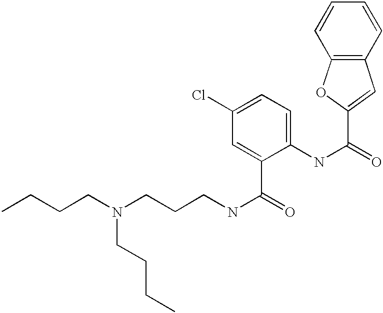 |
| B28 | 2-N- Fmoc- amino-5- chlorobenzoic acid | 3,4- dimethoxybenzylamine | Picolinic Acid | 426.6 (M + H)⁺ | 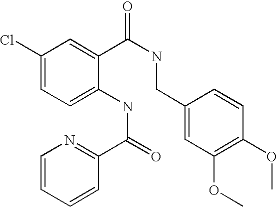 |
| 31 | 2-N- Fmoc- amino-5- chlorobenzoic acid | 3-(Di-N- butylamino)propyl amine | Picolinic Acid | 447.0 (M + H)⁺ | 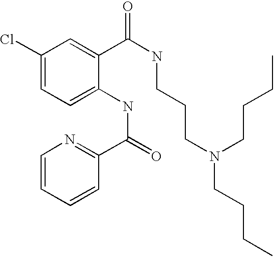 |
| 32 | 2-N- Fmoc- amino-5- chlorobenzoic acid | 2-aminoindane | 3-Cyano- benzoic acid | 417.8 (M + H)⁺ | 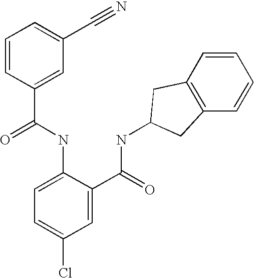 |

We claim:

1. A probe comprising: a framework and an input fragment wherein the probe comprises a recognition element.

2. The probe of claim 1 wherein the framework, the input fragment and the recognition element collectively comprise one of the following molecular formula: 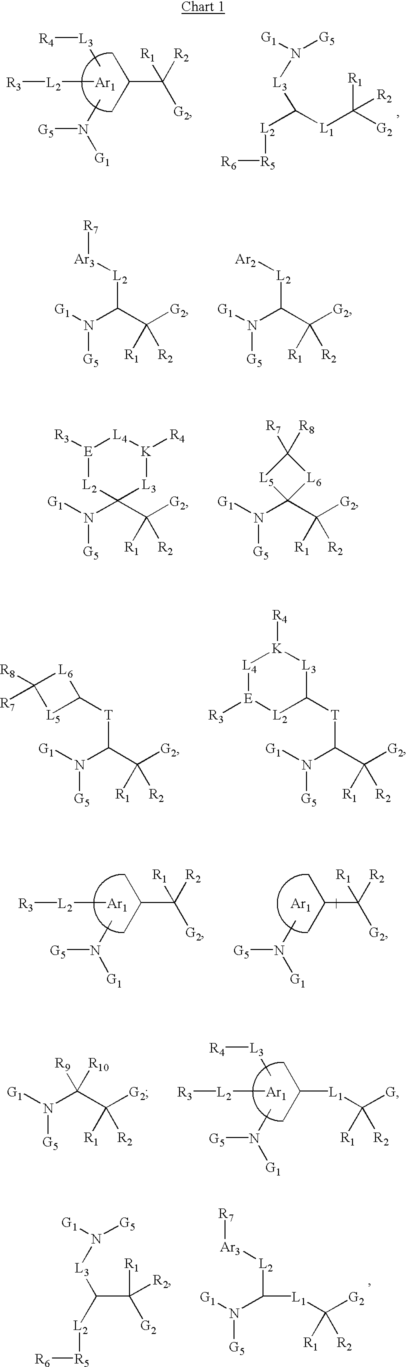
Ar₁ comprises aryl, heteroaryl, fused cycloalkylaryl, fused cycloakylheteroaryl, fused heterocyclylaryl, or fused heterocyclylheteroaryl;
L₁ comprises alkylene;
L₂ and L₃ independently comprise alkylene, alkenylene, alkynylene, or a direct bond;
R₁ and R₂ independently comprise alkyl, alkenyl, alkynyl, cycloalkyl, heterocyclyl, aryl, heteroaryl, or hydrogen;
R₁ and R₂ may be taken together to constitute an oxo group;
R₃ and R₄ independently comprise alkyl, alkenyl, alkynyl, cycloalkyl, heterocyclyl, aryl, heteroaryl, hydrogen, —O-G₃, —O-G₄, -G₃, -G₄, —N(G₆)G₃, or —N(G₆)G₄;
R₃ and R₄ may be taken together to constitute a cycloalkyl or heterocyclyl ring, or, where L₄ is a direct bond, R₃ and R₄ may be taken together to constitute a fused aryl or heteroaryl ring;
R₅ comprises alkylene, alkenylene, alkynylene, cycloalkylene, heterocyclylene, arylene, or heteroarylene;
R₆ comprises alkyl, alkenyl, alkynyl, cycloalkyl, heterocyclyl, aryl, heteroaryl, or hydrogen;
Ar₂ comprises arylene, heteroarylene, fused arylene, or fused heteroarylene;
Ar₃ comprises arylene, heteroarylene, fused arylene, or fused heteroarylene;
T comprises alkylene, alkenylene, alkynylene or a direct bond;
E and K independently comprise N or CH;
L₄ comprises alkylene, —O—, —C(O)—, —S—, —S(O)—, —S(O)₂—, or a direct single or double bond;
L₅ and L₆ are, independently, alkylene or a direct bond, with the proviso that both L₅ and L₆ are not both a direct bond;
R₇ and R₈ indpendently comprise alkyl, cycloalkyl, heterocyclyl, aryl, heteroaryl, alkoxy, alkylaryl, -alkylene-aryl, -alkylene-heteroaryl, —O-aryl, —O-heteroaryl, or hydrogen;
R₇ and R₈ may further be taken together to constitute a cycloalkyl or heterocyclyl ring;
R₉ comprises alkyl, alkenyl, alkynyl, cycloalkyl, heterocyclyl, aryl, heteroaryl, alkylaryl, alkylheteroaryl, or hydrogen;
R₁₀ comprises alkyl, alkenyl, alkynyl, cycloalkyl, heterocyclyl, aryl, heteroaryl, alkylaryl, alkylheteroaryl, or the side chain of a natural or non-natural alpha-amino acid in which any functional groups may be protected;
G₁, G₃, G₄ and G₁₄ independently comprise 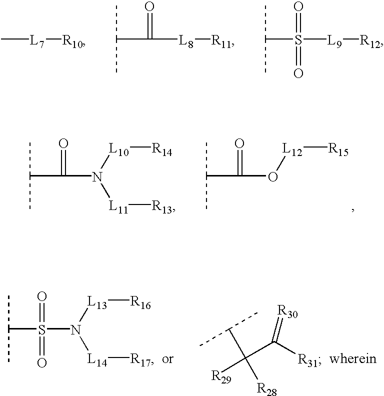
L₇, L₈, L₉, L₁₀, L₁₁, L₁₂, L₁₃, and L₁₄ independently comprise alkylene, alkenylene, alkynylene, cycloalkylene, cycloalkenylene, arylene, heterocyclylene, heteroarylene, fused cycloalkylarylene, fused cycloakylheteroarylene, fused heterocyclylarylene, fused heterocyclylheteroarylene, or a direct bond; and
R₁₁, R₁₂, R₁₃, R₁₄, R₁₅, R₁₆, and R₁₇ independently comprise alkyl, alkenyl, alkynyl, cycloalkyl, cycloalkenyl, heterocyclyl, heteroaryl, aryl, fused cycloalkylaryl, fused cycloakylheteroaryl, fused heterocyclylaryl, fused heterocyclylheteroaryl, NR₁₈R₁₉, OR₁₈, SR₁₈, or hydrogen, where R₁₈ and R₁₉ are as defined below;
R₂₈ comprises alkyl, alkenyl, alkynyl, aryl, heteroaryl, -alkenylene-aryl, or -alkenylene-heteroaryl;
R₂₉ comprises H, alkyl, alkenyl, alkynyl, -alkylene-aryl, or -alkylene-heteroaryl;
R₃₀ comprises O or H/OH;
R₃₁ comprises H, alkyl, or aryl;
G₂ comprises 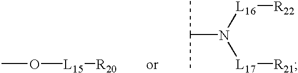
wherein
L₁₅, L₁₆, and L₁₇ independently comprise alkylene, alkenylene, alkynylene, cycloalkylene, cycloalkenylene, arylene, heterocyclylene, heteroarylene, fused cycloalkylarylene, fused cycloakylheteroarylene, fused heterocyclylarylene, fused heterocyclylheteroarylene, or a direct bond; and
R₂₀, R₂₁, and R₂₂ independently comprise alkyl, alkenyl, alkynyl, cycloalkyl, cycloalkenyl, heterocyclyl, heteroaryl, aryl, fused cycloalkylaryl, fused cycloakylheteroaryl, fused heterocyclylaryl, fused heterocyclylheteroaryl, NR₂₃R₂₄, OR₂₃, SR₂₃, or hydrogen, wherein R₂₃ and R₂₄ are as defined below;
G₅, G₆, and G₁₃ independently comprise 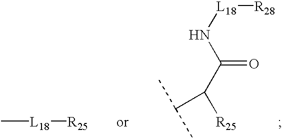
wherein L₁₈ comprises alkylene, alkenylene, alkynylene, cycloalkylene, cycloalkenylene, arylene, heterocyclylene, heteroarylene, fused cycloalkylarylene, fused cycloakylheteroarylene, fused heterocyclylarylene, fused heterocyclylheteroarylene, -alkylene-(aryl)₂, or a direct bond; and
R₂₅ comprises alkyl, alkenyl, alkynyl, cycloalkyl, cycloalkenyl, heterocyclyl, heteroaryl, aryl, fused cycloalkylaryl, fused cycloakylheteroaryl, fused heterocyclylaryl, fused heterocyclylheteroaryl, NR₂₆R₂₇, OR₂₆, SR₂₆, or hydrogen, where R₂₆ and R₂₇ are as defined below;
R₁₈, R₁₉, R₂₃, R₂₄, R₂₆, and R₂₇ independently comprise hydrogen, alkyl, alkynyl, alkenyl, cycloalkyl, cycloalkenyl, aryl, heterocyclyl, or heteroaryl;
optionally, G₁ and G₅ may be taken together in combination to constitute a heterocyclic or heteroaryl ring, wherein said heterocyclic or heteroaryl ring may be optionally substituted by a group 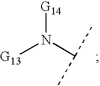
optionally, G₂ and one of G₁ or G₅ may be taken together in combination to constitute a heterocyclic ring;
optionally, G₂ of one probe and one of G₁, G₃, G₄, G₅ or G₆ of another probe may be taken together in combination to constitute a direct bond;
optionally, G₂ of a first probe and G₁ of a second probe may be taken together in combination to constitute a direct bond, where also G₂ of that second probe is taken in combination with G₁ of that first probe to constitute a direct bond;
optionally, one of G₁, G₃, G₄, G₅ or G₆ of one probe and one of G₁, G₃, G₄, G₅ or G₆ of another probe may be taken together in combination to constitute a group comprising;

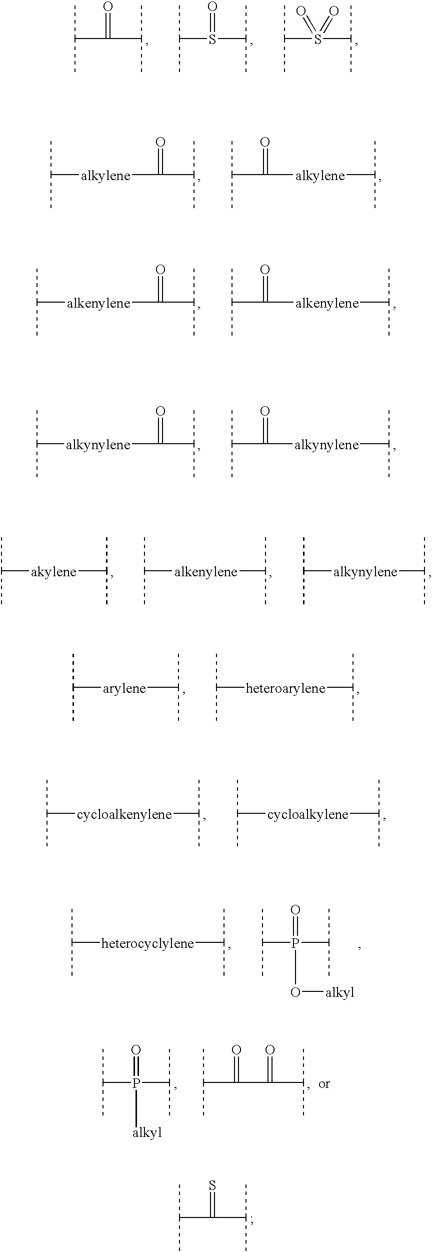

3. The probe of claim 2 wherein the probe comprises a molecular weight less than 1000 MW.

4. A probe of claim 2 wherein the probe comprises one of the following molecular formula: 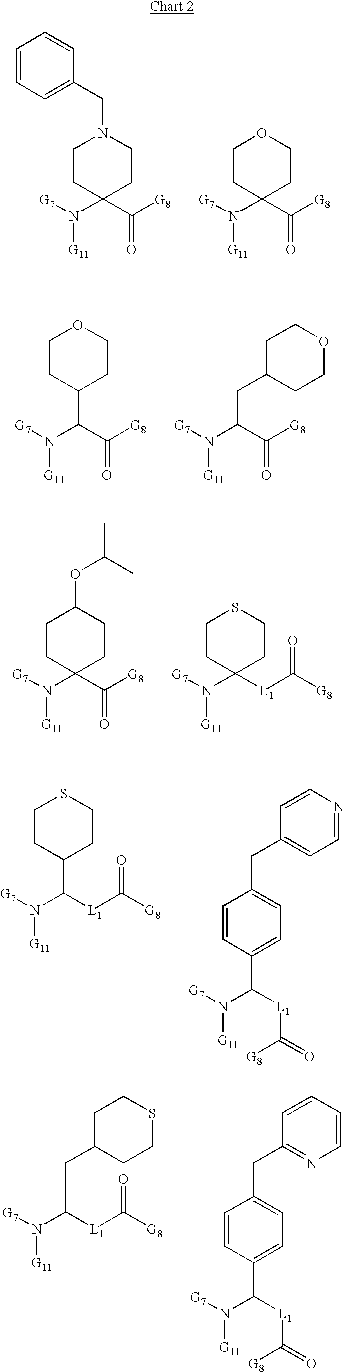
G₇, G₉, and G₁₀ independently comprise 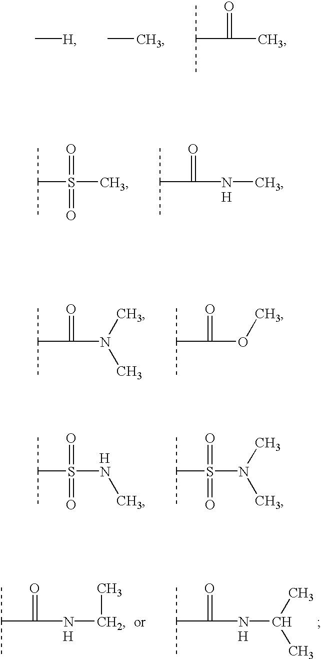
G₈ comprises 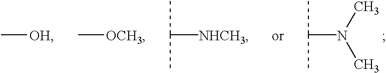
G₁₁ and G₁₂ independently comprise hydrogen or —CH₃;
Optionally, G₈ of one probe and one of G₇, G₉, or G₁₀ of another probe may be taken together in combination to constitute a direct bond.

4. A set of probes, each probe individually comprising a probe of claim 2.

5. A set of probes, each probe individually comprising a probe of claim 3.

6. A probe of claim 3, wherein the probe comprises:

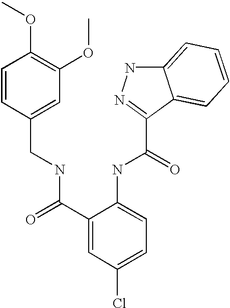

7. A probe of claim 3, wherein the probe comprises:

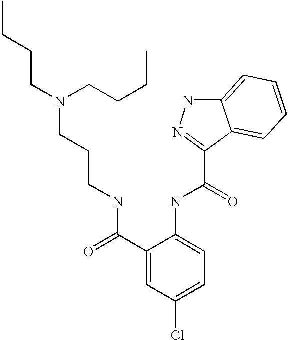

8. A probe of claim 3, wherein the probe comprises:

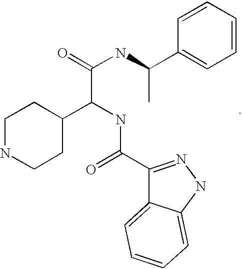

9. A pharmaceutical composition comprising a probe of claim 2.

10. A pharmaceutical composition comprising a probe of claim 6.

11. A pharmaceutical composition comprising a probe of claim 7.

12. A pharmaceutical composition comprising a probe of claim 8.

13. A system for drug discovery comprising:
a set of probes, each probe comprising a framework, an input fragment wherein the probe comprises a recognition element;
means for attempting to associate a probe from the set of probes with a binding site on a therapeutic target;
means for evaluating the association between the probe and the binding site; and
means for selecting probes with a desired association to the binding site.

14. The system of claim 13 further comprising means for creating a set of probes.

15. The system of claim 13 wherein each probe comprises a probe of claim 2.

16. The system of claim 15 wherein at least one of the means for attempting to associate a probe; the means for evaluating the association; and/or the means for selecting probes comprises computer software.

17. The system of claim 14 wherein at least one of the means for creating a set of probes; means for attempting to associate a probe; the means for evaluating the association; and/or the means for selecting probes comprises computer software.

18. The method of claim 17 wherein the means iteratively interact.

19. A method of drug discovery comprising:
attempting to associate a probe from a set of probes with a binding site on a therapeutic target;
evaluating the association between the probe and the binding site; and
selecting probes with a desired association to the binding site.

20. The method of claim 19 further comprising creating a set of probes.

21. The method of claim 20 wherein each probe comprises a probe of claim 2.

22. The method of claim 19 wherein at least a part of one of the steps of attempting to associate a probe; evaluating the association; and/or selecting probes is performed utilizing computer software.

23. The method of claim 21 wherein at least part of one of the steps of creating a set of probes; attempting to associate a probe; evaluating the association; and/or selecting probes is performed utilizing computer software.

24. The method of claim 23 wherein the computer software iteratively interacts among method steps.
